(19) United States
(12) Patent Application Publication
(10) Pub. No.: US 2007/0026076 A1
(43) Pub. Date: Feb. 1, 2007

(54) Molecular vaccines employing nucleic acid encoding anti-apoptotic proteins

(76) Inventors: Tzyy-Choou Wu, Stevenson, MD (US); Chien Fu Hung, Timonium, MD (US); Tae-Woo Kim, Baltimore, MD (US)

Correspondence Address: MCKENNA LONG & ALDRIDGE LLP, 1900 K STREET, NW, WASHINGTON, DC, 20006, US

(21) Appl. No.: 10/546,810

(22) PCT Filed: Feb. 24, 2004
(86) PCT No.: PCT/US04/05292

§ 371 Date: Sep. 27, 2006

Related U.S. Application Data
(60) Provisional application No. 60/449,429, filed on Feb. 24, 2003.
(60) Provisional application No. 60/488,527, filed on Jul. 18, 2003.
(60) Provisional application No. 60/533,792, filed on Dec. 31, 2003.

(51) Int. Cl. A61K 48/00; C12N 15/09; C12N 15/87; A61K 9/14

(52) U.S. Cl. 424/489; 514/44; 435/459; 977/906

(57) ABSTRACT

T cell immune responses are enhanced by presentation of antigen to CD8⁺ T cells using a chimeric nucleic acid immunogen or vaccine that links DNA encoding an antigen with DNA encoding a polypeptide that targets or translocates the antigenic polypeptide to which it is fused (immunogenicity-potentiating polypeptides or “IPP”). By inhibiting apoptosis in the vicinity of a T cell responses to such a nucleic acid immunogen, even more potent immune responses are attained. The present strategy prolongs the survival of DNA-transduced cells, including dendritic cells (DCs), thereby enhancing the priming of antigen-specific T cells and increase potency. Co-delivery of DNA encoding an inhibitor of apoptosis, including (a) BCL-xL, (b) BCL-2, (c) XIAP, (d) dominant negative caspase-9, or (e) dominant negative caspase-8, or (f) serine protease inhibitor 6 (SPI-6) which inhibits granzyme B, with DNA encoding an antigen, prolongs the survival of transduced DCs and results in significant enhancement of antigenspecific T cell immune responses that provide potent antitumor effects. Thus, co-administration of a DNA vaccine encoding antigen linked to an IPP along with one or more DNA constructs encoding an anti-apoptotic protein provides a novel way to enhance vaccine potency.

### BACKGROUND OF THE INVENTION

1. Field of the Invention

The present invention in the fields of molecular biology, immunology and medicine relates to combinations or mixtures of nucleic acid molecules and chimeric nucleic acid molecules that encode an antigen and an anti-apoptotic protein, and their uses a immunogenic compositions to induce and enhance immune responses, primarily cytotoxic T lymphocyte (CTL) responses to specific antigens such as tumor or viral antigens. The optionally chimeric antigen-encoding nucleic acids also encode a fusion protein comprising an antigenic polypeptide fused to an immunogenicity-potentiating polypeptide (“IPP”) that promotes processing via the MHC class I pathway and selective induction of immunity mediated by CD8+ antigen-specific CTL.

2. Description of the Background Art

Cytotoxic T lymphocytes (CTL) are critical effectors of anti-viral and antitumor responses (reviewed in Chen, C H et al., J Biomed Sci. 5: 231-252, 1998; Pardoll, D M. Nat Med. 4: 525-531, 1998; Wang, R F et al, Immunol Rev. 170: 85-100, 1999). Activated CTL are effector cells that mediate antitumor immunity by direct lysis of their target tumor cells or virus-infected cells and by releasing of cytokines that orchestrate immune and inflammatory responses that interfere with tumor growth or metastasis, or viral spread. Depletion of CD8⁺ CTL leads to the loss of antitumor effects of several cancer vaccines (Lin, K-Y et al., Canc Res. 56: 21-26, 1996; Chen, C-H et al., Canc Res. 60: 1035-42, 2000). Therefore, the enhancement of antigen presentation through the MHC class I pathway to CD8⁺ T cells has been a primary focus of cancer immunotherapy.

Naked DNA vaccines have emerged recently as attractive approaches for vaccine development (reviewed in Hoffman, S L et al., Ann NY Acad Sci. 772: 88-94, 1995; Robinson, H L. Vaccine. 15: 785-787, 1997; Donnelly, J J et al., Annu Rev Immunol. 15: 617-648, 1997; Klinman, D M et al., Immunity. 11: 123-129, 1999; Restifo, N P et al., Gene Ther. 7. 89-92, 2000; Gurunathan, S et al., Annu Rev Immunol. 18: 927-974, 2000). DNA vaccines generated long-term cell-mediated immunity (reviewed in Gurunathan, S et al, Curr Opin Immunol. 12: 442-447, 2000). In addition, DNA vaccines can generate CD8⁺ T cell responses in vaccinated humans (Wang, R et al. Science. 282: 476-480, 1998).

However, one limitation of these vaccines is their lack of potency, since the DNA vaccine vectors generally do not have the intrinsic ability to be amplified and to spread in vivo as do some replicating viral vaccine vectors. Furthermore, some tumor antigens such as the E7 protein of human papillomavirus-16 (“HPV-16”) are weak immunogens (Chen et al., 2000, supra). Therefore, there is a need in the art for strategies to enhance DNA vaccine potency, particularly for more effective cancer and viral immunotherapy.

The present inventors and their colleagues demonstrated that linkage of HPV-16 E7 antigen to a number of immunogenicity-potentiating polypeptides, such as Mycobacterium tuberculosis(Mtb) heat shock protein 70 (Hsp70) led to the enhancement of DNA vaccine potency (Chen et al., supra; Wu et al., WO 01/29233). This followed the discovery that immunization with HSP complexes isolated from tumor or virus-infected cells potentiated anti-tumor immunity (Janetzki, S et al., 1998. J Immunother 21:269-76) or antiviral immunity (Heikema, A E et al., Immunol Lett 57:69-74). Immunogenic HSP-peptide complexes could be reconstituted in vitro by mixing the peptides with HSPs (Ciupitu, A M et al., 1998. J Exp Med 187:685-91). Furthermore, HSP-based protein vaccines have been created by fusing antigens to HSPs (Suzue, K et al., 1996. J Immunol 156:873-9). The results of these investigations point to HSPs one attractive candidate for use in immunotherapy. However, prior to the present inventors' work, HSP vaccines were peptide/protein-based vaccines. The present inventors and their colleagues were the first to provide naked DNA and self-replicating RNA vaccines that incorporated HSP70 and other immunogenicity-potentiating polypeptides. The present inventors and their colleagues also demonstrated that linking antigen to intracellular targeting moeities calreticulin (CRT), domain II of Pseudomonas aeruginosa exotoxin A (ETA(dII)), or the sorting signal of the lysosome-associated membrane protein type 1 (Sig/LAMP-1) enhanced DNA vaccine potency compared to compositions comprising only DNA encoding the antigen of interest. To enhance MHC class II antigen processing, one of the present inventors and colleagues (Lin, K Y et al., 1996, Canc Res 56: 21-26) linked the sorting signals of the lysosome-associated membrane protein (LAMP-1) to the cytoplasmic/nuclear human papilloma virus (HPV-16) E7 antigen, creating a chimera (Sig/E7/LAMP-1). Expression of this chimera in vitro and in vivo with a recombinant vaccinia vector had targeted E7 to endosomal and lysosomal compartments and enhanced MHC class II presentation to CD4+ T cells. This vector was found to induce in vivo protection against an E7+tumor, TC-1 so that 80% of mice vaccinated with the chimeric Sig/E7/LAMP1 vaccinia remained tumor free 3 months after tumor injection. Treatment with the Sig/E7/LAMP-1 vaccinia vaccine cured mice with small established TC-1 tumors, whereas the wild-type E7-vaccinia showed no effect on this established tumor burden. These findings point to the importance of adding an “element” to an antigenic composition to enhance in vivo potency of a recombinant vaccine: in this case, a polypeptide that rerouted a cytosolic tumor antigen to the endosomal/lysosomal compartment

Intradermal administration of DNA vaccines via gene gun in vivo have proven to be an effective means to deliver such vaccines into professional antigen-presenting cells (APCs), primarily dendritic cells (DCs), which function in the uptake, processing, and presentation of antigen to T cells. The interaction between APCs and T cells is crucial for developing a potent specific immune response. However, various of the strategies noted above lead to apoptosis of APCs. For example, DNA-based alphaviral RNA replicon vectors, also called suicidal DNA vectors, have an advantage of greatly reducing the risk of that the vaccine DNA molecule(s) will integrate into the DNA of a host cell and further transform the cell. Suicidal DNA vectors do so because they eventually cause apoptosis of any transfected cells. The disadvantage is that expression of inserted genes in these vectors is transient, as apoptotic cell death of those cells expressing the immungenic proteins may compromise the potency of a suicidal DNA vaccine.

Therefore, a strategy to prolong the survival of APCs is expected to enhance antigen-specific T cell immune responses even more then the some of the chimeric DNA vaccines that simply combine antigen with a immunogenicity-potentiating polypeptide.

### SUMMARY OF THE INVENTION

The present inventors have designed and disclose herein an immunotherapeutic strategy that combines antigen-encoding DNA vaccine compositions with additional DNA vectors comprising anti-apoptotic genes including bcl-2, bc-lxL, XIAP, dominant negative mutants of caspase-8 and caspase-9, the products of which are known to inhibit apoptosis. Serine protease inhibitor 6 (SPI-6) which inhibits granzyme B, is also employed in novel compositions and methods to delay apoptotic cell death of DCs.

The growing understanding of the antigen presentation pathway creates the potential for designing novel strategies to enhance vaccine potency. One strategy taken by the present inventors in the present invention to enhance the presentation of antigen through the MHC class I pathway to CD8⁺ T cells is the exploitation of the features of certain polypeptides to target or translocate the antigenic polypeptide to which they are fused. Such polypeptide are referred to collectively herein as “immunogenicity-potentiating (or -promoting) polypeptide” or “IPP” to reflect this general property, even though these IPP's may act by any of a number of cellular and molecular mechanisms that may or may not share common steps. This designation is intended to be interchangeable with the term “targeting polypeptide.” Inclusion of nucleic acid sequences that encode polypeptides that modify the way the antigen encoded by molecular vaccine is “received” or “handled” by the immune system serve as a basis for enhancing vaccine potency. All of these polypeptides in some way, contribute to the augumentation of the specific immune response to an antigen to which they are linked by one or another means that these molecules “employ” to affect the way in which the cells of the immune system handle the antigen or respond in terms of cell proliferation or survival. IPP's may be produced as fusion or chimeric polypeptides with the antigen, or may be expressed from the same nucleic acid vector but produced as distinct expression products.

In addition to the strategy of including DNA encoding such IPPs in their vaccine constructs, the present inventors have now discovered that the harnessing of an additional biological mechanism, that of inhibiting apoptosis, significantly enhances T cell responses to DNA vaccine comprising antigen-coding sequences as well as linked sequences encoding such IPPs.

Intradermal vaccination by gene gun efficiently delivers a DNA vaccine into DCs of the skin, resulting in the activation and priming of antigen-specific T cells in vivo. DCs, however, have a limited life span, hindering their long-term ability to prime antigen-specific T cells. According to the present invention, a strategy that prolongs the survival of DNA-transduced DCs enhances priming of antigen-specific T cells and thereby, increase DNA vaccine potency. As described herein (see Example I) co-delivery of DNA encoding inhibitors of apoptosis (BCL-xL, BCL-2, XIAP, dominant negative caspase-9, or dominant negative caspase-8) with DNA encoding an antigen (exemplified as HPV-16 E7 protein) prolongs the survival of transduced DCs. More importantly, vaccinated subjects exhibited significant enhancement in antigen-specific CD8+ T cell immune responses, resulting in a potent antitumor effect against antigen-expressing tumors. Among these anti-apoptotic factors, BCL-XL demonstrated the greatest enhancement of both antigen-specific immune responses and antitumor effects. Thus, co-administration of a DNA vaccine with one or more DNA constructs encoding anti-apoptotic proteins provides a novel way to enhance DNA vaccine potency.

The combination of a strategy to prolong DC life with intracellular targeting strategies effected by certain IPPs produce a more effective DNA vaccine against HPV E7. Co-administration of DNA encoding Bcl-xL with DNA encoding E7 linked to HSP70, CRT, or Sig/E7/LAMP-1 resulted in further enhancement of the E7-specific CD8+ T cell response for all three constructs. This combination increased CD8+ T cell functional avidity, and increased the E7-specific CD4+ Th1 cell response, enhanced tumor therapeutic effect, and yielded more durable tumor protection when compared with mice vaccinated without Bcl-xL DNA. Therefore, DNA vaccines that combine strategies to enhance intracellular Ag processing and prolong DC life have clinical utility for control of viral infection and neoplasia.

Serine protease inhibitor 6 (SPI-6), also called Serpinb9, inhibits granzyme B, and may thereby delay apoptotic cell death in DCs. Intradermal co-administration of DNA encoding SPI-6 with DNA constructs encoding E7 linked to various IPPs significantly increased E7-specific CD8+ T cell and CD4+ Th1 cell responses and enhanced anti-tumor effects when compared to vaccination without SPI-6. Thus it is preferred to combine methods that enhance MHC class I and II antigen processing with delivery of SPI-6 to potentiate immunity

A similar approach employs DNA-based alphaviral RNA replicon vectors, also called suicidal DNA vectors. To enhance the immune response to an antigen, e.g., HPV E7, a DNA-based Semliki Forest virus vector, pSCA1, the antigen DNA is fused with DNA encoding an anti-apoptotic polypeptide such BCL-xL, a member of the BCL-2 family. pSCA1 encoding a fusion protein of an antigen polypeptide and/BCL-xL delays cell death in transfected DCs and generates significantly higher antigen-specific CD8+ T-cell-mediated immunity. The antiapoptotic function of BCL-xL is important for the enhancement of antigen-specific CD8+ T-cell responses. Thus, in one embodiment, delaying cell death induced by an otherwise desirable suicidal DNA vaccine enhances its potency.

Thus, the present invention is directed to a nucleic acid composition useful as an immunogen, comprising a combination of:
(a) first nucleic acid vector comprising a first sequence encoding an antigenic polypeptide or peptide, which first vector optionally comprises a second sequence linked to the first sequence, which second sequence encodes an immunogenicity-potentiating polypeptide (IPP);
b) a second nucleic acid vector encoding an anti-apoptotic polypeptide,
   wherein, when the second vector is administered with the first vector to a subject, a T cell-mediated immune response to the antigenic polypeptide or peptide is induced that is greater in magnitude and/or duration than an immune response induced by administration of the first vector alone. The first vector above may comprises a promoter operatively linked the first and/or the second sequence.

Also provided is a nucleic acid composition useful as an immunogen comprising
(a) a first nucleic acid sequence that encodes an antigenic polypeptide or peptide.
(b) optionally, fused in frame with the first nucleic acid sequence, a linker nucleic acid sequence encoding a linker peptide;
(c) a second nucleic acid sequence that is linked in frame to the first nucleic acid sequence or to the linker nucleic acid sequence and that encodes an IPP; and
(d) a third nucleic acid sequence encoding an anti-apoptotic polypeptide.
   This may comprise a promoter operatively linked to one or more of the first, second and sequences.

In the above composition the IPP preferablyi acts in potentiating an immune response by promoting:
(a) processing of the linked antigenic polypeptide via the MHC class I or class II pathway or targeting of a cellular compartment that increases the processing;
(b) development, accumulation or activity of antigen presenting cells or targeting of antigen to compartments of the antigen presenting cells leading to enhanced antigen presentation;
(c) intercellular transport and spreading of the antigen; or
(d) any combination of (a)-(c).

The IPP is preferably
(a) the sorting signal of the lysosome-associated membrane protein type 1 (Sig/LAMP-1)
(b) a mycobacterial HSP70 polypeptide, the C-terminal domain thereof, or a functional homologue or derivative of the polypeptide or domain;
(c) a viral intercellular spreading protein selected from the group of herpes simplex virus-1 VP22 protein, Marek's disease virus VP22 protein or a functional homologue or derivative thereof;
(d) an endoplasmic reticulum chaperone polypeptide selected from the group of calreticulin, ER60, GRP94, gp96, or a functional homologue or derivative thereof
(e) a cytoplasmic translocation polypeptide domains of a pathogen toxin selected from the group of domain II of Pseudomonas exotoxin ETA or a functional homologue or derivative thereof;
(f) a polypeptide that targets the centrosome compartment of a cell selected from γ-tubulin or a functional homologue or derivative thereof; or
(g) a polypeptide that stimulates dendritic cell precursors or activates dendritic cell activity selected from the group of GM-CSF, Flt3-ligand extracellular domain, or a functional homologue or derivative thereof.

In the above composition the anti-apoptotic polypeptide is preferably selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or derivative of any of (a)-(g).

In the above composition, the antigenic peptide may comprise an epitope that binds to and is presented on the cell surface by MHC class I proteins and the epitope is preferably between about 8 and about 11 amino acid residues in length.

The antigenic polypeptide or peptide may be one that:
(i) is derived from a pathogen selected from the group consisting of a mammalian cell, a microorganism or a virus;
(ii) cross-reacts with an antigen of the pathogen; or
(iii) is expressed on the surface of a pathogenic cell, such as a tumor-specific or tumor-associated antigen.

In a preferred composition the virus is a human papilloma virus and the antigen is an HPV-16 E6 or E7 peptide.

Also provided is a particle comprising a material is suitable for introduction into a cell or an animals by particle bombardment to which is bound the first vector, the second vector, or both the first and the second vectors of the first composition above.

The particle may have bound thereto any of the foregoing compositions.

Also provided is a pharmaceutical composition capable of inducing or enhancing an antigen specific immune response, comprising the above composition or particle and a pharmaceutically acceptable carrier or excipient.

The invention is directed to a method of inducing or enhancing an antigen specific immune response in a subject, preferably a human, comprising administering to the subject an effective amount of the above composition (or particles), thereby inducing or enhancing the antigen specific immune response.

In a preferred embodiment, the antigen specific immune response is mediated at least in part by CD8⁺ cytotoxic T lymphocytes (CTL).

In the above method, the composition or particles are preferably administered intradermally or, in the case of a tumor, intratumorally or peritumorally.

Also included is a method of increasing the numbers of CD8⁺ CTLs specific for a selected desired antigen in a subject comprising administering an effective amount of the above composition wherein the antigenic peptide comprises an epitope that binds to and is presented on the cell surface by MHC class I proteins, thereby increasing the numbers of antigen-specific CD8⁺ CTLs.

In another embodiment, the method comprises increasing the numbers of CD4⁺ Th cells specific for a selected desired antigen in a subject comprising administering an effective amount of the above composition wherein the antigenic peptide comprises an epitope that binds to and is presented on the cell surface by MHC class II proteins, thereby increasing the numbers of antigen-specific CD4⁺ Th cells.

Also provided is a method of inhibiting the growth of a tumor in a subject comprising administering an effective amount of the above composition or particles, thereby inhibiting growth of the tumor.

### BRIEF DESCRIPTION OF THE DRAWINGS

FIGS. 1A-1E show E7-specific CD8+ T cell immune responses and antitumor effect induced by vaccination with E7 DNA co-administered mixed with DNA encoding anti-apoptotic or pro-apoptotic proteins. pcDNA3 (no insert) mixed with pSG5-BCL-xL was a negative control. FIG. 1A shows representative flow-cytometric results from one of three studies. FIG. 1B is a bar graph depicting the mean (±SD) number of antigen-specific IFNγ-secreting CD8+ T cell precursors (per 3×10⁵ splenocytes). FIG. 1C is a graph showing results of a tumor growth prevention study. Mice were immunized with pcDNA3-E7 mixed with pSG5 encoding BCL-xL, caspase-3, or no insert. The pcDNA3 (no insert) mixed with pSG5-BCL-xL was the negatife antigen control. One week after the last vaccination, mice were challenged subcutaneously (s.c.) with 5×104 TC-1 cells in the right leg. FIG. 1D is a grap showing effect of in vivo depletion of cell populations using mAb depletion to determine the contribution of various lymphocyte subsets to tumor protection. Depletion of CD4+, CD8+, and NK1.1+ cells was initiated 1 week before tumor challenge. FIG. 1E is a graph showing results of a tumor therapy study. Mice were implanted with 10⁴ TC-1 tumor challenge and were treated 3 days later with pcDNA3-E7 mixed with pSG5 encoding (i) BCL-XL, (ii) caspase-3, or (iii) no insert. Experiments of the type shown in FIGS. 1C-1E were repeated three times. Casp=caspace.

FIGS. 2A and 2B show antigen-specific CD8+ T cell precursors in mice vaccinated with DNA encoding HA or OVA co-administered with DNA encoding an anti-apoptotic protein. Mice (3/group) were immunized with pcDNA3 encoding HA or OVA mixed with pSG5 that included the anti-apoptotic gene (BCL-xL) or no insert. The pcDNA3 (no insert) mixed with pSG5-BCL-xL was a negative control. FIG. 1A shows representative flow-cytometry results (from 1 of 3 studies). FIG. 1B is a bar graph depicting the mean (±SD) number of antigen-specific IFNγ-secreting CD8+ T cell precursors induced by two different antigen vectors co-administered with a control or an anti-apoptotic vector.

FIG. 3A-3E show E7-specific CD8+ T cell immune responses in mice vaccinated with Sig/E7/LAMP-1 DNA co-administered with DNA encoding anti-apoptotic (or pro-apoptotic) proteins. Mice (3/group) were immunized with pcDNA3-Sig/E7/LAMP-1 mixed with pSG5 encoding one of several anti-apoptotic proteins: BCL-xL, XIAP, BCL-2, dn caspase-9, dn caspase-8); a proapoptotic protein (caspase-3); or no insert. The pcDNA3 (no insert) mixed with pSG5-BCL-xL was a negative control. The number of E7-specific IFNγ-secreting CD8+ T cell precursors was analyzed by intracellular cytokine staining followed by flow cytometry analysis. FIG. 3A shows representative flow-cytometric results from one of three studies. FIG. 3B is a bar graph depicting the mean (±SD) number of antigen-specific IFNγ-secreting CD8+ T cell precursors (per 3×10⁵ splenocytes) FIG. 3C shows representative flow-cytometric results from one of three studies in which mice (3/group) were immunized with pcDNA3-Sig/E7/LAMP-1 mixed with pSG5 encoding BCL-xL, caspase-3, mt BCL-xL, mt caspase-3, or no insert. The pcDNA3 (no insert) mixed with pSG5-BCL-xL was a negative control. FIG. 3D is a bar graph depicting the mean (±SD) number of antigen-specific IFNγ-secreting CD8+ T cell precursors (per 3×10⁵ splenocytes). FIG. 3E is a graph depicting the number of antigen-specific IFNγ-secreting CD8+ T cell precursors enumerated at 1, 7, 12, and 14 weeks after co-administration of pcDNA-Sig/E7/LAMP-1 with pSG5-BCL-XL, pSG5-caspase-3, or pSG5 (no insert). casp, caspase.

FIGS. 4A and 4B provide a characterization of DNA-transfected DCs in the inguinal lymph nodes (LNs) of vaccinated mice. Mice (3/group) were immunized with pcDNA3-E7/GFP DNA mixed with pSG5-BCL-xL, pSG5-mt BCL-xL, pSG5-caspase-3, or pSG5. The pcDNA3 mixed with pSG5-BCL-xL was a negative control. DCs were enriched using CD11c microbeads from a single-cell suspension of inguinal LN cells harvested 1 and 5 days after gene gun vaccination. Enriched CD11c+ cells were analyzed for forward versus side scatter; the gated area represents the monocyte population. FIG. 4A shows representative flow-cytometry results (3 total experiments) indicating the percentage of E7/GFP-transfected CD11c+ cells among the gated monocytes. FIG. 4B is a bar graph depicting the percentage of CD11c+ GFP+ monocytes among the gated monocytes (mean±SD). FIG. 4C is a bar graph depicting the percentage of apoptotic cells in CD11c+ GFP+ cells (mean±SD). casp, caspase; FSC, forward scatter; SSC, side scatter.

FIGS. 5A and 5B show activation of E7-specific CD8+ T cells by CD11c-enriched cells isolated from the draining LN of vaccinated mice. Mice (3/group) were immunized and CD11c+ cells were enriched as described in the legend for FIGS. 4A-4B. CD11c-enriched cells were incubated with cells of an E7-specific CD8+ T cell line. Cells were then stained for both CD8 and intracellular IFNγ to enumerate the E7-specific, CD8+, IFNγ-secreting T cells. FIG. 5A shows representative flow-cytometry results (one of three experiments). FIG. 5B is a bar graph depicting the number of E7-specific, CD8+ T IFNγ-secreting T cells (mean±SD).

JI Paper Figures

FIGS. 6A-6B show results of E7-specific CD8+ T cell response in mice vaccinated with DNA encoding antigen plus intracellular targeting moieties along with DNA encoding the anti-apoptotic polypeptide Bcl-xL. Mice were immunized with pcDNA3, pcDNA3-E7, pcDNA3-E7/HSP70, pcDNA3-Sig/E7/LAMP-1, or pcDNA3-CRT/E7 co-administered with pSG5 or with pSG5-Bcl-xL. Splenocytes from vaccinated mice were harvested 7 days after a booster, cultured in vitro with the MHC class I-restricted E7 peptide (aa 49-57) overnight, and stained for both CD8 and IFNγ and analyzed by flow cytometry. FIG. 6A provides representative flow cytometry results (one experiment of two). FIG. 6B is a bar graph depicting the number of E7-specific CD8+ IFNγ-secreting T cells. pcDNA3 empty vectors mixed with pSG5 or pSG5-Bcl-xL were used as negative controls.

FIG. 7 is a graph showing the functional avidity of E7-specific CD8+ T cells in mice vaccinated with pcDNA3-Sig/E7/LAMP-1 mixed with pSG5-Bcl-xL or pSG5 control. Mice were immunized with pcDNA3-Sig/E7/LAMP-1 mixed with pSG5-Bcl-xL, pSG5-mtBcl-xL, or pSG5. Splenocytes were collected 1 wk after vaccination and incubated with different concentrations of E7 peptide (aa 49-57) for 20 h. pcDNA3 mixed with pSG5 encoding Bcl-xL was used as a negative control. A line indicating 50% of maximum response is shown and curves are compared for the concentration of E7 peptide needed to attain this 50% level.

FIGS. 8A and 8B show Th1- and Th2-type CD4+ T cell responses induced by vaccination with pcDNA3-Sig/E7/LAMP-1 co-administered with pSG5-Bcl-xL or pSG5 control. Splenocytes from vaccinated mice were harvested 7 days after a booster vaccination, cultured with MHC class II-restricted E7 peptide (aa 30-67) overnight, and stained for CD4, IFNγ, and IL-4. FIG. 8A is a bar graph depicting the number of E7-specific IFNγ-secreting CD4+ T cell precursors/3×10⁵ splenocytes. FIG. 8B is a bar graph depicting the number of E7-specific IL-4-secreting CD4+ T cell precursors/3×10⁵ splenocytes.

FIGS. 9A-9B show E7-specific CD8+ T lymphocyte response in CD4KO mice vaccinated with pcDNA3-Sig/E7/LAMP-1 co-administered pSG5-Bcl-xL or pSGF control (no insert). Wild type C57BL/6 and C57BL/6/CD4KO mice were immunized and splenocytes were collected and prepared as above. The number of E7-specific CD8+ T IFNγ-secreting cell precursors was analyzed by intracellular cytokine staining and flow cytometry. FIG. 9A shows flow cytometry results (from one of two experiments) depicting numbers of E7-specific IFNγ-secreting CD8+ T cells in mice after vaccination. FIG. 9B is a bar graph showing the number of E7-specific IFNγ-secreting CD8+ T cell precursors/3×10⁵ splenocytes in the various treatment groups.

FIGS. 10A-10B show results of treating tumors in vivo and analysis of cell substrates by in vivo depletion using mAbs. Mice were vaccinated with pcDNA3-Sig/E7/LAMP-1 mixed with pSG5-Bcl-xL or pSG5 control. FIG. 10A is a graph showing the number of tumor nodules in the lungs of mice inoculated i.v. with 10⁵ TC-1 tumor cells and treated 3 days later with the various combinations. FIG. 10B shows tumor protection with depletion to determine the contribution of various lymphocyte subsets. All results are expressed as mean number of pulmonary nodules with SE indicated.

FIGS. 11A and 11B show the duration of E7-specific CD8+ T cell memory and long-term tumor protection in mice vaccinated in conjunction with Bcl-xL DNA or empty vectors. Mice were immunized with pcDNA3-Sig/E7/LAMP-1 mixed with pSG5 with the Bcl-xL insert or no insert. pcDNA3 mixed with pSG5 was used as a control. FIG. 11A is a bar graph depicting number of E7-specific CD8+ IFNγ-secreting CD8+ T lymphocytes/3×10⁵ splenocytes 1 and 7 wk after immunization. FIG. 11B depicts longer-term tumor protection as the number pulmonary nodules in vaccinated mice over time. Mice were challenged with 10⁴ TC-1 tumor cells 7 wk after immunization. Results are expressed as mean number of pulmonary tumor nodules; bars±SE.

FIGS. 12A-12C show results of experiments in which pcDNA3-SPI-6 co-administration with pcDNA3-E7 potentiates T cell responses and anti-tumor immunity. Mice were immunized with pcDNA3-E7 plus pcDNA3-SPI-6 or control pcDNA3 and received a booster of the same composition one week later. FIG. 12A is a bar graph depicting the number of E7-specific IFN-γ-secreting CD8+ T cell precursors (mean±SD). FIG. 12B shows results of a tumor protection study in which experiment mice were challenged with 5×10⁴ TC-1 tumor cells one week after the last vaccination. FIG. 12C shows results of a study of in vivo antibody depletion to determine the contribution of lymphocyte subsets to tumor protection. Depletion was initiated 1 week before tumor challenge.

FIGS. 13A-13B show results of experiments in which pcDNA3-SPI-6 co-administration with vectors linking E7 to intracellular targeting polypeptides potentiate T cell responses. Mice were immunized with pcDNA3 (negative control), pcDNA3-E7, pcDNA3-Sig/E7/LAMP-1, pcDNA3-ETA(dII)/E7, pcDNA3-E7/HSP70, or pcDNA3-CRT/E7 co-administered with pcDNA3-SPI-6 or control DNA. FIG. 13A shows representative flow cytometry results (one experiment of two. FIG. 13B is a bar graph depicting the number of antigen-specific IFN-γ-secreting CD8⁺ T cell precursors (mean±SD).

FIGS. 14A-14B characterize Th1 and Th2 E7-specific CD4⁺ T cell precursors after vaccinationd with E7 DNA linked to intracellular targeting polypeptides molecules co-adminstered with pcDNA3-SPI-6 or control DNA. Mice were immunized with pcDNA3, pcDNA3-E7, pcDNA3-Sig/E7/LAMP-1, pcDNA3-ETA(dII)/E7, pcDNA3-E7/HSP70, or pcDNA3-CRT/E7 co-administered with pcDNA3 or with pcDNA3-SPI-6. Splenocytes harvested 7 days after a booster vaccination were cultured in vitro with MHC class II-restricted E7 peptide (aa 30-67) overnight and stained for CD4, IFNγ, and IL-4. FIG. 14A is a bar graph depicting the number of E7-specific IFNγ-secreting CD4+ T cell precursors (mean±SD). FIG. 14B is a bar graph depicting the number of E7-specific IL-4-secreting CD4⁺ T lymphocytes (mean±SD).

FIG. 15 is a graph showing tumor growth in vaccinated mice receiving pcDNA3-Sig/E7/LAMP-1 co-administered with pcDNA3-SPI-6 or control DNA. Data are expressed as the mean number of lung nodules ±SE.

FIGS. 16A-16B are bar graphs showing numbers of E7-specific CD8⁺ T cell precursors in vivo and non-apoptotic DCs s in vitro after co-administration of antigen-encoding DNA with DNA encoding SPI-6 or mutant mtSPI-6. In FIG. 16A, mice were immunized with pcDNA3-Sig/E7/LAMP-1 mixed with pcDNA3-SPI-6, pcDNA3-mtSPI-6, or pcDNA3. The graph depicts the number of antigen-specific IFN-γ-secreting CD8⁺ T cell precursors (mean±SD). In FIG. 16B, DCs were transfected in vitro with pcDNA3-E7/GFP mixed with pcDNA3-SPI-6, pcDNA3-mtSPI-6, or pcDNA3. Annexin V staining and flow cytometry was performed after gating around a GFP+cell population. DCs were co-cultured with an E7-specific CD8+ T cell line. The graph depicts the meand (±SD) percent of Annexin V-negative (non-apoptotic), GFP+ DCs (results from one representative experiment of two).

FIGS. 17A-17B show results of transfection of DC's with various suicidal DNA vectors. DC-V cells were co-transfected with 2 μg of pcDNA3-GFP (label) mixed with 2 μg of suicide DNA vectors, pSCA1 encoding (i) E7, (ii) BCL-xL, (iii) E7/BCL-xL, (iv) E7/mt BCL-xL, or (v) no insert. The percentage of dead cells among the gated GFP+ cells was determined by flow cytometry after staining with propidium iodide (PI). FIG. 17A is a graph depicting the percentage of dead cells among the gated GFP+ cells as a function of time. FIG. 17B is a histogram depicting percentage of dead DCs among the gated GFP+ cells 4 days after co-transfection (mean±SEM).

FIGS. 18A and 18B evaluate the T cell response to various suicidal DNA vectors. Flow cytometry was used to determine the number of E7-specific IFNγg-secreting CD8+ T cells. Mice (3/group) were immunized with pSCA1 encoding BCL-XL, E7, E7/BCL-xL, or E7/mt BCL-xL. The negative control was pSCA1 (no insert). Splenocytes from vaccinated mice were harvested 7 days after a booster vaccination, cultured in vitro with MHC class I-restricted E7(aa 49-57) peptide overnight, and stained for CD8 and intracellular IFNγ. FIG. 18A shows representative flow cytometry results. FIG. 18B is a bar graph depicting the number of antigen-specific IFNγ-secreting CD8+ T cells (mean±SEM).

FIG. 19A-19C show anti-tumor responses in mice immunized with suicidal DNA vectors as above. In vivo tumor protection, antibody depletion, and tumor treatment experiments using E7-expressing TC-1 tumor cells. FIG. 19A shows in vivo tumor protection against the growth of TC -1 tumors in mice immunized with the indicated vector and subcutaneously challenged with tumor cells in the right leg. 100% of mice vaccinated with pSCA1-E7/BCL-xL remained tumor-free 42 days after TC-1 challenge. FIG. 19B is a graph shows the results of antibody depletion in mice given mAbs to deplete CD4, CD8, and NK1.1 cells. FIG. 19C shows the results of treatment of tumors using the suicidal DNA vaccines, in which mice were first inoculated with tumor cells and later immunized with one of the various vector types. Mice were sacrificed after 35 day and the numbers of pulmonary nodules determined (mean±SEM).

### DESCRIPTION OF THE PREFERRED EMBODIMENTS

### Partial List of Abbreviations Used

APC, antigen presenting cell; CMV, cytomegalovirus; CTL, cytotoxic T lymphocyte; DC, dendritic cell; ECD, extracellular domain; E6, HPV oncoprotein E6; E7, HPV oncoprotein E7; ELISA, enzyme-linked immunosorbent assay; FL, Flt3 ligand; GFP, green fluorescent protein; HPV, human papillomavirus; HSP, heat shock protein; Hsp70, mycobacterial heat shock protein 70; IFNγ, interferon-γ; i.m., intramuscular(ly); i.v., intravenous(ly); MHC, major histocompatibility complex; PBS, phosphate-buffered saline; PCR, polymerase chain reaction; β-gal, β-galactosidase

The present invention is directed to one of two fundamental approaches to the improvement of molecular vaccine potency. As the present inventors discovered, in addition to DNA encoding an antigen, the concomitant administration of a second DNA molecule encoding an anti-apoptotic polypeptide (termed “anti-apoptotic DNA” for simplicity), enhances the magnitude and/or duration of a T cell mediated immune response, and potentiates a desired clinical effect—such as eradication of an existing tumor or prevention of the spread or metastasis of a tumor.

The anti-apoptotic DNA may be physically linked to the antigen-encoding DNA. Examples of this are provided, primarily in the form of suicidal DNA vaccine vectors. Alternatively, the anti-apoptotic DNA may be administered separately from, but in combination with the antigen-endcoding DNA molecule. Even more examples of the co-administration of these two types of vectors is provided.

This strategy may be combined with an additional strategy pioneered by the present inventors and colleagues, that involve linking DNA encoding another protein, generically termed a “targeting polypeptide, to the antigen-encoding DNA. Again, for the sake of simplicity, the DNA encoding such a targeting polypeptide will be referred to herein as a “targeting DNA.” That strategy has been shown to be effective in enhancing the potency of the vectors carrying only antigen-encoding DNA. See for example: Wu et al., WO 01/29233; Wu et al., WO 02/009645; Wu et al., WO 02/061113; Wu et al., WO 02/074920; Wu et al., WO 02/12281, all of which are incorporated by reference in their entirety.

The details of the various targeting polypeptide strategies will not be discussed in detail herein, although such vectors are used in the present examples, and their sequences are provided below. The preferred “targeting polypeptide” include the sorting signal of the lysosome-associated membrane protein type 1 (Sig/LAMP-1), the translocation domain (domain II or dII) of Pseudomonas aeruginosa exotoxin A (ETA(dII) (or from similar toxins from Diptheria, Clostridium, Botulinum, Bacillus, Yersinia, Vibrio cholerae, or Bordetella), an endoplasmic reticulum chaperone polypeptide exemplified by calreticulin (CRT) but also including ER60, GRP94 or gp96, well-characterized ER chaperone polypeptide that representatives of the HSP90 family of stress-induced proteins (see WO 02/012281), VP22 protein from herpes simplex virus and related herpes viruses such as Marek's disease virus (see WO 02/09645), mycobacterial heat shock protein HSP70, and γ-tubulin. DNA encoding each of these polypeptides, or fragments or variants thereof with substantially the same biological activity, when linked to an antigen-endcoding or epitope-encoding DNA molecule, result in more potent T cell mediate responses to the antigen compared to immunization with the antigen-encoding DNA alone. These polypeptide can be considered as “molecular adjuvants.” These effects are manifest primarily with CD8+ T cells, although some of these approaches induce potent CD4+ T cell mediated effects as well.

The results presented herein prove that molecular vaccination with

(a) a combination of an antigen-encoding DNA and an anti-apoptotic DNA; or

(b) a combination of a chimeric DNA encoding antigen and a targeting DNA sequence; or

(c) a chimeric DNA comprising

(i) an antigen-encoding DNA sequence linked to an antiapoptotic DNA sequence; or
(ii) an antigen-encoding DNA sequence linked to both an antiapoptotic DNA and a targeting DNA;
   or a combination of any of the above, will results in a stronger and more durable immune response which can be protective and/or therapeutic.

The vectors may also comprise DNA encoding an immunostimulatory cytokine, preferably those that target APCs, preferably DC's, such as granulocyte macrophage colony stimulating factor (GM-CSF), or active fragments or domains thereof, and/or DNA encoding a costimulatory signal, such as a B7 family protein, including B7-DC (see U.S. Ser. No. 09/794,210), B7.1, B7.2, soluble CD40, etc.).

The vectors used to deliver the foregoing DNA sequences include naked DNA vectors, DNA-based alphaviral RNA replicons (“suicidal DNA vectors”) as disclosed herein, and self replicating RNA replicons. y be similar pathogenic bacterial toxins pertussis, or active fragments or domains of any of the foregoing polypeptides.

The order in which the two (or more) components of a chimeric DNA construct are arranged, and therefore, the order of the encoding nucleic acid fragments in the nucleic acid vector, can be altered without affecting immunogenicity of the fusion polypeptides proteins and the utility of the composition.

The experiments described herein demonstrate that the methods of the invention can enhance a cellular immune response, particularly, tumor-destructive CTL reactivity, induced by a DNA vaccine encoding an epitope of a human pathogen. Human HPV-16 E7 was used as a model antigen for vaccine development because human papillomaviruses (HPVs), particularly HPV-16, are associated with most human cervical cancers. The oncogenic HPV proteins E7 and E6 are important in the induction and maintenance of cellular transformation and co-expressed in most HPV-containing cervical cancers and their precursor lesions. Therefore, cancer vaccines, such as the compositions of the invention, that target E7 can be used to control of HPV-associated neoplasms (Wu (1994) Curr. Opin. Immunol. 6:746-754).

However, the present invention is not limited to the exemplified antigen(s). Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Thus, in one embodiment, the antigen (e.g., the MHC class I-binding peptide epitope) is derived from a pathogen, e.g., it comprises a peptide expressed by a pathogen. The pathogen can be a virus, such as, e.g., a papilloma virus, a herpesvirus, a retrovirus (e.g., an immunodeficiency virus, such as HIV-1), an adenovirus, and the like. The papilloma virus can be a human papilloma virus; for example, the antigen (e.g., the Class I-binding peptide) can be derived from an HPV-16 E6 or E7 polypeptide. In one embodiment, the HPV-16 E6 or E7 polypeptide used as an immunogen is substantially non-oncogenic, i.e., it does not bind retinoblastoma polypeptide (pRB) or binds pRB with such low affinity that the HPV-16 E7 polypeptide is effectively non-oncogenic when expressed or delivered in vivo.

In alternative embodiments, the pathogen is a bacteria, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum.

In another embodiment, the MHC class I-binding peptide epitope is derived from a tumor cell. The tumor cell-derived peptide epitope can comprise a tumor associated antigen, e.g., a tumor specific antigen, such as, e.g., a HER-2/neu antigen, or one of a number of known melanoma antigens, etc.

In one embodiment, the isolated or recombinant nucleic acid molecule is operatively linked to a promoter, such as, e.g., a constitutive, an inducible or a tissue-specific promoter. The promoter can be expressed in any cell, including cells of the immune system, including, e.g., antigen presenting cells (APCs), e.g., in a constitutive, an inducible or a tissue-specific manner.

In alternative embodiments, the APCs are dendritic cells, keratinocytes, astrocytes, monocytes, macrophages, B lymphocytes, a microglial cell, or activated endothelial cells, and the like.

Unless defined otherwise, all technical and scientific terms used herein have the meaning commonly understood by a person skilled in the art of this invention. As used herein, the following terms have the meanings ascribed to them unless specified otherwise.

The term “antigen” or “immunogen” as used herein refers to a compound or composition comprising a peptide, polypeptide or protein which is “antigenic” or “immunogenic” when administered (or expressed in vivo by an administered nucleic acid, e.g., a DNA vaccine) in an appropriate amount (an “immunogenically effective amount”), i.e., capable of inducing, eliciting, augmenting or boosting a cellular and/or humoral immune response either alone or in combination or linked or fused to another substance (which can be administered at once or over several intervals). An immunogenic composition can comprise an antigenic peptide of at least about 5 amino acids, a peptide of 10 amino acids in length, a polypeptide fragment of 15 amino acids in length, 20 amino acids in length or longer. Smaller immunogens may require presence of a “carrier” polypeptide e.g., as a fusion protein, aggregate, conjugate or mixture, preferably linked (chemically or otherwise) to the immunogen. The immunogen can be recombinantly expressed from a vaccine vector, which can be naked DNA comprising the immunogen's coding sequence operably linked to a promoter, e.g., an expression cassette as described herein. The immunogen includes one or more antigenic determinants or epitopes which may vary in size from about 3 to about 15 amino acids.

The term “epitope” as used herein refers to an antigenic determinant or antigenic site that interacts with an antibody or a T cell receptor (TCR), e.g., the MHC class I-binding peptide compositions (or expressed products of the nucleic acid compositions of the invention) used in the methods of the invention. An “antigen” is a molecule or chemical structure that either induces an immune response or is specifically recognized or bound by the product or mediator of an immune response, such as an antibody or a CTL. The specific conformational or stereochemical “domain” to which an antibody or a TCR bind is an “antigenic determinant” or “epitope.” TCRs bind to peptide epitopes which are physically associated with a third molecule, a major histocompatibility complex (MHC) class I or class II protein.

The term “recombinant” refers to (1) a nucleic acid or polynucleotide synthesized or otherwise manipulated in vitro, (2) methods of using recombinant DNA technology to produce gene products in cells or other biological systems, or (3) a polypeptide encoded by a recombinant nucleic acid. For example, the ETA(dII)-encoding nucleic acid or polypeptide, the nucleic acid encoding an MHC class I-binding peptide epitope (antigen) or the peptide itself can be recombinant. “Recombinant means” includes ligation of nucleic acids having various coding regions or domains or promoter sequences from different sources into a single unit in the form of an expression cassette or vector for expression of the coding sequences in the vectors resulting in production of the encoded polypeptide.

The term “self-replicating RNA replicon” refers to a construct based on an RNA viruses, such as alphavirus genome RNAs (e.g., Sindbis virus, Semliki Forest virus, etc.), that have been engineered to allow expression of heterologous RNAs and proteins. These recombinant vectors are self-replicating (“replicons”) which can be introduced into cells as naked RNA or DNA, as described in detail in co-pending, commonly assigned U.S. and PCT patent applications by several of the present inventors (U.S. Ser. No. 10/060,274, and WO 02/061113).

Sequences of Polypeptides and Nucleic Acids

Plasmid and Vector Sequences

The sequence of the pcDNA3 plasmid vector (SEQ ID NO:1) is:

GACGGATCGG GAGATCTCCC GATCCCCTAT GGTCGACTCT CAGTACAATC TGCTCTGATG CCGCATAGTT AAGCCAGTAT CTGCTCCCTG
CTTGTGTGTT GGAGGTCGCT GAGTAGTGCG CGAGCAAAAT TTAAGCTACA ACAAGGCAAG GCTFGACCGA CAATTGCATG AAGAATCTGC
TTAGGGTTAG GCGTTTTGCG CTGCTTCGCG ATGTACGGGC CAGATATACG CGTTGACATT GATTATTGAC TAGTTATThA TAGTAATCAA
TTACGGGGTC ATTAGTTCAT AGCCCATATA TGGAGTTCCG CGTTACATAA CTTACGGTAA ATGGCCCGCC TGGCTGACCG CCCAACGACC
CCCGCCCATT GACGTCAATA ATGACGTATG TTCCCATAGT AACGCCAATA GGGACTTTCC ATTGACGTCA ATGGGTGGAC TATTTACGGT
AAACTGCCCA CTTGGCAGTA CATCAAGTGT ATCATATGCC AAGTACGCCC CCTATTGACG TCAATGACGG TAAATGGCCC GCCTGGCATT
ATGCCCAGTA CATGACCTTA TGGGACTTTC CTACTTGGCA GTACATCTAC GTATTAGTCA TCGCTATTAC CATGGTGATG CGGTTTTGGC
AGTACATCAA TGGGCGTGGA TAGCGGTTTG ACTCACGGGG ATTTCCAAGT CTCCACCCCA TTGACGTCAA TGGGAGTFTG TTTTGGCACC
AAAATCAACG GGACTTTCCA AAATGTCGTA ACAACTCCGC CCCATTGACG CAAATGGGCG GTAGGCGTGT ACGGTGGGAG GTCTATATAA
GCAGAGCTCT CTGGCTAACT AGAGAACCCA CTGCTTACTG GCTTATCGAA ATTAATACGA CTCACTATAG GGAGACCCAA GCTGGCTAGC
GTTTAAACGG GCCCTCTAGA CTCGAGCGGC CGCCACTGTG CTGGATATCT GCAGAATTCC ACCACACTGG ACTAGTGGAT CCGAGCTCGG
TACCAAGCTT AAGTTTAAAC CGCTGATCAG CCTCGACTGT GCCTTCTAGT TGCCAGCCAT CTGTTGTTTG CCCCTCCCCC GTGCCTTCCT
TGACCCTGGA AGGTGCCACT CCCACTGTCC TTTCCTAATA AAATGAGGAA ATTGCATCGC ATTGTCTGAG TAGGTGTCAT TCTATTCTGG
GGGGTGGGGT GGGGCAGGAC AGCAAGGGGG AGGATTGGGA AGACAATAGC AGGCATGCTG GGGATGCGGT GGGCTCTATG GCTTCTGAGG
CGGAAAGAAC CAGCTGGGGC TCTAGGGGGT ATCCCCACGC GCCCTGTAGC GGCGCATTAA GCGCGGCGGG TGTGGTGGTT ACGCGCAGCG
TGACCGCTAC ACTTGCCAGC GCCCTAGCGC CCGCTCCTTT CGCTTTCTTC CCTTCCTTTC TCGCCACGTT CGCCGGCTTT CCCCGTCAAG
CTCTAAATCG GGGCATCCCT TTAGGGTTCC GATTTAGTGC TTTACGGCAC CTCGACCCCA AAAAACTTGA TTAGGGTGAT GGTTCACGTA
GTGGGCCATC GCCCTGATAG ACGGTTTTTC GCCCTTTGAC GTTGGAGTCC ACGTTCTTTA ATAGTGGACT CTTGTTCCAA ACTGGAACAA
CACTCAACCC TATCTCGGTC TATTCTTTTG ATTTATAAGG GATTTTGGGG ATTTCGGCCT ATTGGTTAAA AAATGAGCTG ATTTAACAAA
AATTTAACGC GAATTAATTC TGTGGAATGT GTGTCAGTTA GGGTGTGGAA AGTCCCCAGG CTCCCCAGGC AGGCAGAAGT ATGCAAAGCA
TGCATCTCAA TTAGTCAGCA ACCAGGTGTG GAAAGTCCCC AGGCTCCCCA GCAGGCAGAA GTATGCAAAG CATGCATCTC AATTAGTCAG
CAACCATAGT CCCGCCCCTA ACTCCGCCCA TCCCGCCCCT AACTCCGCCC AGTTCCGCCC ATTCTCCGCC CCATGGCTGA CTAATTTTTT
TTATTTATGC AGAGGCCGAG GCCGCCTCTG CCTCTGAGCT ATTCCAGAAG TAGTGAGGAG GCTTTTTTGG AGGCCTAGGC TTTTGCAAAA
AGCTCCCGGG AGCTTGTATA TCCATTTTCG GATCTGATCA AGAGACAGGA TGAGGATCGT TTCGCATGAT TGAACAAGAT GGATTGCACG
CAGGTTCTCC GGCCGCTTGG GTGGAGAGGC TATTCGGCTA TGACTGGGCA CAACAGACAA TCGGCTGCTC TGATGCCGCC GTGTTCCGGC
TGTCAGCGCA GGGGCGCCCG GTTCTTTTTG TCAAGACCGA CCTGTCCGGT GCCCTGAATG AACTGCAGGA CGAGGCAGCG CGGCTATCGT
GGCTGGCCAC GACGGGCGTT CCTTGCGCAG CTGTGCTCGA CGTTGTCACT GAAGCGGGAA GGGACTGGCT GCTATTGGGC GAAGTGCCGG
GGCAGGATCT CCTGTCATCT CACCTTGCTC CTGCCGAGAA AGTATCCATC ATGGCTGATG CAATGCGGCG GCTGCATACG CTTGATCCGG
CTACCTGCCC ATTCGACCAC CAAGCGAAAC ATCGCATCGA GCGAGCACGT ACTCGGATGG AAGCCGGTCT TGTCGATCAG GATGATCTGG
ACGAAGAGCA TCAGGGGCTC GCGCCAGCCG AACTGTTCGC CAGGCTCAAG GCGCGCATGC CCGACGGCGA GGATCTCGTC GTGACCCATG
GCGATGCCTG CTTGCCGAAT ATCATGGTGG AAAATGGCCG CTTTTCTGGA TTCATCGACT GTGGCCGGCT GGGTGTGGCG GACCGCTATC
AGGACATAGC GTTGGCTACC CGTGATATTG CTGAAGAGCT TGGCGGCGAA TGGGCTGACC GCTTCCTCGT GCTTTACGGT ATCGCCGCTC
CCGATTCGCA GCGCATCGCC TTCTATCGCC TTCTTGACGA GTTCTTCTGA GCGGGACTCT GGGGTTCGAA ATGACCGACC AAGCGACGCC
CAACCTGCCA TCACGAGATT TCGATTCCAC CGCCGCCTTC TATGAAAGGT TGGGCTTCGG AATCGTTTTC CGGGACGCCG GCTGGATGAT
CCTCCAGCGC GGGGATCTCA TGCTGGAGTT CTTCGCCCAC CCCAACTTGT TTATTGCAGC TTATAATGGT TACAAATAAA GCAATAGCAT
CACAAATTTC ACAAATAAAG CATTTTTTTC ACTGCATTCT AGTTGTGGTT TGTCCAAACT CATCAATG7A TCTTATCATG TCTGTATACC
GTCGACCTCT AGCTAGAGCT TGGCGTAATC ATGGTCATAG CTGTTTCCTG TGTGAAATTG TTATCCGCTC ACAATTCCAC ACAACATACG
AGCCGGAAGC ATAAAGTGTA AAGCCTGGGG TGCCTAATGA GTGAGCTAAC TCACATTAAT TGCGTTGCGC TCACTGCCCG CTTTCCAGTC
GGGAAACCTG TCGTGCCAGC TGCATTAATG AATCGGCCAA CGCGCGGGGA GAGGCGGTTT GCGTATTGGG CGCTCTTCCG CTTCCTCGCT
CACTGACTCG CTGCGCTCGG TCGTTCGGCT GCGGCGAGCG GTATCAGCTC ACTCAAAGGC GGTAATACGG TTATCCACAG AATCAGGGGA
TAACGCAGGA AAGAACATGT GAGCAAAAGG CCAGCAAAAG GCCAGGAACC GTAAAAAGGC CGCGTTGCTG GCGTTTTTCC ATAGGCTCCG
CCCCCCTGAC GAGCATCACA AAAATCGACG CTCAAGTCAG AGGTGGCGAA ACCCGACAGG ACTATAAAGA TACCAGGCGT TTCCCCCTGG
AAGCTCCCTC GTGCGCTCTC CTGTTCCGAC CCTGCCGCTT ACCGGATACC TGTCCGCCTT TCTCCCTTCG GGAAGCGTGG CGCTTTCTCA
ATGCTCACGC TGTAGGTATC TCAGTTCGGT GTAGGTCGTT CGCTCCAAGC TGGGCTGTGT GCACGAACCC CCCGTTCAGC CCGACCGCTG
CGCCTTATCC GGTAACTATC GTCTTGAGTC CAACCCGGTA AGACACGACT TATCGCCACT GGCAGCAGCC ACTGGTAACA GGATTAGCAG
AGCGAGGTAT GTAGGCGGTG CTACAGAGTT CTTGAAGTGG TGGCCTAACT ACGGCTACAC TAGAAGGACA GTATTTGGTA TCTGCGCTCT
GCTGAAGCCA GTTACCTTCG GAAAAAGAGT TGGTAGCTCT TGATCCGGCA AACAAACCAC CGCTGGTAGC GGTGGTTTTT TTGTTTGCAA
GCAGCAGATT ACGCGCAGAA AAAAAGGATC TCAAGAAGAT CCTTTGATCT TTTCTACGGG GTCTGACGCT CAGTGGAACG AAAACTCACG
TTAAGGGATT TTGGTCATGA GATTATCAAA AAGGATCTTC ACCTAGATCC TTTTAAATTA AAAATGAAGT TTTAAATCAA TCTAAAGTAT
ATATGAGTAA ACTTGGTCTG ACAGTTACCA ATGCTTAATC AGTGAGGCAC CTATCTCAGC GATCTGTCTA TTTCGTTCAT CCATAGTTGC
CTGACTCCCC GTCGTGTAGA TAACTACGAT ACGGGAGGGC TTACCATCTG GCCCCAGTGC TGCAATGATA CCGCGAGACC CACGCTCACC
GGCTCCAGAT TTATCAGCAA TAAACCAGCC AGCCGGAAGG GCCGAGCGCA GAAGTGGTCC TGCAACTTTA TCCGCCTCCA TCCAGTCTAT
TAATTGTTGC CGGGAAGCTA GAGTAAGTAG TTCGCCAGTT AATAGTTTGC GCAACGTTGT TGCCATTGCT ACAGGCATCG TGGTGTCACG
CTCGTCGTTT GGTATGGCTT CATTCAGCTC CGGTTCCCAA CGATCAAGGC GAGTTACATG ATCCCCCATG TTGTGCAAAA AAGCGGTTAG
CTCCTTCGGT CCTCCGATCG TTGTCAGAAG TAAGTTGGCC GCAGTGTTAT CACTCATGGT TATGGCAGCA CTGCATAATT CTCTTACTGT
CATGCCATCC GTAAGATGCT TTTCTGTGAC TGGTGAGTAC TCAACCAAGT CATTCTGAGA ATAGTGTATG CGGCGACCGA GTTGCTCTTG
CCCGGCGTCA ATACGGGATA ATACCGCGCC ACATAGCAGA ACTTTAAAAG TGCTCATCAT TGGAAAACGT TCTTCGGGGC GAAAACTCTC
AAGGATCTTA CCGCTGTTGA GATCCAGTTC GATGTAACCC ACTCGTGCAC CCAACTGATC TTCAGCATCT TTTACTTTCA CCAGCGTTTC
TGGGTGAGCA AAAACAGGAA GGCAAAATGC CGCAAAAAAG GGAATAAGGG CGACACGGAA ATGTTGAATA CTCATACTCT TCCTTTTTCA
ATATTATTGA AGCATTTATC AGGGTTATTG TCTCATGAGC GGATACATAT TTGAATGTAT TTAGAAAAAT AAACAAATAG GGGTTCCGCG
CACATTTCCC CGAAAAGTGC CACCTGACGT C

The pSCA1 suicide DNA vector has the sequence [SEQ ID NO:2]:

(includes cloning sites

ATGGCGGATG TGTGACATAC ACGACGCCAA AAGATTTTGT TCCAGCTCCT GCCACCTCCG CTACGCGAGA GATTAACCAC
CCACGATGGC CGCCAAAGTG CATGTTGATA TTGAGGCTGA CAGCCCATTC ATCAAGTCTT TGCAGAAGGC ATTTCCGTCG
TTCGAGGTGG AGTCATTGCA GGTCACACCA AATGACCATG CAAATGCCAG AGCATTTTCG CACCTGGCTA CCAAATTGAT
CGAGCAGGAG ACTGACAAAG ACACACTCAT CTTGGATATC GGCAGTGCGC CTTCCAGGAG AATGATGTCT ACGCACAAAT
ACCACTGCGT ATGCCCTATG CGCAGCGCAG AAGACCCCGA AAGGCTCGAT AGCTACGCAA AGAAACTGGC AGCGGCCTCC
GGGAAGGTGC TGGATAGAGA GATCGCAGGA AAAATCACCG ACCTGCAGAC CGTCATGGCT ACGCCAGACG CTGAATCTCC
TACCTTTTGC CTGCATACAG ACGTCACGTG TCGTACGGCA GCCGAAGTGG CCGTATACCA GGACGTGTAT GCTGTACATG
CACCAACATC GCTGTACCAT CAGGCGATGA AAGGTGTCAG AACGGCGTAT TGGATTGGGT TTGACACCAC CCCGTTTATG
TTTGACGCGC TAGCAGGCGC GTATCCAACC TACGCCACAA ACTGGGCCGA CGAGCAGGTG TTACAGGCCA GGAACATAGG
ACTGTGTGCA GCATCCTTGA CTGAGGGAAG ACTCGGCAAA CTGTCCATTC TCCGCAAGAA GCAATTGAAA CCTTGCGACA
CAGTCATGTT CTCGGTAGGA TCTACATTGT ACACTGAGAG CAGAAAGCTA CTGAGGAGCT GGCACTTACC CTCCGTATTC
CACCTGAAAG GTAAACAATC CTTTACCTGT AGGTGCGATA CCATCGTATC ATGTGAAGGG TACGTAGTTA AGAAAATCAC
TATGTGCCCC GGCCTGTACG GTAAAACGGT AGGGTACGCC GTGACGTATC ACGCGGAGGG ATTCCTAGTG TGCAAGACCA
CAGACACTGT CAAAGGAGAA AGAGTCTCAT TCCCTGTATG CACCTACGTC CCCTCAACCA TCTGTGATCA AATGACTGGC
ATACTAGCGA CCGACGTCAC ACCGGAGGAC GCACAGAAGT TGTTAGTGGG ATTGAATCAG AGGATAGTTG TGAACGGAAG
AACACAGCGA AACACTAACA CGATGAAGAA CTATCTGCTT CCGATTGTGG CCGTCGCATT TAGCAAGTGG GCGAGGGAAT
ACAAGGCAGA CCTTGATGAT GAAAAACCTC TGGGTGTCCG AGAGAGGTCA CTTACTTGCT GCTGCTTGTG GGCATTTAAA
ACGAGGAAGA TGCACACCAT GTACAAGAAA CCAGACACCC AGACAATAGT GAAGGTGCCT TCAGAGTTTA ACTCGTTCGT
CATCCCGAGC CTATGGTCTA CAGGCCTCGC AATCCCAGTC AGATCACGCA TTAAGATGCT TTTGGCCAAG AAGACCAAGC
GAGAGTTAAT ACCTGTTCTC GACGCGTCGT CAGCCAGGGA TGCTGAACAA GAGGAGAAGG AGAGGTTGGA GGCCGAGCTG
ACTAGAGAAG CCTTACCACC CCTCGTCCCC ATCGCGCCGG CGGAGACGGG AGTCGTCGAC GTCGACGTTG AAGAACTAGA
GTATCACGCA GGTGCAGGGG TCGTGGAAAC ACCTCGCAGC GCGTTGAAAG TCACCGCACA GCCGAACGAC GTACTACTAG
GAAATTACGT AGTTCTGTCC CCGCAGACCG TGCTCAAGAG CTCCAAGTTG GCCCCCGTGC ACCCTCTAGC AGAGCAGGTG
AAAATAATAA CACATAACGG GAGGGCCGGC GGTTACCAGG TCGACGGATA TGACGGCAGG GTCCTACTAC CATGTGGATC
GGCCATTCCG GTCCCTGAGT TTCAAGCTTT GAGCGAGAGC GCCACTATGG TGTACAACGA AAGGGAGTTC GTCAACAGGA
AACTATACCA TATTGCCGTT CACGGACCGT CGCTGAACAC CGACGAGGAG AACTACGAGA AAGTCAGAGC TGAAAGAACT
GACGCCGAGT ACGTGTTCGA CGTAGATAAA AAATGCTGCG TCAAGAGAGA GGAAGCGTCG GGTTTGGTGT TGGTGGGAGA
GCTAACCAAC CCCCCGTTCC ATGAATTCGC CTACGAAGGG CTGAAGATCA GGCCGTCGGC ACCATATAAG ACTACAGTAG
TAGGAGTCTT TGGGGTTCCG GGATCAGGCA AGTCTGCTAT TATTAAGAGC CTCGTGACCA AACACGATCT GGTCACCAGC
GGCAAGAAGG AGAACTGCCA GGAAATAGTT AACGACGTGA AGAAGCACCG CGGGAAGGGG ACAAGTAGGG AAAACAGTGA
CTCCATCCTG CTAAACGGGT GTCGTCGTGC CGTGGACATC CTATATGTGG ACGAGGCTTT CGCTaGCCAT TCCGGTACTC
TGCTGGCCCT AATTGCTCTT GTTAAACCTC GGAGCAAAGT GGTGTTATGC GGAGACCCCA AGCAATGCGG ATTCTTCAAT
ATGATGCAGC TTAAGGTGAA CTTCAACCAC AACATCTGCA CTGAAGTATG TCATAAAAGT ATATCCAGAC GTTGCACGCG
TCCAGTCACG GCCATCGTGT CTACGTTGCA CTACGGAGGC AAGATGCGCA CGACCAACCC GTGCAACAAA CCCATAATCA
TAGACACCAC AGGACAGACC AAGCCCAAGC CAGGAGACAT CGTGTTAACA TGCTTCCGAG GCTGGGCAAA GCAGCTGGAG
TTGGACTACC GTGGACACGA AGTCATGACA GCAGCAGCAT CTCAGGGCCT CACCCGCAAA GGGGTATACG CCGTAAGGCA
GAAGGTGAAT GAAAATCCCT TGTATGCCCC TGCGTCGGAG CACGTGAATG TACTGCTGAC GCGCACTGAG GATAGGCTGG
TGTGGAAAAC GCTGGCCGGC GATCCCTGGA TTAAGGTCCT ATCAAACATT CCACAGGGTA ACTTTACGGC CACATTGGAA
GAATGGCAAG AAGAACACGA CAAAATAATG AAGGTGATTG AAGGACCGGC TGCGCCTGTG GACGCGTTCC AGAACAAAGC
GAACGTGTGT TGGGCGAAAA GCCTGGTGCC TGTCCTGGAC ACTGCCGGAA TCAGATTGAC AGCAGAGGAG TGGAGCACCA
TAATTACAGC ATTTAAGGAG GACAGAGCTT ACTCTCCAGT GGTGGCCTTG AATGAAATTT GCACCAAGTA CTATGGAGTT
GACCTGGACA GTGGCCTGTT TTCTGCCCCG AAGGTGTCCC TGTATTACGA GAACAACCAC TGGGATAACA GACCTGGTGG
AAGGATGTAT GGATTCAATG CCGCAACAGC TGCCAGGCTG GAAGCTAGAC ATACCTTCCT GAAGGGGCAG TGGCATACGG
GCAAGCAGGC AGTTATCGCA GAAAGAAAAA TCCAACCGCT TTCTGTGCTG GACAATGTAA TTCCTATCAA CCGCAGGCTG
CCGCACGCCC TGGTGGCTGA GTACAAGACG GTTAAAGGCA GTAGGGTTGA GTGGCTGGTC AATAAAGTAA GAGGGTACCA
CGTCCTGCTG GTGAGTGAGT ACAACCTGGC TTTGCCTCGA CGCAGGGTCA CTTGGTTGTC ACCGCTGAAT GTCAGAGGCG
CCGATAGGTG CTACGACCTA AGTTTAGGAC TGCCGGCTGA CGCCGGCAGG TTCGACTTGG TCTTTGTGAA CATTCACACG
GAATTCAGAA TCCACCACTA CCAGCAGTGT GTCGACCACG CCATGAAGCT GCAGATGCTT GGGGGAGATG CGCTACGACT
GCTAAAACCC GGCGGCATCT TGATGAGAGC TTACGGATAC GCCGATAAAA TCAGCGAAGC CGTTGTTTCC TCCTTAAGCA
GAAAGTTCTC GTCTGCAAGA GTGTTGCGCC CGGATTGTGT CACCAGCAAT ACAGAAGTGT TCTTGCTGTT CTCCAACTTT
GACAACGGAA AGAGACCCTC TACGCTACAC CAGATGAATA CCAAGCTGAG TGCCGTGTAT GCCGGAGAAG CCATGCACAC
GGCCGGGTGT GCACCATCCT ACAGAGTTAA GAGAGCAGAC ATAGCCACGT GCACAGAAGC GGCTGTGGTT AACGCAGCTA
ACGCCCGTGG AACTGTAGGG GATGGCGTAT GCAGGGCCGT GGCGAAGAAA TGGCCGTCAG CCTTTAAGGG AGCAGCAACA
CCAGTGGGCA CAATTAAAAC AGTCATGTGC GGCTCGTACC CCGTCATCCA CGCTGTAGCG CCTAATTTCT CTGCCACGAC
TGAAGCGGAA GGGGACCGCG AATTGGCCGC TGTCTACCGG GCAGTGGCCG CCGAAGTAAA CAGACTGTCA CTGAGCAGCG
TAGCCATCCC GCTGCTGTCC ACAGGAGTGT TCAGCGGCGG AAGAGATAGG CTGCAGCAAT CCCTCAACCA TCTATTCACA
GCAATGGACG CCACGGACGC TGACGTGACC ATCTACTGCA GAGACAAAAG TTGGGAGAAG AAAATCCAGG AAGCCATTGA
CATGAGGACG GCTGTGGAGT TGCTCAATGA TGACGTGGAG CTGACCACAG ACTTGGTGAG AGTGCACCCG GACAGCAGCC
TGGTGGGTCG TAAGGGCTAC AGTACCACTG ACGGGTCGCT GTACTCGTAC TTTGAAGGTA CGAAATTCAA CCAGGCTGCT
ATTGATATGG CAGAGATACT GACGTTGTGG CCCAGACTGC AAGAGGCAAA CGAACAGATA TGCCTATACG CGCTGGGCGA
AACAATGGAC AACATCAGAT CCAAATGTCC GGTGAACGAT TCCGATTCAT CAACACCTCC CAGGACAGTG CCCTGCCTGT
GCCGCTACGC AATGACAGCA GAACGGATCG CCCGCCTTAG GTCACACCAA GTTAAAAGCA TGGTGGTTTG CTCATCTTTT
CCCCTCCCGA AATACCATGT AGATGGGGTG CAGAAGGTAA AGTGCGAGAA GGTTCTCCTG TTCGACCCGA CGGTACCTTC
AGTGGTTAGT CCGCGGAAGT ATGCCGCATC TACGACGGAC CACTCAGATC GGTCGTTACG AGGGTTTGAC TTGGACTGGA
CCACCGACTC GTCTTCCACT GCCAGCGATA CCATGTCGCT ACCCAGTTTG CAGTCGTGTG ACATCGACTC GATCTACGAG
CCAATGGCTC CCATAGTAGT GACGGCTGAC GTACACCCTG AACCCGCAGG CATCGCGGAC CTGGCGGCAG ATGTGCACCC
TGAACCCGCA GACCATGTGG ACCTCGAGAA CCCGATTCCT CCACCGCGCC CGAAGAGAGC TGCATACCTT GCCTCCCGCG
CGGCGGAGCG ACCGGTGCCG GCGCCGAGAA AGCCGACGCC TGCCCCAAGG ACTGCGTTTA GGAACAAGCT GCCTTTGACG
TTCGGCGACT TTGACGAGCA CGAGGTCGAT GCGTTGGCCT CCGGGATTAC TTTCGGAGAC TTCGACGACG TCCTGCGACT
AGGCCGCGCG GGTGCATATA TTTTCTCCTC GGACACTGGC AGCGGACATT TACAACAAAA ATCCGTTAGG CAGCACAATC
TCCAGTGCGC ACAACTGGAT GCGGTCCAGG AGGAGAAAAT GTACCCGCCA AAATTGGATA CTGAGAGGGA GAAGCTGTTG
CTGCTGAAAA TGCAGATGCA CCCATCGGAG GCTAATAAGA GTCGATACCA GTCTCGCAAA GTGGAGAACA TGAAAGCCAC
GGTGGTGGAC AGGCTCACAT CGGGGGCCAG ATTGTACACG GGAGCGGACG TAGGCCGCAT ACCAACATAC GCGGTTCGGT
ACCCCCGCCC CGTGTACTCC CCTACCGTGA TCGAAAGATT CTCAAGCCCC GATGTAGCAA TCGCAGCGTG CAACGAATAC
CTATCCAGAA ATTACCCAAC AGTGGCGTCG TACCAGATAA CAGATGAATA CGACGCATAC TTGGACATGG TTGACGGGTC
GGATAGTTGC TTGGACAGAG CGACATTCTG CCCGGCGAAG CTCCGGTGCT ACCCGAAACA TCATGCGTAC CACCAGCCGA
CTGTACGCAG TGCCGTCCCG TCACCCTTTC AGAACACACT ACAGAACGTG CTAGCGGCCG CCACCAAGAG AAACTGCAAC
GTCACGCAAA TGCGAGAACT ACCCACCATG GACTCGGCAG TGTTCAACGT GGAGTGCTTC AAGCGCTATG CCTGCTCCGG
AGAATATTGG GAAGAATATG CTAAACAACC TATCCGGATA ACCACTGAGA ACATCACTAC CTATGTGACC AAATTGAAAG
GCCCGAAAGC TGCTGCCTTG TTCGCTAAGA CCCACAACTT GGTTCCGCTG CAGGAGGTTC CCATGGACAG ATTCACGGTC
GACATGAAAC GAGATGTCAA AGTCACTCCA GGGACGAAAC ACACAGAGGA AAGACCCAAA GTCCAGGTAA TTCAAGCAGC
GGAGCCATTG GCGACCGCTT ACCTGTGCGG CATCCACAGG GAATTAGTAA GGAGACTAAA TGCTGTGTTA CGCCCTAACG
TGCACACATT GTTTGATATG TCGGCCGAAG ACTTTGACGC GATCATCGCC TCTCACTTCC ACCCAGGAGA CCCGGTTCTA
GAGACGGACA TTGCATCATT CGACAAAAGC CAGGACGACT CCTTGGCTCT TACAGGTTTA ATGATCCTCG AAGATCTAGG
GGTGGATCAG TACCTGCTGG ACTTGATCGA GGCAGCCTTT GGGGAAATAT CCAGCTGTCA CCTACCAACT GGCACGCGCT
TCAAGTTCGG AGCTATGATG AAATCGGGCA TGTTTCTGAC TTTGTTTATT AACACTGTTT TGAACATCAC CATAGCAAGC
AGGGTACTGG AGCAGAGACT CACTGACTCC GCCTGTGCGG CCTTCATCGG CGACGAGAAC ATCGTTCACG GAGTGATCTC
CGACAAGCTG ATGGCGGAGA GGTGCGCGTC GTGGGTCAAC ATGGAGGTGA AGATCATTGA CGCTGTCATG GGCGAAAAAC
CCCCATATTT TTGTGGGGGA TTCATAGTTT TTGACAGCGT CACACAGACC GCCTGCCGTG TTTCAGACCC ACTTAAGCGC
CTGTTCAAGT TGGGTAAGCC GCTAACAGCT GAAGACAAGC AGGACGAAGA CAGGCGACGA GCACTGAGTG ACGAGGTTAG
CAAGTGGTTC CGGACAGGCT TGGGGGCCGA ACTGGAGGTG GCACTAACAT CTAGGTATGA GGTAGAGGGC TGCAAAAGTA
TCCTCATAGC CATGGCCACC TTGGCGAGGG ACATTAAGGC GTTTAAGAAA TTGAGAGGAC CTGTTATACA CCTCTACGGC
GGTCCTAGAT TGGTGCGTTA ATACACAGAA TTCTGATTgg atccCGGGTA ATTAATTGAA TTACATCCCT ACGCAAACGT
TTTACGGCCG CCGGTGGCGC CCGCGCCCGG CGGCCCGTCC TTGGCCGTTG CAGGCCACTC CGGTGGCTCC CGTCGTCCCC
GACTTCGAGG CCCAGCAGAT GCAGCAACTC ATCAGCGCCG TAAATGCGCT GACAATGAGA CAGAACGCAA TTGCTCCTGC
TAGGCCTCCC AAACCAAAGA AGAAGAAGAC AACCAAACCA AAGCCGAAAA CGCAGCCCAA GAAGATCAAC GGAAAAACGC
AGCAGCAAAA GAAGAAAGAC AAGCAAGCCG ACAAGAAGAA GAAGAAACCC GGAAAAAGAG AAAGAATGTG CATGAAGATT
GAAAATGACT GTATCTTCGT ATGCGGCTAG CCACAGTAAC GTAGTGTTTC CAGACATGTC GGGCACCGCA CTATCATGGG
TGCAGAAAAT CTCGGGTGGT CTGGGGGCCT TCGCAATCGG CGCTATCCTG GTGCTGGTTG TGGTCACTTG CATTGGGCTC
CGCAGATAAG TTAGGGTAGG CAATGGCATT GATATAGCAA GAAAATTGAA AACAGAAAAA GTTAGGGTAA GCAATGGCAT
ATAACCATAA CTGTATAACT TGTAACAAAG CGCAACAAGA CCTGCGCAAT TGGCCCCGTG GTCCGCCTCA CGGAAACTCG
GGGCAACTCA TATTGACACA TTAATTGGCA ATAATTGGAA GCTTACATAA GCTTAATTCG ACGAATAATT GGATTTTTAT
TTTATTTTGC AATTGGTTTT TAATATTTCC AAAAAAAAAA AAAAAAAAAA AAAAAAAAAA AAAAAAAAAA AAAAAAAAAA
AAAAAAAAAA AAAAAAAAAA CTAGTgatca taatcagcca taccacattt gtagaggttt tacttgcttt aaaaaacctc
ccacacctcc ccctgaacct gaaacataaa atgaatgcaa ttgttgttgt taacttgttt attgcagctt ataatggtta
caaataaagc aatagcatca caaatttcac aaataaagca tttttttcac tgcattctag ttgtggtttg tccaaactca
tcaatgtatc ttatcatgtc tggaTCTAGT CTGCATTAAT GAATCGGCCA ACGCGCGGGG AGAGGCGGTT TGCGTATTGG
GCGCTCTTCC GCTTCCTCGC TCACTGACTC GCTGCGCTCG GTCGTTCGGC TGCGGCGAGC GGTATCAGCT CACTCAAAGG
CGGTAATACG GTTATCCACA GAATCAGGGG ATAACGCAGG AAAGAACATG TGAGCAAAAG GCCAGCAAAA GGCCAGGAAC
CGTAAAAAGG CCGCGTTGCT GGCGTTTTTC CATAGGCTCC GCCCCCCTGA CGAGCATCAC AAAAATCGAC GCTCAAGTCA
GAGGTGGCGA AACCCGACAG GACTATAAAG ATACCAGGCG TTTCCCCCTG GAAGCTCCCT CGTGCGCTCT CCTGTTCCGA
CCCTGCCGCT TACCGGATAC CTGTCCGCCT TTCTCCCTTC GGGAAGCGTG GCGCTTTCTC AATGCTCGCG CTGTAGGTAT
CTCAGTTCGG TGTAGGTCGT TCGCTCCAAG CTGGGCTGTG TGCACGAACC CCCCGTTCAG CCCGACCGCT GCGCCTTATC
CGGTAACTAT CGTCTTGAGT CCAACCCGGT AAGACACGAC TTATCGCCAC TGGCAGCAGC CACTGGTAAC AGGATTAGCA
GAGCGAGGTA TGTAGGCGGT GCTACAGAGT TCTTGAAGTG GTGGCCTAAC TACGGCTACA CTAGAAGGAC AGTATTTGGT
ATCTGCGCTC TGCTGAAGCC AGTTACCTTC GGAAAAAGAG TTGGTAGCTC TTGATCCGGC AAACAAACCA CCGCTGGTAG
CGGTGGTTTT TTTGTTTGCA AGCAGCAGAT TACGCGCAGA AAAAAAGGAT CTCAAGAAGA TCCTTTGATC TTTTCTACGG
GGcatTCTGA CGCTCAGTGG AACGAAAACT CACGTTAAGG GATTTTGGTC ATGAGATTAT CAAAAAGGAT CTTCACCTAG
ATCCTTTTAA ATTAAAAATG AAGTTTTAAA TCAATCTAAA GTATATATGA GTAAACTTGG TCTGACAGTT ACCAATGCTT
AATCAGTGAG GCACCTATCT CAGCGATCTG TCTATTTCGT TCATCCATAG TTGCCTGACT CCCCGTCGTG TAGATAACTA
CGATACGGGA GGGCTTACCA TCTGGCCCCA GTGCTGCAAT GATACCGCGA GACCCACGCT CACCGGCTCC AGATTTATCA
GCAATAAACC AGCCAGCCGG AAGGGCCGAG CGCAGAAGTG GTCCTGCAAC TTTATCCGCC TCCATCCAGT CTATTAATTG
TTGCCGGGAA GCTAGAGTAA GTAGTTCGCC AGTTAATAGT TTGCGCAACG TTGTTGCCAT TGCTACAGGC ATCGTGGTGT
CACGCTCGTC GTTTGGTATG GCTTCATTCA GCTCCGGTTC CCAACGATCA AGGCGAGTTA CATGATCCCC CATGTTGTGC
AAAAAAGCGG TTAGCTCCTT CGGTCCTCCG ATCGTTGTCA GAAGTAAGTT GGCCGCAGTG TTATCACTCA TGGTTATGGC
AGCACTGCAT AATTCTCTTA CTGTCATGCC ATCCGTAAGA TGCTTTTCTG TGACTGGTGA GTACTCAACC AAGTCATTCT
GAGAATAGTG TATGCGGCGA CCGAGTTGCT CTTGCCCGGC GTCAATACGG GATAATACCG CGCCACATAG CAGAACTTTA
AAAGTGCTCA TCATTGGAAA ACGTTCTTCG GGGCGAAAAC TCTCAAGGAT CTTACCGCTG TTGAGATCCA GTTCGATGTA
ACCCACTCGT GCACCCAACT GATCTTCAGC ATCTTTTACT TTCACCAGCG TTTCTGGGTG AGCAAAAACA GGAAGGCAAA
ATGCCGCAAA AAAGGGAATA AGGGCGACAC GGAAATGTTG AATACTCATA CTCTTCCTTT TTCAATATTA TTGAAGCATT
TATCAGGGTT ATTGTCTCAT GAGCGGATAC ATATTTGAAT GTATTTAGAA AAATAAACAA ATAGGGGTTC CGCGCACATT
TCCCCGAAAA GTGCCACCTG ACGTCTAAGA AACCATTATT ATCATGACAT TAACCTATAA AAATAGGCGT ATCACGAGGC
CCTTTCGTCT CGCGCGTTTC GGTGATGACG GTGAAAACCT CTGACACATG CAGCTCCCGG AGACGGTCAC AGCTTCTGTC
TAAGCGGATG CCGGGAGCAG ACAAGCCCGT CAGGGCGCGT CAGCGGGTGT TGGCGGGTGT CGGGGCTGGC TTAACTATGC
GGCATCAGAG CAGATTGTAC TGAGAGTGCA CCATATCGAC GCTCTCCCTT ATGCGACTCC TGCATTAGGA AGCAGCCCAG
TACTAGGTTG AGGCCGTTGA GCACCGCCGC CGCAAGGAAT GGTGCATGCG TAATCAATTA CGGGGTCATT AGTTCATAGC
CCATATATGG AGTTCCGCGT TACATAACTT ACGGTAAATG GCCCGCCTGG CTGACCGCCC AACGACCCCC GCCCATTGAC
GTCAATAATG ACGTATGTTC CCATAGTAAC GCCAATAGGG ACTTTCCATT GACGTCAATG GGTGGAGTAT TTACGGTAAA
CTGCCCACTT GGCAGTACAT CAAGTGTATC ATATGCCAAG TACGCCCCCT ATTGACGTCA ATGACGGTAA ATGGCCCGCC
TGGCATTATG CCCAGTACAT GACCTTATGG GACTTTCCTA CTTGGCAGTA CATCTACGTA TTAGTCATCG CTATTACCAT
GGTGATGCGG TTTTGGCAGT ACATCAATGG GCGTGGATAG CGGTTTGACT CACGGGGATT TCCAAGTCTC CACCCCATTG
ACGTCAATGG GAGTTTGTTT TGGCACCAAA ATCAACGGGA CTTTCCAAAA TGTCGTAACA ACTCCGCCCC ATTGACGCAA
ATGGGCGGTA GGCGTGTACG GTGGGAGGTC TATATAAGCA GAGCTCTCTG GCTAACTAGA GAACCCACTG CTTAACTGGC
TTATCGAAAT TAATACGACT CACTATAGGG AGACCGGAAG CTTGAATTC

The PSG5 vector has the sequence [SEQ ID NO:3]

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCAGATCTTATTAAAGCAGAACTTGTTTATTGCAGCTTATAATGGTTACAAATAAAGCAATAGCATCA
CAAATTTCACAAATAAAGCATTTTTTTCACTGCATTCTAGTTGTGGTTTGTCCAAACTCATCAATGTATCTTATCATGTC
TGGTCGACTCTAGACTCTTCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGTGAGCAAAAGGCCAGCAAA
AGGCCAGGAACCGTAAAAAGGCCGCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGCGTGGCGCTTTCTCAATGCTCAC
GCTGTAGGTATCTCAGTTCGGTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGCCTAACTACGGCTACACTAGAAGGA
CAGTATTTGGTATCTGCGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAACTTGGTCTGACAGTTAC
CAATGCTTAATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGCGAGTTACATGATCCCCCA
TGTTGTGCAAAAAAGCGGTTAGCTCCTTCGGTCCTCCGATCGTTGTCAGAAGTAAGTTGGCCGCAGTGTTATCACTCATG
GTTATGGCAGCACTGCATAATTCTCTTACTGTCATGCCATCCGTAAGATGCTTTTCTGTGACTGGTGAGTACTCAACCAA
GTCATTCTGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCTTTTTTCAATATTATT
GAAGCATTTATCAGGGTTATTGTCTCATGAGCGGATACATATTTGAATGTATTTAGAAAAATAAACAAATAGGGGTTCCG
CGCACATTTCCCCGAAAAGTGCCACCTGACGTCTAAGAAACCATTATTATCATGACATTAACCTATAAAAATAGGCGTAT
CACGAGGCCCCTTTCGTCTCGCGCGTTTCGGTGATGACGGTGAAAACCTCTGACACATGCAGCTCCCGGAGACGGTCACA
GCTTGTCTGTAAGCGGATGCCGGGAGCAGACAAGCCCGTCAGGGCGCGTCAGCGGGTGTTGGCGGGTGTCGGGGCTGGCT
TAACTATGCGGCATCAGAGCAGATTGTACTGAGAGTGCACCATATGCGGTGTGAAATACCGCACAGATGCGTAAGGAGAA
AATACCGCATCAGGAAATTGTAAACGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGATAGGGTTGAGTGTTGTTCCAGTTTGG
AACAAGAGTCCACTATTAAAGAACGTGGACTCCAACGTCAAAGGGCGAAAAACCGTCTATCAGGGCGATGGCCCACTACG
TGAACCATCACCCTAATCAAGTTTTTTGGGGTCGAGGTGCCGTAAAGCACTAAATCGGAACCCTAAAGGGAGCCCCCGAT
TTAGAGCTTGACGGGGAAAGCCGGCGAACGTGGCGAGAAAGGAAGGGAAGAAAGCGAAAGGAGCGGGCGCTAGGGCGCTG
GCAAGTGTAGCGGTCACGCTGCGCGTAACCACCACACCCGCCGCGCTTAATGCGCCGCTACAGGGCGCGTCGCGCCATTC
GCCATTCAGGTACGCAACTGTTGGGAAGGGCGATCGGTGCGGGCCTCTTCGCTATTACGCCAGCTGGCGAAGGGGGGGAT
GTGCTGCAAGGCGATTAAGTTGGGTAACGCCAGGGTTTTCCCAGTCACGACGTTGTAAAACGACGGCCAGTGAATT

Antigen Sequences

The HPV E7 sequence (nucleotide sequence is SEQ ID NO:4 used in the present vectors and amino acid sequence is SEQ ID NO:5) is shown below:

1/1 31/11
atg cat gga gat aca cct aca ttg cat gaa tat atg tta gat ttg caa cca gag aca act
Met His Gly Asp Thr Pro Thr Leu His Glu Tyr Met Leu Asp Leu Gln Pro Glu Thr Thr
61/21 91/31
gat ctc tac tgt tat gag caa tta aat gac agc tca gag gag gag gat gaa ata gat ggt
Asp Leu Tyr Cys Tyr Glu Gln Leu Asn Asp Ser Ser Glu Glu Glu Asp Glu Ile Asp Gly
121/41 151/51
cca gct gga caa gca gaa ccg gac aga gcc cat tac aat att gta acc ttt tgt tgc aag
Pro Ala Gly Gln Ala Glu Pro Asp Arg Ala His Tyr Asn Ile Val Thr Phe Cys Cys Lys
181/61 211/71
tgt gac tct acg ctt cgg ttg tgc gta caa agc aca cac gta gac att cgt act ttg gaa
Cys Asp Ser Thr Leu Arg Leu Cys Val Gln Ser Thr His Val Asp Ile Arg Thr Leu Glu
241/81 271/91
gac ctg tta atg ggc aca cta gga att gtg tgc ccc atc tgt tct cag gat aag ctt
Asp Leu Leu Met Gly Thr Leu Gly Ile Val Cys Pro Ile Cys Ser Gln Asp Lys Leu

This differs from the GENEBANK Accession Number NC_{—}001526 for the E7 protein which is:

(SEQ ID NO:6)
MHGDTPTLHE YMLDLQPETT DLYCYEQLND SSEEEDEIDG PAGQAEPDRA HYNIVTFCCK
CDSTLRLCVQ STHVDIRTLE DLLMGTLGIV CPICSQKP 97

The HPV E6 protein amino acid sequence GENEBANK Accession Number NC_{—}001526

[SEQ ID NO:7]
MHQKRTAMFQ DPQERPRKLP QLCTELQTTI HDIILECVYC KQQLLRREVY DFAFRDLCIV
YRDGNPYAVC DKCLKFYSKI SEYRHYCYSL YGTTLEQQYN KPLCDLLIRC INCQKPLCPE
EKQRHLDKKQ RFHNIRGRWT GRCMSCCRSS RTRRETQL 168

Any nucleotide sequence encoding this protein can be used in the present vectors.

Two additional antigens used in the studies described herein, OVA and HA have the following coding sequences:

1. Influenza hemagglutinin (HA) [SEQ ID NO:8]

atgaaggcaaacctactggtcctgttaagtgcacttgcagctgcagatgcagacacaatatgtataggctaccatgcgaa
caattcaaccgacactgttgacacagtactcgagaagaatgtgacagtgacacactctgttaacctgctcgaagacagcc
acaacggaaaactatgtagattaaaaggaatagccccactacaattggggaaatgtaacatcgccggatggctcttggga
aacccagaatgcgacccactgcttccagtgagatcatggtcctacattgtagaaacaccaaactctgagaatggaatatg
ttatccaggagatttcatcgactatgaggagctgagggagcaattgagctcagtgtcatcattcgaaagattcgaaatat
ttcccaaagaaagctcatggcccaaccacaacacaaacggagtaacggcagcatgctcccatgaggggaaaagcagtttt
tacagaaatttgctatggctgacggagaaggagggctcatacccaaagctgaaaaattcttatgtgaacaaaaaagggaa
agaagtccttgtactgtggggtattcatcacccgcctaacagtaaggaacaacagaatatctatcagaatgaaaatgctt
atgtctctgtagtgacttcaaattataacaggagatttaccccggaaatagcagaaagacccaaagtaagagatcaagct
gggaggatgaactattactggaccttgctaaaacccggagacacaataatatttgaggcaaatggaaatctaatagcacc
aatgtatgctttcgcactgagtagaggctttgggtccggcatcatcacctcaaacgcatcaatgcatgagtgtaacacga
agtgtcaaacacccctgggagctataaacagcagtctcccttaccagaatatacacccagtcacaataggagagtgccca
aaatacgtcaggagtgccaaattgaggatggttacaggactaaggaacactccgtccattcaatccagaggtctatttgg
agccattgccggttttattgaagggggatggactggaatgatagatggatggtatggttatcatcatcagaatgaacagg
gatcaggctatgcagcggatcaaaaaagcacacaaaatgccattaacgggattacaaacaaggtgaacactgttatcgag
aaaatgaacattcaattcacagctgtgggtaaagaattcaacaaattagaaaaaaggatggaaaatttaaataaaaaagt
tgatgatggatttctggacatttggacatataatgcagaattgttagttctactggaaaatgaaaggactctggatttcc
atgactcaaatgtgaagaatctgtatgagaaagtaaaaagccaattaaagaataatgccaaagaaatcggaaatggatgt
tttgagttctaccacaagtgtgacaatgaatgcatggaaagtgtaagaaatgggacttatgattatcccaaatattcaga
agagtcaaagttgaacagggaaaaggtagatggagtgaaattggaatcaatggggatctatcagattctggcgatctact
caactgtcgccagttcactggtgcttttggtctccctgggggcaatcagtttctggatgtgttctaatggatctttgcag
tgcagaatatgcatctga

The amino acid sequence of HA [SEQ ID NO:9] is

MKANLLVLLS ALAAADADTI CIGYHANNST DTVDTVLEKN VTVTHSVNLL EDSHNGKLCR
LKGIAPLQLG KCNIAGWLLG NPECDPLLPV RSWSYIVETP NSENGICYPG DFIDYEELRE
QLSSVSSFER FEIFPKESSW PNHNTNGVTA ACSHEGKSSF YRNLLWLTEK EGSYPKLKNS
YVNKKGKEVL VLWGIHHPPN SKEQQNIYQN ENAYVSVVTS NYNRRFTPEI AERPKVRDQA
GRMNYYWTLL KPGDTIIFEA NGNLIAPMYA FALSRGFGSG IITSNASMHE CNTKCQTPLG
AINSSLPYQN IHPVTIGECP KYVRSAKLRM VTGLRNTPSI QSRGLFGAIA GFIEGGWTGM
IDGWYGYHHQ NEQGSGYAAD QKSTQNAING ITNKVNTVIE KMNIQFTAVG KEFNKLEKRM
ENLNKKVDDG FLDIWTYNAE LLVLLENERT LDFHDSNVKN LYEKVKSQLK NNAKEIGNGC
FEFYHKCDNE CMESVRNGTY DYPKYSEESK LNREKVDGVK LESMGIYQIL AIYSTVASSL
VLLVSLGAIS FWMCSNGSLQ CRICI

2. Ovalbumin (OVA) [SEQ ID NO:10]

atgggctccatcggcgcagcaagcatggaattttgttttgatgtattcaaggagctcaaagtccaccatgcca
atgagaacatcttctactgccccattgccatcatgtcagctctagccatggtatacctgggtgcaaaagacag
caccaggacacagataaataaggttgttcgctttgataaacttccaggattcggagacagtattgaagctcag
tgtggcacatctgtaaacgttcactcttcacttagagacatcctcaaccaaatcaccaaaccaaatgatgttt
attcgttcagccttgccagtagactttatgctgaagagagatacccaatcctgccagaatacttgcagtgtgt
gaaggaactgtatagaggaggcttggaacctatcaactttcaaacagctgcagatcaagccagagagctcatc
aattcctgggtagaaagtcagacaaatggaattatcagaaatgtccttcagccaagctccgtggattctcaaa
ctgcaatggttctggttaatgccattgtcttcaaaggactgtgggagaaaacatttaaggatgaagacacaca
agcaatgcctttcagagtgactgagcaagaaagcaaacctgtgcagatgatgtaccagattggtttatttaga
gtggcatcaatggcttctgagaaaatgaagatcctggagcttccatttgccagtgggacaatgagcatgttgg
tgctgttgcctgatgaagtctcaggccttgagcagcttgagagtataatcaactttgaaaaactgactgaatg
gaccagttctaatgttatggaagagaggaagatcaaagtgtacttacctcgcatgaagatggaggaaaaatac
aacctcacatctgtcttaatggctatgggcattactgacgtgtttagctcttcagccaatctgtctggcatct
cctcagcagagagcctgaagatatctcaagctgtccatgcagcacatgcagaaatcaatgaagcaggcagaga
ggtggtagggtcagcagaggctggagtggatgctgcaagcgtctctgaagaattt

The amino acid sequence of OVA [SEQ ID NO:11] is:

MGSIGAASME FCFDVFKELK VHHANENIFY CPIAIMSALA MVYLGAKDST RTQINKVVRF
DKLPGFGDSI EAQCGTSVNV HSSLRDILNQ ITKPNDVYSF SLASRLYAEE RYPILPEYLQ
CVKELYRGGL EPINFQTAAD QARELINSWV ESQTNGIIRN VLQPSSVDSQ TAMVLVNAIV
FKGLWEKTFK DEDTQAMPFR VTEQESKPVQ MMYQIGLFRV ASMASEKMKI LELPFASGTM
SMLVLLPDEV SGLEQLESII NFEKLTEWTS SNVMEERKIK VYLPRMKMEE KYNLTSVLMA
MGITDVFSSS ANLSGISSAE SLKISQAVHA AHAEINEAGR EVVGSAEAGV DAASVSEEF

The vectors that include these inserts are:

(a) pcDNA3-HA [SEQ ID NO:12] in which the HA sequence is lower case, underscored:

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCC
ACCACACTGGACTAGTGGATCCatgaaggcaaacctactggtcctgttaagtgcacttgcagctgcagatgcagacacaa
tatgtataggctaccatgcgaacaattcaaccgacactgttgacacagtactcgagaagaatgtgacagtgacacactct
gttaacctgctcgaagacagccacaacggaaaactatgtagattaaaaggaatagccccactacaattggggaaatgtaa
catcgccggatggctcttgggaaacccagaatgcgacccactgcttccagtgagatcatggtcctacattgtagaaacac
caaactctgagaatggaatatgttatccaggagatttcatcgactatgaggagctgagggagcaattgagctcagtgtca
tcattcgaaagattcgaaatatttcccaaagaaagctcatggcccaaccacaacacaaacggagtaacggcagcatgctc
ccatgaggggaaaagcagtttttacagaaatttgctatggctgacggagaaggagggctcatacccaaagctgaaaaatt
cttatgtgaacaaaaaagggaaagaagtccttgtactgtggggtattcatcacccgcctaacagtaaggaacaacagaat
atctatcagaatgaaaatgcttatgtctctgtagtgacttcaaattataacaggagatttaccccggaaatagcagaaag
acccaaagtaagagatcaagctgggaggatgaactattactggaccttgctaaaacccggagacacaataatatttgagg
caaatggaaatctaatagcaccaatgtatgctttcgcactgagtagaggctttgggtccggcatcatcacctcaaacgca
tcaatgcatgagtgtaacacgaagtgtcaaacacccctgggagctataaacagcagtctcccttaccagaatatacaccc
agtcacaataggagagtgcccaaaatacgtcaggagtgccaaattgaggatggttacaggactaaggaacactccgtcca
ttcaatccagaggtctatttggagccattgccggttttattgaagggggatggactggaatgatagatggatggtatggt
tatcatcatcagaatgaacagggatcaggctatgcagcggatcaaaaaagcacacaaaatgccattaacgggattacaaa
caaggtgaacactgttatcgagaaaatgaacattcaattcacagctgtgggtaaagaattcaacaaattagaaaaaagga
tggaaaatttaaataaaaaagttgatgatggatttctggacatttggacatataatgcagaattgttagttctactggaa
aatgaaaggactctggatttccatgactcaaatgtgaagaatctgtatgagaaagtaaaaagccaattaaagaataatgc
caaagaaatcggaaatggatgttttgagttctaccacaagtgtgacaatgaatgcatggaaagtgtaagaaatgggactt
atgattatcccaaatattcagaagagtcaaagttgaacagggaaaaggtagatggagtgaaattggaatcaatggggatc
tatcagattctggcgatctactcaactgtcgccagttcactggtgcttttggtctccctgggggcaatcagtttctggat
gtgttctaatggatctttgcagtgcagaatatgcatctgaAAGCTTAAGTTTAAACCGCTGATCAGCCTCGACTGTGCCT
TCTAGTTGCCAGCCATCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATGGCTTCTGAGGCGGAAAGAACCAGC
TGGGGCTCTAGGGGGTATCCCCACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCCGGCTTTCCCC
GTCAAGCTCTAAATCGGGGCATCCCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACGTTGGAGTCCACGTTCTTTAATAG
TGGACTCTTGTTCCAAACTGGAACAACACTCAACCCTATCTCGGTCTATTCTTTTGATTTATAAGGGATTTTGGGGATTT
CGGCCTATTGGTTAAAAAATGAGCTGATTTAACAAAAATTTAACGCGAATTAATTCTGTGGAATGTGTGTCAGTTAGGGT
GTGGAAAGTCCCCAGGCTCCCCAGGCAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCAGCAACCATAGTCCCGCCCCTAACTC
CGCCCATCCCGCCCCTAACTCCGCCCAGTTCCGCCCATTCTCCGCCCCATGGCTGACTAATTTTTTTTATTTATGCAGAG
GCCGAGGCCGCCTCTGCCTCTGAGCTATTCCAGAAGTAGTGAGGAGGCTTTTTTGGAGGCCTAGGCTTTTGCAAAAAGCT
CCCGGGAGCTTGTATATCCATTTTCGGATCTGATCAAGAGACAGGATGAGGATCGTTTCGCATGATTGAACAAGATGGAT
TGCACGCAGGTTCTCCGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGTGCCCTGAATGAACT
GCAGGACGAGGCAGCGCGGCTATCGTGGCTGGCCACGACGGGCGTTCCTTGCGCAGCTGTGCTCGACGTTGTCACTGAAG
CGGGAAGGGACTGGCTGCTATTGGGCGAAGTGCCGGGGCAGGATCTCCTGTCATCTCACCTTGCTCCTGCCGAGAAAGTA
TCCATCATGGCTGATGCAATGCGGCGGCTGCATACGCTTGATCCGGCTACCTGCCCATTCGACCACCAAGCGAAACATCG
CATCGAGCGAGCACGTACTCGGATGGAAGCCGGTCTTGTCGATCAGGATGATCTGGACGAAGAGCATCAGGGGCTCGCGC
CAGCCGAACTGTTCGCCAGGCTCAAGGCGCGCATGCCCGACGGCGAGGATCTCGTCGTGACCCATGGCGATGCCTGCTTG
CCGAATATCATGGTGGAAAATGGCCGCTTTTCTGGATTCATCGACTGTGGCCGGCTGGGTGTGGCGGACCGCTATCAGGA
CATAGCGTTGGCTACCCGTGATATTGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGAGCGGGACTCTGGGGTTCGAAATGA
CCGACCAAGCGACGCCCAACCTGCCATCACGAGATTTCGATTCCACCGCCGCCTTCTATGAAAGGTTGGGCTTCGGAATC
GTTTTCCGGGACGCCGGCTGGATGATCCTCCAGCGCGGGGATCTCATGCTGGAGTTCTTCGCCCACCCCAACTTGTTTAT
TGCAGCTTATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGTATACCGTCGACCTCTAGCTAGAGCTTGGCGTAATCATGG
TCATAGCTGTTTCCTGTGTGAAATTGTTATCCGCTCACAATTCCACACAACATACGAGCCGGAAGCATAAAGTGTAAAGC
CTGGGGTGCCTAATGAGTGAGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCTCTTCCGCTTCCTCGCTCACT
GACTCGCTGCGCTCGGTCGTTCGGCTGCGGCGAGCGGTATCAGCTCACTCAAAGGCGGTAATACGGTTATCCACAGAATC

(b) pcDNA3-OVA [SEQ ID NO:13] in which the OVA sequence is lower case, underscored:

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCa
tgggctccatcggcgcagcaagcatggaattttgttttgatgtattcaaggagctcaaagtccaccatgccaatgagaac
atcttctactgccccattgccatcatgtcagctctagccatggtatacctgggtgcaaaagacagcaccaggacacagat
aaataaggttgttcgctttgataaacttccaggattcggagacagtattgaagctcagtgtggcacatctgtaaacgttc
actcttcacttagagacatcctcaaccaaatcaccaaaccaaatgatgtttattcgttcagccttgccagtagactttat
gctgaagagagatacccaatcctgccagaatacttgcagtgtgtgaaggaactgtatagaggaggcttggaacctatcaa
ctttcaaacagctgcagatcaagccagagagctcatcaattcctgggtagaaagtcagacaaatggaattatcagaaatg
tccttcagccaagctccgtggattctcaaactgcaatggttctggttaatgccattgtcttcaaaggactgtgggagaaa
acatttaaggatgaagacacacaagcaatgcctttcagagtgactgagcaagaaagcaaacctgtgcagatgatgtacca
gattggtttatttagagtggcatcaatggcttctgagaaaatgaagatcctggagcttccatttgccagtgggacaatga
gcatgttggtgctgttgcctgatgaagtctcaggccttgagcagcttgagagtataatcaactttgaaaaactgactgaa
tggaccagttctaatgttatggaagagaggaagatcaaagtgtacttacctcgcatgaagatggaggaaaaatacaacct
cacatctgtcttaatggctatgggcattactgacgtgtttagctcttcagccaatctgtctggcatctcctcagcagaga
gcctgaagatatctcaagctgtccatgcagcacatgcagaaatcaatgaagcaggcagagaggtggtagggtcagcagag
gctggagtggatgctgcaagcgtctctgaagaatttGGATCCGAGCTCGGTACCAAGCTTAAGTTTAAACCGCTGATCAG
CCTCGACTGTGCCTTCTAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGGGATGCGGTGGGCTCTATGGCTTCTGAGG
CGGAAAGAACCAGCTGGGGCTCTAGGGGGTATCCCCACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTCTTCCCTTCCTTTCTCGCCACGTT
CGCCGGCTTTCCCCGTCAAGCTCTAAATCGGGGCATCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCGGTCTATTCTTTTGATTTATAAGG
GATTTTGGGGATTTCGGCCTATTGGTTAAAAAATGAGCTGATTTAACAAAAATTTAACGCGAATTAATTCTGTGGAATGT
GTGTCAGTTAGGGTGTGGAAAGTCCCCAGGCTCCCCAGGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGCATCTCAATTAGTCAGCAACCATAGT
CCCGCCCCTAACTCCGCCCATCCCGCCCCTAACTCCGCCCAGTTCCGCCCATTCTCCGCCCCATGGCTGACTAATTTTTT
TTATTTATGCAGAGGCCGAGGCCGCCTCTGCCTCTGAGCTATTCCAGAAGTAGTGAGGAGGCTTTTTTGGAGGCCTAGGC
TTTTGCAAAAAGCTCCCGGGAGCTTGTATATCCATTTTCGGATCTGATCAAGAGACAGGATGAGGATCGTTTCGCATGAT
TGAACAAGATGGATTGCACGCAGGTTCTCCGGCCGCTTGGGTGGAGAGGCTATTCGGCTATGACTGGGCACAACAGACAA
TCGGCTGCTCTGATGCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATCCGGCTACCTGCCCATTCGACCAC
CAAGCGAAACATCGCATCGAGCGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGCGCATGCCCGACGGCGAGGATCTCGTCGTGACCCATG
GCGATGCCTGCTTGCCGAATATCATGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAGCTTGGCGGCGAATGGGCTGACCGCTTCCTCGT
GCTTTACGGTATCGCCGCTCCCGATTCGCAGCGCATCGCCTTCTATCGCCTTCTTGACGAGTTCTTCTGAGCGGGACTCT
GGGGTTCGAAATGACCGACCAAGCGACGCCCAACCTGCCATCACGAGATTTCGATTCCACCGCCGCCTTCTATGAAAGGT
TGGGCTTCGGAATCGTTTTCCGGGACGCCGGCTGGATGATCCTCCAGCGCGGGGATCTCATGCTGGAGTTCTTCGCCCAC
CCCAACTTGTTTATTGCAGCTTATAATGGTTACAAATAAAGCAATAGCATCACAAATTTCACAAATAAAGCATTTTTTTC
ACTGCATTCTAGTTGTGGTTTGTCCAAACTCATCAATGTATCTTATCATGTCTGTATACCGTCGACCTCTAGCTAGAGCT
TGGCGTAATCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTTTCCAGTC
GGGAAACCTGTCGTGCCAGCTGCATTAATGAATCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAGCGGTATCAGCTCACTCAAAGGCGGTAATACGG
TTATCCACAGAATCAGGGGATAACGCAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCAATGCTCACGCTGTAGGTATCTCAGTTCGGT
GTAGGTCGTTCGCTCCAAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGACAGTATTTGGTATCTGCGCTCT
GCTGAAGCCAGTTACCTTCGGAAAAAGAGTTGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTCACGCTCGTCGTTT
GGTATGGCTTCATTCAGCTCCGGTTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACTTTAAAAGTGCTCATCAT
TGGAAAACGTTCTTCGGGGCGAAAACTCTCAAGGATCTTACCGCTGTTGAGATCCAGTTCGATGTAACCCACTCGTGCAC
CCAACTGATCTTCAGCATCTTTTACTTTCACCAGCGTTTCTGGGTGAGCAAAAACAGGAAGGCAAAATGCCGCAAAAAAG
GGAATAAGGGCGACACGGAAATGTTGAATACTCATACTCTTCCTTTTTCAATATTATTGAAGCATTTATCAGGGTTATTG
TCTCATGAGCGGATACATATTTGAATGTATTTAGAAAAATAAACAAATAGGGGTTCCGCGCACATTTCCCCGAAAAGTGC
CACCTGACGTC

Sequences of Anti-Apoptotic DNA and Vectors

The coding sequence for BCL-xL [SEQ ID NO:14] as present in the pcDNA3 vector of the present invention is:

atggcgtacccatacgatgttccagattacgctagcttgagatctaccat
gtctcagagcaaccgggagctggtggttgactttctctcctacaagcttt
cccagaaaggatacagctggagtcagtttagtgatgtggaagagaacagg
actgaggccccagaagggactgaatcggagatggagacccccagtgccat
caatggcaacccatcctggcacctggcagacagccccgcggtgaatggag
ccactgcgcacagcagcagtttggatgcccgggaggtgatccccatggca
gcagtaaagcaagcgctgagggaggcaggcgacgagtttgaactgcggta
ccggcgggcattcagtgacctgacatcccagctccacatcaccccaggga
cagcatatcagagctttgaacaggtagtgaatgaactcttccgggatggg
gtaaactggggtcgcattgtggcctttttctccttcggcggggcactgtg
cgtggaaagcgtagacaaggagatgcaggtattggtgagtcggatcgcag
cttggatggccacttacctgaatgaccacctagagccttggatccaggag
aacggcggctgggatacttttgtggaactctatgggaacaatgcagcagc
cgagagccgaaagggccaggaacgcttcaaccgctggttcctgacgggca
tgactgtggccggcgtggttctgctgggctcactcttcagtcggaaatga

The amino acid sequence of BCL-xL is [SEQ ID NO:15]:

MAYPYDVPDY ASLRSTMSQS NRELVVDFLS YKLSQKGYSW SQFSDVEENR TEAPEGTESE
METPSAINGN PSWHLADSPA VNGATAHSSS LDAREVIPMA AVKQALREAG DEFELRYRRA
FSDLTSQLHI TPGTAYQSFE QVVNELFRDG VNWGRIVAFF SFGGALCVES VDKEMQVLVS
RIAAWMATYL NDHLEPWIQE NGGWDTFVEL YGNNAAAESR KGQERFNRWF LTGMTVAGVV
LLGSLFSRK

The sequence pcDNA3-BCL-xL [SEQ ID NO:16] is shown below (the BCL-xL coding sequence is lower case and underscored

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCC
ACCACACTGGACTAGTGGATCTatggcgtacccatacgatgttccagattacgctagcttgagatctaccatgtctcaga
gcaaccgggagctggtggttgactttctctcctacaagctttcccagaaaggatacagctggagtcagtttagtgatgtg
gaagagaacaggactgaggccccagaagggactgaatcggagatggagacccccagtgccatcaatggcaacccatcctg
gcacctggcagacagccccgcggtgaatggagccactgcgcacagcagcagtttggatgcccgggaggtgatccccatgg
cagcagtaaagcaagcgctgagggaggcaggcgacgagtttgaactgcggtaccggcgggcattcagtgacctgacatcc
cagctccacatcaccccagggacagcatatcagagctttgaacaggtagtgaatgaactcttccgggatggggtaaactg
gggtcgcattgtggcctttttctccttcggcggggcactgtgcgtggaaagcgtagacaaggagatgcaggtattggtga
gtcggatcgcagcttggatggccacttacctgaatgaccacctagagccttggatccaggagaacggcggctgggatact
tttgtggaactctatgggaacaatgcagcagccgagagccgaaagggccaggaacgcttcaaccgctggttcctgacggg
catgactgtggccggcgtggttctgctgggctcactcttcagtcggaaatgaAGATCCGAGCTCGGTACCAAGCTTAAGT
TTAAACCGCTGATCAGCCTCGACTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAAATGCATCGCATTGTCTGAGTAGGTGTCATTCTA
TTCTGGGGGGTGGGGTGGGGCAGGACAGCAAGGGGGAGGATTGGGAAGACAATAGCAGGCATGCTGGGGATGCGGTGGGC
TCTATGGCTTCTGAGGCGGAAAGAACCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGCATCCCTTTAGGGTTCCGATTTAGTGCTTTA
CGGCACCTCGACCCCAAAAAACTTGATTAGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACAACACTCAACCCTATCTCGGTCTATT
CTTTTGATTTATAAGGGATTTTGGGGATTTCGGCCTATTGGTTAAAAAATGAGCTGATTTAACAAAAATTTAACGCGAAT
TAATTCTGTGGAATGTGTGTCAGTTAGGGTGTGGAAAGTCCCCAGGCTCCCCAGGCAGGCAGAAGTATGCAAAGCATGCA
TCTCAATTAGTCAGCAACCAGGTGTGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCAGTTCCGCCCATTCTCCGCCCCAT
GGCTGACTAATTTTTTTTATTTATGCAGAGGCCGAGGCCGCCTCTGCCTCTGAGCTATTCCAGAAGTAGTGAGGAGGCTT
TTTTGGAGGCCTAGGCTTTTGCAAAAAGCTCCCGGGAGCTTGTATATCCATTTTCGGATCTGATCAAGAGACAGGATGAG
GATCGTTTCGCATGATTGAACAAGATGGATTGCACGCAGGTTCTCCGGCCGCTTGGGTGGAGAGGCTATTCGGCTATGAC
TGGGCACAACAGACAATCGGCTGCTCTGATGCCGCCGTGTTCCGGCTGTCAGCGCAGGGGCGCCCGGTTCTTTTTGTCAA
GACCGACCTGTCCGGTGCCCTGAATGAACTGCAGGACGAGGCAGCGCGGCTATCGTGGCTGGCCACGACGGGCGTTCCTT
GCGCAGCTGTGCTCGACGTTGTCACTGAAGCGGGAAGGGACTGGCTGCTATTGGGCGAAGTGCCGGGGCAGGATCTCCTG
TCATCTCACCTTGCTCCTGCCGAGAAAGTATCCATCATGGCTGATGCAATGCGGCGGCTGCATACGCTTGATCCGGCTAC
CTGCCCATTCGACCACCAAGCGAAACATCGCATCGAGCGAGCACGTACTCGGATGGAAGCCGGTCTTGTCGATCAGGATG
ATCTGGACGAAGAGCATCAGGGGCTCGCGCCAGCCGAACTGTTCGCCAGGCTCAAGGCGCGCATGCCCGACGGCGAGGAT
CTCGTCGTGACCCATGGCGATGCCTGCTTGCCGAATATCATGGTGGAAAATGGCCGCTTTTCTGGATTCATCGACTGTGG
CCGGCTGGGTGTGGCGGACCGCTATCAGGACATAGCGTTGGCTACCCGTGATATTGCTGAAGAGCTTGGCGGCGAATGGG
CTGACCGCTTCCTCGTGCTTTACGGTATCGCCGCTCCCGATTCGCAGCGCATCGCCTTCTATCGCCTTCTTGACGAGTTC
TTCTGAGCGGGACTCTGGGGTTCGAAATGACCGACCAAGCGACGCCCAACCTGCCATCACGAGATTTCGATTCCACCGCC
GCCTTCTATGAAAGGTTGGGCTTCGGAATCGTTTTCCGGGACGCCGGCTGGATGATCCTCCAGCGCGGGGATCTCATGCT
GGAGTTCTTCGCCCACCCCAACTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTATCATGTCTGTATACCGTCG
ACCTCTAGCTAGAGCTTGGCGTAATCATGGTCATAGCTGTTTCCTGTGTGAAATTGTTATCCGCTCACAATTCCACACAA
CATACGAGCCGGAAGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTCTCAATGCTCACGCTGTA
GGTATCTCAGTTCGGTGTAGGTCGTTCGCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTACACTAGAAGGACAGTAT
TTGGTATCTGCGCTCTGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGATCCTTTGATCTTTTC
TACGGGGTCTGACGCTCAGTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGGTCTGACAGTTACCAATGC
TTAATCAGTGAGGCACCTATCTCAGCGATCTGTCTATTTCGTTCATCCATAGTTGCCTGACTCCCCGTCGTGTAGATAAC
TACGATACGGGAGGGCTTACCATCTGGCCCCAGTGCTGCAATGATACCGCGAGACCCACGCTCACCGGCTCCAGATTTAT
CAGCAATAAACCAGCCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGAGTTACATGATCCCCCATGTTGT
GCAAAAAAGCGGTTAGCTCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAGTACTCAACCAAGTCATT
CTGAGAATAGTGTATGCGGCGACCGAGTTGCTCTTGCCCGGCGTCAATACGGGATAATACCGCGCCACATAGCAGAACTT
TAAAAGTGCTCATCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAAACAGGAAGGCA
AAATGCCGCAAAAAAGGGAATAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAACAAATAGGGGTTCCGCGCACA
TTTCCCCGAAAAGTGCCACCTGACGTC

A pcDNA3 vector combining E7 and BCL-xL, designated pcDNA3-E7/BCL-xL (SEQ ID NO:17) is shown below with E7 sequence is lower case, not underscored; and BCL-xL is lower case and underscored

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCa
tgcatggagatacacctacattgcatgaatatatgttagatttgcaaccagagacaactgatctctactgttatgagcaa
ttaaatgacagctcagaggaggaggatgaaatagatggtccagctggacaagcagaaccggacagagcccattacaatat
tgtaaccttttgttgcaagtgtgactctacgcttcggttgtgcgtacaaagcacacacgtagacattcgtactttggaag
acctgttaatgggcacactaggaattgtgtgccccatctgttctcagaaaccaGGATCTatggcgtacccatacgatgtt
ccagattacgctagcttgagatctaccatgtctcagagcaaccgggagctggtggttgactttctctcctacaagctttc
ccagaaaggatacagctggagtcagtttagtgatgtggaagagaacaggactgaggccccagaagggactgaatcggaga
tggagacccccagtgccatcaatggcaacccatcctggcacctggcagacagccccgcggtgaatggagccactgcgcac
agcagcagtttggatgcccgggaggtgatccccatggcagcagtaaagcaagcgctgagggaggcaggcgacgagtttga
actgcggtaccggcgggcattcagtgacctgacatcccagctccacatcaccccagggacagcatatcagagctttgaac
aggtagtgaatgaactcttccgggatggggtaaactggggtcgcattgtggcctttttctccttcggcggggcactgtgc
gtggaaagcgtagacaaggagatgcaggtattggtgagtcggatcgcagcttggatggccacttacctgaatgaccacct
agagccttggatccaggagaacggcggctgggatacttttgtggaactctatgggaacaatgcagcagccgagagccgaa
agggccaggaacgcttcaaccgctggttcctgacgggcatgactgtggccggcgtggttctactgggctcactcttcagt
cggaaatgaAGATCCAAGCTTAAGTTTAAACCGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGAGGATTGGGAAGACAATAG
CAGGCATGCTGGGGATGCGGTGGGCTCTATGGCTTCTGAGGCGGAAAGAACCAGCTGGGGCTCTAGGGGGTATCCCCACG
CGCCCTGTAGCGGCGCATTAAGCGCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCAAGCTCTAAATCGGGGCATCCC
TTTAGGGTTCCGATTTAGTGCTTTACGGCACCTCGACCCCAAAAAACTTGATTAGGGTGATGGTTCACGTAGTGGGCCAT
CGCCCTGATAGACGGTTTTTCGCCCTTTGACGTTGGAGTCCACGTTCTTTAATAGTGGACTCTTGTTCCAAACTGGAACA
ACACTCAACCCTATCTCGGTCTATTCTTTTGATTTATAAGGGATTTTGGGGATTTCGGCCTATTGGTTAAAAAATGAGCT
GATTTAACAAAAATTTAACGCGAATTAATTCTGTGGAATGTGTGTCAGTTAGGGTGTGGAAAGTCCCCAGGCTCCCCAGG
CAGGCAGAAGTATGCAAAGCATGCATCTCAATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCTGCCTCTGAGC
TATTCCAGAAGTAGTGAGGAGGCTTTTTTGGAGGCCTAGGCTTTTGCAAAAAGCTCCCGGGAGCTTGTATATCCATTTTC
GGATCTGATCAAGAGACAGGATGAGGATCGTTTCGCATGATTGAACAAGATGGATTGCACGCAGGTTCTCCGGCCGCTTG
GGTGGAGAGGCTATTCGGCTATGACTGGGCACAACAGACAATCGGCTGCTCTGATGCCGCCGTGTTCCGGCTGTCAGCGC
AGGGGCGCCCGGTTCTTTTTGTCAAGACCGACCTGTCCGGTGCCCTGAATGAACTGCAGGACGAGGCAGCGCGGCTATCG
TGGCTGGCCACGACGGGCGTTCCTTGCGCAGCTGTGCTCGACGTTGTCACTGAAGCGGGAAGGGACTGGCTGCTATTGGG
CGAAGTGCCGGGGCAGGATCTCCTGTCATCTCACCTTGCTCCTGCCGAGAAAGTATCCATCATGGCTGATGCAATGCGGC
GGCTGCATACGCTTGATCCGGCTACCTGCCCATTCGACCACCAAGCGAAACATCGCATCGAGCGAGCACGTACTCGGATG
GAAGCCGGTCTTGTCGATCAGGATGATCTGGACGAAGAGCATCAGGGGCTCGCGCCAGCCGAACTGTTCGCCAGGCTCAA
GGCGCGCATGCCCGACGGCGAGGATCTCGTCGTGACCCATGGCGATGCCTGCTTGCCGAATATCATGGTGGAAAATGGCC
GCTTTTCTGGATTCATCGACTGTGGCCGGCTGGGTGTGGCGGACCGCTATCAGGACATAGCGTTGGCTACCCGTGATATT
GCTGAAGAGCTTGGCGGCGAATGGGCTGACCGCTTCCTCGTGCTTTACGGTATCGCCGCTCCCGATTCGCAGCGCATCGC
CTTCTATCGCCTTCTTGACGAGTTCTTCTGAGCGGGACTCTGGGGTTCGAAATGACCGACCAAGCGACGCCCAACCTGCC
ATCACGAGATTTCGATTCCACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCTTATAATGGTTACAAATAA
AGCAATAGCATCACAAATTTCACAAATAAAGCATTTTTTTCACTGCATTCTAGTTGTGGTTTGTCCAAACTCATCAATGT
ATCTTATCATGTCTGTATACCGTCGACCTCTAGCTAGAGCTTGGCGTAATCATGGTCATAGCTGTTTCCTGTGTGAAATT
GTTATCCGCTCACAATTCCACACAACATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGCTCGGTCGTTCGGC
TGCGGCGAGCGGTATCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCTCCCTTCGGGAAGCGTG
GCGCTTTCTCAATGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTACAGAGTTCTTGAAGTGGTGGCCTAAC
TACGGCTACACTAGAAGGACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGTATATATGAGTA
AACTTGGTCTGACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCGATCGTTGTCAGAAGTAAGTTGGC
CGCAGTGTTATCACTCATGGTTATGGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTTGAATACTCATACTC
TTCCTTTTTCAATATTATTGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTC

The amino acid sequence of the E7-BCL-xL chimeric or fusion polypeptide [SEQ ID NO:8] is:

MHGDTPTLHE YMLDLQPETT DLYCYEQLND SSEEEDEIDG PAGQAEPDRA HYNIVTFCCK
CDSTLRLCVQ STHVDIRTLE DLLMGTLGIV CPICSQKPGS MAYPYDVPDY ASLRSTMSQS
NRELVVDFLS YKLSQKGYSW SQFSDVEENR TEAPEGTESE METPSAINGN PSWHLADSPA
VNGATAHSSS LDAREVIPMA AVKQALREAG DEFELRYRRA FSDLTSQLHI TPGTAYQSFE
QVVNELFRDG VNWGRIVAFF SFGGALCVES VDKEMQVLVS RIAAWMATYL NDHLEPWIQE
NGGWDTFVEL YGNNAAAESR KGQERFNRWF LTGMTVAGVV LLGSLFSRK

The mutant BCL-xL (“mtBCL-xL”) DNA sequence is shown below [SEQ ID NO:19]

atggcgtacccatacgatgttccagattacgctagcttgagatctaccat
gtctcagagcaaccgggagctggtggttgactttctctcctacaagcttt
cccagaaaggatacagctggagtcagtttagtgatgtggaagagaacagg
actgaggccccagaagggactgaatcggagatggagacccccagtgccat
caatggcaacccatcctggcacctggcagacagccccgcggtgaatggag
ccactgcgcacagcagcagtttggatgcccgggaggtgatccccatggca
gcagtaaagcaagcgctgagggaggcaggcgacgagtttgaactgcggta
ccggcgggcattcagtgacctgacatcccagctccacatcaccccaggga
cagcatatcagagctttgaacaggtagtgaatgaactcttccgggatggg
gtagccattcttcgcattgtggcctttttctccttcggcggggcactgtg
cgtggaaagcgtagacaaggagatgcaggtattggtgagtcggatcgcag
cttggatggccacttacctgaatgaccacctagagccttggatccaggag
aacggcggctgggatacttttgtggaactctatgggaacaatgcagcagc
cgagagccgaaagggccaggaacgcttcaaccgctggttcctgacgggca
tgactgtggccggcgtggttctgctgggctcactcttcagtcggaaatga

The amino acid sequence of MtBCL-xL [SEQ ID NO:20] is:

MAYPYDVPDY ASLRSTMSQS NRELVVDFLS YKLSQKGYSW SQFSDVEENR TEAPEGTESE
METPSAINGN PSWHLADSPA VNGATAHSSS LDAREVIPMA AVKQALREAG DEFELRYRRA
FSDLTSQLHI TPGTAYQSFE QVVNELFRDG VAILRIVAFF SFGGALCVES VDKEMQVLVS
RIAAWMATYL NDHLEPWIQE NGGWDTFVEL YGNNAAAESR KGQERFNRWF LTGMTVAGVV
LLGSLFSRK

The amino acid sequence of the E7-mtBCL-xL chimeric or fusion polypeptide [SEQ ID NO:21] is:

MHGDTPTLHE YMLDLQPETT DLYCYEQLND SSEEEDEIDG PAGQAEPDRA HYNIVTFCCK
CDSTLRLCVQ STHVDIRTLE DLLMGTLGIV CPICSQKPGS MAYPYDVPDY ASLRSTMSQS
NRELVVDFLS YKLSQKGYSW SQFSDVEENR TEAPEGTESE METPSAINGN PSWHLADSPA
VNGATAHSSS LDAREVIPMA AVKQALREAG DEFELRYRRA FSDLTSQLHI TPGTAYQSFE
QVVNELFRDG VAILRIVAFF SFGGALCVES VDKEMQVLVS RIAAWMATYL NDHLEPWIQE
NGGWDTFVEL YGNNAAAESR KGQERFNRWF LTGMTVAGVV LLGSLFSRK

In the pcDNA-mtBCL-xL [SEQ ID NO:22] vector, this mutant sequence is inserted in the same position that BCL-xL is inserted in SEQ ID NO:16 and in the pcDNA-E7/mtBCL-XL [SEQ ID NO:23], this sequence is inserted in the same position as the BCL-xL sequence is in SEQ ID NO:17, above.

The sequence of the suicidal DNA vector pSCA1-BCL-[SEQ ID NO:24] is shown below, with the BCL-xL in lower case and underscored:

ATGGCGGATGTGTGACATACACGACGCCAAAAGATTTTGTTCCAGCTCCTGCCACCTCCGCTACGCGAGAGATTAACCAC
CCACGATGGCCGCCAAAGTGCATGTTGATATTGAGGCTGACAGCCCATTCATCAAGTCTTTGCAGAAGGCATTTCCGTCG
TTCGAGGTGGAGTCATTGCAGGTCACACCAAATGACCATGCAAATGCCAGAGCATTTTCGCACCTGGCTACCAAATTGAT
CGAGCAGGAGACTGACAAAGACACACTCATCTTGGATATCGGCAGTGCGCCTTCCAGGAGAATGATGTCTACGCACAAAT
ACCACTGCGTATGCCCTATGCGCAGCGCAGAAGACCCCGAAAGGCTCGATAGCTACGCAAAGAAACTGGCAGCGGCCTCC
GGGAAGGTGCTGGATAGAGAGATCGCAGGAAAAATCACCGACCTGCAGACCGTCATGGCTACGCCAGACGCTGAATCTCC
TACCTTTTGCCTGCATACAGACGTCACGTGTCGTACGGCAGCCGAAGTGGCCGTATACCAGGACGTGTATGCTGTACATG
CACCAACATCGCTGTACCATCAGGCGATGAAAGGTGTCAGAACGGCGTATTGGATTGGGTTTGACACCACCCCGTTTATG
TTTGACGCGCTAGCAGGCGCGTATCCAACCTACGCCACAAACTGGGCCGACGAGCAGGTGTTACAGGCCAGGAACATAGG
ACTGTGTGCAGCATCCTTGACTGAGGGAAGACTCGGCAAACTGTCCATTCTCCGCAAGAAGCAATTGAAACCTTGCGACA
CAGTCATGTTCTCGGTAGGATCTACATUGTACACTGAGAGCAGAAAGCTACTGAGGAGCTGGCACTTACCCTCCGTATTC
CACCTGAAAGGTAAACAATCCTTTACCTGTAGGTGCGATACCATCGTATCATGTGAAGGGTACGTAGTTAAGAAAATCAC
TATGTGCCCCGGCCTGTACGGTAAAACGGTAGGGTACGCCGTGACGTATCACGCGGAGGGATTCCTAGTGTGCAAGACCA
CAGACACTGTCAAAGGAGAAAGAGTCTCATTCCCTGTATGCACCTACGTCCCCTCAACCATCTGTGATCAAATGACTGGC
ATACTAGCGACCGACGTCACACCGGAGGACGCACAGAAGTTGTTAGTGGGATTGAATCAGAGGATAGTTGTGAACGGAAG
AACACAGCGAAACACTAACACGATGAAGAACTATCTGCTTCCGATTGTGGCCGTCGCATTTAGCAAGTGGGCGAGGGAAT
ACAAGGCAGACCTTGATGATGAAAAACCTCTGGGTGTCCGAGAGAGGTCACTTACTTGCTGCTGCTTGTGGGCATTTAAA
ACGAGGAAGATGCACACCATGTACAAGAAACCAGACACCCAGACAATAGTGAAGGTGCCTTCAGAGTTTAACTCGTTCGT
CATCCCGAGCCTATGGTCTACAGGCCTCGCAATCCCAGTCAGATCACGCATTAAGATGCTTTTGGCCAAGAAGACCAAGC
GAGAGTTAATACCTGTTCTCGACGCGTCGTCAGCCAGGGATGCTGAACAAGAGGAGAAGGAGAGGTTGGAGGCCGAGCTG
ACTAGAGAAGCCTTACCACCCCTCGTCCCCATCGCGCCGGCGGAGACGGGAGTCGTCGACGTCGACGTTGAAGAACTAGA
GTATCACGCAGGTGCAGGGGTCGTGGAAACACCTCGCAGCGCGTTGAAAGTCACCGCACAGCCGAACGACGTACTACTAG
GAAATTACGTAGTTCTGTCCCCGCAGACCGTGCTCAAGAGCTCCAAGTTGGCCCCCGTGCACCCTCTAGCAGAGCAGGTG
AAAATAATAACACATAACGGGAGGGCCGGCGGTTACCAGGTCGACGGATATGACGGCAGGGTCCTACTACCATGTGGATC
GGCCATTCCGGTCCCTGAGTTTCAAGCTTTGAGCGAGAGCGCCACTATGGTGTACAACGAAAGGGAGTTCGTCAACAGGA
AACTATACCATATTGCCGTTCACGGACCGTCGCTGAACACCGACGAGGAGAACTACGAGAAAGTCAGAGCTGAAAGAACT
GACGCCGAGTACGTGTTCGACGTAGATAAAAAATGCTGCGTCAAGAGAGAGGAAGCGTCGGGTTTGGTGTTGGTGGGAGA
GCTAACCAACCCCCCGTTCCATGAATTCGCCTACGAAGGGCTGAAGATCAGGCCGTCGGCACCATATAAGACTACAGTAG
TAGGAGTCTTTGGGGTTCCGGGATCAGGCAAGTCTGCTATTATTAAGAGCCTCGTGACCAAACACGATCTGGTCACCAGC
GGCAAGAAGGAGAACTGCCAGGAAATAGTTAACGACGTGAAGAAGCACCGCGGGAAGGGGACAAGTAGGGAAAACAGTGA
CTCCATCCTGCTAAACGGGTGTCGTCGTGCCGTGGACATCCTATATGTGGACGAGGCTTTCGCTAGCCATTCCGGTACTC
TGCTGGCCCTAATTGCTCTTGTTAAACCTCGGAGCAAAGTGGTGTTATGCGGAGACCCCAAGCAATGCGGATTCTTCAAT
ATGATGCAGCTTAAGGTGAACTTCAACCACAACATCTGCACTGAAGTATGTCATAAAAGTATATCCAGACGTTGCACGCG
TCCAGTCACGGCCATCGTGTCTACGTTGCACTACGGAGGCAAGATGCGCACGACCAACCCGTGCAACAAACCCATAATCA
TAGACACCACAGGACAGACCAAGCCCAAGCCAGGAGACATCGTGTTAACATGCTTCCGAGGCTGGGCAAAGCAGCTGCAG
TTGGACTACCGTGGACACGAAGTCATGACAGCAGCAGCATCTCAGGGCCTCACCCGCAAAGGGGTATACGCCGTAAGGCA
GAAGGTGAATGAAAATCCCTTGTATGCCCCTGCGTCGGAGCACGTGAATGTACTGCTGACGCGCACTGAGGATAGGCTGG
TGTGGAAAACGCTGGCCGGCGATCCCTGGATTAAGGTCCTATCAAACATTCCACAGGGTAACTTTACGGCCACATTGGAA
GAATGGCAAGAAGAACACGACAAAATAATGAAGGTGATTGAAGGACCGGCTGCGCCTGTGGACGCGTTCCAGAACAAAGC
GAACGTGTGTTGGGCGAAAAGCCTGGTGCCTGTCCTGGACACTGCCGGAATCAGATTGACAGCAGAGGAGTGGAGCACCA
TAATTACAGCATTTAAGGAGGACAGAGCTTACTCTCCAGTGGTGGCCTTGAATGAAATTTGCACCAAGTACTATGGAGTT
GACCTGGACAGTGGCCTGTTTTCTGCCCCGAAGGTGTCCCTGTATTACGAGAACAACCACTGGGATAACAGACCTGGTGG
AAGGATGTATGGATTCAATGCCGCAACAGCTGCCAGGCTGGAAGCTAGACATACCTTCCTGAAGGGGCAGTGGCATACGG
GCAAGCAGGCAGTTATCGCAGAAAGAAAAATCCAACCGCTTTCTGTGCTGGACAATGTAATTCCTATCAACCGCAGGCTG
CCGCACGCCCTGGTGGCTGAGTACAAGACGGTTAAAGGCAGTAGGGTTGAGTGGCTGGTCAATAAAGTAAGAGGGTACCA
CGTCCTGCTGGTGAGTGAGTACAACCTGGCTTTGCCTCGACGCAGGGTCACTTGGTTGTCACCGCTGAATGTCACAGGCG
CCGATAGGTGCTACGACCTAAGTTTAGGACTGCCGGCTGACGCCGGCAGGTTCGACTTGGTCTTTGTGAACATTCACACG
GAATTCAGAATCCACCACTACCAGCAGTGTGTCGACCACGCCATGAAGCTGCAGATGCTTGGGGGAGATGCGCTACGACT
GCTAAAACCCGGCGGCATCTTGATGAGAGCTTACGGATACGCCGATAAAATCAGCGAAGCCGTTGTTTCCTCCTTAAGCA
GAAAGTTCTCGTCTGCAAGAGTGTTGCGCCCGGATTGTGTCACCAGCAATACAGAAGTGTTCTTGCTGTTCTCCAACTTT
GACAACGGAAAGAGACCCTCTACGCTACACCAGATGAATACCAAGCTGAGTGCCGTGTATGCCGGAGAAGCCATGCACAC
GGCCGGGTGTGCACCATCCTACAGAGTTAAGAGAGCAGACATAGCCACGTGCACAGAAGCGGCTGTGGTTAACGCAGCTA
ACGCCCGTGGAACTGTAGGGGATGGCGTATGCAGGGCCGTGGCGAAGAAATGGCCGTCAGCCTTTAAGGGAGCAGCAACA
CCAGTGGGCACAATTAAAACAGTCATGTGCGGCTCGTACCCCGTCATCCACGCTGTAGCGCCTAATTTCTCTGCCACGAC
TGAAGCGGAAGGGGACCGCGAATTGGCCGCTGTCTACCGGGCAGTGGCCGCCGAAGTAAACAGACTGTCACTGAGCAGCG
TAGCCATCCCGCTGCTGTCCACAGGAGTGTTCAGCGGCGGAAGAGATAGGCTGCAGCAATCCCTCAACCATCTATTCACA
GCAATGGACGCCACGGACGCTGACGTGACCATCTACTGCAGAGACAAAAGTTGGGAGAAGAAAATCCAGGAAGCCATTGA
CATGAGGACGGCTGTGGAGTTGCTCAATGATGACGTGGAGCTGACCACAGACTTGGTGAGAGTGCACCCGGACAGCAGCC
TGGTGGGTCGTAAGGGCTACAGTACCACTGACGGGTCGCTGTACTCGTACTTTGAAGGTACGAAATTCAACCAGGCTGCT
ATTGATATGGCAGAGATACTGACGTTGTGGCCCAGACTGCAAGAGGCAAACGAACAGATATGCCTATACGCGCTGGGCGA
AACAATGGACAACATCAGATCCAAATGTCCGGTGAACGATTCCGATTCATCAACACCTCCCAGGACAGTGCCCTGCCTGT
GCCGCTACGCAATGACAGCAGAACGGATCGCCCGCCTTAGGTCACACCAAGTTAAAAGCATGGTGGTTTGCTCATCTTTT
CCCCTCCCGAAATACCATGTAGATGGGGTGCAGAAGGTAAAGTGCGAGAAGGTTCTCCTGTTCGACCCGACGGTACCTTC
AGTGGTTAGTCCGCGGAAGTATGCCGCATCTACGACGGACCACTCAGATCGGTCGTTACGAGGGTTTGACTTGGACTGGA
CCACCGACTCGTCTTCCACTGCCAGCGATACCATGTCGCTACCCAGTTTGCAGTCGTGTGACATCGACTCGATCTACGAG
CCAATGGCTCCCATAGTAGTGACGGCTGACGTACACCCTGAACCCGCAGGCATCGCGGACCTGGCGGCAGATGTGCACCC
TGAACCCGCAGACCATGTGGACCTCGAGAACCCGATTCCTCCACCGCGCCCGAAGAGAGCTGCATACCTTGCCTCCCGCG
CGGCGGAGCGACCGGTGCCGGCGCCGAGAAAGCCGACGCCTGCCCCAAGGACTGCGTTTAGGAACAAGCTGCCTTTGACG
TTCGGCGACTTTGACGAGCACGAGGTCGATGCGTTGGCCTCCGGGATTACTTTCGGAGACTTCGACGACGTCCTGCGACT
AGGCCGCGCGGGTGCATATATTTTCTCCTCGGACACTGGCAGCGGACATTTACAACAAAAATCCGTTAGGCAGCACAATC
TCCAGTGCGCACAACTGGATGCGGTCCAGGAGGAGAAAATGTACCCGCCAAAATTGGATACTGAGAGGGAGAAGCTGTTG
CTGCTGAAAATGCAGATGCACCCATCGGAGGCTAATAAGAGTCGATACCAGTCTCGCAAAGTGGAGAACATGAAAGCCAC
GGTGGTGGACAGGCTCACATCGGGGGCCAGATTGTACACGGGAGCGGACGTAGGCCGCATACCAACATACGCGGTTCGGT
ACCCCCGCCCCGTGTACTCCCCTACCGTGATCGAAAGATTCTCAAGCCCCGATGTAGCAATCGCAGCGTGCAACGAATAC
CTATCCAGAAATTACCCAACAGTGGCGTCGTACCAGATAACAGATGAATACGACGCATACTTGGACATGGTTGACGGGTC
GGATAGTTGCTTGGACAGAGCGACATTCTGCCCGGCGAAGCTCCGGTGCTACCCGAAACATCATGCGTACCACCAGCCGA
CTGTACGCAGTGCCGTCCCGTCACCCTTTCAGAACACACTACAGAACGTGCTAGCGGCCGCCACCAAGAGAAACTGCAAC
GTCACGCAAATGCGAGAACTACCCACCATGGACTCGGCAGTGTTCAACGTGGAGTGCTTCAAGCGCTATGCCTGCTCCGG
AGAATATTGGGAAGAATATGCTAAACAACCTATCCGGATAACCACTGAGAACATCACTACCTATGTGACCAAATTGAAAG
GCCCGAAAGCTGCTGCCTTGTTCGCTAAGACCCACAACTTGGTTCCGCTGCAGGAGGTTCCCATGGACAGATTCACGGTC
GACATGAAACGAGATGTCAAAGTCACTCCAGGGACGAAACACACAGAGGAAAGACCCAAAGTCCAGGTAATTCAAGCAGC
GGAGCCATTGGCGACCGCTTACCTGTGCGGCATCCACAGGGAATTAGTAAGGAGACTAAATGCTGTGTTACGCCCTAACG
TGCACACATTGTTTGATATGTCGGCCGAAGACTTTGACGCGATCATCGCCTCTCACTTCCACCCAGGAGACCCGGTTCTA
GAGACGGACATTGCATCATTCGACAAAAGCCAGGACGACTCCTTGGCTCTTACAGGTTTAATGATCCTCGAAGATCTAGG
GGTGGATCAGTACCTGCTGGACTTGATCGAGGCAGCCTTTGGGGAAATATCCAGCTGTCACCTACCAACTGGCACGCGCT
TCAAGTTCGGAGCTATGATGAAATCGGGCATGTTTCTGACTTTGTTTATTAACACTGTTTTGAACATCACCATAGCAAGC
AGGGTACTGGAGCAGAGACTCACTGACTCCGCCTGTGCGGCCTTCATCGGCGACGACAACATCGTTCACGGAGTGATCTC
CGACAAGCTGATGGCGGAGAGGTGCGCGTCGTGGGTCAACATGGAGGTGAAGATCATTGACGCTGTCATGGGCGAAAAAC
CCCCATATTTTTGTGGGGGATTCATAGTTTTTGACAGCGTCACACAGACCGCCTGCCGTGTTTCAGACCCACTTAAGCGC
CTGTTCAAGTTGGGTAAGCCGCTAACAGCTGAAGACAAGCAGGACGAAGACAGGCGACGAGCACTGAGTGACGAGGTTAG
CAAGTGGTTCCGGACAGGCTTGGGGGCCGAACTGGAGGTGGCACTAACATCTAGGTATGAGGTAGAGGGCTGCAAAAGTA
TCCTCATAGCCATGGCCACCTTGGCGAGGGACATTAAGGCGTTTAAGAAATTGAGAGGACCTGTTATACACCTCTACGGC
GGTCCTAGATTGGTGCGTTAATACACAGAATTCTGATTGGATCCCAAACGGGCCCTCTAGACTCGAGCGGCCGCCACTGT
GCTGGATATCTGCAGAATTCCACCACACTGGACTAGTGGATCTatggcgtacccatacgatgttccagattacgctagct
tgagatctaccatgtctcagagcaaccgggagctggtggttgactttctctcctacaagctttcccagaaaggatacagc
tggagtcagtttagtgatgtggaagagaacaggactgaggccccagaagggactgaatcggagatggagacccccagtgc
catcaatggcaacccatcctggcacctggcagacagccccgcggtgaatggagccactgcgcacagcagcagtttggatg
cccgggaggtgatccccatggcagcagtaaagcaagcgctgagggaggcaggcgacgagtttgaactgcggtaccggcgg
gcattcagtgacctgacatcccagctccacatcaccccagggacagcatatcagagctttgaacaggtagtgaatgaact
cttccgggatggggtaaactggggtcgcattgtggcctttttctccttcggcggggcactgtgcgtggaaagcgtagaca
aggagatgcaggtattggtgagtcggatcgcagcttggatggccacttacctgaatgaccacctagagccttggatccag
gagaacggcggctgggatacttttgtggaactctatgggaacaatgcagcagccgagagccgaaagggccaggaacgctt
caaccgctggttcctgacgggcatgactgtggccggcatggttctactgggctcactcttcagtcggaaatgaAGATCCG
AGCTCGGTACCAAGCTTAAGTTTGGGTAATTAATTGAATTACATCCCTACGCAAACGTTTTACGGCCGCCGGTGGCGCCC
GCGCCCGGCGGCCCGTCCTTGGCCGTTGCAGGCCACTCCGGTGGCTCCCGTCGTCCCCGACTTCCAGGCCCAGCAGATGC
AGCAACTCATCAGCGCCGTAAATGCGCTGACAATGAGACAGAACGCAATTGCTCCTGCTAGGCCTCCCAAACCAAAGAAG
AAGAAGACAACCAAACCAAAGCCGAAAACGCAGCCCAAGAAGATCAACGGAAAAACGCAGCAGCAAAAGAAGAAAGACAA
GCAAGCCGACAAGAAGAAGAAGAAACCCGGAAAAAGAGAAAGAATGTGCATGAAGATTGAAAATGACTGTATCTTCGTAT
GCGGCTAGCCACAGTAACGTAGTGTTTCCAGACATGTCGGGCACCGCACTATCATGGGTGCAGAAAATCTCGGGTGGTCT
GGGGGCCTTCGCAATCGGCGCTATCCTGGTGCTGGTTGTGGTCACTTGCATTGGGCTCCGCAGATAAGTTAGGGTAGGCA
ATGGCATTGATATAGCAAGAAAATTGAAAACAGAAAAAGTTAGGGTAAGCAATGGCATATAACCATAACTGTATAACTTG
TAACAAAGCGCAACAAGACCTGCGCAATTGGCCCCGTGGTCCGCCTCACGGAAACTCGGGGCAACTCATATTGACACATT
AATTGGCAATAATTGGAAGCTTACATAAGCTTAATTCGACGAATAATTGGATTTTTATTTTATTTTGCAATTGGTTTTTA
ATATTTCCAAAAAAAAAAAAAAAAAAAAAAAAAAAAAAAAAAAAAAAAAAAAAAAAAAAAAAAAAAAAAAAAAAAAAACT
AGTGATCATAATCAGCCATACCACATTTGTAGAGGTTTTACTTGCTTTAAAAAACCTCCCACACCTCCCCCTGAACCTGA
AACATAAAATGAATGCAATTGTTGTTGTTAACTTGTTTATTGCAGCTTATAATGGTTACAAATAAAGCAATAGCATCACA
AATTTCACAAATAAAGCATTTTTTTCACTGCATTCTAGTTGTGGTTTGTCCAAACTCATCAATGTATCTTATCATGTCTG
GATCTAGTCTGCATTAATGAATCGGCCAACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTCAAAGGCGGTAATACGGTTATCCACAGA
ATCAGGGGATAACGCAGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCAATGCTCGCGCTGTAGGTATCTCAGTTCGGTGTAGGTCGTTC
GCTCCAAGCTGGGCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGGACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACGGGGCATTCTGACGCTCAGTGGAA
CGAAAACTCACGTTAAGGGATTTTGGTCATGAGATTATCAAAAAGGATCTTCACCTAGATCCTTTTAAATTAAAAATGAA
GTTTTAAATCAATCTAAAGTATATATGAGTAAACTTGGTCTGACAGTTACCAATGCTTAATCAGTGAGGCACCTATCTCA
GCGATCTGTCTATTTCGTTCATCCATAGTTGCCTGACTCCCCGTCGTGTAGATAACTACGATACGGGAGGGCTTACCATC
TGGCCCCAGTGCTGCAATGATACCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGTCACGCTCGTCGTTTGGTATGGC
TTCATTCAGCTCCGGTTCCCAACGATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGCCGCAAAAAAGGGAATAAG
GGCGACACGGAAATGTTGAATACTCATACTCTTCCTTTTTCAATATTATTGAAGCATTTATCAGGGTTATTGTCTCATGA
GCGGATACATATTTGAATGTATTTAGAAAAATAAACAAATAGGGGTTCCGCGCACATTTCCCCGAAAAGTGCCACCTGAC
GTCTAAGAAACCATTATTATCATGACATTAACCTATAAAAATAGGCGTATCACGAGGCCCTTTCGTCTCGCGCGTTTCGG
TGATGACGGTGAAAACCTCTGACACATGCAGCTCCCGGAGACGGTCACAGCTTCTGTCTAAGCGGATGCCGGGAGCAGAC
AAGCCCGTCAGGGCGCGTCAGCGGGTGTTGGCGGGTGTCGGGGCTGGCTTAACTATGCGGCATCAGAGCAGATTGTACTG
AGAGTGCACCATATCGACGCTCTCCCTTATGCGACTCCTGCATTAGGAAGCAGCCCAGTACTAGGTTGAGGCCGTTGAGC
ACCGCCGCCGCAAGGAATGGTGCATGCGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATTGACGTCAATAATGACGTATGTTCCC
ATAGTAACGCCAATAGGGACTTTCCATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCATGGTGATGCGGTTTTGGCAGTAC
ATCAATGGGCGTGGATAGCGGTTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAATGGGCGGTAGGCGTGTACGGT
GGGAGGTCTATATAAGCAGAGCTCTCTGGCTAACTAGAGAACCCACTGCTTAACTGGCTTATCGAAATTAATACGACTCA
CTATAGGGAGACCGGAAGCTTGAATTC

The sequence of the “combined” vector, pSCA1-E7/BCL-xL [SEQ ID NO:25] is shown below with the sequence of E7 in lower case, not underscored, while the BCL-xL sequence is lower case and underscored.

ATGGCGGATGTGTGACATACACGACGCCAAAAGATTTTGTTCCAGCTCCTGCCACCTCCGCTACGCGAGAGATTAACCAC
CCACGATGGCCGCCAAAGTGCATGTTGATATTGAGGCTGACAGCCCATTCATCAAGTCTTTGCAGAAGGCATTTCCGTCG
TTCGAGGTGGAGTCATTGCAGGTCACACCAAATGACCATGCAAATGCCAGAGCATTTTCGCACCTGGCTACCAAATTGAT
CGAGCAGGAGACTGACAAAGACACACTCATCTTGGATATCGGCAGTGCGCCTTCCAGGAGAATGATGTCTACGCACAAAT
ACCACTGCGTATGCCCTATGCGCAGCGCAGAAGACCCCGAAAGGCTCGATAGCTACGCAAAGAAACTGGCAGCGGCCTCC
GGGAAGGTGCTGGATAGAGAGATCGCAGGAAAAATCACCGACCTGCAGACCGTCATGGCTACGCCAGACGCTGAATCTCC
TACCTTTTGCCTGCATACAGACGTCACGTGTCGTACGGCAGCCGAAGTGGCCGTATACCAGGACGTGTATGCTGTACATG
CACCAACATCGCTGTACCATCAGGCGATGAAAGGTGTCAGAACGGCGTATTGGATTGGGTTTGACACCACCCCGTTTATG
TTTGACGCGCTAGCAGGCGCGTATCCAACCTACGCCACAAACTGGGCCGACGAGCAGGTGTTACAGGCCAGGAACATAGG
ACTGTGTGCAGCATCCTTGACTGAGGGAAGACTCGGCAAACTGTCCATTCTCCGCAAGAAGCAATTGAAACCTTGCGACA
CAGTCATGTTCTCGGTAGGATCTACATTGTACACTGAGAGCAGAAAGCTACTGAGGAGCTGGCACTTACCCTCCGTATTC
CACCTGAAAGGTAAACAATCCTTTACCTGTAGGTGCGATACCATCGTATCATGTGAAGGGTACGTAGTTAAGAAAATCAC
TATGTGCCCCGGCCTGTACGGTAAAACGGTAGGGTACGCCGTGACGTATCACGCGGAGGGATTCCTAGTGTGCAAGACCA
CAGACACTGTCAAAGGAGAAAGAGTCTCATTCCCTGTATGCACCTACGTCCCCTCAACCATCTGTGATCAAATGACTGGC
ATACTAGCGACCGACGTCACACCGGAGGACGCACAGAAGTTGTTAGTGGGATTGAATCAGAGGATAGTTGTGAACGGAAG
AACACAGCGAAACACTAACACGATGAAGAACTATCTGCTTCCGATTGTGGCCGTCGCATTTAGCAAGTGGGCGAGGGAAT
ACAAGGCAGACCTTGATGATGAAAAACCTCTGGGTGTCCGAGAGAGGTCACTTACTTGCTGCTGCTTGTGGGCATTTAAA
ACGAGGAAGATGCACACCATGTACAAGAAACCAGACACCCAGACAATAGTGAAGGTGCCTTCAGAGTTTAACTCGTTCGT
CATCCCGAGCCTATGGTCTACAGGCCTCGCAATCCCAGTCAGATCACGCATTAAGATGCTTTTGGCCAAGAAGACCAAGC
GAGAGTTAATACCTGTTCTCGACGCGTCGTCAGCCAGGGATGCTGAACAAGAGGAGAAGGAGAGGTTGGAGGCCGAGCTG
ACTAGAGAAGCCTTACCACCCCTCGTCCCCATCGCGCCGGCGGAGACGGGAGTCGTCGACGTCGACGTTGAAGAACTAGA
GTATCACGCAGGTGCAGGGGTCGTGGAAACACCTCGCAGCGCGTTGAAAGTCACCGCACAGCCGAACGACGTACTACTAG
GAAATTACGTAGTTCTGTCCCCGCAGACCGTGCTCAAGAGCTCCAAGTTGGCCCCCGTGCACCCTCTAGCAGAGCAGGTG
AAAATAATAACACATAACGGGAGGGCCGGCGGTTACCAGGTCGACGGATATGACGGCAGGGTCCTACTACCATGTGGATC
GGCCATTCCGGTCCCTGAGTTTCAAGCTTTGAGCGAGAGCGCCACTATGGTGTACAACGAAAGGGAGTTCGTCAACAGGA
AACTATACCATATTGCCGTTCACGGACCGTCGCTGAACACCGACGAGGAGAACTACGAGAAAGTCAGAGCTGAAAGAACT
GACGCCGAGTACGTGTTCGACGTAGATAAAAAATGCTGCGTCAAGAGAGAGGAAGCGTCGGGTTTGGTGTTGGTGGGAGA
GCTAACCAACCCCCCGTTCCATGAATTCGCCTACGAAGGGCTGAAGATCAGGCCGTCGGCACCATATAAGACTACAGTAG
TAGGAGTCTTTGGGGTTCCGGGATCAGGCAAGTCTGCTATTATTAAGAGCCTCGTGACCAAACACGATCTGGTCACCAGC
GGCAAGAAGGAGAACTGCCAGGAAATAGTTAACGACGTGAAGAAGCACCGCGGGAAGGGGACAAGTAGGGAAAACAGTGA
CTCCATCCTGCTAAACGGGTGTCGTCGTGCCGTGGACATCCTATATGTGGACGAGGCTTTCGCTAGCCATTCCGGTACTC
TGCTGGCCCTAATTGCTCTTGTTAAACCTCGGAGCAAAGTGGTGTTATGCGGAGACCCCAAGCAATGCGGATTCTTCAAT
ATGATGCAGCTTAAGGTGAACTTCAACCACAACATCTGCACTGAAGTATGTCATAAAAGTATATCCAGACGTTGCACGCG
TCCAGTCACGGCCATCGTGTCTACGTTGCACTACGGAGGCAAGATGCGCACGACCAACCCGTGCAACAAACCCATAATCA
TAGACACCACAGGACAGACCAAGCCCAAGCCAGGAGACATCGTGTTAACATGCTTCCGAGGCTGGGCAAAGCAGCTGCAG
TTGGACTACCGTGGACACGAAGTCATGACAGCAGCAGCATCTCAGGGCCTCACCCGCAAAGGGGTATACGCCGTAAGGCA
GAAGGTGAATGAAAATCCCTTGTATGCCCCTGCGTCGGAGCACGTGAATGTACTGCTGACGCGCACTGAGGATAGGCTGG
TGTGGAAAACGCTGGCCGGCGATCCCTGGATTAAGGTCCTATCAAACATTCCACAGGGTAACTTTACGGCCACATTGGAA
GAATGGCAAGAAGAACACGACAAAATAATGAAGGTGATTGAAGGACCGGCTGCGCCTGTGGACGCGTTCCAGAACAAAGC
GAACGTGTGTTGGGCGAAAAGCCTGGTGCCTGTCCTGGACACTGCCGGAATCAGATTGACAGCAGAGGAGTGGAGCACCA
TAATTACAGCATTTAAGGAGGACAGAGCTTACTCTCCAGTGGTGGCCTTGAATGAAATTTGCACCAAGTACTATGGAGTT
GACCTGGACAGTGGCCTGTTTTCTGCCCCGAAGGTGTCCCTGTATTACGAGAACAACCACTGGGATAACAGACCTGGTGG
AAGGATGTATGGATTCAATGCCGCAACAGCTGCCAGGCTGGAAGCTAGACATACCTTCCTGAAGGGGCAGTGGCATACGG
GCAAGCAGGCAGTTATCGCAGAAAGAAAAATCCAACCGCTTTCTGTGCTGGACAATGTAATTCCTATCAACCGCAGGCTG
CCGCACGCCCTGGTGGCTGAGTACAAGACGGTTAAAGGCAGTAGGGTTGAGTGGCTGGTCAATAAAGTAAGAGGGTACCA
CGTCCTGCTGGTGAGTGAGTACAACCTGGCTTTGCCTCGACGCAGGGTCACTTGGTTGTCACCGCTGAATGTCACAGGCG
CCGATAGGTGCTACGACCTAAGTTTAGGACTGCCGGCTGACGCCGGCAGGTTCGACTTGGTCTTTGTGAACATTCACACG
GAATTCAGAATCCACCACTACCAGCAGTGTGTCGACCACGCCATGAAGCTGCAGATGCTTGGGGGAGATGCGCTACGACT
GCTAAAACCCGGCGGCATCTTGATGAGAGCTTACGGATACGCCGATAAAATCAGCGAAGCCGTTGTTTCCTCCTTAAGCA
GAAAGTTCTCGTCTGCAAGAGTGTTGCGCCCGGATTGTGTCACCAGCAATACAGAAGTGTTCTTGCTGTTCTCCAACTTT
GACAACGGAAAGAGACCCTCTACGCTACACCAGATGAATACCAAGCTGAGTGCCGTGTATGCCGGAGAAGCCATGCACAC
GGCCGGGTGTGCACCATCCTACAGAGTTAAGAGAGCAGACATAGCCACGTGCACAGAAGCGGCTGTGGTTAACGCAGCTA
ACGCCCGTGGAACTGTAGGGGATGGCGTATGCAGGGCCGTGGCGAAGAAATGGCCGTCAGCCTTTAAGGGAGCAGCAACA
CCAGTGGGCACAATTAAAACAGTCATGTGCGGCTCGTACCCCGTCATCCACGCTGTAGCGCCTAATTTCTCTGCCACGAC
TGAAGCGGAAGGGGACCGCGAATTGGCCGCTGTCTACCGGGCAGTGGCCGCCGAAGTAAACAGACTGTCACTGAGCAGCG
TAGCCATCCCGCTGCTGTCCACAGGAGTGTTCAGCGGCGGAAGAGATAGGCTGCAGCAATCCCTCAACCATCTATTCACA
GCAATGGACGCCACGGACGCTGACGTGACCATCTACTGCAGAGACAAAAGTTGGGAGAAGAAAATCCAGGAAGCCATTGA
CATGAGGACGGCTGTGGAGTTGCTCAATGATGACGTGGAGCTGACCACAGACTTGGTGAGAGTGCACCCGGACAGCAGCC
TGGTGGGTCGTAAGGGCTACAGTACCACTGACGGGTCGCTGTACTCGTACTTTGAAGGTACGAAATTCAACCAGGCTGCT
ATTGATATGGCAGAGATACTGACGTTGTGGCCCAGACTGCAAGAGGCAAACGAACAGATATGCCTATACGCGCTGGGCGA
AACAATGGACAACATCAGATCCAAATGTCCGGTGAACGATTCCGATTCATCAACACCTCCCAGGACAGTGCCCTGCCTGT
GCCGCTACGCAATGACAGCAGAACGGATCGCCCGCCTTAGGTCACACCAAGTTAAAAGCATGGTGGTTTGCTCATCTTTT
CCCCTCCCGAAATACCATGTAGATGGGGTGCAGAAGGTAAAGTGCGAGAAGGTTCTCCTGTTCGACCCGACGGTACCTTC
AGTGGTTAGTCCGCGGAAGTATGCCGCATCTACGACGGACCACTCAGATCGGTCGTTACGAGGGTTTGACTTGGACTGGA
CCACCGACTCGTCTTCCACTGCCAGCGATACCATGTCGCTACCCAGTTTGCAGTCGTGTGACATCGACTCGATCTACGAG
CCAATGGCTCCCATAGTAGTGACGGCTGACGTACACCCTGAACCCGCAGGCATCGCGGACCTGGCGGCAGATGTGCACCC
TGAACCCGCAGACCATGTGGACCTCGAGAACCCGATTCCTCCACCGCGCCCGAAGAGAGCTGCATACCTTGCCTCCCGCG
CGGCGGAGCGACCGGTGCCGGCGCCGAGAAAGCCGACGCCTGCCCCAAGGACTGCGTTTAGGAACAAGCTGCCTTTGACG
TTCGGCGACTTTGACGAGCACGAGGTCGATGCGTTGGCCTCCGGGATTACTTTCGGAGACTTCGACGACGTCCTGCGACT
AGGCCGCGCGGGTGCATATATTTTCTCCTCGGACACTGGCAGCGGACATTTACAACAAAAATCCGTTAGGCAGCACAATC
TCCAGTGCGCACAACTGGATGCGGTCCAGGAGGAGAAAATGTACCCGCCAAAATTGGATACTGAGAGGGAGAAGCTGTTG
CTGCTGAAAATGCAGATGCACCCATCGGAGGCTAATAAGAGTCGATACCAGTCTCGCAAAGTGGAGAACATGAAAGCCAC
GGTGGTGGACAGGCTCACATCGGGGGCCAGATTGTACACGGGAGCGGACGTAGGCCGCATACCAACATACGCGGTTCGGT
ACCCCCGCCCCGTGTACTCCCCTACCGTGATCGAAAGATTCTCAAGCCCCGATGTAGCAATCGCAGCGTGCAACGAATAC
CTATCCAGAAATTACCCAACAGTGGCGTCGTACCAGATAACAGATGAATACGACGCATACTTGGACATGGTTGACGGGTC
GGATAGTTGCTTGGACAGAGCGACATTCTGCCCGGCGAAGCTCCGGTGCTACCCGAAACATCATGCGTACCACCAGCCGA
CTGTACGCAGTGCCGTCCCGTCACCCTTTCAGAACACACTACAGAACGTGCTAGCGGCCGCCACCAAGAGAAACTGCAAC
GTCACGCAAATGCGAGAACTACCCACCATGGACTCGGCAGTGTTCAACGTGGAGTGCTTCAAGCGCTATGCCTGCTCCGG
AGAATATTGGGAAGAATATGCTAAACAACCTATCCGGATAACCACTGAGAACATCACTACCTATGTGACCAAATTGAAAG
GCCCGAAAGCTGCTGCCTTGTTCGCTAAGACCCACAACTTGGTTCCGCTGCAGGAGGTTCCCATGGACAGATTCACGGTC
GACATGAAACGAGATGTCAAAGTCACTCCAGGGACGAAACACACAGAGGAAAGACCCAAAGTCCAGGTAATTCAAGCAGC
GGAGCCATTGGCGACCGCTTACCTGTGCGGCATCCACAGGGAATTAGTAAGGAGACTAAATGCTGTGTTACGCCCTAACG
TGCACACATTGTTTGATATGTCGGCCGAAGACTTTGACGCGATCATCGCCTCTCACTTCCACCCAGGAGACCCGGTTCTA
GAGACGGACATTGCATCATTCGACAAAAGCCAGGACGACTCCTTGGCTCTTACAGGTTTAATGATCCTCGAAGATCTAGG
GGTGGATCAGTACCTGCTGGACTTGATCGAGGCAGCCTTTGGGGAAATATCCAGCTGTCACCTACCAACTGGCACGCGCT
TCAAGTTCGGAGCTATGATGAAATCGGGCATGTTTCTGACTTTGTTTATTAACACTGTTTTGAACATCACCATAGCAAGC
AGGGTACTGGAGCAGAGACTCACTGACTCCGCCTGTGCGGCCTTCATCGGCGACGACAACATCGTTCACGGAGTGATCTC
CGACAAGCTGATGGCGGAGAGGTGCGCGTCGTGGGTCAACATGGAGGTGAAGATCATTGACGCTGTCATGGGCGAAAAAC
CCCCATATTTTTGTGGGGGATTCATAGTTTTTGACAGCGTCACACAGACCGCCTGCCGTGTTTCAGACCCACTTAAGCGC
CTGTTCAAGTTGGGTAAGCCGCTAACAGCTGAAGACAAGCAGGACGAAGACAGGCGACGAGCACTGAGTGACGAGGTTAG
CAAGTGGTTCCGGACAGGCTTGGGGGCCGAACTGGAGGTGGCACTAACATCTAGGTATGAGGTAGAGGGCTGCAAAAGTA
TCCTCATAGCCATGGCCACCTTGGCGAGGGACATTAAGGCGTTTAAGAAATTGAGAGGACCTGTTATACACCTCTACGGC
GGTCCTAGATTGGTGCGTTAATACACAGAATTCTGATTGGATCCCAAACGGGCCCTCTAGACTCGAGCGGCCGCCACTGT
GCTGGATATCTGCAGAATTCatgcatggagatacacctacattgcatgaatatatgttagatttgcaaccagagacaact
gatctctactgttatgagcaattaaatgacagctcagaggaggaggatgaaatagatggtccagctggacaagcagaacc
ggacagagcccattacaatattgtaaccttttgttgcaagtgtgactctacgcttcggttgtgcgtacaaagcacacacg
tagacattcgtactttggaagacctgttaatgggcacactaggaattgtgtgccccatctgttctcagaaaccaGGATCT
atggcgtacccatacgatgttccagattacgctagcttgagatctaccatgtctcagagcaaccgggagctggtggttga
ctttctctcctacaagctttcccagaaaggatacagctggagtcagtttagtgatgtggaagagaacaggactgaggccc
cagaagggactgaatcggagatggagacccccagtgccatcaatggcaacccatcctggcacctggcagacagccccgcg
gtgaatggagccactgcgcacagcagcagtttggatgcccgggaggtgatccccatggcagcagtaaagcaagcgctgag
ggaggcaggcgacgagtttgaactgcggtaccggcgggcattcagtgacctgacatcccagctccacatcaccccaggga
cagcatatcagagctttgaacaggtagtgaatgaactcttccgggatggggtaaactggggtcgcattgtggcctttttc
tccttcggcggggcactgtgcgtggaaagcgtagacaaggagatgcaggtattggtgagtcggatcgcagcttggatggc
cacttacctgaatgaccacctagagccttggatccaggagaacggcggctgggatacttttgtggaactctatgggaaca
atgcagcagccgagagccgaaagggccaggaacgcttcaaccgctggttcctgacgggcatgactgtggccggcgtggtt
ctgctgggctcactcttcagtcggaaatgaAGATCCAAGCTTAAGTTTGGGTAATTAATTGAATTACATCCCTACGCAAA
CGTTTTACGGCCGCCGGTGGCGCCCGCGCCCGGCGGCCCGTCCTTGGCCGTTGCAGGCCACTCCGGTGGCTCCCGTCGTC
CCCGACTTCCAGGCCCAGCAGATGCAGCAACTCATCAGCGCCGTAAATGCGCTGACAATGAGACAGAACGCAATTGCTCC
TGCTAGGCCTCCCAAACCAAAGAAGAAGAAGACAACCAAACCAAAGCCGAAAACGCAGCCCAAGAAGATCAACGGAAAAA
CGCAGCAGCAAAAGAAGAAAGACAAGCPAGCCGACAAGAAGAAGAAGAAACCCGGAAAAAGAGAAAGAATGTGCATGAAG
ATTGAAAATGACTGTATCTTCGTATGCGGCTAGCCACAGTAACGTAGTGTTTCCAGACATGTCGGGCACCGCACTATCAT
GGGTGCAGAAAATCTCGGGTGGTCTGGGGGCCTTCGCAATCGGCGCTATCCTGGTGCTGGTTGTGGTCACTTGCATTGGG
CTCCGCAGATAAGTTAGGGTAGGCAATGGCATTGATATAGCAAGAAAATTGAAAACAGAAAAAGTTAGGGTAAGCAATGG
CATATAACCATAACTGTATAACTTGTAACAAAGCGCAACAAGACCTGCGCAATTGGCCCCGTGGTCCGCCTCACGGAAAC
TCGGGGCAACTCATATTGACACATTAATTGGCAATAATTGGAAGCTTACATAAGCTTAATTCGACGAATAATTGGATTTT
TATTTTATTTTGCAATTGGTTTTTAATATTTCCAAAAAAAAAAAAAAAAAAAAAAAAAAAAAAAAAAAAAAAAAAAAAAA
AAAAAAAAAAAAAAAAAAAAAAACTAGTGATCATAATCAGCCATACCACATTTGTAGAGGTTTTACTTGCTTTAAAAAAC
CTCCCACACCTCCCCCTGAACCTGAAACATAAAATGAATGCAATTGTTGTTGTTAACTTGTTTATTGCAGCTTATAATGG
TTACAAATAAAGCAATAGCATCACAAATTTCACAAATAAAGCATTTTTTTCACTGCATTCTAGTTGTGGTTTGTCCAAAC
TCATCAATGTATCTTATCATGTCTGGATCTAGTCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAGGCCAGCAAAAGGCCAGG
AACCGTAAAAAGGCCGCGTTGCTGGCGTTTTTCCATAGGCTCCGCCCCCCTGACGAGCATCACAAAAATCGACGCTCAAG
TCAGAGGTGGCGAAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCAATGCTCGCGCTGTAGG
TATCTCAGTTCGGTGTAGGTCGTTCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGCTACACTAGAAGGACAGTATTT
GGTATCTGCGCTCTGCTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTTTGATCTTTTCTA
CGGGGCATTCTGACGCTCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGACTCCCCGTCGTGTAGATAA
CTACGATACGGGAGGGCTTACCATCTGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACTTTATCCGCCTCCATCCAGTCTATTAA
TTGTTGCCGGGAAGCTAGAGTAAGTAGTTCGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAGTACTCAACCAAGTCAT
TCTGAGAATAGTGTATGCGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTGTTGAGATCCAGTTCGAT
GTAACCCACTCGTGCACCCAACTGATCTTCAGCATCTTTTACTTTCACCAGCGTTTCTGGGTGAGCAAAAACAGGAAGGC
AAAATGCCGCAAAAAAGGGAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACAAATAGGGGTTCCGCGCAC
ATTTCCCCGAAAAGTGCCACCTGACGTCTAAGAAACCATTATTATCATGACATTAACCTATAAAAATAGGCGTATCACGA
GGCCCTTTCGTCTCGCGCGTTTCGGTGATGACGGTGAAAACCTCTGACACATGCAGCTCCCGGAGACGGTCACAGCTTCT
GTCTAAGCGGATGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATATCGACGCTCTCCCTTATGCGACTCCTGCATTAGGAAGCAGCC
CAGTACTAGGTTGAGGCCGTTGAGCACCGCCGCCGCAAGGAATGGTGCATGCGTAATCAATTACGGGGTCATTAGTTCAT
AGCCCATATATGGAGTTCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTATTAGTCATCGCTATTAC
CATGGTGATGCGGTTTTGGCAGTACATCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGAGCTCTCTGGCTAACTAGAGAACCCACTGCTTAACT
GGCTTATCGAAATTAATACGACTCACTATAGGGAGACCGGAAGCTTGAATTC

The sequence of pSCA1-mtBCL-xL [SEQ ID NO:26] is the same as that for the wild type BCL-xL except that the mtBCL-xL sequence is inserted in the same position as the wild type sequence in the pSCA1-mtBCL-xL vecvot

The sequence pSCA1-E7/mtBCL-xL [SEQ ID NO:27] is the same as that for the wild type pSCA1-E7/BCL-xL above, except that the mtBCL-xL sequence is inserted in the same position as the wild type sequence.

The sequenced of the vector pSG5-BCL-xL [SEQ ID NO:28] is shown below, with the BCL-xL coding sequence in lower case underscored:

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCAGATCTatggcgtacccatacgatgttccagattacgctagcttgagatctaccatgtctcagagc
aaccgggagctggtggttgactttctctcctacaagctttcccagaaaggatacagctggagtcagtttagtgatgtgga
agagaacaggactgaggccccagaagggactgaatcggagatggagacccccagtgccatcaatggcaacccatcctggc
acctggcagacagccccgcggtgaatggagccactgcgcacagcagcagtttggatgcccgggaggtgatccccatggca
gcagtaaagcaagcgctgagggaggcaggcgacgagtttgaactgcggtaccggcgggcattcagtgacctgacatccca
gctccacatcaccccagggacagcatatcagagctttgaacaggtagtgaatgaactcttccgggatggggtaaactggg
gtcgcattgtggcctttttctccttcggcggggcactgtgcgtggaaagcgtagacaaggagatgcaggtattggtgagt
cggatcgcagcttggatggccacttacctgaatgaccacctagagccttggatccaggagaacggcggctgggatacttt
tgtggaactctatgggaacaatgcagcagccgagagccgaaagggccaggaacgcttcaaccgctggttcctgacgggca
tgactgtggccggcgtggttctgctgggctcactcttcagtcggaaatgaAGATCTTATTAAAGCAGAACTTGTTTATTG
CAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGGTCGACTCTAGACTCTTCCGCTTCCTCGCTCACTGACTCGCT
GCGCTCGGTCGTTCGGCTGCGGCGAGCGGTATCAGCTCACTCAAAGGCGGTAATACGGTTATCCACAGAATCAGGGGATA
ACGCAGGAAAGAACATGTGAGCAAAGGCCAGCAAAAGGCCAGGAACCGTAAAAAGGCCGCGTTGCTGGCGTTTTTTCCAT
AGGCTCCGCCCCCCTGACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGATACCTGTCCGCCTTTC
TCCCTTCGGGAAGCGTGGCGCTTTCTCAATGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTAAAAATGAAGTTTTAAATCAATC
TAAAGTATATATGAGTAAACTTGGTCTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAGGGCTTACCATCTGGCCCCAGTGCTG
CAATGATACCGCGAGACCCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCTTACTGTCATGCCATCCGT
AAGATGCTTTTCTGTGACTGGTGAGTACTCAACCAAGTCATTCTGAGAATAGTGTATGCGGCGACCGAGTTGCTCTTGCC
CGGCGTCAATACGGGATAATACCGCGCCACATAGCAGAACTTTAAAAGTGCTCATCATTGGAAAACGTTCTTCGGGGCGA
AAACTCTCAAGGATCTTACCGCTGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAAAGGGAATAAGGGCGACACGGAAAT
GTTGAATACTCATACTCTTCTTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCCTTTCGTCTCGCGCGTTTCGGTGATGACGGTGAA
AACCTCTGACACATGCAGCTCCCGGAGACGGTCACAGCTTGTCTGTAAGCGGATGCCGGGAGCAGACAAGCCCGTCAGGG
CGCGTCAGCGGGTGTTGGCGGGTGTCGGGGCTGGCTTAACTATGCGGCATCAGAGCAGATTGTACTGAGAGTGCACCATA
TGCGGTGTGAAATACCGCACAGATGCGTAAGGAGAAAATACCGCATCAGGAAATTGTAAACGTTAATATTTTGTTAAAAT
TCGCGTTAAATTTTTGTTAAATCAGCTCATTTTTTAACCAATAGGCCGAAATCGGCAAAATCCCTTATAAATCAAAAGAA
TAGACCGAGATAGGGTTGAGTGTTGTTCCAGTTTGGAACAAGAGTCCACTATTAAAGAACGTGGACTCCAACGTCAAAGG
GCGAAAAACCGTCTATCAGGGCGATGGCCCACTACGTGAACCATCACCCTAATCAAGTTTTTTGGGGTCGAGGTGCCGTA
AAGCACTAAATCGGAACCCTAAAGGGAGCCCCCGATTTAGAGCTTGACGGGGAAAGCCGGCGAACGTGGCGAGAAAGGAA
GGGAAGAAAGCGAAAGGAGCGGGCGCTAGGGCGCTGGCAAGTGTAGCGGTCACGCTGCGCGTAACCACCACACCCGCCGC
GCTTAATGCGCCGCTACAGGGCGCGTCGCGCCATTCGCCATTCAGGCTACGCAACTGTTGGGAAGGGCGATCGGTGCGGG
CCTCTTCGCTATTACGCCAGCTGGCGAAGGGGGGATGTGCTGCAAGGCGATTAAGTTGGGTAACGCCAGGGTTTTCCCAG
TCACGACGTTGTAAAACGACGGCCAGTGAATT

The sequenced of the vector pSG5-mtBCL-xL [SEQ ID NO:29] with the mutant BCL-xL sequence has the mtBCL-xL, shown above, inserted in the same location as for the wild type vector immediately above.

The nucleotide sequence of the DNA [SEQ ID NO:30] encoding the XIAP anti-apoptotic protein is:

ATGACTTTTAACAGTTTTGAAGGATCTAAAACTTGTGTACCTGCAGACATCAATAAGGAAGAAGAATTTGTAGAAGAGTT
TAATAGATTAAAAACTTTTGCTAATTTTCCAAGTGGTAGTCCTGTTTCAGCATCAACACTGGCACGAGCAGGGTTTCTTT
ATACTGGTGAAGGAGATACCGTGCGGTGCTTTAGTTGTCATGCAGCTGTAGATAGATGGCAATATGGAGACTCAGCAGTT
GGAAGACACAGGAAAGTATCCCCAAATTGCAGATTTATCAACGGCTTTTATCTTGAAAATAGTGCCACGCAGTCTACAAA
TTCTGGTATCCAGAATGGTCAGTACAAAGTTGAAAACTATCTGGGAAGCAGAGATCATTTTGCCTTAGACAGGCCATCTG
AGACACATGCAGACTATCTTTTGAGAACTGGGCAGGTTGTAGATATATCAGACACCATATACCCGAGGAACCCTGCCATG
TATTGTGAAGAAGCTAGATTAAAGTCCTTTCAGAACTGGCCAGACTATGCTCACCTAACCCCAAGAGAGTTAGCAAGTGC
TGGACTCTACTACACAGGTATTGGTGACCAAGTGCAGTGCTTTTGTTGTGGTGGAAAACTGAAAAATTGGGAACCTTGTG
ATCGTGCCTGGTCAGAACACAGGCGACACTTTCCTAATTGCTTCTTTGTTTTGGGCCGGAATCTTAATATTCGAAGTGAA
TCTGATGCTGTGAGTTCTGATAGGAATTTCCCAAATTCAACAAATCTTCCAAGAAATCCATCCATGGCAGATTATGAAGC
ACGGATCTTTACTTTTGGGACATGGATATACTCAGTTAACAAGGAGCAGCTTGCAAGAGCTGGATTTTATGCTTTAGGTG
AAGGTGATAAAGTAAAGTGCTTTCACTGTGGAGGAGGGCTAACTGATTGGAAGCCCAGTGAAGACCCTTGGGAACAACAT
GCTAAATGGTATCCAGGGTGCAAATATCTGTTAGAACAGAAGGGACAAGAATATATAAACAATATTCATTTAACTCATTC
ACTTGAGGAGTGTCTGGTAAGAACTACTGAGAAAACACCATCACTAACTAGAAGAATTGATGATACCATCTTCCAAAATC
CTATGGTACAAGAAGCTATACGAATGGGGTTCAGTTTCAAGGACATTAAGAAAATAATGGAGGAAAAAATTCAGATATCT
GGGAGCAACTATAAATCACTTGAGGTTCTGGTTGCAGATCTAGTGAATGCTCAGAAAGACAGTATGCAAGATGAGTCAAG
TCAGACTTCATTACAGAAAGAGATTAGTACTGAAGAGCAGCTAAGGCGCCTGCAAGAGGAGAAGCTTTGCAAAATCTGTA
TGGATAGAAATATTGCTATCGTTTTTGTTCCTTGTGGACATCTAGTCACTTGTAAACAATGTGCTGAAGCAGTTGACAAG
TGTCCCATGTGCTACACAGTCATTACTTTCAAGCAAAAAATTTTTATGTCTTAATCTAA

The amino acid of the vector comprising the XIAP anti-apoptotic protein coding sequence [SEQ ID NO:31] is:

MTFNSFEGSK TCVPADINKE EEFVEEFNRL KTFANFPSGS PVSASTLARA GFLYTGEGDT
VRCFSCHAAV DRWQYGDSAV GRHRKVSPNC RFINGFYLEN SATQSTNSGI QNGQYKVENY
LGSRDHFALD RPSETHADYL LRTGQVVDIS DTIYPRNPAM YCEEARLKSF QNWPDYAHLT
PRELASAGLY YTGIGDQVQC FCCGGKLKNW EPCDRAWSEH RRHFPNCFFV LGRNLNIRSE
SDAVSSDRNF PNSTNLPRNP SMADYEARIF TFGTWIYSVN KEQLARAGFY ALGEGDKVKC
FHCGGGLTDW KPSEDPWEQH AKWYPGCKYL LEQKGQEYIN NIHLTHSLEE CLVRTTEKTP
SLTRRIDDTI FQNPMVQEAI RMGFSFKDIK KIMEEKIQIS GSNYKSLEVL VADLVNAQKD
SMQDESSQTS LQKEISTEEQ LRRLQEEKLC KICMDRNIAI VFVPCGHLVT CKQCAEAVDK
CPMCYTVITF KQKIFMS

The nucleotide sequence of the vector comprising the XIAP anti-apoptotic protein coding sequence, designated PSG5-XIAP [SEQ ID NO:32] is shown below (with the XIAP in lower case, underscored:

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATAATCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCatgacttttaacagttttgaaggatctaaaacttgtgtacctgcagacatcaataaggaagaagaa
tttgtagaagagtttaatagattaaaaacttttgctaattttccaagtggtagtcctgtttcagcatcaacactggcacg
agcagggtttctttatactggtgaaggagataccgtgcggtgctttagttgtcatgcagctgtagatagatggcaatatg
gagactcagcagttggaagacacaggaaagtatccccaaattgcagatttatcaacggcttttatcttgaaaatagtgcc
acgcagtctacaaattctggtatccagaatggtcagtacaaagttgaaaactatctgggaagcagagatcattttgcctt
agacaggccatctgagacacatgcagactatcttttgagaactgggcaggttgtagatatatcagacaccatatacccga
ggaaccctgccatgtattgtgaagaagctagattaaagtcctttcagaactggccagactatgctcacctaaccccaaga
gagttagcaagtgctggactctactacacaggtattggtgaccaagtgcagtgcttttgttgtggtggaaaactgaaaaa
ttgggaaccttgtgatcgtgcctggtcagaacacaggcgacactttcctaattgcttctttgttttgggccggaatctta
atattcgaagtgaatctgatgctgtgagttctgataggaatttcccaaattcaacaaatcttccaagaaatccatccatg
gcagattatgaagcacggatctttacttttgggacatggatatactcagttaacaaggagcagcttgcaagagctggatt
ttatgctttaggtgaaggtgataaagtaaagtgctttcactgtggaggagggctaactgattggaagcccagtgaagacc
cttgggaacaacatgctaaatggtatccagggtgcaaatatctgttagaacagaagggacaagaatatataaacaatatt
catttaactcattcacttgaggagtgtctggtaagaactactgagaaaacaccatcactaactagaagaattgatgatac
catcttccaaaatcctatggtacaagaagctatacgaatggggttcagtttcaaggacattaagaaaataatggaggaaa
aaattcagatatctgggagcaactataaatcacttgaggttctggttgcagatctagtgaatgctcagaaagacagtatg
caagatgagtcaagtcagacttcattacagaaagagattagtactgaagagcagctaaggcgcctgcaagaggagaagct
ttgcaaaatctgtatggatagaaatattgctatcgtttttgttccttgtggacatctagtcacttgtaaacaatgtgctg
aagcagttgacaagtgtcccatgtgctacacagtcattactttcaagcaaaaaatttttatgtcttaatctaaAGATCTT
ATTAAAGCAGAACTTGTTTATTGCAGCTTATAATGGTTACAAATAAAGCAATAGCATCACAAATTTCACAAATAAAGCAT
TTTTTTCACTGCATTCTAGTTGTGGTTTGTCCAAACTCATCAATGTATCTTATCATGTCTGGTCGACTCTAGACTCTTCC
GCTTCCTCGCTCACTGACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGTCAGAGGTGGCGA
AACCCGACAGGACTATAAAGATACCAGGCGTTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTCAATGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGGACAGTATTTGGTATCTGCGCTC
TGCTGAAGCCAGTTACCTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAGCAATAAACCAGC
CAGCCGGAAGGGCCGAGCGCAGAAGTGGTCCTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGCTACAGGCATCGTGGTGTCACGCTCGTCGTT
TGGTATGGCTTCATTCAGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCAAAATGCCGCAAAAAA
GGGAATAAGGGCGACACGGAAATGTTGAATACTCATACTCTTCTTTTTTCAATATTATTGAAGCATTTATCAGGGTTATT
GTCTCATGAGCGGATACATATTTGAATGTATTTAGAAAAATAAACAAATAGGGGTTCCGCGCACATTTCCCCGAAAAGTG
CCACCTGACGTCTAAGAAACCATTATTATCATGACATTAACCTATAAAAATAGGCGTATCACGAGGCCCCTTTCGTCTCG
CGCGTTTCGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCATCAGAGCA
GATTGTACTGAGAGTGCACCATATGCGGTGTGAAATACCGCACAGATGCGTAAGGAGAAAATACCGCATCAGGAAATTGT
AAACGTTAATATTTTGTTAAAATTCGCGTTAAATTTTTGTTAAATCAGCTCATTTTTTAACCAATAGGCCGAAATCGGCA
AAATCCCTTATAAATCAAAAGAATAGACCGAGATAGGGTTGAGTGTTGTTCCAGTTTGGAACAAGAGTCCACTATTAAAG
AACGTGGACTCCAACGTCAAAGGGCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCGATTTAGAGCTTGACGGGGAAAGC
CGGCGAACGTGGCGAGAAAGGAAGGGAAGAAAGCGAAAGGAGCGGGCGCTAGGGCGCTGGCAAGTGTAGCGGTCACGCTG
CGCGTAACCACCACACCCGCCGCGCTTAATGCGCCGCTACAGGGCGCGTCGCGCCATTCGCCATTCAGGCTACGCAACTG
TTGGGAAGGGCGATCGGTGCGGGCCTCTTCGCTATTACGCCAGCTGGCGAAGGGGGGATGTGCTGCAAGGCGATTAAGTT
GGGTAACGCCAGGGTTTTCCCAGTCACGACGTTGTAAAACGACGGCCAGTGAATT

The sequence of DNA encoding the anti-apoptotic protein FLICEc-s [SEQ ID NO:33] is shown below:

ATGGACTTCAGCAGAAATCTTTATGATATTGGGGAACAACTGGACAGTGAAGATCTGGCCTCCCTCAAGTTCC
TGAGCCTGGACTACATTCCGCAAAGGAAGCAAGAACCCATCAAGGATGCCTTGATGTTATTCCAGAGACTCCA
GGAAAAGAGAATGTTGGAGGAAAGCAATCTGTCCTTCCTGAAGGAGCTGCTCTTCCGAATTAATAGACTGGAT
TTGCTGATTACCTACCTAAACACTAGAAAGGAGGAGATGGAAAGGGAACTTCAGACACCAGGCAGGGCTCAAA
TTTCTGCCTACAGGGTCATGCTCTATCAGATTTCAGAAGAAGTGAGCAGATCAGAATTGAGGTCTTTTAAGTT
TCTTTTGCAAGAGGAAATCTCCAAATGCAAACTGGATGATGACATGAACCTGCTGGATATTTTCATAGAGATG
GAGAAGAGGGTCATCCTGGGAGAAGGAAAGTTGGACATCCTGAAAAGAGTCTGTGCCCAAATCAACAAGAGCC
TGCTGAAGATAATCAACGACTATGAAGAATTCAGCAAAGGGGAGGAGTTGTGTGGGGTAATGACAATCTCGGA
CTCTCCAAGAGAACAGGATAGTGAATCACAGACTTTGGACAAAGTTTACCAAATGAAAAGCAAACCTCGGGGA
TACTGTCTGATCATCAACAATCACAATTTTGCAAAAGCACGGGAGAAAGTGCCCAAACTTCACAGCATTAGGG
ACAGGAATGGAACACACTTGGATGCAGGGGCTTTGACCACGACCTTTGAAGAGCTTCATTTTGAGATCAAGCC
CCACGATGACTGCACAGTAGAGCAAATCTATGAGATTTTGAAAATCTACCAACTCATGGACCACAGTAACATG
GACTGCTTCATCTGCTGTATCCTCTCCCATGGAGACAAGGGCATCATCTATGGCACTGATGGACAGGAGGCCC
CCATCTATGAGCTGACATCTCAGTTCACTGGTTTGAAGTGCCCTTCCCTTGCTGGAAAACCCAAAGTGTTTTT
TATTCAGGCTTGTCAGGGGGATAACTACCAGAAAGGTATACCTGTTGAGACTGATTCAGAGGAGCAACCCTAT
TTAGAAATGGATTTATCATCACCTCAAACGAGATATATCCCGGATGAGGCTGACTTTCTGCTGGGGATGGCCA
CTGTGAATAACTGTGTTTCCTACCGAAACCCTGCAGAGGGAACCTGGTACATCCAGTCACTTTGCCAGAGCCT
GAGAGAGCGATGTCCTCGAGGCGATGATATTCTCACCATCCTGACTGAAGTGAACTATGAAGTAAGCAACAAG
GATGACAAGAAAAACATGGGGAAACAGATGCCTCAGCCTACTTTCACACTAAGAAAAAAACTTGTCTTCCCTT
CTGATTGA

The amino acid sequence of the anti-apoptotic protein FLICEc-s [SEQ ID NO:34] is:

MDFSRNLYDI GEQLDSEDLA SLKFLSLDYI PQRKQEPIKD ALMLFQRLQE KRMLEESNLS
FLKELLFRIN RLDLLITYLN TRKEEMEREL QTPGRAQISA YRVMLYQISE EVSRSELRSF
KFLLQEEISK CKLDDDMNLL DIFIEMEKRV ILGEGKLDIL KRVCAQINKS LLKIINDYEE
FSKGEELCGV MTISDSPREQ DSESQTLDKV YQMKSKPRGY CLIINNHNFA KAREKVPKLH
SIRDRNGTHL DAGALTTTFE ELHFEIKPHD DCTVEQIYEI LKIYQLMDHS NMDCFICCIL
SHGDKGIIYG TDGQEAPIYE LTSQFTGLKC PSLAGKPKVF FIQACQGDNY QKGIPVETDS
EEQPYLEMDL SSPQTRYIPD EADFLLGMAT VNNCVSYRNP AEGTWYIQSL CQSLRERCPR
GDDILTILTE VNYEVSNKDD KKNMGKQMPQ PTFTLRKKLV FPSD

The PSG5 vector encoding the anti-apoptotic protein FLICEc-s, designated PSG5-FLICEc-s, has the sequence shown below [SEQ ID NO:35] (with the FLICEc-s sequence in lower case, underscored):

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCatggacttcagcagaaatctttatgatattggggaacaactggacagtgaagatctggcctccctcaagttc
ctgagcctggactacattccgcaaaggaagcaagaacccatcaaggatgccttgatgttattccagagactccaggaaaa
gagaatgttggaggaaagcaatctgtccttcctgaaggagctgctcttccgaattaatagactggatttgctgattacct
acctaaacactagaaaggaggagatggaaagggaacttcagacaccaggcagggctcaaatttctgcctacagggtcatg
ctctatcagatttcagaagaagtgagcagatcagaattgaggtcttttaagtttcttttgcaagaggaaatctccaaatg
caaactggatgatgacatgaacctgctggatattttcatagagatggagaagagggtcatcctgggagaaggaaagttgg
acatcctgaaaagagtctgtgcccaaatcaacaagagcctgctgaagataatcaacgactatgaagaattcagcaaaggg
gaggagttgtgtggggtaatgacaatctcggactctccaagagaacaggatagtgaatcacagactttggacaaagttta
ccaaatgaaaagcaaacctcgggatactgtctgatcatcaacaatcacaattttgcaaaagcacgggagaaagtgcccca
aacttcacagcattagggacaggaatggaacacacttggatgcaggggctttgaccacgacctttgaagagcttcatttt
gagatcaagccccacgatgactgcacagtagagcaaatctatgagattttgaaaatctaccaactcatggaccacagtaa
catggactgcttcatctgctgtatcctctcccatggagacaagggcatcatctatggcactgatggacaggaggccccca
tctatgagctgacatctcagttcactggtttgaagtgcccttcccttgctggaaaacccaaagtgttttttattcaggct
tgtcagggggataactaccagaaaggtatacctgttgagactgattcagaggagcaaccctatttagaaatggatttatc
atcacctcaaacgagatatatcccggatgaggctgactttctgctggggatggccactgtgaataactgtgtttcctacc
gaaaccctgcagagggaacctggtacatccagtcactttgccagagcctgagagagcgatgtcctcgaggcgatgatatt
ctcaccatcctgactgaagtgaactatgaagtaagcaacaaggatgacaagaaaaacatggggaaacagatgcctcagcc
tactttcacactaagaaaaaaacttgtcttcccttctgattgaGGATCCAGATCTTATTAAAGCAGAACTTGTTTATTGC
AGCTTATAATGGTTACAAATAAAGCAATAGCATCACAAATTTCACAAATAAAGCATTTTTTTCACTGCATTCTAGTTGTG
GTTTGTCCAAACTCATCAATGTATCTTATCATGTCTGGTCGACTCTAGACTCTTCCGCTTCCTCGCTCACTGACTCGCTG
CGCTCGGTCGTTCGGCTGCGGCGAGCGGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCGGATACCTGTCCGCCTTTCT
CCCTTCGGGAAGCGTGGCGCTTTCTCAATGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTATGTAGGCGGTGCTACAGAGTTCTT
GAAGTGGTGGCCTAACTACGGCTACACTAGAAGGACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGCTTACCATCTGGCCCCAGTGCTGC
AATGATACCGCGAGACCCACGCTCACCGGCTCCAGATTTATCAGCAATAAACCAGCCAGCCGGAAGGGCCGAGCGCAGAA
GTGGTCCTGCAACTTTATCCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTTCTTCGGGGCGAA
AACTCTCAAGGATCTTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGGGCGACACGGAAATG
TTGAATACTCATACTCTTCTTTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCCACCTGACGTCTAAGAAACCATT
ATTATCATGACATTAACCTATAAAAATAGGCGTATCACGAGGCCCCTTTCGTCTCGCGCGTTTCGGTGATGACGGTGAAA
ACCTCTGACACATGCAGCTCCCGGAGACGGTCACAGCTTGTCTGTAAGCGGATGCCGGGAGCAGACAAGCCCGTCAGGGC
GCGTCAGCGGGTGTTGGCGGGTGTCGGGGCTGGCTTAACTATGCGGCATCAGAGCAGATTGTACTGAGAGTGCACCATAT
GCGGTGTGAAATACCGCACAGATGCGTAAGGAGAAAATACCGCATCAGGAAATTGTAAACGTTAATATTTTGTTAAAATT
CGCGTTAAATTTTTGTTAAATCAGCTCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAAGAACGTGGACTCCAACGTCAAAGGG
CGAAAAACCGTCTATCAGGGCGATGGCCCACTACGTGAACCATCACCCTAATCAAGTTTTTTGGGGTCGAGGTGCCGTAA
AGCACTAAATCGGAACCCTAAAGGGAGCCCCCGATTTAGAGCTTGACGGGGAAAGCCGGCGAACGTGGCGAGAAAGGAAG
GGAAGAAAGCGAAAGGAGCGGGCGCTAGGGCGCTGGCAAGTGTAGCGGTCACGCTGCGCGTAACCACCACACCCGCCGCG
CTTAATGCGCCGCTACAGGGCGCGTCGCGCCATTCGCCATTCAGGCTACGCAACTGTTGGGAAGGGCGATCGGTGCGGGC
CTCTTCGCTATTACGCCAGCTGGCGAAGGGGGGATGTGCTGCAAGGCGATTAAGTTGGGTAACGCCAGGGTTTTCCCAGT
CACGACGTTGTAAAACGACGGCCAGTGAATT

The sequence of DNA encoding the anti-apoptotic protein Bcl2 [SEQ ID NO:36] is shown below:

ATGGCGCACGCTGGGAGAACAGGGTACGATAACCGGGAGATAGTGATGAA
GTACATCCATTATAAGCTGTCGCAGAGGGGCTACGAGTGGGATGCGGGAG
ATGTGGGCGCCGCGCCCCCGGGGGCCGCCCCCGCACCGGGCATCTTCTCC
TCCCAGCCCGGGCACACGCCCCATCCAGCCGCATCCCGGGACCCGGTCGC
CAGGACCTCGCCGCTGCAGACCCCGGCTGCCCCCGGCGCCGCCGCGGGGC
CTGCGCTCAGCCCGGTGCCACCTGTGGTCCACCTGACCCTCCGCCAGGCC
GGCGACGACTTCTCCCGCCGCTACCGCCGCGACTTCGCCGAGATGTCCAG
CCAGCTGCACCTGACGCCCTTCACCGCGCGGGGACGCTTTGCCACGGTGG
TGGAGGAGCTCTTCAGGGACGGGGTGAACTGGGGGAGGATTGTGGCCTTC
TTTGAGTTCGGTGGGGTCATGTGTGTGGAGAGCGTCAACCGGGAGATGTC
GCCCCTGGTGGACAACATCGCCCTGTGGATGACTGAGTACCTGAACCGGC
ACCTGCACACCTGGATCCAGGATAACGGAGGCTGGGTAGGTGCACTTGGT
GATGTGAGTCTGGGCTGA

The amino acid sequence of Bcl2 [SEQ ID NO:37] is:

MAHAGRTGYD NREIVMKYIH YKLSQRGYEW DAGDVGAAPP GAAPAPGIFS SQPGHTPHPA
ASRDPVARTS PLQTPAAPGA AAGPALSPVP PVVHLTLRQA GDDFSRRYRR DFAEMSSQLH
LTPFTARGRF ATVVEELFRD GVNWGRIVAF FEFGGVMCVE SVNREMSPLV DNIALWMTEY
LNRHLHTWIQ DNGGWVGALG DVSLG

The PSG5 vector encoding Bcl2, designated PSG5-BCL2, has the sequence shown below [SEQ ID NO:38] (with the Bcl2 sequence in lower case, underscored):

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCAGATCTatggcgcacgctgggagaacagggtacgataaccgggagatagtgatgaagtacatccat
tataagctgtcgcagaggggctacgagtgggatgcgggagatgtgggcgccgcgcccccgggggccgcccccgcaccggg
catcttctcctcccagcccgggcacacgccccatccagccgcatcccgggacccggtcgccaggacctcgccgctgcaga
ccccggctgcccccggcgccgccgcggggcctgcgctcagcccggtgccacctgtggtccacctgaccctccgccaggcc
ggcgacgacttctcccgccgctaccgccgcgacttcgccgagatgtccagccagctgcacctgacgcccttcaccgcgcg
gggacgctttgccacggtggtggaggagctcttcagggacggggtgaactgggggaggattgtggccttctttgagttcg
gtggggtcatgtgtgtggagagcgtcaaccgggagatgtcgcccctggtggacaacatcgccctgtggatgactgagtac
ctgaaccggcacctgcacacctggatccaggataacggaggctgggtaggtgcacttggtgatgtgagtctgggctgaAG
ATCTTATTAAAGCAGAACTTGTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTATCATGTCTGGTCGACTCTAGACT
CTTCCGCTTCCTCGCTCACTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGCCAGCAAAAGGCCAGG6ACCGTAA
AAAGGCCGCGTTGCTGGCGTTTTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCAATGCTCACGCTGTAGGTATCTCAG
TTCGGTGTAGGTCGTTCGCTCCAAGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTACCAATGCTTAATCAGTG
AGGCACCTATCTCAGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCATGTTGTGCAAAAAAGC
GGTTAGCTCCTTCGGTCCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCTTTTTTCAATATTATTGAAGCATTTATCAGGG
TTATTGTCTCATGAGCGGATACATATTTGAATGTATTTAGAAAAATAAACAAATAGGGGTTCCGCGCACATTTCCCCGAA
AAGTGCCACCTGACGTCTAAGAAACCATTATTATCATGACATTAACCTATAAAAATAGGCGTATCACGAGGCCCCTTTCG
TCTCGCGCGTTTCGGTGATGACGGTGAAAACCTCTGACACATGCAGCTCCCGGAGACGGTCACAGCTTGTCTGTAAGCGG
ATGCCGGGAGCAGACAAGCCCGTCAGGGCGCGTCAGCGGGTGTTGGCGGGTGTCGGGGCTGGCTTAACTATGCGGCATCA
GAGCAGATTGTACTGAGAGTGCACCATATGCGGTGTGAAATACCGCACAGATGCGTAAGGAGAAAATACCGCATCAGGAA
ATTGTAAACGTTAATATTTTGTTAAAATTCGCGTTAAATTTTTGTTAAATCAGCTCATTTTTTAACCAATAGGCCGAAAT
CGGCAAAATCCCTTATAAATCAAAAGAATAGACCGAGATAGGGTTGAGTGTTGTTCCAGTTTGGAACAAGAGTCCACTAT
TAAAGAACGTGGACTCCAACGTCAAAGGGCGAAAAACCGTCTATCAGGGCGATGGCCCACTACGTGAACCATCACCCTAA
TCAAGTTTTTTGGGGTCGAGGTGCCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCTAGGGCGCTGGCAAGTGTAGCGGTCA
CGCTGCGCGTAACCACCACACCCGCCGCGCTTAATGCGCCGCTACAGGGCGCGTCGCGCCATTCGCCATTCAGGCTACGC
AACTGTTGGGAAGGGCGATCGGTGCGGGCCTCTTCGCTATTACGCCAGCTGGCGAAGGGGGGATGTGCTGCAAGGCGATT
AAGTTGGGTAACGCCAGGGTTTTCCCAGTCACGACGTTGTAAAACGACGGCCAGTGAATT

The pSG5-dn-caspase-8 vector [SEQ ID NO:39] encoding the dominant-negative caspase-8 is shown below with the dn-caspase-8 sequence in lower case, underscored:

| | |
|---|---|
| GTCGACTTCTGAGGCGGAAAGAACCAGCTGTGGAATGTGTGTCAGTTAGGGTGTGGAAAGTCCCCAGGCTCCCCAGCAGG | |
| CAGAAGTATGCAAAGCATGCATCTCAATTAGTCAGCAACCAGGTGTGGAAAGTCCCCAGGCTCCCCAGCAGGCAGAAGTA | |
| TGCAAAGCATGCATCTCAATTAGTCAGCAACCATAGTCCCGCCCCTAACTCCGCCCATCCCGCCCCTAACTCCGCCCAGT | |
| TCCGCCCATTCTCCGCCCCATGGCTGACTAATTTTTTTTATTTATGCAGAGGCCGAGGCCGCCTCGGCCTCTGAGCTATT | |
| CCAGAAGTAGTGAGGAGGCTTTTTTGGAGGCCTAGGCTTTTGCAAAAAGCTGGATCGATCCTGAGAACTTCAGGGTGAGT | |
| TTGGGGACCCTTGATTGTTCTTTCTTTTTCGCTATTGTAAAATTCATGTTATATGGAGGGGGCAAAGTTTTCAGGGTGTT | |
| GTTTAGAATGGGAAGATGTCCCTTGTATCACCATGGACCCTCATGATAATTTTGTTTCTTTCACTTTCTACTCTGTTGAC | |
| AACCATTGTCTCCTCTTATTTTCTTTTCATTTTCTGTAACTTTTTCGTTAAACTTTAGCTTGCATTTGTAACGAATTTTT | |
| AAATTCACTTTTGTTTATTTGTCAGATTGTAAGTACTTTCTCTAATCACTTTTTTTTCAAGGCAATCAGGGTATATTATA | |
| TTGTACTTCAGCACAGTTTTAGAGAACAATTGTTATAATTAAATGATAAGGTAGAATATTTCTGCATATAAATTCTGGCT | |
| GGCGTGGAAATATTCTTATTGGTAGAAACAACTACATCCTGGTCATCATCCTGCCTTTCTCTTTATGGTTACAATGATAT | |
| ACACTGTTTGAGATGAGGATAAAATACTCTGAGTCCAAACCGGGCCCCTCTGCTAACCATGTTCATGCCTTCTTCTTTTT | |
| CCTACAGCTCCTGGGCAACGTGCTGGTTATTGTGCTGTCTCATCATTTTGGCAAAGAATTGTAATACGACTCACTATAGG | |
| GCGAATTCGGATCCatggacttcagcagaaatctttatgatattggggaacaactggacagtgaagatctggcctccctc | |
| aagttcctgagcctggactacattccgcaaaggaagcaagaacccatcaaggatgccttgatgttattccagagactcca | |
| ggaaaagagaatgttggaggaaagcaatctgtccttcctgaaggagctgctcttccgaattaatagactggatttgctga | |
| ttacctacctaaacactagaaaggaggagatggaaagggaacttcagacaccaggcagggctcaaatttctgcctacagg | |
| gtcatgctctatcagatttcagaagaagtgagcagatcagaattgaggtcttttaagtttcttttgcaagaggaaatctc | |
| caaatgcaaactggatgatgacatgaacctgctggatattttcatagagatggagaagagggtcatcctgggagaaggaa | |
| agttggacatcctgaaaagagtctgtgcccaaatcaacaagagcctgctgaagataatcaacgactatgaagaattcagc | |
| aaaggggaggagttgtgtggggtaatgacaatctcggactctccaagagaacaggatagtgaatcacagactttggacaa | |
| agtttaccaaatgaaaagcaaacctcggggatactgtctgatcatcaacaatcacaattttgcaaaagcacgggagaaag | |
| tgcccaaacttcacagcattagggacaggaatggaacacacttggatgcaggggctttgaccacgacctttgaagagctt | |
| cattttgagatcaagccccacgatgactgcacagtagagcaaatctatgagattttgaaaatctaccaactcatggacca | |
| cagtaacatggactgcttcatctgctgtatcctctcccatggagacaagggcatcatctatggcactgatggacaggagg | |
| cccccatctatgagctgacatctcagttcactggtttgaagtgcccttcccttgctggaaaacccaaagtgttttttatt | |
| caggcttctcagggggataactaccagaaaggtatacctgttgagactgattcagaggagcaaccctatttagaaatgga | |
| tttatcatcacctcaaacgagatatatcccggatgaggctgactttctgctggggatggccactgtgaataactgtgttt | |
| cctaccgaaaccctgcagagggaacctggtacatccagtcactttgccagagcctgagagagcgatgtcctcgaggcgat | |
| gatattctcaccatcctgactgaagtgaactatgaagtaagcaacaaggatgacaagaaaaacatggggaaacagatgcc | |
| tcagcctactttcacactaagaaaaaaacttgtcttcccttctgattgaAGATCTTATTAAAGCAGAACTTGTTTATTGC | |
| AGCTTATAATGGTTACAAATAAAGCAATAGCATCACAAATTTCACAAATAAAGCATTTTTTTCACTGCATTCTAGTTGTG | |
| GTTTGTCCAAACTCATCAATGTATCTTATCATGTCTGGTCGACTCTAGACTCTTCCGCTTCCTCGCTCACTGACTCGCTG | |
| CGCTCGGTCGTTCGGCTGCGGCGAGCGGTATCAGCTCACTCAAAGGCGGTAATACGGTTATCCACAGAATCAGGGGATAA | |
| CGCAGGAAAGAACATGTGAGCAAAAGGCCAGCAAAAGGCCAGGAACCGTAAAAAGGCCGCGTTGCTGGCGTTTTTCCATA | |
| GGCTCCGCCCCCCTGACGAGCATCACAAAAATCGACGCTCAAGTCAGAGGTGGCGAAACCCGACAGGACTATAAAGATAC | |
| CAGGCGTTTCCCCCTGGAAGCTCCCTCGTGCGCTCTCCTGTTCCGACCCTGCCGCTTACCGGATACCTGTCCGCCTTTCT | |
| CCCTTCGGGAAGCGTGGCGCTTTCTCAATGCTCACGCTGTAGGTATCTCAGTTCGGTGTAGGTCGTTCGCTCCAAGCTGG | |
| GCTGTGTGCACGAACCCCCCGTTCAGCCCGACCGCTGCGCCTTATCCGGTAACTATCGTCTTGAGTCCAACCCGGTAAGA | |
| CACGACTTATCGCCACTGGCAGCAGCCACTGGTAACAGGATTAGCAGAGCGAGGTATGTAGGCGGTGCTACAGAGTTCTT | |
| GAAGTGGTGGCCTAACTACGGCTACACTAGAAGGACAGTATTTGGTATCTGCGCTCTGCTGAAGCCAGTTACCTTCGGAA | |
| AAAGAGTTGGTAGCTCTTGATCCGGCAAACAAACCACCGCTGGTAGCGGTGGTTTTTTTGTTTGCAAGCAGCAGATTACG | |
| CGCAGAAAAAAAGGATCTCAAGAAGATCCTTTGATCTTTTCTACGGGGTCTGACGCTCAGTGGAACGAAAACTCACGTTA | |
| AGGGATTTTGGTCATGAGATTATCAAAAAGGATCTTCACCTAGATCCTTTTAAATTAAAAATGAAGTTTTAAATCAATCT | |
| AAAGTATATATGAGTAAACTTGGTCTGACAGTTACCAATGCTTAATCAGTGAGGCACCTATCTCAGCGATCTGTCTATTT | |
| CGTTCATCCATAGTTGCCTGACTCCCCGTCGTGTAGATAACTACGATACGGGAGGGCTTACCATCTGGCCCCAGTGCTGC | |
| AATGATACCGCGAGACCCACGCTCACCGGCTCCAGATTTATCAGCAATAAACCAGCCAGCCGGAAGGGCCGAGCGCAGAA | |
| GTGGTCCTGCAACTTTATCCGCCTCCATCCAGTCTATTAATTGTTGCCGGGAAGCTAGAGTAAGTAGTTCGCCAGTTAAT | |
| AGTTTGCGCAACGTTGTTGCCATTGCTACAGGCATCGTGGTGTCACGCTCGTCGTTTGGTATGGCTTCATTCAGCTCCGG | |
| TTCCCAACGATCAAGGCGAGTTACATGATCCCCCATGTTGTGCAAAAAAGCGGTTAGCTCCTTCGGTCCTCCGATCGTTG | |
| TCAGAAGTAAGTTGGCCGCAGTGTTATCACTCATGGTTATGGCAGCACTGCATAATTCTCTTACTGTCATGCCATCCGTA | |
| AGATGCTTTTCTGTGACTGGTGAGTACTCAACCAAGTCATTCTGAGAATAGTGTATGCGGCGACCGAGTTGCTCTTGCCC | |
| GGCGTCAATACGGGATAATACCGCGCCACATAGCAGAACTTTAAAAGTGCTCATCATTGGAAAACGTTCTTCGGGGCGAA | ~ |
| AACTCTCAAGGATCTTACCGCTGTTGAGATCCAGTTCGATGTAACCCACTCGTGCACCCAACTGATCTTCAGCATCTTTT | |
| ACTTTCACCAGCGTTTCTGGGTGAGCAAAAACAGGAAGGCAAAATGCCGCAAAAAAGGGAATAAGGGCGACACGGAAATG | ~ |
| TTGAATACTCATACTCTTCTTTTTTCAATATTATTGAAGCATTTATCAGGGTTATTGTCTCATGAGCGGATACATATTTG | |
| AATGTATTTAGAAAAATAAACAAATAGGGGTTCCGCGCACATTTCCCCGAAAAGTGCCACCTGACGTCTAAGAAACCATT | |
| ATTATCATGACATTAACCTATAAAAATAGGCGTATCACGAGGCCCCTTTCGTCTCGCGCGTTTCGGTGATGACGGTGAAA | |
| ACCTCTGACACATGCAGCTCCCGGAGACGGTCACAGCTTGTCTGTAAGCGGATGCCGGGAGCAGACAAGCCCGTCAGGGC | |
| GCGTCAGCGGGTGTTGGCGGGTGTCGGGGCTGGCTTAACTATGCGGCATCAGAGCAGATTGTACTGAGAGTGCACCATAT | |
| GCGGTGTGAAATACCGCACAGATGCGTAAGGAGAAAATACCGCATCAGGAAATTGTAAACGTTAATATTTTGTTAAAATT | |
| CGCGTTAAATTTTTGTTAAATCAGCTCATTTTTTAACCAATAGGCCGAAATCGGCAAAATCCCTTATAAATCAAAAGAAT | |
| AGACCGAGATAGGGTTGAGTGTTGTTCCAGTTTGGAACAAGAGTCCACTATTAAAGAACGTGGACTCCAACGTCAAAGGG | |
| CGAAAAACCGTCTATCAGGGCGATGGCCCACTACGTGAACCATCACCCTAATCAAGTTTTTTGGGGTCGAGGTGCCGTAA | |
| AGCACTAAATCGGAACCCTAAAGGGAGCCCCCGATTTAGAGCTTGACGGGGAAAGCCGGCGAACGTGGCGAGAAAGGAAG | |
| GGAAGAAAGCGAAAGGAGCGGGCGCTAGGGCGCTGGCAAGTGTAGCGGTCACGCTGCGCGTAACCACCACACCCGCCGCG | |
| CTTAATGCGCCGCTACAGGGCGCGTCGCGCCATTCGCCATTCAGGCTACGCAACTGTTGGGAAGGGCGATCGGTGCGGGC | |
| CTCTTCGCTATTACGCCAGCTGGCGAAGGGGGGATGTGCTGCAAGGCGATTAAGTTGGGTAACGCCAGGGTTTTCCCAGT | |
| CACGACGTTGTAAAACGACGGCCAGTGAATT | |

The amino acid sequence of dn-caspase-8 [SEQ ID NO:40] is:

MDFSRNLYDI GEQLDSEDLA SLKFLSLDYI PQRKQEPIKD ALMLFQRLQE KRMLEESNLS
FLKELLFRIN RLDLLITYLN TRKEEMEREL QTPGRAQISA YRVMLYQISE EVSRSELRSF
KFLLQEEISK CKLDDDMNLL DIFIEMEKRV ILGEGKLDIL KRVCAQINKS LLKIINDYEE
FSKGEELCGV MTISDSPREQ DSESQTLDKV YQMKSKPRGY CLIINNHNFA KAREKVPKLH
SIRDRNGTHL DAGALTTTFE ELHFEIKPHD DCTVEQIYEI LKIYQLMDHS NMDCFICCIL
SHGDKGIIYG TDGQEAPIYE LTSQFTGLKC PSLAGKPKVF FIQASQGDNY QKGIPVETDS
EEQPYLEMDL SSPQTRYIPD EADFLLGMAT VNNCVSYRNP AEGTWYIQSL CQSLRERCPR
GDDILTILTE VNYEVSNKDD KKNMGKQMPQ PTFTLRKKLV FPSD

The pSG5-dn-caspase-9 vector [SEQ ID NO:41] encoding the dominant-negative caspase-9 is shown below with the dn-caspase-9 sequence in lower case, underscored:

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCatggacgaagcggatcggcggctcctgcggcggtgccggctgcggctggtggaagagctgcaggtg
gaccagctctgggacgccctgctgagccgcgagctgttcaggccccatatgatcgaggacatccagcgggcaggctctgg
atctcggcgggatcaggccaggcagctgatcatagatctggagactcgagggagtcaggctcttcctttgttcatctcct
gcttagaggacacaggccaggacatgctggcttcgtttctgcgaactaacaggcaagcagcaaagttgtcgaagccaacc
ctagaaaaccttaccccagtggtgctcagaccagagattcgcaaaccagaggttctcagaccggaaacacccagaccagt
ggacattggttctggaggatttggtgatgtcggtgctcttgagagtttgaggggaaatgcagatttggcttacatcctga
gcatggagccctgtggccactgcctcattatcaacaatgtgaacttctgccgtgagtccgggctccgcacccgcactggc
tccaacatcgactgtgagaagttgcggcgtcgcttctcctcgctgcatttcatggtggaggtgaagggcgacctgactgc
caagaaaatggtgctggctttgctggagctggcgcagcaggaccacggtgctctggactgctgcgtggtggtcattctct
ctcacggctgtcaggccagccacctgcagttcccaggggctgtctacggcacagatggatgccctgtgtcggtcgagaag
attgtgaacatcttcaatgggaccagctgccccagcctgggagggaagcccaagctctttttcatccaggcctctggtgg
ggagcagaaagaccatgggtttgaggtggcctccacttcccctgaagacgagtcccctggcagtaaccccgagccagatg
ccaccccgttccaggaaggtttgaggaccttcgaccagctggacgccatatctagtttgcccacacccagtgacatcttt
gtgtcctactctactttcccaggttttgtttcctggagggaccccaagagtggctcctggtacgttgagaccctggacga
catctttgagcagtgggctcactctgaagacctgcagtccctcctgcttagggtcgctaatgctgtttcggtgaaaggga
tttataaacagatgcctggttgctttaatttcctccggaaaaaacttttctttaaaacatcataaAGATCTTATTAAAGC
AGAACTTGTTTATTGCAGCTTATAATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGGTCGACTCTAGACTCTTCCGCTTCCTC
GCTCACTGACTCGCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCAATGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTGACGCTCAGTGGA
ACGAAAACTCACGTTAAGGGATTTTGGTCATGAGATTATCAAAAAGGATCTTCACCTAGATCCTTTTAAATTAAAAATGA
AGTTTTAAATCAATCTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGTTTGGTATGG
CTTCATTCAGCTCCGGTTCCCAACGATCAAGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCTTTTTTCAATATTATTGAAGCATTTATCAGGGTTATTGTCTCATG
AGCGGATACATATTTGAATGTATTTAGAAAAATAAACAAATAGGGGTTCCGCGCACATTTCCCCGAAAAGTGCCACCTGA
CGTCTAAGAAACCATTATTATCATGACATTAACCTATAAAAATAGGCGTATCACGAGGCCCCTTTCGTCTCGCGCGTTTC
GGTGATGACGGTGAAAACCTCTGACACATGCAGCTCCCGGAGACGGTCACAGCTTGTCTGTAAGCGGATGCCGGGAGCAG
ACAAGCCCGTCAGGGCGCGTCAGCGGGTGTTGGCGGGTGTCGGGGCTGGCTTAACTATGCGGCATCAGAGCAGATTGTAC
TGAGAGTGCACCATATGCGGTGTGAAATACCGCACAGATGCGTAAGGAGAAAATACCGCATCAGGAAATTGTAAACGTTA
ATATTTTGTTAAAATTCGCGTTAAATTTTTGTTAAATCAGCTCATTTTTTAACCAATAGGCCGAAATCGGCAAAATCCCT
TATAAATCAAAAGAATAGACCGAGATAGGGTTGAGTGTTGTTCCAGTTTGGAACAAGAGTCCACTATTAAAGAACGTGGA
CTCCAACGTCAAAGGGCGAAAAACCGTCTATCAGGGCGATGGCCCACTACGTGAACCATCACCCTAATCAAGTTTTTTGG
GGTCGAGGTGCCGTAAAGCACTAAATCGGAACCCTAAAGGGAGCCCCCGATTTAGAGCTTGACGGGGAAAGCCGGCGAAC
GTGGCGAGAAAGGAAGGGAAGAAAGCGAAAGGAGCGGGCGCTAGGGCGCTGGCAAGTGTAGCGGTCACGCTGCGCGTAAC
CACCACACCCGCCGCGCTTAATGCGCCGCTACAGGGCGCGTCGCGCCATTCGCCATTCAGGCTACGCAACTGTTGGGAAG
GGCGATCGGTGCGGGCCTCTTCGCTATTACGCCAGCTGGCGAAGGGGGGATGTGCTGCAAGGCGATTAAGTTGGGTAACG
CCAGGGTTTTCCCAGTCACGACGTTGTAAAACGACGGCCAGTGAATT

The amino acid sequence of dn-caspase-9 [SEQ ID NO:42] is:

MDEADRRLLR RCRLRLVEEL QVDQLWDALL SRELFRPHMI EDIQRAGSGS RRDQARQLII
DLETRGSQAL PLFISCLEDT GQDMLASFLR TNRQAAKLSK PTLENLTPVV LRPEIRKPEV
LRPETPRPVD IGSGGFGDVG ALESLRGNAD LAYILSMEPC GHCLIINNVN FCRESGLRTR
TGSNIDCEKL RRRFSSLHFM VEVKGDLTAK KMVLALLELA QQDHGALDCC VVVILSHGCQ
ASHLQFPGAV YGTDGCPVSV EKIVNIFNGT SCPSLGGKPK LFFIQASGGE QKDHGFEVAS
TSPEDESPGS NPEPDATPFQ EGLRTFDQLD AISSLPTPSD IFVSYSTFPG FVSWRDPKSG
SWYVETLDDI FEQWAHSEDL QSLLLRvANA VSVKGIYKQM PGCFNFLRKK LFFKTS

The nucleotide sequence of murine serine protease inhibitor 6 (SPI-6, deposited in GENEBANK as NM_{—}009256 is shown below [SEQ ID NO:43]

```
      1 gaattccggg ctggattgag aagccgcaac tgtgactctg catcatgaat actctgtctg
    61 aaggaaatgg cacctttgcc atccatcttt tgaagatgct atgtcaaagc aacccttcca
  121 aaaatgtatg ttattctcct gcgagcatct cctctgctct agctatggtt ctcttgggtg
  181 caaagggaca gacggcagtc cagatatctc aggcacttgg tttgaataaa gaggaaggca
  241 tccatcaggg tttccagttg cttctcagga agctgaacaa gccagacaga aagtactctc
  301 ttagagtggc caacaggctc tttgcagaca aaacttgtga agtcctccaa acctttaagg
  361 agtcctctct tcacttctat gactcagaga tggagcagct ctcctttgct gaagaagcag
  421 aggtgtccag gcaacacata aacacatggg tctccaaaca aactgaaggt aaaattccag
  481 agttgttgtc aggtggctcc gtcgattcag aaaccaggct ggttctcatc aatgccttat
  541 attttaaagg aaagtggcat caaccattta acaaagagta cacaatggac atgcccttta
  601 aaataaacaa ggatgagaaa aggccagtgc agatgatgtg tcgtgaagac acatataacc
  661 tcgcctatgt gaaggaggtg caggcgcaag tgctggtgat gccatatgaa ggaatggagc
  721 tgagcttggt ggttctgctc ccagatgagg gtgtggacct cagcaaggtg gaaaacaatc
  781 tcacttttga gaagttaaca gcctggatgg aagcagattt tatgaagagc actgatgttg
  841 aggttttcct tccaaaattt aaactccaag aggattatga catggagtct ctgtttcagc
  901 gcttgggagt ggtggatgtc ttccaagagg acaaggctga cttatcagga atgtctccag
  961 agagaaacct gtgtgtgtcc aagtttgttc accagagtgt agtggagatc aatgaggaag
1021 gcacagaggc tgcagcagcc tctgccatca tagaattttg ctgtgcctct tctgtcccaa
1081 cattctgtgc tgaccacccc ttccttttct tcatcaggca caacaaagca aacagcatcc
1141 tgttctgtgg caggttctca tctccataaa gacacatata ctacacaggg agagttctct
1201 cttcagtatc cctaccactc ctacagctct gtcaagatgg gcaagtaggg ggaagtcatg
1261 ttctaagatg aagacacttt ccttctctgt cagcctgatc ttataatgcc tgcattcaac
1321 tctccctgtc ttgaatgcat ctatgccctt taccaggtta tgtctaatga tgccaaatac
1381 cttctgctat gctattgatt gatagcctag ccagtaattt atagccagtt agaactgact
1441 tgactgtgca agaatgctat aatggagcta gagagaaggc acaaacacta ggaaaggttg
1501 ctgtttttgc agaggacaca gggacatttc ccaccactca catggctgct tacaacctct
1561 ggaaattcca gtttctgtcc atgacttgat tcctttcttt ggcttctact ggctccagca
1621 tcctgcacat acatgtatcg tcattcagtt acacacaaac aagtaaaatt ttaaaaataa
1681 ataaaaattt aaagagagag tctaaaattt tagtaatggt tagataatag ctgctattgt
1741 gcctttttca ggttttaatg tcattattct tgtgtataaa gtcaataatt tataggaaaa
1801 catcagtgcc ccggaattc
```

The amino acid sequence of the SPI-6 protein [SEQ ID NO:44] is:

MNTLSEGNGTFAIHLLKMLCQSNPSKNVCYSPASISSALAMVLLGAKGQTAVQISQALGLNKEEGIHQGFQLL
LRKLNKPDRKYSLRVANRLFADKTCEVLQTFKESSLHFYDSEMEQLSFAEEAEVSRQHINTWVSKQTEGKIPE
LLSGGSVDSETRLVLINALYFKGKWHQPFMKEYTMDMPFKINKDEKRPVQMMCREDTYNLAYVKEVQAQVLVM
PYEGMELSLVVLLPDEGVDLSKVENNLTFEKLTAWMEADFMKSTDVEVFLPKFKLQEDYDMESLFQRLGVVDV
FQEDKADLSGMSPERNLCVSKFVHQSVVEINEEGTEAAAASAIIEFCCASSVPTFCADHPFLFFIRHNKANSI
LFCGRFSSP

The nucleic acid sequence of the mutant SPI-6 (mtSPI6) is shown below [SEQ ID NO:45]

atgaatactctgtctgaaggaaatggcacctttgccatccatcttttgaagatgctatgtcaaagcaaccctt
ccaaaaatgtatgttattctcctgcgagcatctcctctgctctagctatggttctcttgggtgcaaagggaca
gacggcagtccagatatctcaggcacttggtttgaataaagaggaaggcatccatcagggtttccagttgctt
ctcaggaagctgaacaagccagacagaaagtactctcttagagtggccaacaggctctttgcagacaaaactt
gtgaagtcctccaaacctttaaggagtcctctcttcacttctatgactcagagatggagcagctctcctttgc
tgaagaagcagaggtgtccaggcaacacataaacacatgggtctccaaacaaactgaaggtaaaattccagag
ttgttgtcaggtggctccgtcgattcagaaaccaggctggttctcatcaatgccttatattttaaaggaaagt
ggcatcaaccatttaacaaagagtacacaatggacatgccctttaaaataaacaaggatgagaaaaggccagt
gcagatgatgtgtcgtgaagacacatataacctcgcctatgtgaaggaggtgcaggcgcaagtgctggtgatg
ccatatgaaggaatggagctgagcttggtggttctgctcccagatgagggtgtggacctcagcaaggtggaaa
acaatctcacttttgagaagttaacagcctggatggaagcagattttatgaagagcactgatgttgaggtttt
ccttccaaaatttaaactccaagaggattatgacatggagtctctgtttcagcgcttgggagtggtggatgtc
ttccaagaggacaaggctgacttatcaggaatgtctccagagagaaacctgtgtgtgtccaagtttgttcacc
agagtgtagtggagatcaatgaggaaggcagagaggctgcagcagcctctgccatcatagaattttgctgtgc
ctcttctgtcccaacattctgtgctgaccaccccttccttttcttcatcaggcacaacaaagcaaacagcatc
ctgttctgtggcaggttctcatctccataa

The amino acid sequence of the mutant SPI-6 protein (mtSPI-6) [SEQ ID NO:46] is:

MNTLSEGNGT FAIHLLKMLC QSNPSKNVCY SPASISSALA MVLLGAKGQT AVQISQALGL
NKEEGIHQGF QLLLRKLNKP DRKYSLRVAN RLFADKTCEV LQTFKESSLH FYDSEMEQLS
FAEEAEVSRQ HINTWVSKQT EGKIPELLSG GSVDSETRLV LINALYFKGK WHQPFNKEYT
MDMPFKINKD EKRPVQMMCR EDTYNLAYVK EVQAQVLVMP YEGMELSLVV LLPDEGVDLS
KVENNLTFEK LTAWMEADFM KSTDVEVFLP KFKLQEDYDM ESLFQRLGVV DVFQEDKADL
SGMSPERNLC VSKFVHQSVV EINEEGREAA AASAIIEFCC ASSVPTFCAD HPFLFFIRHN
KANSILFCGR FSSP

The sequence of the pcDNA3-Spi6 vector [SEQ ID NO:47] is shown below with the SPI-6 in lower case, underscored:

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCa
tgaatactctgtctgaaggaaatggcacctttgccatccatcttttgaagatgctatgtcaaagcaacccttccaaaaat
gtatgttattctcctgcgagcatctcctctgctctagctatggttctcttgggtgcaaagggacagacggcagtccagat
atctcaggcacttggtttgaataaagaggaaggcatccatcagggtttccagttgcttctcaggaagctgaacaagccag
acagaaagtactctcttagagtggccaacaggctctttgcagacaaaacttgtgaagtcctccaaacctttaaggagtcc
tctcttcacttctatgactcagagatggagcagctctcctttgctgaagaagcagaggtgtccaggcaacacataaacac
atgggtctccaaacaaactgaaggtaaaattccagagttgttgtcaggtggctccgtcgattcagaaaccaggctggttc
tcatcaatgccttatattttaaaggaaagtggcatcaaccatttaacaaagagtacacaatggacatgccctttaaaata
aacaaggatgagaaaaggccagtgcagatgatgtgtcgtgaagacacatataacctcgcctatgtgaaggaggtgcaggc
gcaagtgctggtgatgccatatgaaggaatggagctgagcttggtggttctgctcccagatgagggtgtggacctcagca
aggtggaaaacaatctcacttttgagaagttaacagcctggatggaagcagattttatgaagagcactgatgttgaggtt
ttccttccaaaatttaaactccaagaggattatgacatggagtctctgtttcagcgcttgggagtggtggatgtcttcca
agaggacaaggctgacttatcaggaatgtctccagagagaaacctgtgtgtgtccaagtttgttcaccagagtgtagtgg
agatcaatgaggaaggcacagaggctgcagcagcctctgccatcatagaattttgctgtgcctcttctgtcccaacattc
tgtgctgaccaccccttccttttcttcatcaggcacaacaaagcaaacagcatcctgttctgtggcaggttctcatctcc
aGGATCCGAGCTCGGTACCAAGCTTAAGTTTAAACCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGGAGGATTGGGAAGACA
ATAGCAGGCATGCTGGGGATGCGGTGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGTGACCGCTACACTTGCCAGCGCCCT
AGCGCCCGCTCCTTTCGCTTTCTTCCCTTCCTTTCTCGCCACGTTCGCCGGCTTTCCCCGTCAAGCTCTAAATCGGGGCA
TCCCTTTAGGGTTCCGATTTAGTGCTTTACGGCACCTCGACCCCAAAAAACTTGATTAGGGTGATGGTTCACGTAGTGGG
CCATCGCCCTGATAGACGGTTTTTCGCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGGGGATTTCGGCCTATTGGTTAAAAAATG
AGCTGATTTAACAAAAATTTAACGCGAATTAATTCTGTGGAATGTGTGTCAGTTAGGGTGTGGAAAGTCCCCAGGCTCCC
CAGGCAGGCAGAAGTATGCAAAGCATGCATCTCAATTAGTCAGCAACCAGGTGTGGAAAGTCCCCAGGCTCCCCAGCAGG
CAGAAGTATGCAAAGCATGCATCTCAATTAGTCAGCAACCATAGTCCCGCCCCTAACTCCGCCCATCCCGCCCCTAACTC
CGCCCAGTTCCGCCCATTCTCCGCCCCATGGCTGACTAATTTTTTTTATTTATGCAGAGGCCGAGGCCGCCTCTGCCTCT
GAGCTATTCCAGAAGTAGTGAGGAGGCTTTTTTGGAGGCCTAGGCTTTTGCAAAAAGCTCCCGGGAGCTTGTATATCCAT
TTTCGGATCTGATCAAGAGACAGGATGAGGATCGTTTCGCATGATTGAACAAGATGGATTGCACGCAGGTTCTCCGGCCG
CTTGGGTGGAGAGGCTATTCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCAGGACGAGGCAGCGCGGCT
ATCGTGGCTGGCCACGACGGGCGTTCCTTGCGCAGCTGTGCTCGACGTTGTCACTGAAGCGGGAAGGGACTGGCTGCTAT
TGGGCGAAGTGCCGGGGCAGGATCTCCTGTCATCTCACCTTGCTCCTGCCGAGAAAGTATCCATCATGGCTGATGCAATG
CGGCGGCTGCATACGCTTGATCCGGCTACCTGCCCATTCGACCACCAAGCGAAACATCGCATCGAGCGAGCACGTACTCG
GATGGAAGCCGGTCTTGTCGATCAGGATGATCTGGACGAAGAGCATCAGGGGCTCGCGCCAGCCGAACTGTTCGCCAGGC
TCAAGGCGCGCATGCCCGACGGCGAGGATCTCGTCGTGACCCATGGCGATGCCTGCTTGCCGAATATCATGGTGGAAAAT
GGCCGCTTTTCTGGATTCATCGACTGTGGCCGGCTGGGTGTGGCGGACCGCTATCAGGACATAGCGTTGGCTACCCGTGA
TATTGCTGAAGAGCTTGGCGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAGCGGGACTCTGGGGTTCGAAATGACCGACCAAGCGACGCCCAACC
TGCCATCACGAGATTTCGATTCCACCGCCGCCTTCTATGAAAGGTTGGGCTTCGGAATCGTTTTCCGGGACGCCGGCTGG
ATGATCCTCCAGCGCGGGGATCTCATGCTGGAGTTCTTCGCCCACCCCAACTTGTTTATTGCAGCTTATAATGGTTACAA
ATAAAGCAATAGCATCACAAATTTCACAAATAAAGCATTTTTTTCACTGCATTCTAGTTGTGGTTTGTCCAAACTCATCA
ATGTATCTTATCATGTCTGTATACCGTCGACCTCTAGCTAGAGCTTGGCGTAATCATGGTCATAGCTGTTTCCTGTGTGA
AATTGTTATCCGCTCACAATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCCTTTCTCCCTTCGGGAAG
CGTGGCGCTTTCTCAATGCTCACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTCTTGAAGTGGTGGCC
TAACTACGGCTACACTAGAAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAAAGTATATATG
AGTAAACTTGGTCTGACAGTTACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTTCTTCGGGGCGAAAACTCTCAAGGA
TCTTACCGCTGTTGAGATCCAGTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTTGAATGTATTTAGA
AAAATAAACAAATAGGGGTTCCGCGCACATTTCCCCGAAAAGTGCCACCTGACGTC

The sequence of the mutant vector pcDNA3-mtSpi6 vector [SEQ ID NO:48] is the same as that above, except that the mtSPI-6 sequence is inserted in the same location in place of the wilt type SPI-6.

Vectors Encoding of Pro-Apoptotic Proteins

The pSG5-caspase-3 vector [SEQ ID NO:90] is shown below with the caspase-3 sequence in lower case, underscored:

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCatggagaacactgaaaactcagtggattcaaaatccattaaaaatttggaaccaaagatcatacat
ggaagcgaatcaatggactctggaatatccctggacaacagttataaaatggattatcctgagatgggtttatgtataat
aattaataataagaattttcataaaagcactggaatgacatctcggtctggtacagatgtcgatgcagcaaacctcaggg
aaacattcagaaacttgaaatatgaagtcaggaataaaaatgatcttacacgtgaagaaattgtggaattgatgcgtgat
gtttctaaagaagatcacagcaaaaggagcagttttgtttgtgtgcttctgagccatggtgaagaaggaataatttttgg
aacaaatggacctgttgacctgaaaaaaataacaaactttttcagaggggatcgttgtagaagtctaactggaaaaccca
aacttttcattattcaggcctgccgtggtacagaactggactgtggcattgagacagacagtggtgttgatgatgacatg
gcgtgtcataaaataccagtggaggccgacttcttgtatgcatactccacagcacctggttattattcttggcgaaattc
aaaggatggctcctggttcatccagtcgctttgtgccatgctgaaacagtatgccgacaagcttgaatttatgcacattc
ttacccgggttaaccgaaaggtggcaacagaatttgagtccttttcctttgacgctacttttcatgcaaagaaacagatt
ccatgtattgtttccatgctcacaaaagaactctatttttatcactaaAGATCTTATTAAAGCAGAACTTGTTTATTGCA
GCTTATAATGGTTACAAATAAAGCAATAGCATCACAAATTTCACAAATAAAGCATTTTTTTCACTGCATTCTAGTTGTGG
TTTGTCCAAACTCATCAATGTATCTTATCATGTCTGGTCGACTCTAGACTCTTCCGCTTCCTCGCTCACTGACTCGCTGC
GCTCGGTCGTTCGGCTGCGGCGAGCGGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCGGATACCTGTCCGCCTTTCTC
CCTTCGGGAAGCGTGGCGCTTTCTCAATGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGAGTTCTTG
AAGTGGTGGCCTAACTACGGCTACACTAGAAGGACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGGCGACCGAGTTGCTCTTGCCCG
GCGTCAATACGGGATAATACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAGGGAATAAGGGCGACACGGAAATGT
TGAATACTCATACTCTTCTTTTTTCAATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGTGCCACCTGACGTCTAAGAAACCATTA
TTATCATGACATTAACCTATAAAAATAGGCGTATCACGAGGCCCCTTTCGTCTCGCGCGTTTCGGTGATGACGGTGAAAA
CCTCTGACACATGCAGCTCCCGGAGACGGTCACAGCTTGTCTGTAAGCGGATGCCGGGAGCAGACAAGCCCGTCAGGGCG
CGTCAGCGGGTGTTGGCGGGTGTCGGGGCTGGCTTAACTATGCGGCATCAGAGCAGATTGTACTGAGAGTGCACCATATG
CGGTGTGAAATACCGCACAGATGCGTAAGGAGAAAATACCGCATCAGGAAATTGTAAACGTTAATATTTTGTTAAAATTC
GCGTTAAATTTTTGTTAAATCAGCTCATTTTTTAACCAATAGGCCGAAATCGGCAAAATCCCTTATAAATCAAAAGAATA
GACCGAGATAGGGTTGAGTGTTGTTCCAGTTTGGAACAAGAGTCCACTATTAAAGAACGTGGACTCCAACGTCAAAGGGC
GAAAAACCGTCTATCAGGGCGATGGCCCACTACGTGAACCATCACCCTAATCAAGTTTTTTGGGGTCGAGGTGCCGTAAA
GCACTAAATCGGAACCCTAAAGGGAGCCCCCGATTTAGAGCTTGACGGGGAAAGCCGGCGAACGTGGCGAGAAAGGAAGG
GAAGAAAGCGAAAGGAGCGGGCGCTAGGGCGCTGGCAAGTGTAGCGGTCACGCTGCGCGTAACCACCACACCCGCCGCGC
TTAATGCGCCGCTACAGGGCGCGTCGCGCCATTCGCCATTCAGGCTACGCAACTGTTGGGAAGGGCGATCGGTGCGGGCC
TCTTCGCTATTACGCCAGCTGGCGAAGGGGGGATGTGCTGCAAGGCGATTAAGTTGGGTAACGCCAGGGTTTTCCCAGTC
ACGACGTTGTAAAACGACGGCCAGTGAATT

The amino acid sequence of Caspase-3 (SEQ ID NO:49) is:

MENTENSVDS KSIKNLEPKI IHGSESMDSG ISLDNSYKMD YPEMGLCIII NNKNFHKSTG
MTSRSGTDVD AANLRETFRN LKYEVRNKND LTREEIVELM RDVSKEDHSK RSSFVCVLLS
HGEEGIIFGT NGPVDLKKIT NFFRGDRCRS LTGKPKLFII QACRGTELDC GIETDSGVDD
DMACHKIPVE ADFLYAYSTA PGYYSWRNSK DGSWFIQSLC AMLKQYADKL EFMHILTRVN
RKVATEFESF SFDATFHAKK QIPCIVSMLT KELYFYH

The vector encoding mutant caspase-3, pSG5-mt caspase-3 [SEQ ID NO:50] is the same as that of the wild type, except that the mutant caspase-3 sequence is inserted in the same location as the wild type sequence above (indicated in lower case, underscored.

GTCGACTTCTGAGGCGGAAAGAACCAGCTGTGGAATGTGTGTCAGTTAGGGTGTGGAAAGTCCCCAGGCTCCCCAGCAGG
CAGAAGTATGCAAAGCATGCATCTCAATTAGTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTGGATCGATCCTGAGAACTTCAGGGTGAGT
TTGGGGACCCTTGATTGTTCTTTCTTTTTCGCTATTGTAAAATTCATGTTATATGGAGGGGGCAAAGTTTTCAGGGTGTT
GTTTAGAATGGGAAGATGTCCCTTGTATCACCATGGACCCTCATGATAATTTTGTTTCTTTCACTTTCTACTCTGTTGAC
AACCATTGTCTCCTCTTATTTTCTTTTCATTTTCTGTAACTTTTTCGTTAAACTTTAGCTTGCATTTGTAACGAATTTTT
AAATTCACTTTTGTTTATTTGTCAGATTGTAAGTACTTTCTCTAATCACTTTTTTTTCAAGGCAATCAGGGTATATTATA
TTGTACTTCAGCACAGTTTTAGAGAACAATTGTTATAATTAAATGATAAGGTAGAATATTTCTGCATATAAATTCTGGCT
GGCGTGGAAATATTCTTATTGGTAGAAACAACTACATCCTGGTCATCATCCTGCCTTTCTCTTTATGGTTACAATGATAT
ACACTGTTTGAGATGAGGATAAAATACTCTGAGTCCAAACCGGGCCCCTCTGCTAACCATGTTCATGCCTTCTTCTTTTT
CCTACAGCTCCTGGGCAACGTGCTGGTTATTGTGCTGTCTCATCATTTTGGCAAAGAATTGTAATACGACTCACTATAGG
GCGAATTCGGATCCatggagaacactgaaaactcagtggattcaaaatccattaaaaatttggaaccaaagatcatacat
ggaagcgaatcaatggactctggaatatccctggacaacagttataaaatggattatcctgagatgggtttatgtataat
aattaataataagaattttcataaaagcactggaatgacatctcggtctggtacagatgtcgatgcagcaaacctcaggg
aaacattcagaaacttgaaatatgaagtcaggaataaaaatgatcttacacgtgaagaaattgtggaattgatgcgtgat
gtttctaaagaagatcacagcaaaaggagcagttttgtttgtgtgcttctgagccatggtgaagaaggaataatttttgg
aacaaatggacctgttgacctgaaaaaaataacaaactttttcagaggggatcgttgtagaagtctaactggaaaaccca
aacttttcattattcaggccggccgtggtacagaactggactgtggcattgagacagacagtggtgttgatgatgacatg
gcgtgtcataaaataccagtggaggccgacttcttgtatgcatactccacagcacctggttattattcttggcgaaattc
aaaggatggctcctggttcatccagtcgctttgtgccatgctgaaacagtatgccgacaagcttgaatttatgcacattc
ttacccgggttaaccgaaaggtggcaacagaatttgagtccttttcctttgacgctacttttcatgcaaagaaacagatt
ccatgtattgtttccatgctcacaaaagaactctatttttatcactaaAGATCTTATTAAAGCAGAACTTGTTTATTGCA
GCTTATAATGGTTACAAATAAAGCAATAGCATCACAAATTTCACAAATAAAGCATTTTTTTCACTGCATTCTAGTTGTGG
TTTGTCCAAACTCATCAATGTATCTTATCATGTCTGGTCGACTCTAGACTCTTCCGCTTCCTCGCTCACTGACTCGCTGC
GCTCGGTCGTTCGGCTGCGGCGAGCGGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCGGATACCTGTCCGCCTTTCTC
CCTTCGGGAAGCGTGGCGCTTTCTCAATGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGAGTTCTTG
AAGTGGTGGCCTAACTACGGCTACACTAGAAGGACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGGCGACCGAGTTGCTCTTGCCCG
GCGTCAATACGGGATAATACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAGGGAATAAGGGCGACACGGAAATGT
TGAATACTCATACTCTTCTTTTTTCAATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGTGCCACCTGACGTCTAAGAAACCATTA
TTATCATGACATTAACCTATAAAAATAGGCGTATCACGAGGCCCCTTTCGTCTCGCGCGTTTCGGTGATGACGGTGAAAA
CCTCTGACACATGCAGCTCCCGGAGACGGTCACAGCTTGTCTGTAAGCGGATGCCGGGAGCAGACAAGCCCGTCAGGGCG
CGTCAGCGGGTGTTGGCGGGTGTCGGGGCTGGCTTAACTATGCGGCATCAGAGCAGATTGTACTGAGAGTGCACCATATG
CGGTGTGAAATACCGCACAGATGCGTAAGGAGAAAATACCGCATCAGGAAATTGTAAACGTTAATATTTTGTTAAAATTC
GCGTTAAATTTTTGTTAAATCAGCTCATTTTTTAACCAATAGGCCGAAATCGGCAAAATCCCTTATAAATCAAAAGAATA
GACCGAGATAGGGTTGAGTGTTGTTCCAGTTTGGAACAAGAGTCCACTATTAAAGAACGTGGACTCCAACGTCAAAGGGC
GAAAAACCGTCTATCAGGGCGATGGCCCACTACGTGAACCATCACCCTAATCAAGTTTTTTGGGGTCGAGGTGCCGTAAA
GCACTAAATCGGAACCCTAAAGGGAGCCCCCGATTTAGAGCTTGACGGGGAAAGCCGGCGAACGTGGCGAGAAAGGAAGG
GAAGAAAGCGAAAGGAGCGGGCGCTAGGGCGCTGGCAAGTGTAGCGGTCACGCTGCGCGTAACCACCACACCCGCCGCGC
TTAATGCGCCGCTACAGGGCGCGTCGCGCCATTCGCCATTCAGGCTACGCAACTGTTGGGAAGGGCGATCGGTGCGGGCC
TCTTCGCTATTACGCCAGCTGGCGAAGGGGGGATGTGCTGCAAGGCGATTAAGTTGGGTAACGCCAGGGTTTTCCCAGTC
ACGACGTTGTAAAACGACGGCCAGTGAATT

The amino acid sequence of mtCasapase-3 [SEQ ID NO:51] is:

MENTENSVDS KSIKNLEPKI IHGSESMDSG ISLDNSYKMD YPEMGLCIII NNKNFHKSTG
MTSRSGTDVD AANLRETFRN LKYEVRNKND LTREEIVELM RDVSKEDHSK RSSFVCVLLS
HGEEGIIFGT NGPVDLKKIT NFFRGDRCRS LTGKPKLFII QAGRGTELDC GIETDSGVDD
DMACHKIPVE ADFLYAYSTA PGYYSWRNSK DGSWFIQSLC AMLKQYADKL EFMHILTRVN
RKVATEFESF SFDATFHAKK QIPCIVSMLT KELYFYH

Sequences of DNA Encoding “Translocation Polypeptides” and their Vectors

The DNA sequence encoding the E7 protein with the translocation Signal sequence and LAMP-1 domain [SEQ ID NO:52] is:

ATGGCGGCCCCCGGCGCCCGGCGGCCGCTGCTCCTGCTGCTGCTGGCAGGCCTTGCACATGGCGCCTCAGCACTCTTTGA
GGATCTAATCATGCATGGAGATACACCTACATTGCATGAATATATGTTAGATTTGCAACCAGAGACAACTGATCTCTACT
GTTATGAGCAATTAAATGACAGCTCAGAGGAGGAGGATGAAATAGATGGTCCAGCTGGACAAGCAGAACCGGACAGAGCC
CATTACAATATTGTTACCTTTTGTTGCAAGTGTGACTCTACGCTTCGGTTGTGCGTACAAAGCACACACGTAGACATTCG
TACTTTGGAAGACCTGTTAATGGGCACACTAGGAATTGTGTGCCCCATCTGTTCTCAGGATCTTAACAACATGTTGATCC
CCATTGCTGTGGGCGGTGCCCTGGCAGGGCTGGTCCTCATCGTCCTCATTGCCTACCTCATTGGCAGGAAGAGGAGTCAC
GCCGGCTATCAGACCATCTAG

The amino acid sequence of Sig-E7-L1 [SEQ ID NO:53] is:

MAAPGARRPL LLLLLAGLAH GASALFEDLI MHGDTPTLHE YMLDLQPETT DLYCYEQLND
SSEEEDEIDG PAGQAEPDRA HYNIVTFCCK CDSTLRLCVQ STHVDIRTLE DLLMGTLGIV
CPICSQDLNN MLIPIAVGGA LAGLVLIVLI AYLIGRKRSH AGYQTI

The sequence of the vector pcDNA3-sigE7L1 [SEQ ID NO:54] is shown below with the SigE7-L1 sequence in lower case, underscored:

GACGGATCGGGAGATCTCCCGATCCCCTATGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCTACAACAAGGCAAGGCTTGACCGA
CAATTGCATGAAGAATCTGCTTAGGGTTAGGCGTTTTGCGCTGCTTCGCGATGTACGGGCCAGATATACGCGTTGACATT
GATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGACTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGGAGTTTGTTTTGGCACC
AAAATCAACGGGACTTTCCAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCTTACTGGCTTATCGAAATTAATACGACTCACTATAG
GGAGACCCAAGCTGGCTAGCGTTTAAACGGGCCCTCTAGACTCGAGCGGCCGCCACTGTGCTGGATATCTGCAGAATTCa
tggcggcccccggcgcccggcggccgctgctcctgctgctgctggcaggccttgcacatggcgcctcagcactctttgag
gatctaatcatgcatggagatacacctacattgcatgaatatatgttagatttgcaaccagagacaactgatctctactg
ttatgagcaattaaatgacagctcagaggaggaggatgaaatagatggtccagctggacaagcagaaccggacagagccc
attacaatattgttaccttttgttgcaagtgtgactctacgcttcggttgtgcgtacaaagcacacacgtagacattcgt
actttggaagacctgttaatgggcacactaggaattgtgtgccccatctgttctcaggatcttaacaacatgttgatccc
cattgctgtgggcggtgccctggcagggctggtcctcatcgtcctcattgcctacctcattggcaggaagaggagtcacg
ccggctatcagaccatctagGGATCCGAGCTCGGTACCAAGCTTAAGTTTAAACCGCTGATCAGCCTCGACTGTGCCTTC
TAGTTGCCAGCCATCTGTTGTTTGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGGCTTCTGAGGCGGAAAGAACCAGCTG
GGGCTCTAGGGGGTATCCCCACGCGCCCTGTAGCGGCGCATTAAGCGCGGCGGGTGTGGTGGTTACGCGCAGCGTGACCG
CTACACTTGCCAGCGCCCTAGCGCCCGCTCCTTTCGCTTTCTTCCCTTCCTTTCTCGCCACGTTCGCCGGCTTTCCCCGT
CAAGCTCTAAATCGGGGCATCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGTCCACGTTCTTTAATAGTG
GACTCTTGTTCCAAACTGGAACAACACTCAACCCTATCTCGGTCTATTCTTTTGATTTATAAGGGATTTTGGGGATTTCG
GCCTATTGGTTAAAAAATGAGCTGATTTAACAAAAATTTAACGCGAATTAATTCTGTGGAATGTGTGTCAGTTAGGGTGT
GGAAAGTCCCCAGGCTCCCCAGGCAGGCAGAAGTATGCAAAGCATGCATCTCAATTAGTCAGCAACCAGGTGTGGAAAGT
CCCCAGGCTCCCCAGCAGGCAGAAGTATGCAAAGCATGCATCTCAATTAGTCAGCAACCATAGTCCCGCCCCTAACTCCG
CCCATCCCGCCCCTAACTCCGCCCAGTTCCGCCCATTCTCCGCCCCATGGCTGACTAATTTTTTTTATTTATGCAGAGGC
CGAGGCCGCCTCTGCCTCTGAGCTATTCCAGAAGTAGTGAGGAGGCTTTTTTGGAGGCCTAGGCTTTTGCAAAAAGCTCC
CGGGAGCTTGTATATCCATTTTCGGATCTGATCAAGAGACAGGATGAGGATCGTTTCGCATGATTGAACAAGATGGATTG
CACGCAGGTTCTCCGGCCGCTTGGGTGGAGAGGCTATTCGGCTATGACTGGGCACAACAGACAATCGGCTGCTCTGATGC
CGCCGTGTTCCGGCTGTCAGCGCAGGGGCGCCCGGTTCTTTTTGTCAAGACCGACCTGTCCGGTGCCCTGAATGAACTGC
AGGACGAGGCAGCGCGGCTATCGTGGCTGGCCACGACGGGCGTTCCTTGCGCAGCTGTGCTCGACGTTGTCACTGAAGCG
GGAAGGGACTGGCTGCTATTGGGCGAAGTGCCGGGGCAGGATCTCCTGTCATCTCACCTTGCTCCTGCCGAGAAAGTATC
CATCATGGCTGATGCAATGCGGCGGCTGCATACGCTTGATCCGGCTACCTGCCCATTCGACCACCAAGCGAAACATCGCA
TCGAGCGAGCACGTACTCGGATGGAAGCCGGTCTTGTCGATCAGGATGATCTGGACGAAGAGCATCAGGGGCTCGCGCCA
GCCGAACTGTTCGCCAGGCTCAAGGCGCGCATGCCCGACGGCGAGGATCTCGTCGTGACCCATGGCGATGCCTGCTTGCC
GAATATCATGGTGGAAAATGGCCGCTTTTCTGGATTCATCGACTGTGGCCGGCTGGGTGTGGCGGACCGCTATCAGGACA
TAGCGTTGGCTACCCGTGATATTGCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGCGGGACTCTGGGGTTCGAAATGACC
GACCAAGCGACGCCCAACCTGCCATCACGAGATTTCGATTCCACCGCCGCCTTCTATGAAAGGTTGGGCTTCGGAATCGT
TTTCCGGGACGCCGGCTGGATGATCCTCCAGCGCGGGGATCTCATGCTGGAGTTCTTCGCCCACCCCAACTTGTTTATTG
CAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGTATACCGTCGACCTCTAGCTAGAGCTTGGCGTAATCATGGTC
ATAGCTGTTTCCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCTTACCGGATACCTGTCCG
CCTTTCTCCCTTCGGGAAGCGTGGCGCTTTCTCAATGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGGACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCGTGCACCCAACTGATCTTCAGC
ATCTTTTACTTTCACCAGCGTTTCTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGTGCCACCTGACGTC

HSP70 from M. tuberculosis

The nucleotide sequence encoding HSP70 (SEQ ID NO:55) is shown below and is deposited in GENBANK; nucleotides 10633-12510 of the M. tuberculosis genome.

atggctcg tgcggtcggg atcgacctcg ggaccaccaa ctccgtcgtc tcggttctgg aaggtggcga
cccggtcgtc gtcgccaact ccgagggctc caggaccacc ccgtcaattg tcgcgttcgc ccgcaacggt
gaggtgctgg tcggccagcc cgccaagaac caggcagtga ccaacgtcga tcgcaccgtg cgctcggtca
agcgacacat gggcagcgac tggtccatag agattgacgg caagaaatac accgcgccgg agatcagcgc
ccgcattctg atgaagctga agcgcgacgc cgaggcctac ctcggtgagg acattaccga cgcggttatc
acgacgcccg cctacttcaa tgacgcccag cgtcaggcca ccaaggacgc cggccagatc gccggcctca
acgtgctgcg gatcgtcaac gagccgaccg cggccgcgct ggcctacggc ctcgacaagg gcgagaagga
gcagcgaatc ctggtcttcg acttgggtgg tggcactttc gacgtttccc tgctggagat cggcgagggt
gtggttgagg tccgtgccac ttcgggtgac aaccacctcg gcggcgacga ctgggaccag cgggtcgtcg
attggctggt ggacaagttc aagggcacca gcggcatcga tctgaccaag gacaagatgg cgatgcagcg
gctgcgggaa gccgccgaga aggcaaagat cgagctgagt tcgagtcagt ccacctcgat caacctgccc
tacatcaccg tcgacgccga caagaacccg ttgttcttag acgagcagct gacccgcgcg gagttccaac
ggatcactca ggacctgctg gaccgcactc gcaagccgtt ccagtcggtg atcgctgaca ccggcatttc
ggtgtcggag atcgatcacg ttgtgctcgt gggtggttcg acccggatgc ccgcggtgac cgatctggtc
aaggaactca ccggcggcaa ggaacccaac aagggcgtca accccgatga ggttgtcgcg gtgggagccg
ctctgcaggc cggcgtcctc aagggcgagg tgaaagacgt tctgctgctt gatgttaccc cgctgagcct
gggtatcgag accaagggcg gggtgatgac caggctcatc gagcgcaaca ccacgatccc caccaagcgg
tcggagactt tcaccaccgc cgacgacaac caaccgtcgg tgcagatcca ggtctatcag ggggagcgtg
agatcgccgc gcacaacaag ttgctcgggt ccttcgagct gaccggcatc ccgccggcgc cgcgggggat
tccgcagatc gaggtcactt tcgacatcga cgccaacggc attgtgcacg tcaccgccaa ggacaagggc
accggcaagg agaacacgat ccgaatccag gaaggctcgg gcctgtccaa ggaagacatt gaccgcatga
tcaaggacgc cgaagcgcac gccgaggagg atcgcaagcg tcgcgaggag gccgatgttc gtaatcaagc
cgagacattg gtctaccaga cggagaagtt cgtcaaagaa cagcgtgagg ccgagggtgg ttcgaaggta
cctgaagaca cgctgaacaa ggttgatgcc gcggtggcgg aagcgaaggc ggcacttggc ggatcggata
tttcggccat caagtcggcg atggagaagc tgggccagga gtcgcaggct ctggggcaag cgatctacga
agcagctcag gctgcgtcac aggccactgg cgctgcccac cccggcggcg agccgggcgg tgcccacccc
ggctcggctg atgacgttgt ggacgcggag gtggtcgacg acggccggga ggccaagtga

The amino acid sequence of HSP70 [SEQ ID NO:56] is:

MARAVGIDLG TTNSVVSVLE GGDPVVVANS EGSRTTPSIV AFARNGEVLV GQPAKNQAVT NVDRTVRSVK
RHMGSDWSIE IDGKKYTAPE ISARILMKLK RDAEAYLGED ITDAVITTPA YFNDAQRQAT KDAGQIAGLN
VLRIVNEPTA AALAYGLDKG EKEQRILVFD LGGGTFDVSL LEIGEGVVEV RATSGDNHLG GDDWDQRVVD
WLVDKFKGTS GIDLTKDKMA MQRLREAAEK AKIELSSSQS TSINLPYITV DADKNPLFLD EQLTRAEFQR
ITQDLLDRTR KPFQSVIADT GISVSEIDHV VLVGGSTRMP AVTDLVKELT GGKEPNKGVN PDEVVAVGAA
LQAGVLKGEV KDVLLLDVTP LSLGIETKGG VMTRLIERNT TIPTKRSETF TTADDNQPSV QIQVYQGERE
IAAHNKLLGS FELTGIPPAP RGIPQIEVTF DIDANGIVHV TAKDKGTGKE NTIRIQEGSG LSKEDIDRMI
KDAEAHAEED RKRREEADVR NQAETLVYQT EKFVKEQREA EGGSKVPEDT LNKVDAAVAE AKAALGGSDI
SAIKSAMEKL GQESQALGQA IYEAAQAASQ ATGAAHPGGE PGGAHPGSAD DVVDAEVVDD GREAK

E7-Hsp70 Chimera or Fusion (nucleic acid is SEQ ID NO:57; amino acids are SEQ ID NO:58) E7 coding sequence is capitalized and underscored.

1/1 31/11
ATG CAT GGA GAT ACA CCT ACA TTG CAT GAA TAT ATG TTA GAT TTG CAA CCA GAG ACA ACT
Met his gly asp thr pro thr leu his glu tyr met leu asp leu gln pro glu thr thr
61/21 91/31
GAT CTC TAC TGT TAT GAG CAA TTA AAT GAC AGC TCA GAG GAG GAG GAT GAA ATA GAT GGT
asp leu tyr cys tyr glu gln leu asn asp ser ser glu glu glu asp glu ile asp gly
121/41 151/51
CCA GCT GGA CAA GCA GAA CCG GAC AGA GCC CAT TAC AAT ATT GTA ACC TTT TGT TGC AAG
pro ala gly gln ala glu pro asp arg ala his tyr asn lie val thr phe cys cys lys
181/61 211/71
TGT GAC TCT ACG CTT CGG TTG TGC GTA CAA AGC ACA CAC GTA GAC ATT CGT ACT TTG GAA
cys asp ser thr leu arg leu cys val gln ser thr his val asp ile arg thr leu glu
241/81 271/91
GAC CTG TTA ATG GGC ACA CTA GGA ATT GTG TGC CCC ATC TGT TCT CAA GGA TCC atg gct
asp leu leu met gly thr leu gly ile val cys pro ile cys ser gln gly ser met ala
301/101 331/111
cgt gcg gtc ggg atc gac ctc ggg acc acc aac tcc gtc gtc tcg gtt ctg gaa ggt ggc
arg ala val gly ile asp leu gly thr thr asn ser val val ser val leu glu gly gly
361/121 391/131
gac ccg gtc gtc gtc gcc aac tcc gag ggc tcc agg acc acc ccg tca att gtc gcg ttc
asp pro val val val ala asn ser glu gly ser arg thr thr pro ser ile val ala phe
421/141 451/151
gcc cgc aac ggt gag gtg ctg gtc ggc cag ccc gcc aag aac cag gca gtg acc aac gtc
ala arg asn gly glu val leu val gly gln pro ala lys asn gln ala val thr asn val
481/161 511/171
gat cgc acc gtg cgc tcg gtc aag cga cac atg ggc agc gac tgg tcc ata gag att gac
asp arg thr val arg ser val lys arg his met gly ser asp trp ser ile glu ile asp
541/181 571/191
ggc aag aaa tac acc gcg ccg gag atc agc gcc cgc att ctg atg aag ctg aag cgc gac
gly lys lys tyr thr ala pro glu ile ser ala arg ile leu met lys leu lys arg asp
601/201 631/211
gcc gag gcc tac ctc ggt gag gac att acc gac gcg gtt atc acg acg ccc gcc tac ttc
ala glu ala tyr leu gly glu asp ile thr asp ala val ile thr thr pro ala tyr phe
661/221 691/231
aat gac gcc cag cgt cag gcc acc aag gac gcc ggc cag atc gcc ggc ctc aac gtg ctg
asn asp ala gln arg gln ala thr lys asp ala gly gln ile ala gly leu asn val leu
721/241 751/251
cgg atc gtc aac gag ccg acc gcg gcc acg ctg gcc tac ggc ctc gac aag ggc gag aag
arg ile val asn glu pro thr ala ala ala leu ala tyr gly leu asp lys gly glu lys
781/261 811/271
gag cag cga atc ctg gtc ttc gac ttg ggt ggt ggc act ttc gac gtt tcc ctg ctg gag
glu gln arg ile leu val phe asp leu gly gly gly thr phe asp val ser leu leu glu
841/281 871/291
atc ggc gag ggt gtg gtt gag gtc cgt gcc act tcg ggt gac aac cac ctc ggc ggc gac
ile gly glu gly val val glu val arg ala thr ser gly asp asn his leu gly gly asp
901/301 931/311
gac tgg gac cag cgg gtc gtc gat tgg ctg gtg gac aag ttc aag ggc acc agc ggc atc
asp trp asp gln arg val val asp trp leu val asp lys phe lys gly thr ser gly ile
961/321 991/331
gat ctg acc aag gac aag atg gcg atg cag cgg ctg cgg gaa gcc gcc gag aag gca aag
asp leu thr lys asp lys met ala met gln arg leu arg glu ala ala glu lys ala lys
1021/341 1051/351
atc gag ctg agt tcg agt cag tcc acc tcg atc aac ctg ccc tac atc acc gtc gac gcc
ile glu leu ser ser ser gln ser thr ser ile asn leu pro tyr ile thr val asp ala
1081/361 1111/371
gac aag aac ccg ttg ttc tta gac gag cag ctg acc cgc gcg gag ttc caa cgg atc act
asp lys asn pro leu phe leu asp glu gln leu thr arg ala glu phe gln arg ile thr
1141/381 1171/391
cag gac ctg ctg gac cgc act cgc aag ccg ttc cag tcg gtg atc gct gac acc ggc att
gln asp leu leu asp arg thr arg lys pro phe gln ser val ile ala asp thr gly ile
1201/401 1231/411
tcg gtg tcg gag atc gat cac gtt gtg ctc gtg ggt ggt tcg acc cgg atg ccc gcg gtg
ser val ser glu ile asp his val val leu val gly gly ser thr arg met pro ala val
1261/421 1291/431
acc gat ctg gtc aag gaa ctc acc ggc ggc aag gaa ccc aac aag ggc gtc aac ccc gat
thr asp leu val lys glu leu thr gly gly lys glu pro asn lys gly val asn pro asp
1321/441 1351/451
gag gtt gtc gcg gtg gga gcc gct ctg cag gcc ggc gtc ctc aag ggc gag gtg aaa gac
glu val val ala val gly ala ala leu gln ala gly val leu lys gly glu val lys asp
1381/461 1411/471
gtt ctg ctg ctt gat gtt acc ccg ctg agc ctg ggt atc gag acc aag ggc ggg gtg atg
val leu leu leu asp val thr pro leu ser leu gly ile glu thr lys gly gly val met
1441/481 1471/491
acc agg ctc atc gag cgc aac acc acg atc ccc acc aag cgg tcg gag act ttc acc acc
thr arg leu ile glu arg asn thr thr ile pro thr lys arg ser glu thr phe thr thr
1501/501 1531/511
gcc gac gac aac caa ccg tcg gtg cag atc cag gtc tat cag ggg gag cgt gag atc gcc
ala asp asp asn gln pro ser val gln ile gln val tyr gln gly glu arg glu ile ala
1561/521 1591/531
gcg cac aac aag ttg ctc ggg tcc ttc gag ctg acc ggc atc ccg ccg gcg ccg cgg ggg
ala his asn lys leu leu gly ser phe glu leu thr gly ile pro pro ala pro arg gly
1621/541 1651/551
att ccg cag atc gag gtc act ttc gac atc gac gcc aac ggc att gtg cac gtc acc gcc
ile pro gln ile glu val thr phe asp ile asp ala asn gly ile val his val thr ala
1681/561 1711/571
aag gac aag ggc acc ggc aag gag aac acg atc cga atc cag gaa ggc tcg ggc ctg tcc
lys asp lys gly thr gly lys glu asn thr ile arg ile gln glu gly ser gly leu ser
1741/581 1771/591
aag gaa gac att gac cgc atg atc aag gac gcc gaa gcg cac gcc gag gag gat cgc aag
lys glu asp ile asp arg met ile lys asp ala glu ala his ala glu glu asp arg lys
1801/601 1831/611
cgt cgc gag gag gcc gat gtt cgt aat caa gcc gag aca ttg gtc tac cag acg gag aag
arg arg glu glu ala asp val arg asn gln ala glu thr leu val tyr gln thr glu lys
1861/621 1891/631
ttc gtc aaa gaa cag cgt gag gcc gag ggt ggt tcg aag gta cct gaa gac acg ctg aac
phe val lys glu gln arg glu ala glu gly gly ser lys val pro glu asp thr leu asn
1921/641 1951/651
aag gtt gat gcc gcg gtg gcg gaa gcg aag gcg gca ctt ggc gga tcg gat att tcg gcc
lys val asp ala ala val ala glu ala lys ala ala leu gly gly ser asp ile ser ala
1981/661 2011/671
atc aag tcg gcg atg gag aag ctg ggc cag gag tcg cag gct ctg ggg caa gcg atc tac
ile lys ser ala met glu lys leu gly gln glu ser gln ala leu gly gln ala ile tyr
2041/681 2071/691
gaa gca gct cag gct gcg tca cag gcc act ggc gct gcc cac ccc ggc tcg gct gat gaA
glu ala ala gln ala ala ser gln ala thr gly ala ala his pro gly ser ala asp glu
2101/701
AGC a
ser

ETA(dII) from Pseudomonas aeruginosa

The section that follows lists the sequences of the ETA(dII) polypeptides alone or in fusion with E7 antigen, the nucleic acids encoding some of these peptides and nucleic acids of the vectors into which the sequences encoding these polypeptides are cloned. The complete coding sequence for Pseudomonas aeruginosa exotoxin type A (ETA)—SEQ ID NO:59—GenBank Accession No. K01397, is shown below:

```
      1 ctgcagctgg tcaggccgtt tccgcaacgc ttgaagtcct ggccgatata ccggcagggc
    61 cagccatcgt tcgacgaata aagccacctc agccatgatg ccctttccat ccccagcgga
  121 accccgacat ggacgccaaa gccctgctcc tcggcagcct ctgcctggcc gccccattcg
  181 ccgacgcggc gacgctcgac aatgctctct ccgcctgcct cgccgcccgg ctcggtgcac
  241 cgcacacggc ggagggccag ttgcacctgc cactcaccct tgaggcccgg cgctccaccg
  301 gcgaatgcgg ctgtacctcg gcgctggtgc gatatcggct gctggccagg ggcgccagcg
  361 ccgacagcct cgtgcttcaa gagggctgct cgatagtcgc caggacacgc cgcgcacgct
  421 gaccctggcg gcggacgccg gcttggcgag cggccgcgaa ctggtcgtca ccctgggttg
  481 tcaggcgcct gactgacagg ccgggctgcc accaccaggc cgagatggac gccctgcatg
  541 tatcctccga tcggcaagcc tcccgttcgc acattcacca ctctgcaatc cagttcataa
  601 atcccataaa agccctcttc cgctccccgc cagcctcccc gcatcccgca ccctagacgc
  661 cccgccgctc tccgccggct cgcccgacaa gaaaaaccaa ccgctcgatc agcctcatcc
  721 ttcacccatc acaggagcca tcgcgatgca cctgataccc cattggatcc ccctggtcgc
  781 cagcctcggc ctgctcgccg gcggctcgtc cgcgtccgcc gccgaggaag ccttcgacct
  841 ctggaacgaa tgcgccaaag cctgcgtgct cgacctcaag gacggcgtgc gttccagccg
  901 catgagcgtc gacccggcca tcgccgacac caacggccag ggcgtgctgc actactccat
  961 ggtcctggag ggcggcaacg acgcgctcaa gctggccatc gacaacgccc tcagcatcac
1021 cagcgacggc ctgaccatcc gcctcgaagg cggcgtcgag ccgaacaagc cggtgcgcta
1081 cagctacacg cgccaggcgc gcggcagttg gtcgctgaac tggctggtac cgatcggcca
1141 cgagaagccc tcgaacatca aggtgttcat ccacgaactg aacgccggca accagctcag
1201 ccacatgtcg ccgatctaca ccatcgagat gggcgacgag ttgctggcga agctggcgcg
1261 cgatgccacc ttcttcgtca gggcgcacga gagcaacgag atgcagccga cgctcgccat
1321 cagccatgcc ggggtcagcg tggtcatggc ccagacccag ccgcgccggg aaaagcgctg
1381 gagcgaatgg gccagcggca aggtgttgtg cctgctcgac ccgctggacg gggtctacaa
1441 ctacctcgcc cagcaacgct gcaacctcga cgatacctgg gaaggcaaga tctaccgggt
1501 gctcgccggc aacccggcga agcatgacct ggacatcaaa cccacggtca tcagtcatcg
1561 cctgcacttt cccgagggcg gcagcctggc cgcgctgacc gcgcaccagg cttgccacct
1621 gccgctggag actttcaccc gtcatcgcca gccgcgcggc tgggaacaac tggagcagtg
1681 cggctatccg gtgcagcggc tggtcgccct ctacctggcg gcgcggctgt cgtggaacca
1741 ggtcgaccag gtgatccgca acgccctggc cagccccggc agcggcggcg acctgggcga
1801 agcgatccgc gagcagccgg agcaggcccg tctggccctg accctggccg ccgccgagag
1861 cgagcgcttc gtccggcagg gcaccggcaa cgacgaggcc ggcgcggcca acgccgacgt
1921 ggtgagcctg acctgcccgg tcgccgccgg tgaatgcgcg ggcccggcgg acagcggcga
1981 cgccctgctg gagcgcaact atcccactgg cgcggagttc ctcggcgacg gcggcgacgt
2041 cagcttcagc acccgcggca cgcagaactg gacggtggag cggctgctcc aggcgcaccg
2101 ccaactggag gagcgcggct atgtgttcgt cggctaccac ggcaccttcc tcgaagcggc
2161 gcaaagcatc gtcttcggcg gggtgcgcgc gcgcagccag gacctcgacg cgatctggcg
2221 cggtttctat atcgccggcg atccggcgct ggcctacggc tacgcccagg accaggaacc
2281 cgacgcacgc ggccggatcc gcaacggtgc cctgctgcgg gtctatgtgc cgcgctcgag
2341 cctgccgggc ttctaccgca ccagcctgac cctggccgcg ccggaggcgg cgggcgaggt
2401 cgaacggctg atcggccatc cgctgccgct gcgcctggac gccatcaccg gccccgagga
2461 ggaaggcggg cgcctggaga ccattctcgg ctggccgctg gccgagcgca ccgtggtgat
2521 tccctcggcg atccccaccg acccgcgcaa cgtcggcggc gacctcgacc cgtccagcat
2581 ccccgacaag gaacaggcga tcagcgccct gccggactac gccagccagc ccggcaaacc
2641 gccgcgcgag gacctgaagt aactgccgcg accggccggc tcccttcgca ggagccggcc
2701 ttctcggggc ctggccatac atcaggtttt cctgatgcca gcccaatcga atatgaattc
2760
```

The amino acid sequence of ETA (SEQ ID NO:60), GenBank Accession No. K01397, is shown below

MHLIPHWIPL VASLGLLAGG SSASAAEEAF DLWNECAKAC VLVLKDGVRS SRMSVDPAIA 60
DTNGQGVLHY SMVLEGGNDA LKLAIDNALS ITSDGLTIRL EGGVEPNKPV RYSYTRQARG 120
SWSLNWLVPI GHEKPSNIKV FIHELNAGNQ LSHMSPIYTI EMGDELLAKL ARDATFFVRA 180
HESNEMQPTL AISHAGVSVV MAQTQPRREK RWSEWASGKV LCLLDPLDGV YNYLAQQRCN 240
LDDTWEGKIY RVLAGNPAKH DLDIKPTVIS HRLHFPEGGS LAALTAHQAC HLPLETFTRH 300
RQPRGWEQLE QCGYPVQRLV ALYLAARLSW NQVDQVIRNA LASPGSGGDL GEAIREQPEQ 360
ARLALTLAAA ESERFVRQGT GNDEAGAANA DVVSLTCPVA AGECAGPADS GDALLERNYP 420
TGAEFLGDGG DVSFSTRGTQ NWTVERLLQA HRQLEERGYV FVGYHGTFLE AAQSIVFGGV 480
RARSQDLDAI WRGFYIAGDP ALAYGYAQDQ EPDARGRIRN GALLRVYVPR SSLPGFYRTS 540
LTLAAPEAAG EVERLIGHPL PLRLDAITGP EEEGGRLETI LGWPLAERTV VIPSAIPTDP 600
RNVGGDLDPS SIPDKEQAIS ALPDYASQPG KPPREDLK 638

Residues 1-25 (italicized) represent the signal peptide; the start of the mature polypeptide is shown as a bold/underlined. The mature polypeptide is residues 26-638 of SEQ ID NO:60. The ETA(dII) translocation domain (underscored above) spans residues 247-417 of the mature polypeptide (corresponding to residues 272-442 of SEQ ID NO:60) and is presented below separately as SEQ ID NO:61.

RLHFPEGGSL AALTAHQACH LPLETFTRHR QPRGWEQLEQ CGYPVQRLVA LYLAARLSWN 60
QVDQVIRNAL ASPGSGGDLG EAIREQPEQA RLALTLAAAE SERFVRQGTG NDEAGAANAD 120
VVSLTCPVAA GECAGPADSG DALLERNYPT GAEFLGDGGD VSFSTRGTQN W 171

The sequences shown below (nucleotide is SEQ ID NO:62 and amino acid is SEQ ID NO:63) are the construct in which ETA(dII) is fused to the HPV-16 E7 polypeptide. The ETA(dII) sequence appears in plain font, extra codons from pcDNA3 are italicized; those between the ETA(dII) and E7 sequence are also bolded (and result in the interposition of two amino acids between ETA(dII) and E7. The E7 sequence is underscored. The E7 sequence ends in Gln.

1/1 31/11
atg cgc ctg cac ttt ccc gag ggc ggc agc ctg gcc gcg ctg acc gcg cac cag gct tgc
Met arg leu his phe pro glu gly gly ser leu ala ala leu thr ala his gln ala cys
61/21 91/31
cac ctg ccg ctg gag act ttc acc cgt cat cgc cag ccg cgc ggc tgg gaa caa ctg gag
his leu pro leu glu thr phe thr arg his arg gln pro arg gly trp glu gln leu glu
121/41 151/51
cag tgc ggc tat ccg gtg cag cgg ctg gtc gcc ctc tac ctg gcg gcg cgg ctg tcg tgg
gln cys gly tyr pro val gln arg leu val ala leu tyr leu ala ala arg leu ser trp
181/61 211/71
aac cag gtc gac cag gtg atc cgc aac gcc ctg gcc agc ccc ggc agc ggc ggc gac ctg
asn gln val asp gln val ile arg asn ala leu ala ser pro gly ser gly gly asp leu
241/81 271/91
ggc gaa gcg atc cgc gag cag ccg gag cag gcc cgt ctg gcc ctg acc ctg gcc gcc gcc
gly glu ala ile arg glu gln pro glu gln ala arg leu ala leu thr leu ala ala ala
301/101 331/111
gag agc gag cgc ttc gtc cgg cag ggc acc ggc aac gac gag gcc ggc gcg gcc aac gcc
glu ser glu arg phe val arg gln gly thr gly asn asp glu ala gly ala ala asn ala
361/121 391/131
gac gtg gtg agc ctg acc tgc ccg gtc gcc gcc ggt gaa tgc gcg ggc ccg gcg gac agc
asp val val ser leu thr cys pro val ala ala gly glu cys ala gly pro ala asp ser
421/141 451/151
ggc gac gcc ctg ctg gag cgc aac tat ccc act ggc gcg gag ttc ctc ggc gac ggc ggc
gly asp ala leu leu glu arg asn tyr pro thr gly ala glu phe leu gly asp gly gly
481/161 511/171
gac gtc agc ttc agc acc cgc ggc acg cag [symbol] atg cat gga gat aca cct aca
asp val ser phe ser thr arg gly thr gln [symbol] met his gly asp thr pro thr
541/181 571/191
ttg cat gaa tat atg tta gat ttg caa cca gag aca act gat ctc tac tgt tat gag caa
leu his glu tyr met leu asp leu gly pro glu thr thr asp leu tyr cys tyr glu gln
601/201 631/211
tta aat gac agc tca gag gag gag gat gaa ata gat ggt cca gct gga caa gca gaa ccg
leu asn asp ser ser glu glu glu asp glu ile asp gly pro ala gly gln ala glu pro
661/221 691/231
gac aga gcc cat tac aat att gta acc ttt tgt tgc aag tgt gac tct acg ctt cgg ttg
asp arg ala his tyr asn ile val thr phe cys cys lys cys asp ser thr leu arg leu
721/241 751/251
tgc gta caa agc aca cac gta gac att cgt act ttg gaa gac ctg tta atg ggc aca cta
cys val gln ser thr his val asp ile arg thr leu glu asp leu leu met gly thr leu
781/261 811/271
gga att gtg tgc ccc atc tgt tct caa gga tcc gag ctc ggt acc aag ctt aag ttt aaa
gly ile val cys pro ile cys ser gln gly ser glu leu gly thr lys leu lys phe lys
841/281
ccg ctg atc agc ctc gac tgt gcc ttc tag
pro leu ile ser leu asp cys ala phe AMB

Compared to the GenBank sequence of E7 (SEQ ID NO:64 65) shown below, three C-terminal amino acids have been deleted.

pcDNA3-E7-Hsp70 SEQ ID NO:66 The E7-Hsp70 fusion sequence is shown in bold, caps

```
        | 10 | 20 | 30 | 40 | 50 | 60 | 70 | 80 |
      1 gacggatcgg gagatctccc gatcccctat ggtcgactct cagtacaatc tgctctgatg ccgcatagtt aagccagtat 80
    81 ctgctccctg cttgtgtgtt ggaggtcgct gagtagtgcg cgagcaaaat ttaagctaca acaaggcaag gcttgaccga 160
  161 caattgcatg aagaatctgc ttagggttag gcgttttgcg ctgcttcgcg atgtacgggc cagatatacg cgttgacatt 240
  241 gattattgac tagttattaa tagtaatcaa ttacggggtc attagttcat agcccatata tggagttccg cgttacataa 320
  321 cttacggtaa atggcccgcc tggctgaccg cccaacgacc cccgcccatt gacgtcaata atgacgtatg ttcccatagt 400
  401 aacgccaata gggactttcc attgacgtca atgggtggac tatttacggt aaactgccca cttggcagta catcaagtgt 480
  481 atcatatgcc aagtacgccc cctattgacg tcaatgacgg taaatggccc gcctggcatt atgcccagta catgacctta 560
  561 tgggactttc ctacttggca gtacatctac gtattagtca tcgctattac catggtgatg cggttttggc agtacatcaa 640
  641 tgggcgtgga tagcggtttg actcacgggg atttccaagt ctccacccca ttgacgtcaa tgggagtttg ttttggcacc 720
  721 aaaatcaacg ggactttcca aaatgtcgta acaactccgc cccattgacg caaatgggcg gtaggcgtgt acggtgggag 800
  801 gtctatataa gcagagctct ctggctaact agagaaccca ctgcttactg gcttatcgaa attaatacga ctcactatag 880
  881 ggagacccaa gctggctagc gtttaaacgg gccctctaga ctcgagcggc cgccactgtg ctggatatct gcagaattcc 960
  961 accacactgg actagtggat ccATGCATGG AGATACACCT ACATTGCATG AATATATGTT AGATTTGCAA CCAGAGACAA 1040
1041 CTGATCTCTA CTGTTATGAG CAATTAAATG ACAGCTCAGA GGAGGAGGAT GAAATAGATG GTCCAGCTGG ACAAGCAGAA 1120
1121 CCGGACAGAG CCCATTACAA TATTGTAACC TTTTGTTGCA AGTGTGACTC TACGCTTCGG TTGTGCGTAC AAAGCACACA 1200
1201 CGTAGACATT CGTACTTTGG AAGACCTGTT AATGGGCACA CTAGGAATTG TGTGCCCCAT CTGTTCTCAA GGATCCATGG 1280
1281 CTCGTGCGGT CGGGATCGAC CTCGGGACCA CCAACTCCGT CGTCTCGGTT CTGGAAGGTG GCGACCCGGT CGTCGTCGCC 1360
1361 AACTCCGAGG GCTCCAGGAC CACCCCGTCA ATTGTCGCGT TCGCCCGCAA CGGTGAGGTG CTGGTCGGCC AGCCCGCCAA 1440
1441 GAACCAGGCA GTGACCAACG TCGATCGCAC CGTGCGCTCG GTCAAGCGAC ACATGGGCAG CGACTGGTCC ATAGAGATTG 1520
1521 ACGGCAAGAA ATACACCGCG CCGGAGATCA GCGCCCGCAT TCTGATGAAG CTGAAGCGCG ACGCCGAGGC CTACCTCGGT 1600
1601 GAGGACATTA CCGACGCGGT TATCACGACG CCCGCCTACT TCAATGACGC CCAGCGTCAG GCCACCAAGG ACGCCGGCCA 1680
1681 GATCGCCGGC CTCAACGTGC TGCGGATCGT CAACGAGCCG ACCGCGGCCG CGCTGGCCTA CGGCCTCGAC AAGGGCGAGA 1760
1761 AGGAGCAGCG AATCCTGGTC TTCGACTTGG GTGGTGGCAC TTTCGACGTT TCCCTGCTGG AGATCGGCGA GGGTGTGGTT 1840
1841 GAGGTCCGTG CCACTTCGGG TGACAACCAC CTCGGCGGCG ACGACTGGGA CCAGCGGGTC GTCGATTGGC TGGTGGACAA 1920
1921 GTTCAAGGGC ACCAGCGGCA TCGATCTGAC CAAGGACAAG ATGGCGATGC AGCGGCTGCG GGAAGCCGCC GAGAAGGCAA 2000
2001 AGATCGAGCT GAGTTCGAGT CAGTCCACCT CGATCAACCT GCCCTACATC ACCGTCGACG CCGACAAGAA CCCGTTGTTC 2080
2081 TTAGACGAGC AGCTGACCCG CGCGGAGTTC CAACGGATCA CTCAGGACCT GCTGGACCGC ACTCGCAAGC CGTTCCAGTC 2160
2161 GGTGATCGCT GACACCGGCA TTTCGGTGTC GGAGATCGAT CACGTTGTGC TCGTGGGTGG TTCGACCCGG ATGCCCGCGG 2240
2241 TGACCGATCT GGTCAAGGAA CTCACCGGCG GCAAGGAACC CAACAAGGGC GTCAACCCCG ATGAGGTTGT CGCGGTGGGA 2320
2321 GCCGCTCTGC AGGCCGGCGT CCTCAAGGGC GAGGTGAAAG ACGTTCTGCT GCTTGATGTT ACCCCGCTGA GCCTGGGTAT 2400
2401 CGAGACCAAG GGCGGGGTGA TGACCAGGCT CATCGAGCGC AACACCACGA TCCCCACCAA GCGGTCGGAG ACTTTCACCA 2480
2481 CCGCCGACGA CAACCAACCG TCGGTGCAGA TCCAGGTCTA TCAGGGGGAG CGTGAGATCG CCGCGCACAA CAAGTTGCTC 2560
2561 GGGTCCTTCG AGCTGACCGG CATCCCGCCG GCGCCGCGGG GGATTCCGCA GATCGAGGTC ACTTTCGACA TCGACGCCAA 2640
2641 CGGCATTGTG CACGTCACCG CCAAGGACAA GGGCACCGGC AAGGAGAACA CGATCCGAAT CCAGGAAGGC TCGGGCCTGT 2720
2721 CCAAGGAAGA CATTGACCGC ATGATCAAGG ACGCCGAAGC GCACGCCGAG GAGGATCGCA AGCGTCGCGA GGAGGCCGAT 2800
2801 GTTCGTAATC AAGCCGAGAC ATTGGTCTAC CAGACGGAGA AGTTCGTCAA AGAACAGCGT GAGGCCGAGG GTGGTTCGAA 2880
2881 GGTACCTGAA GACACGCTGA ACAAGGTTGA TGCCGCGGTG GCGGAAGCGA AGGCGGCACT TGGCGGATCG GATATTTCGG 2960
2961 CCATCAAGTC GGCGATGGAG AAGCTGGGCC AGGAGTCGCA GGCTCTGGGG CAAGCGATCT ACGAAGCAGC TCAGGCTGCG 3040
3041 TCACAGGCCA CTGGCGCTGC CCACCCCGGC TCGGCTGATG AAAGCTTaag tttaaaccgc tgatcagcct cgactgtgcc 3120
3121 ttctagttgc cagccatctg ttgtttgccc ctcccccgtg ccttccttga ccctggaagg tgccactccc actgtccttt 3200
3201 cctaataaaa tgaggaaatt gcatcgcatt gtctgagtag gtgtcattct attctggggg gtggggtggg gcaggacagc 3280
3281 aagggggagg attgggaaga caatagcagg catgctgggg atgcggtggg ctctatggct tctgaggcgg aaagaaccag 3360
3361 ctggggctct agggggtatc cccacgcgcc ctgtagcggc gcattaagcg cggcgggtgt ggtggttacg cgcagcgtga 3440
3441 ccgctacact tgccagcgcc ctagcgcccg ctcctttcgc tttcttccct tcctttctcg ccacgttcgc cggctttccc 3520
3521 cgtcaagctc taaatcgggg catcccttta gggttccgat ttagtgcttt acggcacctc gaccccaaaa aacttgatta 3600
3601 gggtgatggt tcacgtagtg ggccatcgcc ctgatagacg gtttttcgcc ctttgacgtt ggagtccacg ttctttaata 3680
3681 gtggactctt gttccaaact ggaacaacac tcaaccctat ctcggtctat tcttttgatt tataagggat tttggggatt 3760
3761 tcggcctatt ggttaaaaaa tgagctgatt taacaaaaat ttaacgcgaa ttaattctgt ggaatgtgtg tcagttaggg 3840
3841 tgtggaaagt ccccaggctc cccaggcagg cagaagtatg caaagcatgc atctcaatta gtcagcaacc aggtgtggaa 3920
3921 agtccccagg ctccccagca ggcagaagta tgcaaagcat gcatctcaat tagtcagcaa ccatagtccc gcccctaact 4000
4001 ccgcccatcc cgcccctaac tccgcccagt tccgcccatt ctccgcccca tggctgacta atttttttta tttatgcaga 4080
4081 ggccgaggcc gcctctgcct ctgagctatt ccagaagtag tgaggaggct tttttggagg cctaggcttt tgcaaaaagc 4160
4161 tcccgggagc ttgtatatcc attttcggat ctgatcaaga gacaggatga ggatcgtttc gcatgattga acaagatgga 4240
4241 ttgcacgcag gttctccggc cgcttgggtg gagaggctat tcggctatga ctgggcacaa cagacaatcg gctgctctga 4320
4321 tgccgccgtg ttccggctgt cagcgcaggg gcgcccggtt ctttttgtca agaccgacct gtccggtgcc ctgaatgaac 4400
4401 tgcaggacga ggcagcgcgg ctatcgtggc tggccacgac gggcgttcct tgcgcagctg tgctcgacgt tgtcactgaa 4480
4481 gcgggaaggg actggctgct attgggcgaa gtgccggggc aggatctcct gtcatctcac cttgctcctg ccgagaaagt 4560
4561 atccatcatg gctgatgcaa tgcggcggct gcatacgctt gatccggcta cctgcccatt cgaccaccaa gcgaaacatc 4640
4641 gcatcgagcg agcacgtact cggatggaag ccggtcttgt cgatcaggat gatctggacg aagagcatca ggggctcgcg 4720
4721 ccagccgaac tgttcgccag gctcaaggcg cgcatgcccg acggcgagga tctcgtcgtg acccatggcg atgcctgctt 4800
4801 gccgaatatc atggtggaaa atggccgctt ttctggattc atcgactgtg gccggctggg tgtggcggac cgctatcagg 4880
4881 acatagcgtt ggctacccgt gatattgctg aagagcttgg cggcgaatgg gctgaccgct tcctcgtgct ttacggtatc 4960
4961 gccgctcccg attcgcagcg catcgccttc tatcgccttc ttgacgagtt cttctgagcg ggactctggg gttcgaaatg 5040
5041 accgaccaag cgacgcccaa cctgccatca cgagatttcg attccaccgc cgccttctat gaaaggttgg gcttcggaat 5120
5121 cgttttccgg gacgccggct ggatgatcct ccagcgcggg gatctcatgc tggagttctt cgcccacccc aacttgttta 5200
5201 ttgcagctta taatggttac aaataaagca atagcatcac aaatttcaca aataaagcat ttttttcact gcattctagt 5280
5281 tgtggtttgt ccaaactcat caatgtatct tatcatgtct gtataccgtc gacctctagc tagagcttgg cgtaatcatg 5360
5361 gtcatagctg tttcctgtgt gaaattgtta tccgctcaca attccacaca acatacgagc cggaagcata aagtgtaaag 5440
5441 cctggggtgc ctaatgagtg agctaactca cattaattgc gttgcgctca ctgcccgctt tccagtcggg aaacctgtcg 5520
5521 tgccagctgc attaatgaat cggccaacgc gcggggagag gcggtttgcg tattgggcgc tcttccgctt cctcgctcac 5600
5601 tgactcgctg cgctcggtcg ttcggctgcg gcgagcggta tcagctcact caaaggcggt aatacggtta tccacagaat 5680
5681 caggggataa cgcaggaaag aacatgtgag caaaaggcca gcaaaaggcc aggaaccgta aaaaggccgc gttgctggcg 5760
5761 tttttccata ggctccgccc ccctgacgag catcacaaaa atcgacgctc aagtcagagg tggcgaaacc cgacaggact 5840
5841 ataaagatac caggcgtttc cccctggaag ctccctcgtg cgctctcctg ttccgaccct gccgcttacc ggatacctgt 5920
5921 ccgcctttct cccttcggga agcgtggcgc tttctcaatg ctcacgctgt aggtatctca gttcggtgta ggtcgttcgc 6000
6001 tccaagctgg gctgtgtgca cgaacccccc gttcagcccg accgctgcgc cttatccggt aactatcgtc ttgagtccaa 6080
6081 cccggtaaga cacgacttat cgccactggc agcagccact ggtaacagga ttagcagagc gaggtatgta ggcggtgcta 6160
6161 cagagttctt gaagtggtgg cctaactacg gctacactag aaggacagta tttggtatct gcgctctgct gaagccagtt 6240
6241 accttcggaa aaagagttgg tagctcttga tccggcaaac aaaccaccgc tggtagcggt ggtttttttg tttgcaagca 6320
6321 gcagattacg cgcagaaaaa aaggatctca agaagatcct ttgatctttt ctacggggtc tgacgctcag tggaacgaaa 6400
6401 actcacgtta agggattttg gtcatgagat tatcaaaaag gatcttcacc tagatccttt taaattaaaa atgaagtttt 6480
6481 aaatcaatct aaagtatata tgagtaaact tggtctgaca gttaccaatg cttaatcagt gaggcaccta tctcagcgat 6560
6561 ctgtctattt cgttcatcca tagttgcctg actccccgtc gtgtagataa ctacgatacg ggagggctta ccatctggcc 6640
6641 ccagtgctgc aatgataccg cgagacccac gctcaccggc tccagattta tcagcaataa accagccagc cggaagggcc 6720
6721 gagcgcagaa gtggtcctgc aactttatcc gcctccatcc agtctattaa ttgttgccgg gaagctagag taagtagttc 6800
6801 gccagttaat agtttgcgca acgttgttgc cattgctaca ggcatcgtgg tgtcacgctc gtcgtttggt atggcttcat 6880
6881 tcagctccgg ttcccaacga tcaaggcgag ttacatgatc ccccatgttg tgcaaaaaag cggttagctc cttcggtcct 6960
6961 ccgatcgttg tcagaagtaa gttggccgca gtgttatcac tcatggttat ggcagcactg cataattctc ttactgtcat 7040
7041 gccatccgta agatgctttt ctgtgactgg tgagtactca accaagtcat tctgagaata gtgtatgcgg cgaccgagtt 7120
7121 gctcttgccc ggcgtcaata cgggataata ccgcgccaca tagcagaact ttaaaagtgc tcatcattgg aaaacgttct 7200
7201 tcggggcgaa aactctcaag gatcttaccg ctgttgagat ccagttcgat gtaacccact cgtgcaccca actgatcttc 7280
7281 agcatctttt actttcacca gcgtttctgg gtgagcaaaa acaggaaggc aaaatgccgc aaaaaaggga ataagggcga 7360
7361 cacggaaatg ttgaatactc atactcttcc tttttcaata ttattgaagc atttatcagg gttattgtct catgagcgga 440
7441 tacatatttg aatgtattta gaaaaataaa caaatagggg ttccgcgcac atttccccga aaagtgccac ctgacgtc 7518
        | 10 | 20 | 30 | 40 | 50 | 60 | 70 | 80 |
```

The nucleic acid sequence of plasmid construct pcDNA3-ETA(dII)/E7 (SEQ ID NO:67) is shown below. ETA(dII)/E7 is ligated in the EcoRI/BamHI sites of pcDNA3 vector. The nucleotides encoding ETA(dII)/E7 are shown in lower case bold.

```
        | 10 | 20 | 30 | 40 | 50 | 60 | 70 | 80 |
      1 GACGGATCGG GAGATCTCCC GATCCCCTAT GGTCGACTCT CAGTACAATC TGCTCTGATG CCGCATAGTT AAGCCAGTAT 80
    81 CTGCTCCCTG CTTGTGTGTT GGAGGTCGCT GAGTAGTGCG CGAGCAAAAT TTAAGCTACA ACAAGGCAAG GCTTGACCGA 160
  161 CAATTGCATG AAGAATCTGC TTAGGGTTAG GCGTTTTGCG CTGCTTCGCG ATGTACGGGC CAGATATACG CGTTGACATT 240
  241 GATTATTGAC TAGTTATTAA TAGTAATCAA TTACGGGGTC ATTAGTTCAT AGCCCATATA TGGAGTTCCG CGTTACATAA 320
  321 CTTACGGTAA ATGGCCCGCC TGGCTGACCG CCCAACGACC CCCGCCCATT GACGTCAATA ATGACGTATG TTCCCATAGT 400
  401 AACGCCAATA GGGACTTTCC ATTGACGTCA ATGGGTGGAC TATTTACGGT AAACTGCCCA CTTGGCAGTA CATCAAGTGT 480
  481 ATCATATGCC AAGTACGCCC CCTATTGACG TCAATGACGG TAAATGGCCC GCCTGGCATT ATGCCCAGTA CATGACCTTA 560
  561 TGGGACTTTC CTACTTGGCA GTACATCTAC GTATTAGTCA TCGCTATTAC CATGGTGATG CGGTTTTGGC AGTACATCAA 640
  641 TGGGCGTGGA TAGCGGTTTG ACTCACGGGG ATTTCCAAGT CTCCACCCCA TTGACGTCAA TGGGAGTTTG TTTTGGCACC 720
  721 AAAATCAACG GGACTTTCCA AAATGTCGTA ACAACTCCGC CCCATTGACG CAAATGGGCG GTAGGCGTGT ACGGTGGGAG 800
  801 GTCTATATAA GCAGAGCTCT CTGGCTAACT AGAGAACCCA CTGCTTACTG GCTTATCGAA ATTAATACGA CTCACTATAG 880
  881 GGAGACCCAA GCTGGCTAGC GTTTAAACGG GCCCTCTAGA CTCGAGCGGC CGCCACTGTG CTGGATATCT GCAGAATTCa 960
  961 tgcgcctgca ctttcccgag ggcggcagcc tggccgcgct gaccgcgcac caggcttgcc acctgccgct ggagactttc 1040
1041 acccgtcatc gccagccgcg cggctgggaa caactggagc agtgcggcta tccggtgcag cggctggtcg ccctctacct 1120
1121 ggcggcgcgg ctgtcgtgga accaggtcga ccaggtgatc cgcaacgccc tggccagccc cggcagcggc ggcgacctgg 1200
1201 gcgaagcgat ccgcgagcag ccggagcagg cccgtctggc cctgaccctg gccgccgccg agagcgagcg cttcgtccgg 1280
1281 cagggcaccg gcaacgacga ggccggcgcg gccaacgccg acgtggtgag cctgacctgc ccggtcgccg ccggtgaatg 1360
1361 cgcgggcccg gcggacagcg gcgacgccct gctggagcgc aactatccca ctggcgcgga gttcctcggc gacggcggcg 1440
1441 acgtcagctt cagcacccgc ggcacgcaga acgaattcat gcatggagat acacctacat tcgatgaata tatgttagat 1520
1521 ttgcaaccag agacaactga tctctactgt tatgagcaat taaatgacag ctcagaggag gaggatgaaa tagatggtcc 1600
1601 agctggacaa gcagaaccgg acagagccca ttacaatatt gtaacctttt gttgcaagtg tgactctacg cttcggttgt 1680
1681 gcgtacaaag cacacacgta gacattcgta ctttggaaga cctgttaatg ggcacactag gaattgtgtg ccccatctgt 1760
1761 tctcaaGGAT CCGAGCTCGG TACCAAGCTT AAGTTTAAAC CGCTGATCAG CCTCGACTGT GCCTTCTAGT TGCCAGCCAT 1840
1841 CTGTTGTTTG CCCCTCCCCC GTGCCTTCCT TGACCCTGGA AGGTGCCACT CCCACTGTCC TTTCCTAATA AAATGAGGAA 1920
1921 ATTGCATCGC ATTGTCTGAG TAGGTGTCAT TCTATTCTGG GGGGTGGGGT GGGGCAGGAC AGCAAGGGGG AGGATTGGGA 2000
2001 AGACAATAGC AGGCATGCTG GGGATGCGGT GGGCTCTATG GCTTCTGAGG CGGAAAGAAC CAGCTGGGGC TCTAGGGGGT 2080
2081 ATCCCCACGC GCCCTGTAGC GGCGCATTAA GCGCGGCGGG TGTGGTGGTT ACGCGCAGCG TGACCGCTAC ACTTGCCAGC 2160
2161 GCCCTAGCGC CCGCTCCTTT CGCTTTCTTC CCTTCCTTTC TCGCCACGTT CGCCGGCTTT CCCCGTCAAG CTCTAAATCG 2240
2241 GGGCATCCCT TTAGGGTTCC GATTTAGTGC TTTACGGCAC CTCGACCCCA AAAAACTTGA TTAGGGTGAT GGTTCACGTA 2320
2321 GTGGGCCATC GCCCTGATAG ACGGTTTTTC GCCCTTTGAC GTTGGAGTCC ACGTTCTTTA ATAGTGGACT CTTGTTCCAA 2400
2401 ACTGGAAGAA CACTCAACCC TATCTCGGTC TATTCTTTTG ATTTATAAGG GATTTTGGGG ATTTCGGCCT ATTGGTTAAA 2480
2481 AAATGAGCTG ATTTAACAAA AATTTAACGC GAATTAATTC TGTGGAATGT GTGTCAGTTA GGGTGTGGAA AGTCCCCAGG 2560
2561 CTCCCCAGGC AGGCAGAAGT ATGCAAAGCA TGCATCTCAA TTAGTCAGCA ACCAGGTGTG GAAAGTCCCC AGGCTCCCCA 2640
2641 GCAGGCAGAA GTATGCAAAG CATGCATCTC AATTAGTCAG CAACCATAGT CCCGCCCCTA ACTCCGCCCA TCCCGCCCCT 2720
2721 AACTCCGCCC AGTTCCGCCC ATTCTCCGCC CCATGGCTGA CTAATTTTTT TTATTTATGC AGAGGCCGAG GCCGCCTCTG 2800
2801 CCTCTGAGCT ATTCCAGAAG TAGTGAGGAG GCTTTTTTGG AGGCCTAGGC TTTTGCAAAA AGCTCCCGGG AGCTTGTATA 2880
2881 TCCATTTTCG GATCTGATCA AGAGACAGGA TGAGGATCGT TTCGCATGAT TGAACAAGAT GGAATGCACG CAGGTTCTCC 2960
2961 GGCCGCTTGG GTGGAGAGGC TATTCGGCTA TGACTGGGCA CAACAGACAA TCGGCTGCTC TGATGCCGCC GTGTTCCGGC 3040
3041 TGTCAGCGCA GGGGCGCCCG GTTCTTTTTG TCAAGACCGA CCTGTCCGGT GCCCTGAATG AACTGCAGGA CGAGGCAGCG 3120
3121 CGGCTATCGT GGCTGGCCAC GACGGGCGTT CCTTGCGCAG CTGTGCTCGA CGTTGTCACT GAAGCGGGAA GGGACTGGCT 3200
3201 GCTATTGGGC GAAGTGCCGG GGCAGGATCT CCTGTCATCT CACCTTGCTC CTGCCGAGAA AGTATCCATC ATGGCTGATG 3280
3281 CAATGCGGCG GCTGCATACG CTTGATCCGG CTACCTGCCC ATTCGACGAC CAAGCGAAAC ATCGCATCGA GCGAGCACGT 3360
3361 ACTCGGATGG AAGCCGGTCT TGTCGATCAG GATGATCTGG ACGAAGAGCA TCAGGGGCTC GCGCCAGCCG AACTGTTCGC 3440
3441 CAGGCTCAAG GCGCGCATGC CCGACGGCGA GGATCTCGTC GTGACCCATG GCGATGCCTG CTTGCCGAAT ATCATGGTGG 3520
3521 AAAATGGCCG CTTTTCTGGA TTCATCGACT GTGGCCGGCT GGGTGTGGCG GACCGCTATC AGGACATAGC GTTGGCTACC 3600
3601 CGTGATATTG CTGAAGAGCT TGGCGGCGAA TGGGCTGACC GCTTCCTCGT GCTTTACGGT ATCGCCGCTC CCGATTCGCA 3680
3681 GCGCATCGCC TTCTATCGCC TTCTTGACGA GTTCTTCTGA GCGGGACTCT GGGGTTCGAA ATGACCGACC AAGCGACGCC 3760
3761 CAACCTGCCA TCACGAGATT TCGATTCCAC CGCCGCCTTC TATGAAAGGT TGGGCTTCGG AATCGTTTTC CGGGACGCCG 3840
3841 GCTGGATGAT CCTCCAGCGC GGGGATCTCA TGCTGGAGTT CTTCGCCCAC CCCAACTTGT TTATTGCAGC TTATAATGGT 3920
3921 TACAAATAAA GCAATAGCAT CACAAATTTC ACAAATAAAG CATTTTTTTC ACTGCATTCT AGTTGTGGTT TGTCCAAACT 4000
4001 CATCAATGTA TCTTATCATG TCTGTATACC GTCGACCTCT AGCTAGAGCT TGGCGTAATC ATGGTCATAG CTGTTTCCTG 4080
4081 TGTGAAATTG TTATCCGCTC ACAATTCCAC ACAACATACG AGCCGGAAGC ATAAAGTGTA AAGCCTGGGG TGCCTAATGA 4160
4161 GTGAGCTAAC TCACATTAAT TGCGTTGCGC TCACTGCCCG CTTTCCAGTC GGGAAACCTG TCGTGCCAGC TGCATTAATG 4240
4241 AATCGGCCAA CGCGCGGGGA GAGGCGGTTT GCGTATTGGG CGCTCTTCCG CTTCCTCGCT CACTGACTCG CTGCGCTCGG 4320
4321 TCGTTCGGCT GCGGCGAGCG GTATCAGCTC ACTCAAAGGC GGTAATACGG TTATCCACAG AATCAGGGGA TAACGCAGGA 4400
4401 AAGAACATGT GAGCAAAAGG CCAGCAAAAG GCCAGCAACC GTAAAAAGCC CGCGTTGCTG GCGTTTTTCC ATAGGCTCCG 4480
4481 CCCCCCTGAC GAGCATCACA AAAATCGACG CTCAAGTCAG AGGTGGCGAA ACCCGACAGG ACTATAAAGA TACCAGGCGT 4560
4561 TTCCCCCTGG AAGCTCCCTC GTGCGCTCTC CTGTTCCGAC CCTGCCGCTT ACCGGATACC TGTCCGCCTT TCTCCCTTCG 4640
4641 GGAAGCGTGG CGCTTTCTCA ATGCTCACGC TGTAGGTATC TCAGTTCGGT GTAGGTCGTT CGCTCCAAGC TGGGCTGTGT 4720
4721 GCACGAACCC CCCGTTCAGC CCGACCGCTG CGCCTTATCC GGTAACTATC GTCTTGAGTC CAACCCGGTA AGACACGACT 4800
4801 TATCGCCACT GGCAGCAGCC ACTGGTAACA GGATTAGCAG AGCGAGGTAT GTAGGCGGTG CTACAGAGTT CTTGAAGTGG 4880
4881 TGGCCTAACT ACGGCTACAC TAGAAGGACA GTATTTGGTA TCTGCGCTCT GCTGAAGCCA GTTACCTTCG GAAAAAGAGT 4960
4961 TGGTAGCTCT TGATCCGGCA AACAAACCAC CGCTGGTAGC GGTGGTTTTT TTGTTTGCAA GCAGCAGATT ACGCGCAGAA 5040
5041 AAAAAGGATC TCAAGAAGAT CCTTTGATCT TTTCTACGGG GTCTGACGCT CAGTGGAACG AAAACTCACG TTAAGGGATT 5120
5121 TTGGTCATGA GATTATCAAA AAGGATCTTC ACCTAGATCC TTTTAAATTA AAAATGAAGT TTTAAATCAA TCTAAAGTAT 5200
5201 ATATGAGTAA ACTTGGTCTG ACAGTTACCA ATGCTTAATC AGTGAGGCAC CTATCTCAGC GATCTGTCTA TTTCGTTCAT 5280
5281 CCATAGTTGC CTGACTCCCC GTCGTGTAGA TAACTACGAT ACGGGAGGGC TTACCATCTG GCCCCAGTGC TGCAATGATA 5360
5361 CCGCGAGACC CACGCTCACC GGCTCCAGAT TTATCAGCAA TAAACCAGCC AGCCGGAAGG GCCGAGCGCA GAAGTGGTCC 5440
5441 TGCAACTTTA TCCGCCTCCA TCCAGTCTAT TAATTGTTGC CGGGAAGCTA GAGTAAGTAG TTCGCCAGTT AATAGTTTGC 5520
5521 GCAACGTTGT TGCCATTGCT ACAGGCATCG TGGTGTCACG CTCGTCGTTT GGTATGGCTT CATTCAGCTC CGGTTCCCAA 5600
5601 CGATCAAGGC GAGTTACATG ATCCCCCATG TTGTGCAAAA AAGCGGTTAG CTCCTTCGGT CCTCCGATCG TTGTCAGAAG 5680
5681 TAAGTTGGCC GCAGTGTTAT CACTCATGGT TATGGCAGCA CTGCATAATT CTCTTACTGT CATGCCATCC GTAAGATGCT 5760
5761 TTTCTGTGAC TGGTGAGTAC TCAACCAAGT CATTCTGAGA ATAGTGTATG CGGCGACCGA GTTGCTCTTG CCCGGCGTCA 5840
5841 ATACGGGATA ATACCGCGCC ACATAGCAGA ACTTTAAAAG TGCTCATCAT TGGAAAACGT TCTTCGGGGC GAAAACTCTC 5920
5921 AAGGATCTTA CCGCTGTTGA GATCCAGTTC GATGTAACCC ACTCGTGCAC CCAACTGATC TTCAGCATCT TTTACTTTCA 6000
6001 CCAGCGTTTC TGGGTGAGCA AAAACAGGAA GGCAAAATGC CGCAAAAAAG GGAATAAGGG CGACACGGAA ATGTTGAATA 6080
6081 CTCATACTCT TCCTTTTTCA ATATTATTGA AGCATTTATC AGGGTTATTG TCTCATGAGC GGATACATAT TTGAATGTAT 6160
6161 TTAGAAAAAT AAACAAATAG GGGTTCCGCG CACATTTCCC CGAAAAGTGC CACCTGACGT C 6221
        | 10 | 20 | 30 | 40 | 50 | 60 | 70 | 80 |
```

pSCA1-E7-Hsp70 SEQ ID NO:68 The E7-Hsp70 fusion sequence is shown in bold, caps

```
          | 10 | 20 | 30 | 40 | 50 | 60 | 70 | 80 |
        1 atggcggatg tgtgacatac acgacgccaa aagattttgt tccagctcct gccacctccg ctacgcgaga gattaaccac 80
      81 ccacgatggc cgccaaagtg catgttgata ttgaggctga cagcccattc atcaagtctt tgcagaaggc atttccgtcg 160
    161 ttcgaggtgg agtcattgca ggtcacacca aatgaccatg caaatgccag agcattttcg cacctggcta ccaaattgat 240
    241 cgagcaggag actgacaaag acacactcat cttggatatc ggcagtgcgc cttccaggag aatgatgtct acgcacaaat 320
    321 accactgcgt atgccctatg cgcagcgcag aagaccccga aaggctcgat agctacgcaa agaaactggc agcggcctcc 400
    401 gggaaggtgc tggatagaga gatcgcagga aaaatcaccg acctgcagac cgtcatggct acgccagacg ctgaatctcc 480
    481 taccttttgc ctgcatacag acgtcacgtg tcgtacggca gccgaagtgg ccgtatacca ggacgtgtat gctgtacatg 560
    561 caccaacatc gctgtaccat caggcgatga aaggtgtcag aacggcgtat tggattgggt ttgacaccac cccgtttatg 640
    641 tttgacgcgc tagcaggcgc gtatccaacc tacgccacaa actgggccga cgagcaggtg ttacaggcca ggaacatagg 720
    721 actgtgtgca gcatccttga ctgagggaag actcggcaaa ctgtccattc tccgcaagaa gcaattgaaa ccttgcgaca 800
    801 cagtcatgtt ctcggtagga tctacattgt acactgagag cagaaagcta ctgaggagct ggcacttacc ctccgtattc 880
    881 cacctgaaag gtaaacaatc ctttacctgt aggtgcgata ccatcgtatc atgtgaaggg tacgtagtta agaaaatcac 960
    961 tatgtgcccc ggcctgtacg gtaaaacggt agggtacgcc gtgacgtatc acgcggaggg attcctagtg tgcaagacca 1040
  1041 cagacactgt caaaggagaa agagtctcat tccctgtatg cacctacgtc ccctcaacca tctgtgatca aatgactggc 1120
  1121 atactagcga ccgacgtcac accggaggac gcacagaagt tgttagtggg attgaatcag aggatagttg tgaacggaag 1200
  1201 aacacagcga aacactaaca cgatgaagaa ctatctgctt ccgattgtgg ccgtcgcatt tagcaagtgg gcgagggaat 1280
  1281 acaaggcaga ccttgatgat gaaaaacctc tgggtgtccg agagaggtca cttacttgct gctgcttgtg ggcatttaaa 1360
  1361 acgaggaaga tgcacaccat gtacaagaaa ccagacaccc agacaatagt gaaggtgcct tcagagttta actcgttcgt 1440
  1441 catcccgagc ctatggtcta caggcctcgc aatcccagtc agatcacgca ttaagatgct tttggccaag aagaccaagc 1520
  1521 gagagttaat acctgttctc gacgcgtcgt cagccaggga tgctgaacaa gaggagaagg agaggttgga ggccgagctg 1600
  1601 actagagaag ccttaccacc cctcgtcccc atcgcgccgg cggagacggg agtcgtcgac gtcgacgttg aagaactaga 1680
  1681 gtatcacgca ggtgcagggg tcgtggaaac acctcgcagc gcgttgaaag tcaccgcaca gccgaacgac gtactactag 1760
  1761 gaaattacgt agttctgtcc ccgcagaccg tgctcaagag ctccaagttg gcccccgtgc accctctagc agagcaggtg 1840
  1841 aaaataataa cacataacgg gagggccggc ggttaccagg tcgacggata tgacggcagg gtcctactac catgtggatc 1920
  1921 ggccattccg gtccctgagt ttcaagcttt gagcgagagc gccactatgg tgtacaacga aagggagttc gtcaacagga 2000
  2001 aactatacca tattgccgtt cacggaccgt cgctgaacac cgacgaggag aactacgaga aagtcagagc tgaaagaact 2080
  2081 gacgccgagt acgtgttcga cgtagataaa aaatgctgcg tcaagagaga ggaagcgtcg ggtttggtgt tggtgggaga 2160
  2161 gctaaccaac cccccgttcc atgaattcgc ctacgaaggg ctgaagatca ggccgtcggc accatataag actacagtag 2240
  2241 taggagtctt tggggttccg ggatcaggca agtctgctat tattaagagc ctcgtgacca aacacgatct ggtcaccagc 2320
  2321 ggcaagaagg agaactgcca ggaaatagtt aacgacgtga agaagcaccg cgggaagggg acaagtaggg aaaacagtga 2400
  2401 ctccatcctg ctaaacgggt gtcgtcgtgc cgtggacatc ctatatgtgg acgaggcttt cgctagccat tccggtactc 2480
  2481 tgctggccct aattgctctt gttaaacctc ggagcaaagt ggtgttatgc ggagacccca agcaatgcgg attcttcaat 2560
  2561 atgatgcagc ttaaggtgaa cttcaaccac aacatctgca ctgaagtatg tcataaaagt atatccagac gttgcacgcg 2640
  2641 tccagtcacg gccatcgtgt ctacgttgca ctacggaggc aagatgcgca cgaccaaccc gtgcaacaaa cccataatca 2720
  2721 tagacaccac aggacagacc aagcccaagc caggagacat cgtgttaaca tgcttccgag gctgggcaaa gcagctgcag 2800
  2801 ttggactacc gtggacacga agtcatgaca gcagcagcat ctcagggcct cacccgcaaa ggggtatacg ccgtaaggca 2880
  2881 gaaggtgaat gaaaatccct tgtatgcccc tgcgtcggag cacgtgaatg tactgctgac gcgcactgag gataggctgg 2960
  2961 tgtggaaaac gctggccggc gatccctgga ttaaggtcct atcaaacatt ccacagggta actttacggc cacattggaa 3040
  3041 gaatggcaag aagaacacga caaaataatg aaggtgattg aaggaccggc tgcgcctgtg gacgcgttcc agaacaaagc 3120
  3121 gaacgtgtgt tgggcgaaaa gcctggtgcc tgtcctggac actgccggaa tcagattgac agcagaggag tggagcacca 3200
  3201 taattacagc atttaaggag gacagagctt actctccagt ggtggccttg aatgaaattt gcaccaagta ctatggagtt 3280
  3281 gacctggaca gtggcctgtt ttctgccccg aaggtgtccc tgtattacga gaacaaccac tgggataaca gacctggtgg 3360
  3361 aaggatgtat ggattcaatg ccgcaacagc tgccaggctg gaagctagac ataccttcct gaaggggcag tggcatacgg 3440
  3441 gcaagcaggc agttatcgca gaaagaaaaa tccaaccgct ttctgtgctg gacaatgtaa ttcctatcaa ccgcaggctg 3520
  3521 ccgcacgccc tggtggctga gtacaagacg gttaaaggca gtagggttga gtggctggtc aataaagtaa gagggtacca 3600
  3601 cgtcctgctg gtgagtgagt acaacctggc tttgcctcga cgcagggtca cttggttgtc accgctgaat gtcacaggcg 3680
  3681 ccgataggtg ctacgaccta agtttaggac tgccggctga cgccggcagg ttcgacttgg tctttgtgaa cattcacacg 3760
  3761 gaattcagaa tccaccacta ccagcagtgt gtcgaccacg ccatgaagct gcagatgctt gggggagatg cgctacgact 3840
  3841 gctaaaaccc ggcggcatct tgatgagagc ttacggatac gccgataaaa tcagcgaagc cgttgtttcc tccttaagca 3920
  3921 gaaagttctc gtctgcaaga gtgttgcgcc cggattgtgt caccagcaat acagaagtgt tcttgctgtt ctccaacttt 4000
  4001 gacaacggaa agagaccctc tacgctacac cagatgaata ccaagctgag tgccgtgtat gccggagaag ccatgcacac 4080
  4081 ggccgggtgt gcaccatcct acagagttaa gagagcagac atagccacgt gcacagaagc ggctgtggtt aacgcagcta 4160
  4161 acgcccgtgg aactgtaggg gatggcgtat gcagggccgt ggcgaagaaa tggccgtcag cctttaaggg agcagcaaca 4240
  4241 ccagtgggca caattaaaac agtcatgtgc ggctcgtacc ccgtcatcca cgctgtagcg cctaatttct ctgccacgac 4320
  4321 tgaagcggaa ggggaccgcg aattggccgc tgtctaccgg gcagtggccg ccgaagtaaa cagactgtca ctgagcagcg 4400
  4401 tagccatccc gctgctgtcc acaggagtgt tcagcggcgg aagagatagg ctgcagcaat ccctcaacca tctattcaca 4480
  4481 gcaatggacg ccacggacgc tgacgtgacc atctactgca gagacaaaag ttgggagaag aaaatccagg aagccattga 4560
  4561 catgaggacg gctgtggagt tgctcaatga tgacgtggag ctgaccacag acttggtgag agtgcacccg gacagcagcc 4640
  4641 tggtgggtcg taagggctac agtaccactg acgggtcgct gtactcgtac tttgaaggta cgaaattcaa ccaggctgct 4720
  4721 attgatatgg cagagatact gacgttgtgg cccagactgc aagaggcaaa cgaacagata tgcctatacg cgctgggcga 4800
  4801 aacaatggac aacatcagat ccaaatgtcc ggtgaacgat tccgattcat caacacctcc caggacagtg ccctgcctgt 4880
  4881 gccgctacgc aatgacagca gaacggatcg cccgccttag gtcacaccaa gttaaaagca tggtggtttg ctcatctttt 4960
  4961 cccctcccga aataccatgt agatggggtg cagaaggtaa agtgcgagaa ggttctcctg ttcgacccga cggtaccttc 5040
  5041 agtggttagt ccgcggaagt atgccgcatc tacgacggac cactcagatc ggtcgttacg agggtttgac ttggactgga 5120
  5121 ccaccgactc gtcttccact gccagcgata ccatgtcgct acccagtttg cagtcgtgtg acatcgactc gatctacgag 5200
  5201 ccaatggctc ccatagtagt gacggctgac gtacaccctg aacccgcagg catcgcggac ctggcggcag atgtgcaccc 5280
  5281 tgaacccgca gaccatgtgg acctcgagaa cccgattcct ccaccgcgcc cgaagagagc tgcatacctt gcctcccgcg 5360
  5361 cggcggagcg accggtgccg gcgccgagaa agccgacgcc tgccccaagg actgcgttta ggaacaagct gcctttgacg 5440
  5441 ttcggcgact ttgacgagca cgaggtcgat gcgttggcct ccgggattac tttcggagac ttcgacgacg tcctgcgact 5520
  5521 aggccgcgcg ggtgcatata ttttctcctc ggacactggc agcggacatt tacaacaaaa atccgttagg cagcacaatc 5600
  5601 tccagtgcgc acaactggat gcggtccagg aggagaaaat gtacccgcca aaattggata ctgagaggga gaagctgttg 5680
  5681 ctgctgaaaa tgcagatgca cccatcggag gctaataaga gtcgatacca gtctcgcaaa gtggagaaca tgaaagccac 5760
  5761 ggtggtggac aggctcacat cgggggccag attgtacacg ggagcggacg taggccgcat accaacatac gcggttcggt 5840
  5841 acccccgccc cgtgtactcc cctaccgtga tcgaaagatt ctcaagcccc gatgtagcaa tcgcagcgtg caacgaatac 5920
  5921 ctatccagaa attacccaac agtggcgtcg taccagataa cagatgaata cgacgcatac ttggacatgg ttgacgggtc 6000
  6001 ggatagttgc ttggacagag cgacattctg cccggcgaag ctccggtgct acccgaaaca tcatgcgtac caccagccga 6080
  6081 ctgtacgcag tgccgtcccg tcaccctttc agaacacact acagaacgtg ctagcggccg ccaccaagag aaactgcaac 6160
  6161 gtcacgcaaa tgcgagaact acccaccatg gactcggcag tgttcaacgt ggagtgcttc aagcgctatg cctgctccgg 6240
  6241 agaatattgg gaagaatatg ctaaacaacc tatccggata accactgaga acatcactac ctatgtgacc aaattgaaag 6320
  6321 gcccgaaagc tgctgccttg ttcgctaaga cccacaactt ggttccgctg caggaggttc ccatggacag attcacggtc 6400
  6401 gacatgaaac gagatgtcaa agtcactcca gggacgaaac acacagagga aagacccaaa gtccaggtaa ttcaagcagc 6480
  6481 ggagccattg gcgaccgctt acctgtgcgg catccacagg gaattagtaa ggagactaaa tgctgtgtta cgccctaacg 6560
  6561 tgcacacatt gtttgatatg tcggccgaag actttgacgc gatcatcgcc tctcacttcc acccaggaga cccggttcta 6640
  6641 gagacggaca ttgcatcatt cgacaaaagc caggacgact ccttggctct tacaggttta atgatcctcg aagatctagg 6720
  6721 ggtggatcag tacctgctgg acttgatcga ggcagccttt ggggaaatat ccagctgtca cctaccaact ggcacgcgct 6800
  6801 tcaagttcgg agctatgatg aaatcgggca tgtttctgac tttgtttatt aacactgttt tgaacatcac catagcaagc 6880
  6881 agggtactgg agcagagact cactgactcc gcctgtgcgg ccttcatcgg cgacgacaac atcgttcacg gagtgatctc 6960
  6961 cgacaagctg atggcggaga ggtgcgcgtc gtgggtcaac atggaggtga agatcattga cgctgtcatg ggcgaaaaac 7040
  7041 ccccatattt ttgtggggga ttcatagttt ttgacagcgt cacacagacc gcctgccgtg tttcagaccc acttaagcgc 7120
  7121 ctgttcaagt tgggtaagcc gctaacagct gaagacaagc aggacgaaga caggcgacga gcactgagtg acgaggttag 7200
  7201 caagtggttc cggacaggct tgggggccga actggaggtg gcactaacat ctaggtatga ggtagagggc tgcaaaagta 7280
  7281 tcctcatagc catggccacc ttggcgaggg acattaaggc gtttaagaaa ttgagaggac ctgttataca cctctacggc 7360
  7361 ggtcctagat tggtgcgtta atacacagaa ttctgattgg atCCATGCAT GGAGATACAC CTACATTGCA TGAATATATG 7440
  7441 TTAGATTTGC AACCAGAGAC AACTGATCTC TACTGTTATG AGCAATTAAA TGACAGCTCA GAGGAGGAGG ATGAAATAGA 7520
  7521 TGGTCCAGCT GGACAAGCAG AACCGGACAG AGCCCATTAC AATATTTGTAA CCTTTGTTG CAAGTGTGAC TCTACGCTTC 7600
  7601 GGTTGTGCGT ACAAAGCACA CACGTAGACA TTCGTACTTT GGAAGACCTG TTAATGGGCA CACTAGGAAT TGTGTGCCCC 7680
  7681 ATCTGTTCTC AAGGATCCAT GGCTCGTGCG GTCGGGATCG ACCTCGGGAC CACCAACTCC GTCGTCTCGG TTCTGGAAGG 7760
  7761 TGGCGACCCG GTCGTCGTCG CCAACTCCGA GGGCTCCAGG ACCACCCCGT CAATTGTCGC GTTCGCCCGC AACGGTGAGG 7840
  7841 TGCTGGTCGG CCAGCCCGCC AAGAACCAGG CAGTGACCAA CGTCGATCGC ACCGTGCGCT CGGTCAAGCG ACACATGGGC 7920
  7921 AGCGACTGGT CCATAGAGAT TGACGGCAAG AAATACACCG CGCCGGAGAT CAGCGCCCGC ATTCTGATGA AGCTGAAGCG 8000
  8001 CGACGCCGAG GCCTACCTCG GTGAGGACAT TACCGACGCG GTTATCACGA CGCCCGCCTA CTTCAATGAC GCCCAGCGTC 8080
  8081 AGGCCACCAA GGACGCCGGC CAGATCGCCG GCCTCAACGT GCTGCGGATC GTCAACGAGC CGACCGCGGC CGCGCTGGCC 8160
  8161 TACGGCCTCG ACAAGGGCGA GAAGGAGCAG CGAATCCTGG TCTTCGACTT GGGTGGTGGC ACTTTCGACG TTTCCCTGCT 8240
  8241 GGAGATCGGC GAGGGTGTGG TTGAGGTCCG TGCCACTTCG GGTGACAACC ACCTCGGCGG CGACGACTGG GACCAGCGGG 8320
  8321 TCGTCGATTG GCTGGTGGAC AAGTTCAAGG GCACCAGCGG CATCGATCTG ACCAAGGACA AGATGGCGAT GCAGCGGCTG 8400
  8401 CGGGAAGCCC CCGAGAAGGC AAAGATCGAG CTGAGTTCGA GTCAGTCCAC CTCGATCAAC CTGCCCTACA TCACCGTCGA 8480
  8481 CGCCGACAAG AACCCGTTGT TCTTAGACGA GCAGCTGACC CGCGCGGAGT TCCAACGGAT CACTCAGGAC CTGCTGGACC 8560
  8561 GCACTCGCAA GCCGTTCCAG TCGGTGATCG CTGACACCGG CATTTCGGTG TCGGAGATCG ATCACGTTGT GCTCGTGGGT 8640
  8641 GGTTCGACCC GGATGCCCGC GGTGACCGAT CTGGTCAAGG AACTCACCGG CGGCAAGGAA CCCAACAAGG GCGTCAACCC 8720
  8721 CGATGAGGTT GTCGCGGTGG GAGCCGCTCT GCAGGCCGGC GTCCTCAAGG GCGAGGTGAA AGACGTTCTG CTGCTTGATG 8800
  8801 TTACCCCGCT GAGCCTGGGT ATCGAGACCA AGGGCGGGGT GATGACCAGG CTCATCGAGC GCAACACCAC GATCCCCACC 8880
  8881 AAGCGGTCGG AGACTTTCAC CACCGCCGAC GACAACCAAC CGTCGGTGCA GATCCAGGTC TATCAGGGGG AGCGTGAGAT 8960
  8961 CGCCGCGCAC AACAAGTTGC TCGGGTCCTT CGAGCTGACC GGCATCCCGC CGGCGCCGCG GGGGATTCCG CAGATCGAGG 9040
  9041 TCACTTTCGA CATCGACGCC AACGGCATTG TGCACGTCAC CGCCAAGGAC AAGGGCACCG GCAAGGAGAA CACGATCCGA 9120
  9121 ATCCAGGAAG GCTCGGGCCT GTCCAAGGAA GACATTGACC GCATGATCAA GGACGCCGAA GCGCACGCCG AGGAGGATCG 9200
  9201 CAAGCGTCGC GAGGAGGCCG ATGTTCGTAA TCAAGCCGAG ACATTGGTCT ACCAGACGGA GAAGTTCGTC AAAGAACAGC 9280
  9281 GTGAGGCCGA GGGTGGTTCG AAGGTACCTG AAGACACGCT GAACAAGGTT GATGCCGCGG TGGCGGAAGC GAAGGCGGCA 9360
  9361 CTTGGCGGAT CGGATATTTC GGCCATCAAG TCGGCGATGG AGAAGCTGGG CCAGGAGTCG CAGGCTCTGG GGCAAGCGAT 9440
  9441 CTACGAAGCA GCTCAGGCTG CGTCACAGGC CACTGGCGCT GCCCACCCCG GCTCGGCTGA TGAAAGCTTa agtttgggta 9520
  9521 attaattgaa ttacatccct acgcaaacgt tttacggccg ccggtggcgc ccgcgcccgg cggcccgtcc ttggccgttg 9600
  9601 caggccactc cggtggctcc cgtcgtcccc gacttccagg cccagcagat gcagcaactc atcagcgccg taaatgcgct 9680
  9681 gacaatgaga cagaacgcaa ttgctcctgc taggcctccc aaaccaaaga agaagaagac aaccaaacca aagccgaaaa 9760
  9761 cgcagcccaa gaagatcaac ggaaaaacgc agcagcaaaa gaagaaagac aagcaagccg acaagaagaa gaagaaaccc 9840
  9841 ggaaaaagag aaagaatgtg catgaagatt gaaaatgact gtatcttcgt atgcggctag ccacagtaac gtagtgtttc 9920
  9921 cagacatgtc gggcaccgca ctatcatggg tgcagaaaat ctcgggtggt ctgggggcct tcgcaatcgg cgctatcctg 10000
10001 gtgctggttg tggtcacttg cattgggctc cgcagataag ttagggtagg caatggcatt gatatagcaa gaaaattgaa 10080
10081 aacagaaaaa gttagggtaa gcaatggcat ataaccataa ctgtataact tgtaacaaag cgcaacaaga cctgcgcaat 10160
10161 tggccccgtg gtccgcctca cggaaactcg gggcaactca tattgacaca ttaattggca ataattggaa gcttacataa 10240
10241 gcttaattcg acgaataatt ggatttttat tttattttgc aattggtttt taatatttcc aaaaaaaaaa aaaaaaaaaa 10320
10321 aaaaaaaaaa aaaaaaaaaa aaaaaaaaaa aaaaaaaaaa aaaaaaaaaa ctagtgatca taatcagcca taccacattt 10400
10401 gtagaggttt tacttgcttt aaaaaacctc ccacacctcc ccctgaacct gaaacataaa atgaatgcaa ttgttgttgt 10480
10481 taacttgttt attgcagctt ataatggtta caaataaagc aatagcatca caaatttcac aaataaagca tttttttcac 10560
10561 tgcattctag ttgtggtttg tccaaactca tcaatgtatc ttatcatgtc tggatctagt ctgcattaat gaatcggcca 10640
10641 acgcgcgggg agaggcggtt tgcgtattgg gcgctcttcc gcttcctcgc tcactgactc gctgcgctcg gtcgttcggc 10720
10721 tgcggcgagc ggtatcagct cactcaaagg cggtaatacg gttatccaca gaatcagggg ataacgcagg aaagaacatg 10800
10801 tgagcaaaag gccagcaaaa ggccaggaac cgtaaaaagg ccgcgttgct ggcgtttttc cataggctcc gcccccctga 10880
10881 cgagcatcac aaaaatcgac gctcaagtca gaggtggcga aacccgacag gactataaag ataccaggcg tttccccctg 10960
10961 gaagctccct cgtgcgctct cctgttccga ccctgccgct taccggatac ctgtccgcct ttctcccttc gggaagcgtg 11040
11041 gcgctttctc aatgctcgcg ctgtaggtat ctcagttcgg tgtaggtcgt tcgctccaag ctgggctgtg tgcacgaacc 11120
11121 ccccgttcag cccgaccgct gcgccttatc cggtaactat cgtcttgagt ccaacccggt aagacacgac ttatcgccac 11200
11201 tggcagcagc cactggtaac aggattagca gagcgaggta tgtaggcggt gctacagagt tcttgaagtg gtggcctaac 11280
11281 tacggctaca ctagaaggac agtatttggt atctgcgctc tgctgaagcc agttaccttc ggaaaaagag ttggtagctc 11360
11361 ttgatccggc aaacaaacca ccgctggtag cggtggtttt tttgtttgca agcagcagat tacgcgcaga aaaaaaggat 11440
11441 ctcaagaaga tcctttgatc ttttctacgg ggcattctga cgctcagtgg aacgaaaact cacgttaagg gattttggtc 11520
11521 atgagattat caaaaaggat cttcacctag atccttttaa attaaaaatg aagttttaaa tcaatctaaa gtatatatga 11600
11601 gtaaacttgg tctgacagtt accaatgctt aatcagtgag gcacctatct cagcgatctg tctatttcgt tcatccatag 11680
11681 ttgcctgact ccccgtcgtg tagataacta cgatacggga gggcttacca tctggcccca gtgctgcaat gataccgcga 11760
11761 gacccacgct caccggctcc agatttatca gcaataaacc agccagccgg aagggccgag cgcagaagtg gtcctgcaac 11840
11841 tttatccgcc tccatccagt ctattaattg ttgccgggaa gctagagtaa gtagttcgcc agttaatagt ttgcgcaacg 11920
11921 ttgttgccat tgctacaggc atcgtggtgt cacgctcgtc gtttggtatg gcttcattca gctccggttc ccaacgatca 12000
12001 aggcgagtta catgatcccc catgttgtgc aaaaaagcgg ttagctcctt cggtcctccg atcgttgtca gaagtaagtt 12080
12081 ggccgcagtg ttatcactca tggttatggc agcactgcat aattctctta ctgtcatgcc atccgtaaga tgcttttctg 12160
12161 tgactggtga gtactcaacc aagtcattct gagaatagtg tatgcggcga ccgagttgct cttgcccggc gtcaatacgg 12240
12241 gataataccg cgccacatag cagaacttta aaagtgctca tcattggaaa acgttcttcg gggcgaaaac tctcaaggat 12320
12321 cttaccgctg ttgagatcca gttcgatgta acccactcgt gcacccaact gatcttcagc atcttttact ttcaccagcg 12400
12401 tttctgggtg agcaaaaaca ggaaggcaaa atgccgcaaa aaagggaata agggcgacac ggaaatgttg aatactcata 12480
12481 ctcttccttt ttcaatatta ttgaagcatt tatcagggtt attgtctcat gagcggatac atatttgaat gtatttagaa 12560
12561 aaataaacaa ataggggttc cgcgcacatt tccccgaaaa gtgccacctg acgtctaaga aaccattatt atcatgacat 12640
12641 taacctataa aaataggcgt atcacgaggc cctttcgtct cgcgcgtttc ggtgatgacg gtgaaaacct ctgacacatg 12720
12721 cagctcccgg agacggtcac agcttctgtc taagcggatg ccgggagcag acaagcccgt cagggcgcgt cagcgggtgt 12800
12801 tggcgggtgt cggggctggc ttaactatgc ggcatcagag cagattgtac tgagagtgca ccatatcgac gctctccctt 12880
12881 atgcgactcc tgcattagga agcagcccag tactaggttg aggccgttga gcaccgccgc cgcaaggaat ggtgcatgcg 12960
12961 taatcaatta cggggtcatt agttcatagc ccatatatgg agttccgcgt tacataactt acggtaaatg gcccgcctgg 13040
13041 ctgaccgccc aacgaccccc gcccattgac gtcaataatg acgtatgttc ccatagtaac gccaataggg actttccatt 13120
13121 gacgtcaatg ggtggagtat ttacggtaaa ctgcccactt ggcagtacat caagtgtatc atatgccaag tacgccccct 13200
13201 attgacgtca atgacggtaa atggcccgcc tggcattatg cccagtacat gaccttatgg gactttccta cttggcagta 13280
13281 catctacgta ttagtcatcg ctattaccat ggtgatgcgg ttttggcagt acatcaatgg gcgtggatag cggtttgact 13360
13361 cacggggatt tccaagtctc caccccattg acgtcaatgg gagtttgttt tggcaccaaa atcaacggga ctttccaaaa 13440
13441 tgtcgtaaca actccgcccc attgacgcaa atgggcggta ggcgtgtacg gtgggaggtc tatataagca gagctctctg 13520
13521 gctaactaga gaacccactg cttaactggc ttatcgaaat taatacgact cactataggg agaccggaag cttgaattc 13599
          | 10 | 20 | 30 | 40 | 50 | 60 | 70 | 80 |
```

Calreticulin (CRT)

“Calreticulin” or “CRT” describes the well-characterized ˜46 kDa resident protein of the ER lumen that has lectin activity and participates in the folding and assembly of nascent glycoproteins. CRT acts as a “chaperone” polypeptide and a member of the MHC class I transporter TAP complex; CRT associates with TAP1 and TAP2 transporters, tapasin, MHC Class I heavy chain polypeptide and β2 microglobulin to function in the loading of peptide epitopes onto nascent MHC class I molecules (Jorgensen (2000) Eur. J. Biochem. 267:2945-2954). The term “calreticulin” or “CRT” refers to polypeptides and nucleic acids molecules having substantial identity (defined herein) to the exemplary CRT sequences as described herein. A CRT polypeptide is a polypeptides comprising a sequence identical to or substantially identical (defined herein) to the amino acid sequence of CRT. An exemplary nucleotide and amino acid sequence for a CRT used in the present compositions and methods are presented below. The terms “calreticulin” or “CRT” encompass native proteins as well as recombinantly produced modified proteins that induce an immune response, including a CTL response. The terms “calreticulin” or “CRT” encompass homologues and allelic variants of CRT, including variants of native proteins constructed by in vitro techniques, and proteins isolated from natural sources. The CRT polypeptides of the invention, and sequences encoding them, also include fusion proteins comprising non-CRT sequences, particularly MHC class I-binding peptides; and also further comprising other domains, e.g., epitope tags, enzyme cleavage recognition sequences, signal sequences, secretion signals and the like.

The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof. A polypeptide or peptide can be routinely screened for its activity as an ER chaperone using assays known in the art, such as that set forth in Example 1. While the invention is not limited by any particular mechanism of action, in vivo chaperones promote the correct folding and oligomerization of many glycoproteins in the ER, including the assembly of the MHC class I heterotrimeric molecule (heavy (H) chain, β2m, and peptide). They also retain incompletely assembled MHC class I heterotrimeric complexes in the ER (Hauri (2000) FEBS Lett. 476:32-37).

The sequences of CRT, including human CRT, are well known in the art (McCauliffe (1990) J. Clin. Invest. 86:332-335; Burns (1994) Nature 367:476-480; Coppolino (1998) Int. J. Biochem. Cell Biol. 30:553-558). The nucleic acid sequence appears as GenBank Accession No. NM 004343 and is SEQ ID NO:69

```
      1 gtccgtactg cagagccgct gccggagggt cgttttaaag ggccgcgttg ccgccccctc
    61 ggcccgccat gctgctatcc gtgccgctgc tgctcggcct cctcggcctg gccgtcgccg
  121 agcccgccgt ctacttcaag gagcagtttc tggacggaga cgggtggact tcccgctgga
  181 tcgaatccaa acacaagtca gattttggca aattcgttct cagttccggc aagttctacg
  241 gtgacgagga gaaagataaa ggtttgcaga caagccagga tgcacgcttt tatgctctgt
  301 cggccagttt cgagcctttc agcaacaaag gccagacgct ggtggtgcag ttcacggtga
  361 aacatgagca gaacatcgac tgtgggggcg gctatgtgaa gctgtttcct aatagtttgg
  421 accagacaga catgcacgga gactcagaat acaacatcat gtttggtccc gacatctgtg
  481 gccctggcac caagaaggtt catgtcatct tcaactacaa gggcaagaac gtgctgatca
  541 acaaggacat ccgttgcaag gatgatgagt ttacacacct gtacacactg attgtgcggc
  601 cagacaacac ctatgaggtg aagattgaca acagccaggt ggagtccggc tccttggaag
  661 acgattggga cttcctgcca cccaagaaga taaaggatcc tgatgcttca aaaccggaag
  721 actgggatga gcgggccaag atcgatgatc ccacagactc caagcctgag gactgggaca
  781 agcccgagca tatccctgac cctgatgcta agaagcccga ggactgggat gaagagatgg
  841 acggagagtg ggaaccccca gtgattcaga accctgagta caagggtgag tggaagcccc
  901 ggcagatcga caacccagat tacaagggca cttggatcca cccagaaatt gacaaccccg
  961 agtattctcc cgatcccagt atctatgcct atgataactt tggcgtgctg ggcctggacc
1021 tctggcaggt caagtctggc accatctttg acaacttcct catcaccaac gatgaggcat
1081 acgctgagga gtttggcaac gagacgtggg gcgtaacaaa ggcagcagag aaacaaatga
1141 aggacaaaca ggacgaggag cagaggctta aggaggagga agaagacaag aaacgcaaag
1201 aggaggagga ggcagaggac aaggaggatg atgaggacaa agatgaggat gaggaggatg
1261 aggaggacaa ggaggaagat gaggaggaag atgtccccgg ccaggccaag gacgagctgt
1321 agagaggcct gcctccaggg ctggactgag gcctgagcgc tcctgccgca gagcttgccg
1381 cgccaaataa tgtctctgtg agactcgaga actttcattt ttttccaggc tggttcggat
1441 ttggggtgga ttttggtttt gttcccctcc tccactctcc cccaccccct ccccgccctt
1501 tttttttttt tttttaaact ggtattttat cctttgattc tccttcagcc ctcacccctg
1561 gttctcatct ttcttgatca acatcttttc ttgcctctgt gccccttctc tcatctctta
1621 gctcccctcc aacctggggg gcagtggtgt ggagaagcca caggcctgag atttcatctg
1681 ctctccttcc tggagcccag aggagggcag cagaaggggg tggtgtctcc aaccccccag
1741 cactgaggaa gaacggggct cttctcattt cacccctccc tttctcccct gcccccagga
1801 ctgggccact tctgggtggg gcagtgggtc ccagattggc tcacactgag aatgtaagaa
1861 ctacaaacaa aatttctatt aaattaaatt ttgtgtctc
1899
```

Human CRT protein (GenBank Accession No. NM 004343), (SEQ ID NO:70) is shown below:

```
    1 MLLSVPLLLG LLGLAVAEPA VYFKEQFLDG DGWTSRWIES KHKSDFGKFV LSSGKFYGDE
  61 EKDKGLQTSQ DARFYALSAS FEPFSNKGQT LVVQFTVKHE QNIDCGGGYV KLFPNSLDQT
121 DMHGDSEYNI MFGPDICGPG TKKVHVIFNY KGKNVLINKD IRCKDDEFTH LYTLIVRPDN
181 TYEVKIDNSQ VESGSLEDDW DFLPPKKIKD PDASKPEDWD ERAKIDDPTD SKPEDWDKPE
241 HIPDPDAKKP EDWDEEMDGE WEPPVIQNPE YKGEWKPRQI DNPDYKGTWI HPEIDNPEYS
301 PDPSIYAYDN FGVLGLDLWQ VKSGTIFDNF LITNDEAYAE EFGNETWGVT KAAEKQMKDK
361 QDEEQRLKEE EEDKKRKEEE EAEDKEDDED KDEDEEDEED KEEDEEEDVP GQAKDEL
417
```

For the generation of plasmid encoding the full length of rabbit calreticulin (there is more than 90% homology between rabbit, human, mouse, and rat calreticulin), pcDNA3-CRT, the DNA fragment encoding this protein was first amplified with PCR using conditions as described in Chen (2000) Cancer Res., supra, using rabbit calreticulin cDNA template (Michalak (1999) Biochem J. 344 Pt 2:281-292), provided by Dr. Marek Michalak, University of Alberta, Edmonton, Canada, and a set of primers: 5′-ccggtctagaatgctgctccctgtgccgct-3′ (SEQ ID NO:71) and (SEQ ID NO:72) 5′-ccggagatctcagctcgtccttggcctggc-3′. The amplified product was then digested with the restriction digest enzymes XbaI and BamHI and further cloned into the XbaI and BamHI cloning sites of pcDNA3 vector (Invitrogen, Carlsbad, Calif.). For the generation of pcDNA3-CRT/E7, the E7 DNA was amplified by PCR using pcDNA3-E7 as a DNA template and a set of primers: 5′-ggggaattcatggagatacaccta-3′ (SEQ ID NO:73) and 5′-ggtggatccttgagaacagatgg-3′ (SEQ ID NO:74). The amplified E7 DNA fragment was then digested with BamHI and further cloned into the BamHI cloning sites of pcDNA3-CRT vector. The orientation and accuracy of these constructs was confirmed by DNA sequencing.

Plasmid DNA with CRT, E7 or CRT/E7 gene insert and the “empty” plasmid vector were transfected into subcloning-efficient DH5™ cells (Life Technologies, USA). The DNA was then amplified and purified using double CsCl purification (BioServe Biotechnologies, Laurel, Md.). The integrity of plasmid DNA and the absence of Escherichia coli DNA or RNA were checked in each preparation using 1% agarose gel electrophoresis. DNA concentration was determined by the optical density, measured at 260 nm. The presence of inserted E7 fragment was confirmed by restriction enzyme digestion and gel electrophoresis.

General Recombinant DNA Methods

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, subcloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Amplification of Nucleic Acids

Oligonucleotide primers can be used to amplify nucleic acids to generate fusion protein coding sequences used to practice the invention, to monitor levels of vaccine after in vivo administration (e.g., levels of a plasmid or virus), to confirm the presence and phenotype of activated CTLs, and the like. The skilled artisan can select and design suitable oligonucleotide amplification primers using known sequences. Amplification methods are also well known in the art, and include, e.g., polymerase chain reaction, PCR (PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and, self-sustained sequence replication (Guatelli (1990) Proc. Natl. Acad. Sci. USA 87:1874); Qβ replicase amplification (Smith (1997) J. Clin. Microbiol. 35:1477-1491; Burg (1996) Mol. Cell. Probes 10:257-271) and other RNA polymerase mediated techniques (NASBA, Cangene, Mississauga, Ontario; Berger (1987) Methods Enzymol. 152:307-316; U.S. Pat. Nos. 4,683,195 and 4,683,202; Sooknanan (1995) Biotechnology 13:563-564).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, cDNA molecules encoding the amino acid sequence corresponding to the fusion polypeptide of the present invention or fragments or derivatives thereof can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

This invention includes isolated nucleic acids having a nucleotide sequence encoding the novel fusion polypeptides that comprise a translocation polypeptide and an antigen, fragments thereof or equivalents thereof. The term nucleic acid as used herein is intended to include such fragments or equivalents. The nucleic acid sequences of this invention can be DNA or RNA.

A cDNA nucleotide sequence the fusion polypeptide can be obtained by isolating total mRNA from an appropriate cell line. Double stranded cDNA is prepared from total mRNA. cDNA can be inserted into a suitable plasmid, bacteriophage or viral vector using any one of a number of known techniques.

In reference to a nucleotide sequence, the term “equivalent” is intended to include sequences encoding structurally homologous and/or a functionally equivalent proteins. For example, a natural polymorphism in a nucleotide sequence encoding an anti-apoptotic polypeptide according to the present invention (especially at the third base of a codon) may be manifest as “silent” mutations which do not change the amino acid sequence. Furthermore, there may be one or more naturally occurring isoforms or related, immunologically cross-reactive family members of these proteins. Such isoforms or family members are defined as proteins that share function amino acid sequence similarity to the reference polypeptide.

Fragment of Nucleic Acid

A fragment of the nucleic acid sequence is defined as a nucleotide sequence having fewer nucleotides than the nucleotide sequence encoding the full length translocation polypeptide, antigenic polypeptide or the fusion thereof. This invention includes such nucleic acid fragments that encode polypeptides which retain (1) the ability of the fusion polypeptide to induce increases in frequency or reactivity of T cells, preferably CD8+ T cells, that are specific for the antigen part of the fusion polypeptide.

For example, a nucleic acid fragment as intended herein encodes an anti-apoptotic polypeptide that retains the ability to improve the immunogenicity of an antigen vaccube when administered as a chimeric DNA with antigen-encoding sequence, or when co-administered therewith.

Generally, the nucleic acid sequence encoding a fragment of an anti-apoptotic polypeptide comprises of nucleotides from the sequence encoding the mature protein (or an active fragment thereof).

Nucleic acid sequences of this invention may also include linker sequences, natural or modified restriction endonuclease sites and other sequences that are useful for manipulations related to cloning, expression or purification of encoded protein or fragments. These and other modifications of nucleic acid sequences are described herein or are well-known in the art.

The techniques for assembling and expressing DNA coding sequences for translocation types of proteins, and DNA coding sequences for antigenic polypeptides, include synthesis of oligonucleotides, PCR, transforming cells, constructing vectors, expression systems, and the like; these are well-established in the art such that those of ordinary skill are familiar with standard resource materials, specific conditions and procedures.

Expression Vectors and Host Cells

This invention includes an expression vector comprising a nucleic acid sequence encoding a anti-apoptotic polypeptide or a targeting polypeptide operably linked to at least one regulatory sequence.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons (see Example 1, below), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and includes both the expression and nonexpression plasmids. Where a recombinant microorganism or cell culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and/or the type of protein to be expressed.

The present expression vectors comprise the full range of nucleic acid molecules encoding the various embodiments of the fusion polypeptide and its functional derivatives (defined herein) including polypeptide fragments, variants, etc.

Such expression vectors are used to transfect host cells (in vitro, ex vivo or in vivo) for expression of the DNA and production of the encoded proteins which include fusion proteins or peptides. It will be understood that a genetically modified cell expressing the fusion polypeptide may transiently express the exogenous DNA for a time sufficient for the cell to be useful for its stated purpose.

The present in invention provides methods for producing the fusion polypeptides, fragments and derivatives. For example, a host cell transfected with a nucleic acid vector that encodes the fusion polypeptide is cultured under appropriate conditions to allow expression of the polypeptide.

Host cells may also be transfected with one or more expression vectors that singly or in combination comprise DNA encoding at least a portion of the fusion polypeptide and DNA encoding at least a portion of a second protein, so that the host cells produce yet further fusion polypeptides that include both the portions.

A culture typically includes host cells, appropriate growth media and other byproducts. Suitable culture media are well known in the art. The fusion polypeptide can be isolated from medium or cell lysates using conventional techniques for purifying proteins and peptides, including ammonium sulfate precipitation, fractionation column chromatography (e.g. ion exchange, gel filtration, affinity chromatography, etc.) and/or electrophoresis (see generally, “Enzyme Purification and Related Techniques”, Methods in Enzymology, 22:233-577 (1971)). Once purified, partially or to homogeneity, the recombinant polypeptides of the invention can be utilized in pharmaceutical compositions as described in more detail herein.

The term “isolated” as used herein, when referring to a molecule or composition, such as a translocation polypeptide or a nucleic acid coding therefor, means that the molecule or composition is separated from at least one other compound protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Prokaryotic or eukaryotic host cells transformed or transfected to express the fusion polypeptide or a homologue or functional derivative thereof are within the scope of the invention. For example, the fusion polypeptide may be expressed in bacterial cells such as E. coli, insect cells (baculovirus), yeast, or mammalian cells such as Chinese hamster ovary cells (CHO) or human cells. Other suitable host cells may be found in Goeddel, (1990) supra or are otherwise known to those skilled in the art.

Expression in eukaryotic cells leads to partial or complete glycosylation and/or formation of relevant inter- or intra-chain disulfide bonds of the recombinant protein.

Although preferred vectors are described in the Examples, other examples of expression vectors are provided here. Examples of vectors for expression in yeast S. cerevisiae include pYepSec1 (Baldari et al., (1987) EMBO J. 6:229-234), pMFa (Kurjan et al. (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165,) and the pVL series (Lucklow, V. A., and Summers, M. D., (1989) Virology 170:31-39). Generally, COS cells (Gluzman, Y., (1981) Cell 23:175-182) are used in conjunction with such vectors as pCDM 8 (Aruffo A. and Seed, B., supra, for transient amplification/expression in mammalian cells, while CHO (dhfr-negative CHO) cells are used with vectors such as pMT2PC (Kaufman et al. (1987), EMBO J. 6:187-195) for stable amplification/expression in mammalian cells. The NS0 myeloma cell line (a glutamine synthetase expression system.) is available from Celltech Ltd.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant protein.

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lacO fusion promoter mediated by coexpressed viral RNA polymerase (M7gn1). Th is viral polymerase is supplied by host strains BL21(DE3) or HBS174(DE3) from a resident λ, prophage harboring a T7gn1 under the transcriptional control of the lacUV 5 promoter.

Vector Construction

Construction of suitable vectors comprising the desired coding and control sequences employs standard ligation and restriction techniques which are well understood in the art. Isolated plasmids, DNA sequences, or synthesized oligonucleotides are cleaved, tailored, and re-ligated in the form desired.

The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, for example, Edge, M. D., Nature (1981) 292:756; Nambair, K. P., et al., Science (1984) 223:1299; and Jay, E., J Biol Chem (1984) 259:6311.

Synthetic oligonucleotides are prepared by either the phosphotriester method as described by references cited above or the phosphoramidite method as described by Beaucage, S. L., and Caruthers, M. H., Tet Lett (1981) 22:1859; and Matteucci, M. D., and Caruthers, M. H., J Am Chem Soc (1981) 103:3185 and can be prepared using commercially available automated oligonucleotide synthesizers. Kinase treatment of single strands prior to annealing or for labeling is achieved using an excess, e.g., about 10 units of polynucleotide kinase to 1 nmole substrate in the presence of 50 mM Tris, pH 7.6, 10 mM MgCl₂, 5 mM dithiothreitol, 1-2 mM ATP, 1.7 pmoles γ-³²P-ATP (2.9 mCi/mmole), 0.1 mM spermidine, 0.1 mM EDTA.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. In general, about 1 mg of plasmid or DNA sequence is cleaved by one unit of enzyme in about 20 ml of buffer solution; in the examples herein, typically, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods in Enzymology (1980) 65:499-560.

Restriction cleaved fragments may be blunt ended by treating with the large fragment of E. coli DNA polymerase I (Klenow) in the presence of the four deoxynucleotide triphosphates (dNTPs) using conventional methods and conditions. Ligations are performed using known, conventional methods. In vector construction employing “vector fragments”, the fragment is commonly treated with bacterial alkaline phosphatase (BAP) or calf intestinal alkaline phosphatase (CIAP) in order to remove the 5′ phosphate and prevent self- Alternatively, re-ligation can be prevented in vectors which have been double digested by additional restriction enzyme and separation of the unwanted fragments.

Any of a number of methods are used to introduce mutations into the coding sequence to generate the variants of the invention. These mutations include simple deletions or insertions, systematic deletions, insertions or substitutions of clusters of bases or substitutions of single bases.

For example, modifications anti-apoptotic DNA or the antigen-encoding DNA sequence are created by site-directed mutagenesis, a well-known technique for which protocols and reagents are commercially available (Zoller, M J et al., Nucleic Acids Res (1982) 10:6487-6500 and Adelman, J P et al., DNA (1983) 2:183-193)). Correct ligations for plasmid construction are confirmed, for example, by first transforming E. coli strain MC1061 (Casadaban, M., et al., J Mol Biol (1980) 138:179-207) or other suitable host with the ligation mixture. Using conventional methods, transformants are selected based on the presence of the ampicillin-, tetracycline- or other antibiotic resistance gene (or other selectable marker) depending on the mode of plasmid construction. Plasmids are then prepared from the transformants with optional chloramphenicol amplification optionally following chloramphenicol amplification ((Clewell, D B et al., Proc Natl Acad Sci USA (1969) 62:1159; Clewell, D. B., J Bacteriol (1972) 110:667). Several mini DNA preps are commonly used. See, e.g., Holmes, D S, et al., Anal Biochem (1981) 114:193-197; Birnboim, H C et al., Nucleic Acids Res (1979) 7:1513-1523. The isolated DNA is analyzed by restriction and/or sequenced by the dideoxy nucleotide method of Sanger (Proc Natl Acad Sci USA (1977) 74:5463) as further described by Messing, et al., Nucleic Acids Res (1981) 9:309, or by the method of Maxam et al. Methods in Enzymology (1980) 65:499.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Known fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant protein.

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lacO fusion promoter mediated by coexpressed viral RNA polymerase (T7gn1). Th is viral polymerase is supplied by host strains BL21(DE3) or HMS174(DE3) from a resident λ prophage harboring a T7gn1 under the transcriptional control of the lacUV 5 promoter.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521 (1985)) or in the RSV-LTR (from Rous sarcoma virus) (Gorman, C. M., Proc. Natl. Acad. Sci. USA 79:6777 (1982). Also useful are the promoter of the mouse metallothionein I gene (Harner, D., et al., J. Mol. Appl. Gen. 1:273-288 (1982)); the TK promoter of Herpes virus (McKnight, S., Cell 31:355-365 (1982)); the SV40 early promoter (Benoist, C., et al., Nature 290:304-310 (1981)); and the yeast gal4 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)). Other illustrative descriptions of transcriptional factor association with promoter regions and the separate activation and DNA binding of transcription factors include: Keegan et al., Nature (1986) 231:699; Fields et al., Nature (1989) 340:245; Jones, Cell (1990) 61:9; Lewin, Cell (1990) 61:1161; Ptashne et al., Nature (1990) 346:329; Adams et al., Cell (1993) 72:306. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B. M., Genes IV, Oxford University Press, Oxford, (1990), pp. 552-576. Particularly useful are retroviral enhancers (e.g., viral LTR). The enhancer is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Nucleic acids of the invention can also be chemically synthesized using standard techniques. Various methods of chemically synthesizing polydeoxynucleotides are known, including solid-phase synthesis which, like peptide synthesis, has been fully automated with commercially available DNA synthesizers (See, e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

Proteins and Polypeptides

The terms “polypeptide,” “protein,” and “peptide” when referring to compositions of the invention are meant to include variants, analogues, and mimetics with structures and/or activity that substantially correspond to the polypeptide or peptide from which the variant, etc., was derived.

The present invention includes an “isolated” fusion polypeptide comprising a targeting polypeptide linked to an antigenic polypeptide.

The term “chimeric” or “fusion” polypeptide or protein refers to a composition comprising at least one polypeptide or peptide sequence or domain that is chemically bound in a linear fashion with a second polypeptide or peptide domain. One embodiment of this invention is an isolated or recombinant nucleic acid molecule encoding a fusion protein comprising at least two domains, wherein the first domain comprises an anti-apoptotic polypeptide and the second domain comprising an antigenic epitope, e.g., an MHC class I-binding peptide epitope. Additional domains can comprise a targeting polypeptide or the like. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules of the invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of an anti-apoptotic polypeptide (or its coding sequence) which refers to an amino acid substitution variant, a “fragment,” or a “chemical derivative” of the protein, which terms are defined below. A functional derivative retains measurable anti-apoptotic activity, preferably that is manifest as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous anti-apoptotic proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to a reference nucleic acid molecules. BLAST protein searches can be performed with the XBLAST program, score=50, wordlength=3 to obtain amino acid sequences homologous to HVP22 protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of a particular anti-apoptotic polypeptide as described herein is characterized as having (a) functional activity of the native anti-apoptotic polypeptide and (b) sequence similarity to a native anti-apoptotic polypeptide when determined as above, of at least about 20% (at the amino acid level), preferably at least about 40%, more preferably at least about 70%, even more preferably at least about 90%.

It is within the skill in the art to obtain and express such a protein using DNA probes based on the disclosed sequences.

Then, the chimeric DNA construct or fusion protein's biological activity can be tested readily using art-recognized methods such as those described herein in the Examples. A biological assay of the stimulation of antigen-specific T cell reactivity will indicate whether the homologue has the requisite activity to qualify as a “functional” homologue.

A “variant” refers to a molecule substantially identical to either the fill protein or to a fragment thereof in which one or more amino acid residues have been replaced (substitution variant) or which has one or several residues deleted (deletion variant) or added (addition variant). A “fragment” of the anti-apoptotic polypeptide refers to any subset of the molecule, that is, a shorter polypeptide of the full-length protein.

A number of processes can be used to generate fragments, mutants and variants of the isolated DNA sequence. Small subregions or fragments of the nucleic acid encoding the spreading protein, for example 1-30 bases in length, can be prepared by standard, chemical synthesis. Antisense oligonucleotides and primers for use in the generation of larger synthetic fragment.

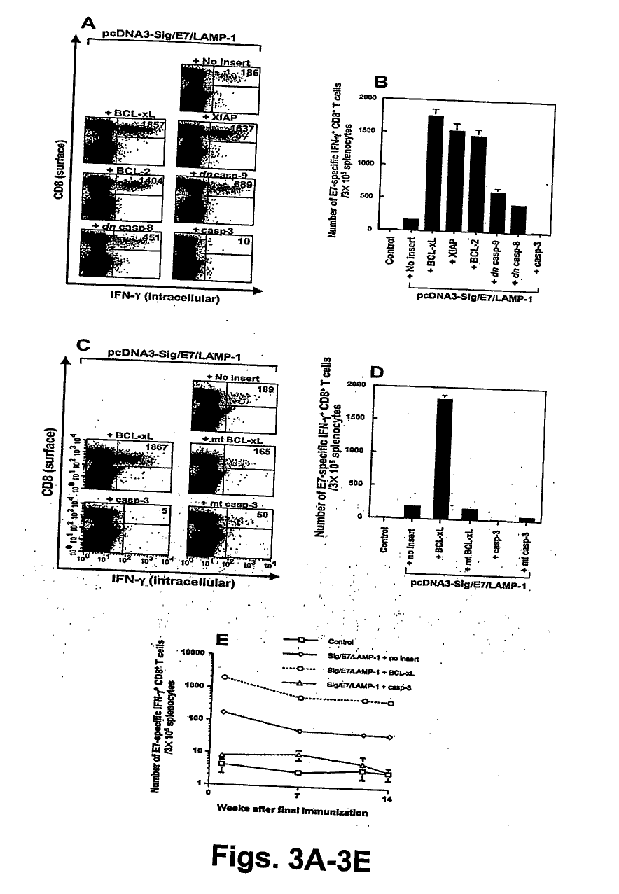

A preferred group of variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | | |
|---|---|---|
| 1 | Small aliphatic, nonpolar or | Ala, Ser, Thr (Pro, Gly); |
| | slightly polar residues | |
| 2 | Polar, negatively charged residues | Asp, Asn, Glu, Gln; |
| | and their amides | |
| 3 | Polar, positively charged residues | His, Arg, Lys; |
| 4 | Large aliphatic, nonpolar residues | Met, Leu, Ile, Val (Cys) |
| 5 | Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bully side chain, e.g., Phe, for (or by) a residue not having such a side chain, e.g., Gly.

Most acceptable deletions, insertions and substitutions according to the present invention are those that do not produce radical changes in the characteristics of the wild-type or native protein in terms of its intercellular spreading activity and its ability to stimulate antigen specific T cell reactivity to an antigenic epitope or epitopes that are fused to the spreading protein. However, when it is difficult to predict the exact effect of the substitution, deletion or insertion in advance of doing so, one skilled in the art will appreciate that the effect can be evaluated by routine screening assays such as those described here, without requiring undue experimentation.

Whereas shorter chain variants can be made by chemical synthesis, for the present invention, the preferred longer chain variants are typically made by site-specific mutagenesis of the nucleic acid encoding the polypeptide, expression of the variant nucleic acid in cell culture, and, optionally, purification of the polypeptide from the cell culture, for example, by immunoaffinity chromatography using specific antibody immobilized to a column (to absorb the variant by binding to at least one epitope).

The term “chemically linked” refers to any chemical bonding of two moieties, e.g., as in one embodiment of the invention, where a translocation polypeptide is chemically linked to an antigenic peptide. Such chemical linking includes the peptide bonds of a recombinantly or in vivo generated fusion protein.

Therapeutic Compositions and their Administration

A vaccine composition comprising the nucleic acid encoding the fusion polypeptide, or a cell expressing this nucleic acid is administered to a mammalian subject, preferably a human. The vaccine composition is administered in a pharmaceutically acceptable carrier in a biologically effective or a therapeutically effective amount, Certain preferred conditions are disclosed in the Examples. The composition may be given alone or in combination with another protein or peptide such as an immunostimulatory molecule. Treatment may include administration of an adjuvant, used in its broadest sense to include any nonspecific immune stimulating compound such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents. I Thus an effective amount is between about 1 nanogram and about 1 gram per kilogram of body weight of the recipient, more preferably between about 0.1 μg/kg and about 10 mg/kg, more preferably between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of cells expressing the nucleic acid is between about 10⁴ and 10⁸ cells. Those skilled in the art of immunotherapy will be able to adjust these doses without undue experimentation.

The active compound may be administered in a convenient manner, e.g. injection by a convenient and effective route. Preferred routes include intradermal “gene gun” delivery, subcutaneous, intravenous and intramuscular routes. Other possible routes include oral administration, intrathecal, inhalation, transdermal application, or rectal administration. For the treatment of existing tumors which have not been completely resected or which have recurred, direct intratumoral injection is also intended.

Depending on the route of administration, the active compound may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol). or in an appropriate carrier such as liposomes (including water-in-oil-in-water emulsions as well as conventional liposomes (Strejan et al., (1984) J. Neuroimmunol 7:27).

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like.

Compositions are preferably formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Other pharmaceutically acceptable carriers for the nucleic acid vaccine compositions according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Antigens Associated with Pathogens

A major use for the present invention is the use of the present nucleic acid compositions in therapeutic vaccine for cancer and for major chronic viral infections that cause morbidity and mortality worldwide. Such vaccines are designed to eliminate infected cells—this requires T cell responses as antibodies are often ineffective. The vaccines of the present invention are designed to meet these needs.

Preferred antigens are epitopes of pathogenic microorganisms against which the host is defended by effector T cells responses, including cytotoxic T lymphocyte (CTL) and delayed type hypersensitivity. These typically include viruses, intracellular parasites such as malaria, and bacteria that grow intracellularly such as Mycobacteria and Listeria species. Thus, the types of antigens included in the vaccine compositions of this invention are any of those associated with such pathogens (in addition, of course, to tumor-specific antigens). It is noteworthy that some viral antigens are also tumor antigens in the case where the virus is a causative factor in cancer.

In fact, the two most common cancers worldwide, hepatoma and cervical cancer, are associated with viral infection. Hepatitis B virus (HBV) (Beasley, R. P. et al., Lancet 2, 1129-1133 (1981) has been implicated as etiologic agent of hepatomas. 80-90% of cervical cancers express the E6 and E7 antigens (exemplified herein) from one of four “high risk” human papillomavirus types: HPV-16, HPV-18, HPV-31 and HPV-45 (Gissmann, L. et al., Ciba Found Symp. 120, 190-207 (1986); Beaudenon, S., et al. Nature 321, 246-249 (1986). The HPV E6 and E7 antigens are the most promising targets for virus associated cancers in immunocompetent individuals because of their ubiquitous expression in cervical cancer. In addition to their importance as targets for therapeutic cancer vaccines, virus associated tumor antigens are also ideal candidates for prophylactic vaccines. Indeed, introduction of prophylactic HBV vaccines in Asia have decreased the incidence of hepatoma (Chang, M. H., et al. New Engl. J. Med. 336, 1855-1859 (1997), representing a great impact on cancer prevention.

Among the most important viruses in chronic human viral infections are HPV, HBV, hepatitis C Virus (HCV), human immunodeficiency virus (HIV-1 and HIV-2), herpesviruses such as Epstein Barr Virus (EBV), cytomegalovirus (CMV) and HSV-1 and HSV-2 and influenza virus. Useful antigens include HBV surface antigen or HBV core antigen; ppUL83 or pp89 of CMV; antigens of gp120, gp41 or p24 proteins of HIV-1; ICP27, gD2, gB of HSV; or influenza nucleoprotein (Anthony, L S et al., Vaccine 1999; 17:373-83). Other antigens associated with pathogens that can be utilized as described herein are antigens of various parasites, includes malaria, preferably malaria peptide (NANP)40.

In addition to its applicability to human cancer and infectious diseases, the present invention is also intended for use in treating animal diseases in the veterinary medicine context. Thus, the approaches described herein may be readily applied by one skilled in the art to treatment of veterinary herpesvirus infections including equine herpesviruses, bovine viruses such as bovine viral diarrhea virus (for example, the E2 antigen), bovine herpesviruses, Marek's disease virus in chickens and other fowl; animal retroviral and lentiviral diseases (e.g., feline leukemia, feline immunodeficiency, simian immunodeficiency viruses, etc.); pseudorabies and rabies; and the like.

As for tumor antigens, any tumor-associated or tumor-specific antigen that can be recognized by T cells, preferably by CTL, can be used. In addition to the HPV-E7 antigen exemplified herein is mutant p53 or HER2/neu or a peptide thereof. Any of a number of melanoma-associated antigens may be used, such as MAGE-1, MAGE-3, MART-1/Melan-A, tyrosinase, gp75, gp100, BAGE, GAGE-1, GAGE-2, GnT-V, and p15 (see, U.S. Pat. No. 6,187,306).

The following references set forth principles and current information in the field of basic, medical and veterinary virology and are incorporated by reference: Fields Virology, Fields, B N et al., eds., Lippincott Williams & Wilkins, NY, 1996; Principles of Virology: Molecular Biology, Pathogenesis, and Control, Flint, S. J. et al., eds., Amer Society for Microbiology, Washington, 1999; Principles and Practice of Clinical Virology, 4th Edition, Zuckerman. A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy: Classification and Nomenclature of Viruses: Seventh Report of the International Committee on Taxonomy of Viruses, by M. H. V. Van Regenmortel, MHV et al., eds., Academic Press; NY, 2000.

Delivery of Vaccine Nucleic Acid to Cells and Animals

The Examples below describe certain preferred approaches to delivery of the vaccines of the present invention. A broader description of other approaches including viral and nonviral vectors and delivery mechanisms follow.

DNA delivery involves introduction of a “foreign” DNA into a cell ex vivo and ultimately, into a live animal or directly into the animal. Several general strategies for gene delivery (=delivery of nucleic acid vectors) for purposes that include “gene therapy” have been studied and reviewed extensively (Yang, N-S., Crit. Rev. Biotechnol. 12:335-356 (1992); Anderson, W. F., Science 256:808-813 (1992); Miller, A. S., Nature 357:455-460 (1992); Crystal, R. G., Amer. J. Med. 92(suppl 6A):44S-52S (1992); Zwiebel, J. A. et al., Ann. N.Y. Acad. Sci. 618:394-404 (1991); McLachlin, J. R. et al., Prog. Nucl. Acid Res. Molec. Biol. 38:91-135 (1990); Kohn, D. B. et al., Cancer Invest. 7:179-192 (1989), which references are herein incorporated by reference in their entirety).

One approach comprises nucleic acid transfer into primary cells in culture followed by autologous transplantation of the ex vivo transformed cells into the host, either systemically or into a particular organ or tissue.

The term “systemic administration” refers to administration of a composition or agent such as a molecular vaccine as described herein, in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. The term “local administration” refers to administration of a composition or drug into a limited, or circumscribed, anatomic space, such as intratumoral injection into a tumor mass, subcutaneous injections, intramuscular injections. One of skill in the art would understand that local administration or regional administration may also result in entry of a composition into the circulatory system.

For accomplishing the objectives of the present invention, nucleic acid therapy would be accomplished by direct transfer of a the functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

The DNA molecules encoding the fusion polypeptides of the present invention may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R. F. et al., Cell 33:153-159 (1983); Miller, A. D. et al., Molec. Cell. Biol. 5:431-437 (1985); Sorge, J., et al., Molec. Cell. Biol. 4:1730-1737 (1984); Hock, R. A. et al., Nature 320:257 (1986); Miller, A. D. et al., Molec. Cell. Biol. 6:2895-2902 (1986). Newer packaging cell lines which are efficient an safe for gene transfer have also been described (Bank et al., U.S. Pat. No. 5,278,056.

This approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M. S., In: Virology, Fields, B N et al., eds, Raven Press, New York, 1990, p. 1679; Berkner, K. L., Biotechniques 6:616 9191988), Strauss, S. E., In: The Adetzoviruses, Ginsberg, H S, ed., Plenum Press, New York, 1984, chapter 11), herpes simplex virus (HSV) for neuron-specific delivery and persistence. Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R. J. et al., EMBO J. 10:3941 (1991) according to the present invention.

Another vector which can express the DNA molecule of the present invention, and is useful in the present therapeutic setting, particularly in humans, is vaccinia virus, which can be rendered non-replicating (U.S. Pat. Nos. 5,225,336; 5,204,243; 5,155,020; 4,769,330; Sutter, G et al., Proc. Natl. Acad. Sci. USA (1992) 89:10847-10851; Fuerst, T. R. et al., Proc. Natl. Acad. Sci. USA (1989) 86:2549-2553; Falkner F. G. et al.; Nucl. Acids Res (1987) 15:7192; Chakrabarti, S et al, Molec. Cell. Biol. (1985) 5:3403-3409). Descriptions of recombinant vaccinia viruses and other viruses containing heterologous DNA and their uses in immunization and DNA therapy are reviewed in: Moss, B., Curr. Opin. Genet. Dev. (1993) 3:86-90; Moss, B. Biotechnology (1992) 20:345-362; Moss, B., Curr Top Microbiol Immunol (1992) 158:25-38; Moss, B., Science (1991) 252:1662-1667; Piccini, A et al., Adv. Virus Res. (1988) 34:43-64; Moss, B. et al., Gene Amplif Anal (1983) 3:201-213.

In addition to naked DNA or RNA, or viral vectors, engineered bacteria may be used as vectors. A number of bacterial strains including Salmonella, BCG and Listeria monocytogenes (LM) Hoiseth & Stocker, Nature 291, 238-239 (1981); Poirier, T P et al. J. Exp. Med. 168, 25-32 (1988); (Sadoff, J. C., et al., Science 240, 336-338 (1988); Stover, C. K., et al., Nature 351, 456-460 (1991); Aldovini, A. et al., Nature 351, 479-482 (1991); Schafer, R., et al., J. Immunol. 149, 53-59 (1992); Ikonomidis, G. et al., J. Exp. Med. 180, 2209-2218 (1994)). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N.-S., et al., Proc. Natl. Acad. Sci. USA 87:9568 (1990); Williams, R. S. et al., Proc. Natl. Acad. Sci. USA 88:2726 (1991); Zelenin, A. V. et al., FEBS Lett. 280:94 (1991); Zelenin, A. V. et al., FEBS Lett. 244:65 (1989); Johnston, S. A. et al., In Vitro Cell. Dev. Biol. 27:11 (1991)). Furthermore, electroporation, a well-known means to transfer genes into cell in vitro, can be used to transfer DNA molecules according to the present invention to tissues in vivo (Titomirov, A. V. et al, Biochim. Biophys. Acta 1088:131 ((1991)).

“Carrier mediated gene transfer” has also been described (Wu, C. H. et al., J. Biol. Chem. 264:16985 (1989); Wu, G. Y. et al., J. Biol. Chem. 263:14621 (1988); Soriano, P. et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc. Natl. Acad. Sci. USA 80:1068 (1983); Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA according to the present invention for transfer.

Plasmid DNA used for transfection or microinjection may be prepared using methods well-known in the art, for example using the Quiagen procedure (Quiagen), followed by DNA purification using known methods, such as the methods exemplified herein.

Having now generally described the invention, the same will be more readily understood through reference to the following examples which are provided by way of illustration, and are not intended to be limiting of the present invention, unless specified.

### EXAMPLE I

### Co-Administration of DNA Encoding Anti-Apoptotic Proteins Enhances DNA Vaccine Potency

(This example incorporates by reference T W Kim et al, J. Clin. Invest. 112:109-117, 2003 July)

A. Materials and Methods

Plasmid DNA constructs and DNA preparation. The generation of pcDNA3-E7 (4), pCMV(neo)-Sig/E7/LAMP-1 (Ji, H., et al., 1999, Hum. Gene Ther. 10:2727-40), and pDNA3-E7/GFP (Hung, C F et al., 2001. Cancer Res. 61:3698-3703) has been described previously. The plasmid containing influenza hemagglutinin (HA), pcDNA3-HA, was provided by Drew Pardoll at the Johns Hopkins School of Medicine. The pEBB-XIAP (Clem, R. J., et al., 2001, J. Biol. Chem. 276:7602-08), pcDNA3-FLICEc-s (Muzio, M. et al., 1996, Cell 85:817-827), and pSG5 plasmids encoding BCL-xL mt 7 (mutant BLC-xL) (Cheng, E H et al., 1996, Nature 379:554-56), BCL-2 (Cheng, E H et al., 1997, Science 278:1966-68), or dn caspase-9 (Stennicke, H R et al., 1999, J. Biol. Chem. 274:8359-62) have been generated in J. Marie Hardwick's lab. To generate pcDNA3-Sig/E7/LAMP-1, Sig/E7/LAMP-1 was isolated from pCMV(neo)-Sig/E7/LAMP-1 (Ji et al., supra) and cloned into the EcoRI/BamHI sites of pcDNA3. For the generation of pcDNA3-OVA, the DNA fragment encoding OVA was amplified by a set of primers, 5′-cccgaattcatgggctccatcggcgcagc-3′ [SEQ ID NO:75] and 5′-cccggatccaaattcttcagagacgcttgc-3′ [SEQ ID NO:76], and OVA cDNA from Michael Bevan of the University of Washington (Seattle, Wash. The amplified product was further cloned into the EcoRI/BamHI sites of pcDNA3. For the generation of pSG5-XIAP, the DNA fragment encoding XIAP was amplified with PCR using pEBB-XIAP as template and a set of primers: 5′-gctaggatccatgacttttaacagttttgaagg-3′ [SEQ ID NO:77] and 5′-gcacggatccttaagacataaaaattttttgct-3′ [SEQ ID NO:78]. The amplified product was further cloned into the BamHI cloning site of pSG5. For the generation of pSG5-dn caspase-8, the DNA fragment of dn caspase-8 was amplified with PCR using pcDNA3-FLICEc-s as a template and a set of primers, 5′-gctaggatccatggacttcagcagaaatcttt-3′ [SEQ ID NO:79] and 5′-gcacggatcctcaatcagaagggaagacaag-3′ [SEQ ID NO:80]. The amplified product was further cloned into the BamHI cloning site of pSG5. For the generation of pSG5-caspase-3 and pSG5-mt caspase-3, the DNA fragments of caspase-3 and its mutant were amplified with PCR using C2P-caspase-3-GFP and C2P-caspase-3AE9(C163)-GFP (Colussi, P A et al., 1998, J. Biol. Chem. 273:26566-70) as a template, respectively, and a set of primers, 5′-ccgtcagatccgctagcgctaccgg-3′ [SEQ ID NO:81] and 5′-gtgcatcccttaggtgataaaaatagagttc-3′ [SEQ ID NO:82]. The amplified product was further cloned into the BamHI sites of pSG5. The accuracy of these constructs was confirmed by DNA sequencing. The DNA was amplified in Escherichia coli DH5x and purified as described previously (Chen C. H. et al., 2000, Cancer Res. 60:1035-42).

Western blot analysis. The expression of pro-apoptotic and anti-apoptotic proteins in COS-7 cells transfected with DNA encoding anti-apoptotic protein was characterized by Western blot analysis. The DNA encoding the various pro-apoptotic and anti-apoptotic proteins also contains an HA epitope (YPYDBPDYA; [SEQ ID NO:83]) at the 5′ end of the encoded protein to serve as a tag. Western blot analysis was performed with 50 μg of the cell lysate derived from COS-7 cells transfected with the various DNA constructs encoding the pro-apoptotic and anti-apoptotic proteins and anti-HA mouse mAb (clone12CA5; Roche Diagnostics Corp., Indianapolis, Ind., USA) using the method described previously (Hung, C. F. et al., supra).

Mice. Six- to eight-week-old female C57BL/6 mice were purchased from the National Cancer Institute (Frederick, Md., USA) and kept in the oncology animal facility of the Johns Hopkins Hospital (Baltimore, Md., USA).

DNA vaccination. DNA-coated gold particles were prepared according to a protocol described previously (Chen et al., supra). DNA-coated gold particles were delivered to the shaved abdominal region of mice using a helium-driven gene gun (Bio-Rad Laboratories Inc., Hercules, Calif.) with a discharge pressure of 400 psi. C57BL/6 mice were immunized with 2 μg of the plasmid encoding E7, Sig/E7/LAMP-1, HA, or OVA mixed with 2 μg of pSG5, pSG5-BCL-xL, pSG5-XIAP, pSG5-BCL-2, pSG5-dn caspse-9, pSG5-dn caspase-8, pSG5-mt BCL-xL, pSG5-caspase-3, or pSG5-mt caspase-3. The mice received a booster with the same dose 1 week later. Intracellular cytokine staining and flow-cytometry analysis. Splenocytes were harvested from mice 1 week after the last vaccination. Prior to intracellular cytokine staining, 4×10⁶ pooled splenocytes from each vaccination group were incubated for 16 hours with either 1 μg/ml of E7 (RAHYNIVTF [SEQ ID NO:84]), HA (IYSTVASSL [SEQ ID NO:85]), or OVA peptide (SIINFEKL [SEQ ID NO:86]) containing an MHC class I epitope for detecting antigen-specific CD8+ T cell precursors. Intracellular IFNγ staining and flow-cytometric analysis were performed as described previously (Chen et al., supra) using a Becton-Dickinson FACScan with CELLQuest software (Becton Dickinson Immunocytometry Systems, Mountain View, Calif.)

In Vivo Tumor Protection and Tumor-Treatment. The HPV-16 E7-expressing murine tumor TC-1, has been described previously (Lin, K Y. et al., 1996, Cancer Res. 56:21-26). In brief, HPV-16 E6, E7, and ras oncogene were used to transform primary C57BL/6 murine lung epithelial cells to generate the TC-1 line. For the tumor-protection, C57BL/6 mice (5/group) were challenged s.c. with 5×10⁴ TC-1 tumor cells per mouse in the right leg one week after the last vaccination. Mice were monitored for evidence of tumor growth by palpation and inspection twice a week. To study the subset of lymphocytes that are important for the antitumor effects, in vivo antibody depletion studies were performed using the method described previously by Lin et al., supra. mAb GK1.5 was used for CD4+ cell depletion, mAb 2.43 for CD8+ cell depletion. mAb PK136 was used for NK cell depletion.

For the tumor-treatment, 10⁴ TC-1 tumor cells were first injected i.v. via the tail vein to simulate hematogenous spread of tumors. Mice were treated with the DNA composition 3 days after tumor inoculation. Mice were monitored twice a week and sacrificed on day 42 after the last vaccination. The mean number of pulmonary nodules per mouse was evaluated by an experimenter blinded to sample identity. In vivo tumor protection, Ab depletion, and tumor-treatment experiments were performed three times and gave reproducible results. Preparation of CD11c+ cells from inguinal lymph nodes (LN) of vaccinated mice. C57BL/6 mice (3/group) received 12 nonoverlapping intradermal inoculations with a gene gun on their abdominal region. Gold particles used for each inoculation were coated with 1 μg of pcDNA3-E7/GFP DNA mixed with 1 μg of pSG5 encoding BCL-xL, mt BCL-xL, caspase-3, or no insert. The pcDNA3 (no insert) mixed with pSG5-BCL-xL served as a negative control. Inguinal LNs were harvested 1 or 5 days later and single cell suspension were prepared from each LN. CD11c+ cells were enriched in these LN cell populations using CD11c (N418) microbeads (Miltenyi Biotec, Auburn, Calif., USA). Enriched CD11c+ cells were analyzed in flow cytometry by forward and side scatter and gated around a population of cells with size and granularity of DCs. The percentage of CD11c+ cells in the gated area was characterized by using phycoerythrin (PE)-conjugated anti-CD11c mAb (PharMingen, San Diego, Calif., USA). GFP-positive cells were analyzed by flow-cytometry using a protocol described previously (Lappin, M B et al., 1999, Immunology. 98:181-88). Data are expressed as percentage of CD11c+ GFP+ cells among gated monocytes. Detection of apoptotic cells in the CD11c+ GFP+ population was performed using an annexin V-PE apoptosis detection Kit-I (BD Bioscience, San Diego, Calif.) according to the vendor's protocol. The percentage of apoptotic cells was analyzed flow-cytometrically by gating CD11c+ GFP+ cells.

Activation of an E7-specific CD8+ T cell line by CD11c enriched cells from vaccinated mice. Mice were vaccinated, and enriched CD11c+ cells were collected as described above. CD11c-enriched cells (2×10⁴) were incubated with 2×10⁶ cells of the E7-specific CD8+ T cell line (Wang, T L et al., 2000, Gene Ther. 7:726-33) for 16 hours. Cells were stained for both surface CD8 and intracellular IFNγ and analyzed by flow-cytometry as above.

Statistical analysis. All data expressed as means±SE are representative of at least two different experiments. Data for intracellular cytokine staining with flow cytometry analysis and tumor treatment experiments were evaluated by ANOVA. Comparisons between individual data points were made using Student's t test.

B. Results

Co-Administration of E7 DNA with DNA Encoding Anti-Apoptotic Factors Significantly Enhanced E7-Specific CD8+ T Cell-Mediated Immune Responses

The inventors hypothesized that DNA encoding anti-apoptotic proteins would enhance E7-specific CD8+ T cell immune responses when co-administered with E7 DNA. They therefore generated DNA constructs encoding anti-apoptotic proteins. Expression of anti-apoptotic proteins was confirmed in transfected COS-7 cells by Western blot analysis, and the expression levels of wild-type and mutant forms of these proteins was equivalent.

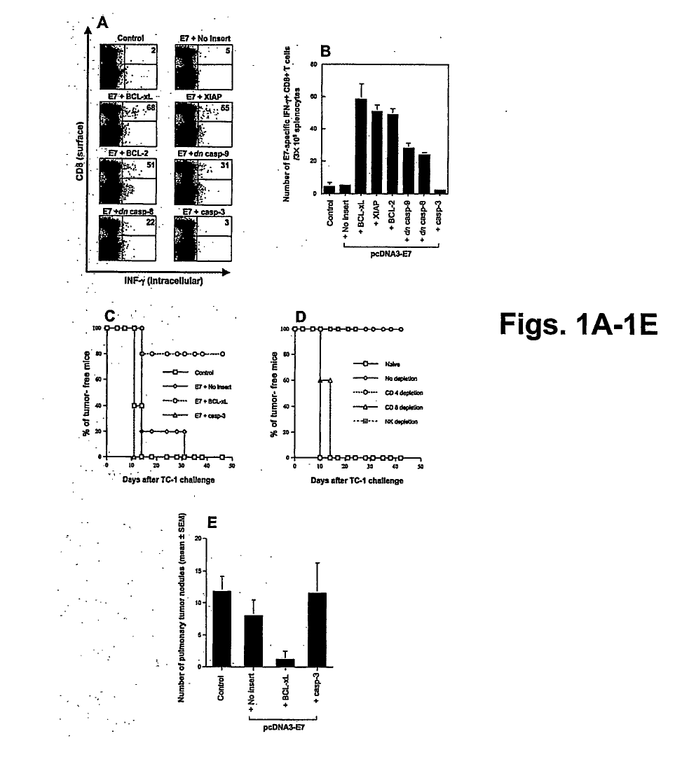

To enumerate E7-specific CD8+ T cell precursors generated by vaccination with E7 DNA mixed with DNA encoding anti-apoptotic or pro-apoptotic proteins, intracellular cytokine staining was performed and the cells analyzed by flow cytometry. As shown in FIGS. 1A and 1B, mice vaccinated with E7 DNA mixed with BCL-xL DNA had the highest frequency of E7-specific IFNγ-secreting CD8+ T cell precursors (58.3±9.5/3×10⁵ splenocytes), more than 11-fold greater than the number of precursors in subjects vaccinated with E7 DNA mixed with control pSG5 vector (no insert) (5.0±1.0/3×10⁵ splenocytes) (P<0.01). Similarly, vaccination with E7 DNA mixed with DNA encoding other anti-apoptotic proteins also led to increased numbers of E7-specific CD8+ T cells (expressed per 3×10⁵ spleen cells: E7+XIAP (50.7±3.8); E7 plus BCL-2 (48.7±3.1); E7 plus dn caspase-9 (28.0±3.0); and E7 plus dn caspase-8 (23.7±1.5). In contrast, co-administering E7 DNA with DNA encoding a pro-apoptotic protein, caspase-3, did not augment the number of E7-specific CD8+ T cell precursors (2.3±0.6). The results also indicated that E7 antigen was required for this immune-enhancing effect since an antigen-negative control, pcDNA3 (no insert) co-administered with BCL-xL did not enhance E7-specific CD8+ T cell activity (4.3±2.1). Thus, co-administration of E7 DNA with DNA encoding anti-apoptotic factors markedly increases the number of antigen-specific CD8+ T cell precursors.

Vaccination with E7 DNA Mixed with DNA Encoding Anti-Apoptotic Protein Leads to Protection Against E7+ Tumors

To determine if the observed enhancement in E7-specific CD8+ T cell-mediated immunity led to a significant E7-specific antitumor effect, an in vivo tumor-protection study was done using a previously described system, TC-1. As shown in FIG. 1C, 80% of mice receiving E7 DNA mixed with BCL-xL DNA remained tumor free 46 days after TC-1 challenge. In contrast, all of the mice receiving E7 DNA mixed with pSG5 (no insert) or caspase-3 (pro-apoptotic) developed tumors by day 46. Similarly, co-administration of DNA encoding either XIAP or BCL-2, like BCL-xL, resulted in significant antitumor effects by inhibiting tumor formation in a subcutaneous tumor model.

In vivo Ab depletion studies were done to determine the subsets of lymphocytes important for these antitumor effects. As shown in FIG. 1D, 100% of the mice depleted of CD8+ T cells grew tumors within 2 weeks after TC-1 challenge. In contrast, 100% of the mice depleted of CD4+ T cells or NK cells remained tumor-free 42 days after TC-1 challenged indicating that CD8⁺ T cells were important for the antitumor effects

Co-Administration of DNA Encoding HA or OVA with DNA Encoding Anti-Apoptotic Protein Leads to Enhanced Antigen-Specific CD8+ T Cell Immune Responses.

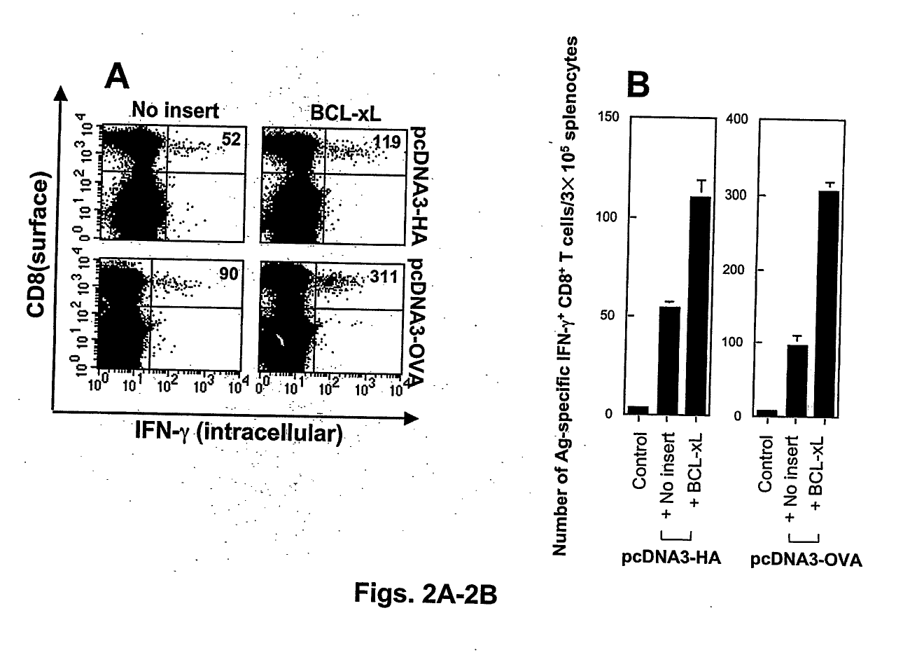

To determine if the observed enhancement of CD8+ T cell-mediated immunity is a general phenomenon that occurs with other antigens, studies were done with different antigen-expressing DNA vaccines in combination with DNA encoding anti-apoptotic proteins. Mice were immunized with pcDNA3 vectors containing DNA encoding the well-characterized antigens HA or OVA, mixed with pSG5 DNA containing no insert or BCL-xL. Using intracellular cytokine staining and flow cytometry, the inventors found that the combination of pcDNA3-HA or pcDNA3-OVA mixed with BCL-xL cells increased the number of antigen-specific CD8+ T cell precursors compared to vaccination of pcDNA3-HA or pcDNA3-OVA mixed with pSG5 (no insert), respectively (FIGS. 2A and 2B). These results suggest by co-administering DNA encoding an anti-apoptotic protein, an DNA encoding any antigen would be rendered more immunogenic as measured by an increase in the number of antigen-specific CD8+ T cell precursors.

Immunogenic compositions that target antigen intracellularly to desired subcellular compartments and enhance MHC class I and/or class II presentation of antigen to CD8+ and CD4+ T cells, respectively were described in the present inventors' earlier publications (Ji et al., supra; Chen et al., supra; W F Cheng et al., J. Clin. Invest. 108:669-678). One such vaccine, Sig/E7/LAMP-1 DNA (signal peptide/E7/lysosome-associated membrane protein) is able to target E7 to the endosomal/lysosomal compartments, which enhances MHC class II presentation of E7 to CD4+ T cells and also increase the number of E7-specific CD8+ T cells resulting in prevention of tumor development (Ji et al., supra).

Studies were conducted to assess the effect of co-administering DNA encoding anti-apoptotic proteins with DNA encoding E7 linked to a targeting polypeptide Mice were vaccinated with Sig/E7/LAMP-1 DNA mixed with DNA encoding different anti-apoptotic or pro-apoptotic proteins. As shown in FIGS. 3A and 3B, co-administration of Sig/E7/LAMP-1 DNA with BCL-xL DNA generated the highest frequency of E7-specific IFNγ-secreting CD8+ T cell precursors (per 3×10⁵ splenocytes): 1,752.7±99.9 which was greater than the number observed in mice vaccinated with Sig/E7/LAMP-1 DNA mixed with pSG5 (no insert) (167.3±16.2; P<0.01, ANOVA) or E7 DNA mixed with pSG5-BCLxL (58.3±9.5 (see FIG. 1A-1C). Similarly, combined vaccination of Sig/E7/LAMP-1 DNA with DNA encoding other anti-apoptotic proteins increased E7-specific CD8+ T cell precursor numbers (per 3×10⁵ splenocytes: Sig/E7/LAMP-1 plus XIAP (1,530.7±115.6), Sig/E7/LAMP-1 plus BCL-2 (1,462.7±99.9), Sig/E7/LAMP-1 plus dn caspase-9 (619.7±62.1), and Sig/E7/LAMP-1 plus dn caspase-8 (430.0±25.9).

Mice vaccinated with pcDNA3-Sig/E7/LAMP-1 mixed with pSG5-BCL-xL demonstrated significantly higher numbers of E7-specific CD4+ T cells (6-fold higher) than mice vaccinated with pcDNA3-Sig/E7/LAMP-1 mixed with pSG5, indicating that the anti-apoptotic DNA als enhanced class II-mediated presentation of antigen to CD4+ T cells.

Gene Gun Co-Administration of Sig/E7/LAMP-1 DNA with DNA Encoding Mutant BCL-xL, Caspase-3, or mt Caspase-3 does not Activate E7-Specific CD8+ T Cell Activity

A mutation abrogating the anti-apoptotic function of BCL-xL was evaluated. Although vaccination with Sig/E7/LAMP-1 DNA mixed with BCL-xL DNA led to a marked increase in the number of E7-specific IFN-γ secreting CD8+ T cell precursors (1,816±54.7), this type of response was not observed when the DNA encoding defective mutant BCL-xL was used (pSG5-mt BCL-xL) (168±16.3; P<0.001, ANOVA) (FIGS. 3C and 3D). In addition, co-administration of pcDNA3-Sig/E7/LA-1 with DNA encoding a wild-type pro-apoptotic protein, caspase-3, or a caspase-3 mutant with somewhat attenuated pro-apoptotic function (Sasaki, S et al., 2001, Nat. Biotechnol. 19:543-47) led to a significant decrease in E7-specific CD8+ T cell precursor numbers (5±1.3/3×10⁵ splenocyte and 52/9.7×10⁵ splenocytes, respectively) compared with mice vaccinated with the mixture of Sig/E7/LAMP-1 DNA and control DNA encoding pSG5 (no insert). The results indicate that the anti-apoptotic function of BCL-xL is critical for the observed immunological enhancement.

Long-Term E7-Specific CD8+ T Cell Memory after Co-Administration of Sig/E7/LAMP-1 DNA and DNA Encoding BCL-xL

The antigen-specific CD8+ T cell immune response was evaluated in mice vaccinated with various combinations of DNA constructs at one, seven, twelve, and fourteen weeks after the last antigen-coding DNA vaccination. As shown in FIG. 3E, mice vaccinated with pcDNA3-Sig/E7/LAMP-1 mixed with pSG5-BCL-xL generated consistently highest numbers of E7 specific CD8+ T cell precursors throughout the duration of the study compared to mice vaccinated with pcDNA3-Sig/E7/LAMP-1 DNA mixed with control pSG5 DNA or pro-apoptotic pSG5-casp-3. This is evidence for the generation of long-term antigen-specific CD8+ T cell memory.

DCs in Inguinal LNs Survive Longer after Transfection by Co-Administration of E7/GFP DNA with DNA Encoding Anti-Apoptotic Protein.

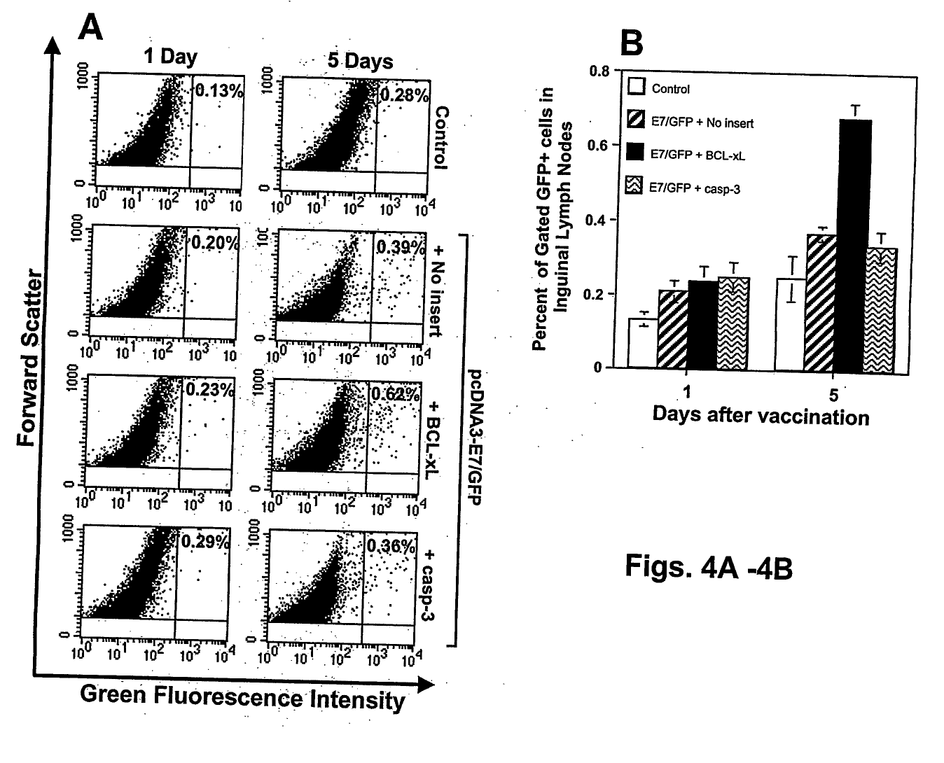

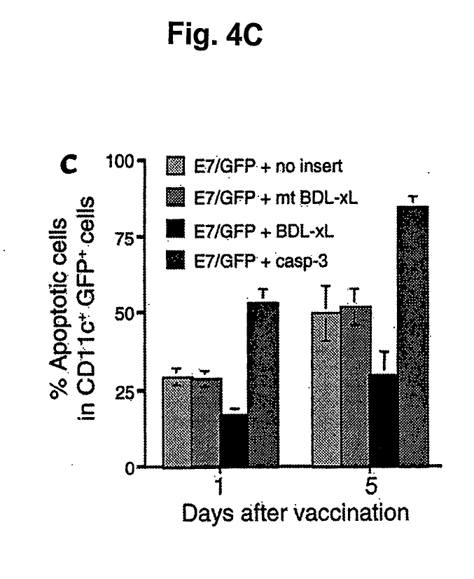

Following intradermal immunization, DCs are known to migrate to draining LNs nodes where they stimulate antigen-specific T cells (Condon, C et al. 1996, Nat. Med. 2:1122-28; Porgador, A et al., 1998, J. Exp. Med. 188:1075-82). The present inventors used GFP linked to E7 as a detectable label for DNA-transfected DCs in LNs draining the site of administration. Inguinal LNs were harvested from mice 1 and 5 days after gene gun vaccination. Because the CD11c+ cell population includes myeloid cells other than DCs (such as NK cells and B and T cell subsets), the gating was directed to a region more consistent with DC size and granular characteristics (Lappin et al., supra) in order to maximize the percentage of GFP+ CD11c+ DC for comparison of groups. Staining for additional DC markers was performed and showed that >90% of the GFP+ CD11c+ cells expressed DC surface markers such as B7.1 and B7.2 and CD40. As shown in FIGS. 4A and 4B, there were no significant differences in the numbers of CD11c+ and GFP+ cells in the inguinal LNs one day after vaccination (with E7 DNA mixed with BCL-xL DNA or control plasmid). By five days, however, a greater percentage of GFP+ CD11c+ cells were found in the LNs of mice vaccinated with the E7/GFP DNA mixed with BCL-xL DNA as compared to mice vaccinated with E7/GFP DNA mixed with DNA encoding pro-apoptotic caspase-3, mt BCL-xL, or no insert (P<0.0005, one-way ANOVA) (FIG. 4B).

The number of apoptotic cells in the CD11c+ GFP+ populations were assessed by staining for annexin V followed by flow-cytometry. As shown in FIG. 4C, mice vaccinated with DNA encoding E7/GFP mixed with DNA encoding BCL-xL demonstrated significantly lower percentages of apoptotic cells when compared to the other groups of vaccinated mice (P<0.0005, one-way ANOVA). Thus, our results suggest that co-administration of E7/GFP DNA with DNA encoding an anti-apoptotic protein may prolong the survival of DNA-transfected DCs.

Activity of CD11c-Enriched Cells from Mice Co-Administered E7/GFP DNA with DNA Encoding BCL-xL

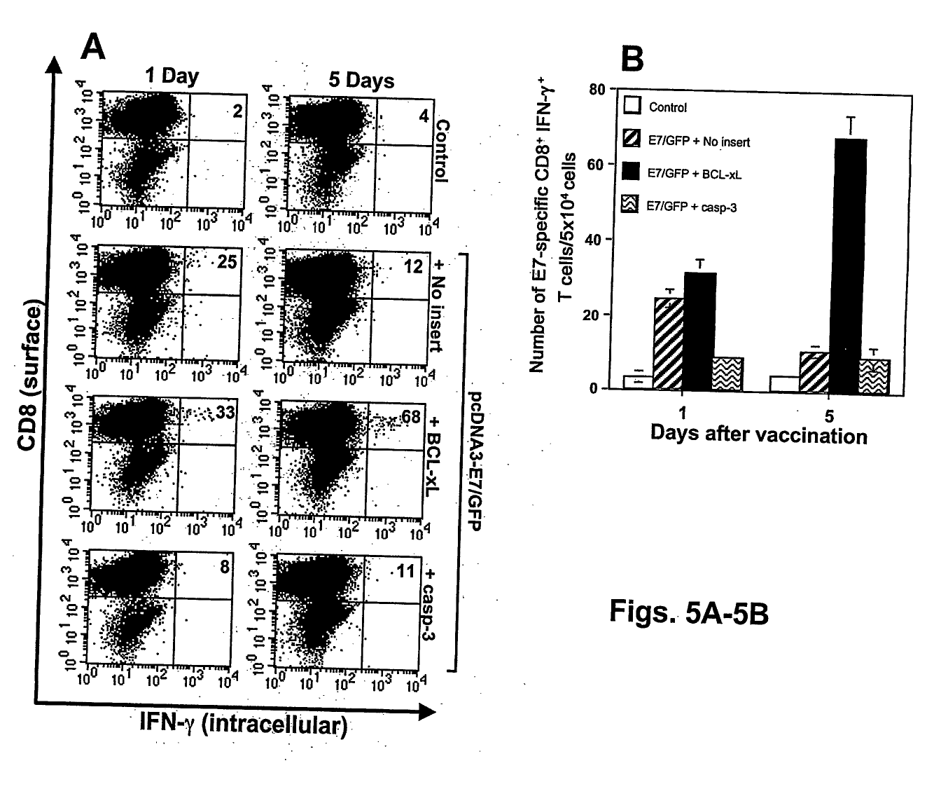

The ability of CD11c-enriched cells from the inguinal LNs of the various groups to stimulate IFNγ secretion from an E7-specific CD8+ T cell line (Wang et al, supra) was tested. The CD11c-enriched cells, isolated 1 or 5 days after the last DNA vaccination, were incubated with an E7-specific T cell line. As shown in FIG. 5, CD11c-enriched cells from mice co-administered E7/GFP DNA mixed and BCL-xL DNA were more effective in activating cells of the T cell line to secrete IFNγ compared with the other DNA constructs, particularly at day 5 (P<0.0005, one-way ANOVA). In comparison, CD11c-enriched cells from mice that had received E7/GFP DNA mixed with DNA encoding caspase-3 (or no insert) obtained on day 5 did significantly activate the antigen-specific CD8+ T cell line. These results indicate that the longer-surviving, transfected DCs, resulting from the co-administration of the DNA encoding BCL-xL, are also more active in antigen-specific T cell stimulation.

C: Discussion and Conclusions

The foregoing study demonstrated that co-administration of antigen-encoding DNA with DNA encoding an anti-apoptotic protein (1) enhances antigen-specific CD8+ T cell-mediated immune responses and (2) increases the survival of DCs in LNs draining the site of administration. This contrasts with previous studies showing that DNA vaccines encoding an antigen, when coexpressed with a proapoptotic agents such as Fas (Chattergoon, M A et al., 2000, Nat. Biotechnol. 18:974-979), mutant caspase with an altered active site (Sasaki et al., supra)), or suicide DNA encoding antigen (Leitner, W W et al, 2000, Cancer Res. 60:51-55) actually enhance antigen-specific T cell responses. This apparent inconsistency may be explained by any of a number of factors, including the expression vector used and the vaccine dose, regimen and route. Among these factors, the route of administration is believed to play a relatively more important role in the effects described above. It is worth noting that the studies cited above that used pro-apoptotic DNA to enhance vaccine potency employed intramuscular immunization.

In contrast, the results presented here were based on intradermal administration DNA encoding anti-apoptotic proteins. Intramuscular immunization should target antigen to myocytes, which are not professional APCs, and lack costimulatory molecules that are important for efficient T cell activation. In this setting, transfection of cells with DNA encoding pro-apoptotic factors may lead to apoptosis or necrosis, and should result in uptake of antigen by APCs through an “exogenous” cross-priming pathway that involves presentation of exogenous antigens through the MHC class I pathway to CD8+ T cells (for review, see Srivastava, P K et al., 1998, Immunity. 8:657-65; Heath, W R et al., 2001, Annu. Rev. Immunol. 19:47-64). In contrast, intradermal immunization can directly target antigen to Langerhans cells and facilitate direct presentation T cells by DNA-transfected DCs. Direct presentation plays an key role with CD8+ T cells after intradermal immunization with a gene gun. The present findings are consistent with this notion and indicate that inhibition of apoptosis prolongs survival of DNA-transfected DCs, resulting in a significant increase in the number of activated antigen-specific T cells. These notions suggest that the route of administration may have a profound impact on the effectiveness of DNA vaccines that employ or that are combined with pro- or anti-apoptotic polypeptides.

The results provided here strongly suggest that an increase in the number and activity of DCs presenting a specific antigen in a draining LN is likely due to inhibition of DC apoptosis. An earlier study also demonstrated that the DCs derived from BCL-2 transgenic mice had increased longevity compared to DCs from normal mice (Nopora, A et al, 2002). There remains a possibility that administration of DNA encoding anti-apoptotic agents may affect DC migration through chemokines or other factors that influence DC homing to the draining LN after encountering an antigen in the periphery. The present results support the idea that an increase in the number of antigen-expressing DCs in a LN contributes to enhancement of antigen-specific T cell activation

The present observation was that co-administration of DNA encoding BCL-xL with DNA encoding antigen generated the most potent enhancement of antigen-specific CD8+ T cell responsiveness among the anti-apoptotic proteins tested. BCL-xL is considered one of the most potent anti-apoptotic proteins and, like BCL-2, localizes to outer mitochondrial membranes and prevents release of pro-apoptotic factors from the mitochondria, including cytochrome c (Kharbanda, S et al., 1997, Proc. Natl. Acad. Sci. USA. 94:6939-42) and Smac/DIABLO (Du, C et al. 2000, Cell 102:33-42; Verhagen, A M et al., 2000, Cell 102:43-53; Sun, X M et al., 2002, J. Biol. Chem. 277:11345-51) by a mechanism that is not yet well understood. In addition, BCL-xL may inhibit apoptosis downstream of caspase-8 (Medema, J P et al., 1998, J. Biol. Chem. 273:3388-93). Thus, BCL-xL may inhibit apoptosis at multiple points along the programmed cell death pathways, which explains why it is one of the most potent anti-apoptotic factors. In summary, the present discovery demonstrates the usefulness of combining DNA encoding anti-apoptotic protein with DNA encoding an antigen as an approach to enhance antigen-specific CD8+ T cell immune responses including those expressed as antitumor effects. This approach can encompass not only antigen-encoding vectors but also chimeric vaccines that comprise DNA encoding antigen and targeting polypeptides. This approach is equally applicable to any antigen, so that it is readily applied with an expectation of success to other types of tumors, infectious agents or any other disease in which heightened antigen-specific immunity is desired.

### EXAMPLE II

### Enhancing DNA Vaccine Potency by Prolong Dendritic Cell Life and Employing Intracellular Targeting

(This example incorporates by reference T W Kim et al., J. Immunol. 171:2970-2976, 2003 Sep. 15)

A. Materials and Methods

Plasmid DNA constructs and DNA preparation. The generation of pcDNA3, pcDNA3-E7, pcDNA3-Sig/E7/LAMP-1, pcDNA3-CRT/E7, and pcDNA3-HSP70/E7 has been described previously (See Example I and Ji et al., supra; Cheng et al., supra; and Chen et al., 2000, supra). pSG5 plasmids encoding Bcl-xL or mt 7 (our mtBcl-xL) were generated as described previously (Cheng, E H, 1996, supra) Cheng, E. H., B. The DNA was amplified and purified according to Chen et al., supra).

Mice: See Example I.

DNA vaccination. See Example I. C57BL/6 mice were immunized with 2 μg of pcDNA3 encoding E7, CRT/E7, E7/HSP70, or Sig/E7/LAMP-1 mixed with 2 μg of pSG5 or pSG5-Bcl-xL. The mice received a booster with the same dose 1 wk later.

Intracellular cytokine staining and flow cytometry analysis. See Example I for most details.

Splenocytes were harvested (5 mice/group) 1 or 7 wks (for memory T cells) after the last vaccination. Before intracellular cytokine staining, 4×10⁶ pooled splenocytes from each vaccination group were incubated overnight with 1 μg/ml E7 (RAHYIVTF) peptide containing an MHC class I epitope (aa 49-57) for detecting E7-specific CD8+ T cell precursors or 1 μg/ml E7 peptide containing an MHC class II epitope (aa 30-67) for detecting E7-specific CD4+ T cell precursors. For the determination of the avidity of E7-specific CD8+ T cells, mice were vaccinated with pcDNA3-Sig/E7/LAMP-1 co-adminstered with pSG5-no insert, with pSG5-Bcl-xL, or with pSG5-mtBcl-xL. Mice were boosted with the same vaccine 1 wk later Splenocytes were collected and pooled 1 wk after the booster and incubated with the following concentrations of E7 peptide (aa 49-57:1, 10⁻¹, 10⁻², 10⁻³, 10⁻⁴, 10⁻⁵, 10⁻⁶, 10⁻⁷, or 10⁻⁸ μg/ml) overnight. The number of E7-specific IFNγ-secreting CD8+ T cells was determined as above.

In vivo tumor treatment and long-term tumor protection. See Example I. To study the subsets of lymphocytes that are important for the antitumor effects, a tumor protection experiment was performed, coupled with in vivo Ab depletion as above. For the long-term tumor protection experiments, 5 mice/group) were challenged i.v. with 10⁴ TC-1 tumor cells 7 wks after the last vaccination. Mice were monitored twice per week and sacrificed on day 42 after tumor challenge.

Statistical analysis. See Example I. In the tumor protection experiment, the principal outcome of interest was time to development of tumor. The event time distributions for different mice were compared by Kaplan and Meier and by log-rank analyses.

B. Results

Combined Anti-Apoptotic and Intracellular Targeting Strategies Further Enhance Antigen-Specific CD8+ T Cell Responses

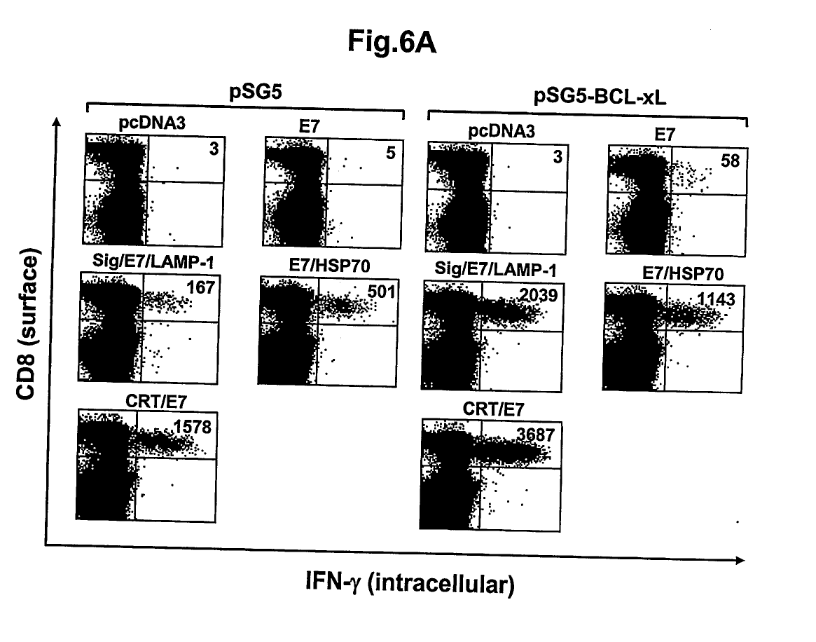

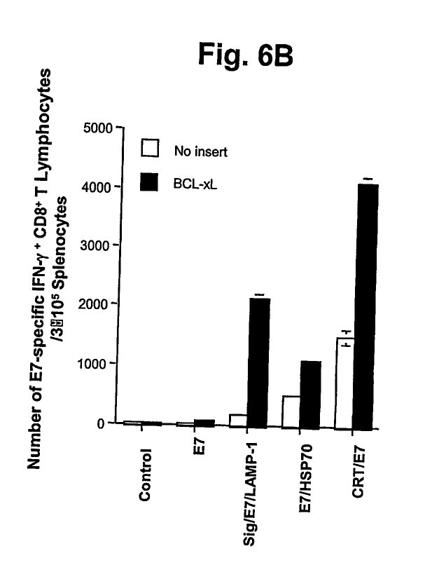

To explore whether DNA encoding Bcl-xL is capable of enhancing responses to DNA vaccines using various intracellular targeting strategies, the present inventors co-administered Bcl-xL with E7 linked to HSP70, CRT, or LAMP-1. As shown in FIGS. 6A and 6B, co-administration of Bcl-xL with any of the three intracellular targeting strategies increased the number of IFNγ-secreting E7-specific CD8+ T cell precursors compared with co-administration with pSG5 empty vector. Although the CRT/E7 vector mixed with Bcl-xL produced the strongest response, Sig/E7/LAMP-1 mixed with Bcl-xL displayed the greatest fold increase (at least 10-fold). The results demonstrate that (1) co-administration of the anti-apoptotic vector Bcl-xL in combination with any of three intracellular targeting strategies further enhances DNA vaccine potency, and (2) the most striking effect of the anti-apoptotic construct occurs when it is combined with Sig/E7/LAMP-1 DNA as the antigen/targeting polypeptide chimeric compositioni.

Co-Administration of pcDNA3-Sig/E7/LAMP-1 with pSG5-Bcl-xL Increases the Average Avidity of the E7-Specific CD8+ T lymphocyte Response

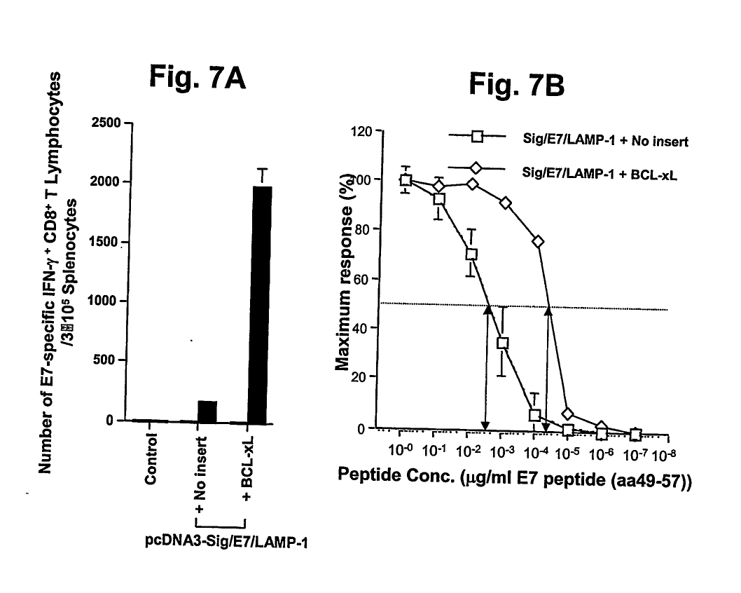

Prior studies have shown that high-avidity CTL provide better protection against viral infection (Derby, M et al., 2001, J. Immunol. 166:1690) and tumor challenge (Cheng, W F et al., 2002, J. Biomed. Sci. 9:675) than do low-avidity CTL. In addition, duration of DC-T cell interaction has been implicated as important in the generation of high avidity T cells (Langenkamp, A. et al., 2002, Eur. J. Immunol. 32:2046). Therefore, a functional avidity assay was performed to determine the avidity of E7-specific CD8+ T cells generated by vaccination of the combination of Sig/E7/LAMP-1 and one of Bcl-xL, mtBcl-xL, or empty vector. The number of IFNγ-secreting CD8+ T cells stimulated by 1 μg/ml E7 peptide (aa 49-57) was defined as the “maximum response” so that the functional avidity of T cells was based on comparisons to mice vaccinated with Bcl-xL, or empty vector at 50% of the maximum. The concentration of E7 peptide required to attain 50% of the maximum IFNγ+ CD8+ T cell response was ˜4×10⁵ μg/ml for mice vaccinated with Sig/E7/LAMP-1 combined with Bcl-xL, and ˜3×10³ μg/ml for mice vaccinated with Sig/E7/LAMP-1 mixed with empty vector or mutant mtBcl-xL (FIG. 7B). It was concluded that co-administration of Sig/E7/LAMP-1 with Bcl-xL generated higher avidity E7-specific CD8+ T cells than did co-administration of Sig/E7/LAMP-1 with empty vector or mutant mtBcl-xL. Furthermore, because the functional avidity of E7-specific CD8+ T cells elicited by co-co-administration with the apoptotically inactive mutant mtBcl-xL was nearly identical to that observed with control with empty vector, it was concluded that the anti-apoptotic function of Bcl-xL encoded by the administered vector was responsible for the observed effect (increased functional avidity).

Co Administration of pSG5-Bcl-xL with pcDNA3-Sig/E7/LAMP-1 Induced an Enhanced Th1 and a Diminished Th2 CD4+ Response

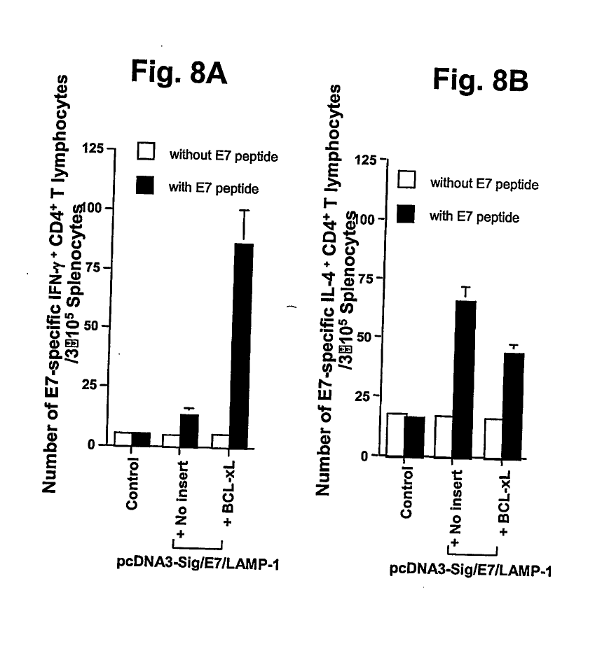

It is known that the LAMP-1 targeting strategy enhances antigen presentation to CD4+ T cells via targeting of expressed antigen to endosomal/lysosomal compartments, important loci for the MHC class II Ag presentation pathway (Wu, T C et al., 1995, Proc. Natl. Acad. Sci. USA 92:11671 and U.S. Pat. No. 5,633,234). To determine the nature of the E7-specific CD4+ T cell response to vaccination with Sig/E7/LAMP-1 combined with Bcl-xL DNA or empty vector, intracellular cytokine staining for IFNγ (secreted by Th1 cells) or IL-4 (secreted by Th2 cells) was performed using mouse splenocytes taken 1 wk after the last vaccination. As shown in FIGS. 8A and 8B, vaccination with Sig/E7/LAMP-1 mixed with Bcl-xL generated significantly more (expressed per 3×10⁵ splenocytes) E7-specific Th1 CD4+ T cells lymphocytes: 86.3±14.3 vs 13.5±2.5, and fewer E7-specific Th2 CD4+ lymphocytes (43.4±3.8 vs 65.2±6.4) than vaccination with Sig/E7/LAMP-1 mixed with empty vector. Thus, co-administration with DNA encoding Bcl-xL potentiates an antigen-specific CD4+ Th1 cell response and diminishes an antigen-specific CD4+ Th2 cell response.

Co-Administration of pSG5-Bcl-xL with pcDNA3-Sig/E71LAMP-1 Vaccine Induces a Stronger E7-Specific CD8+ T Cell Response in CD4 Knockout Mice

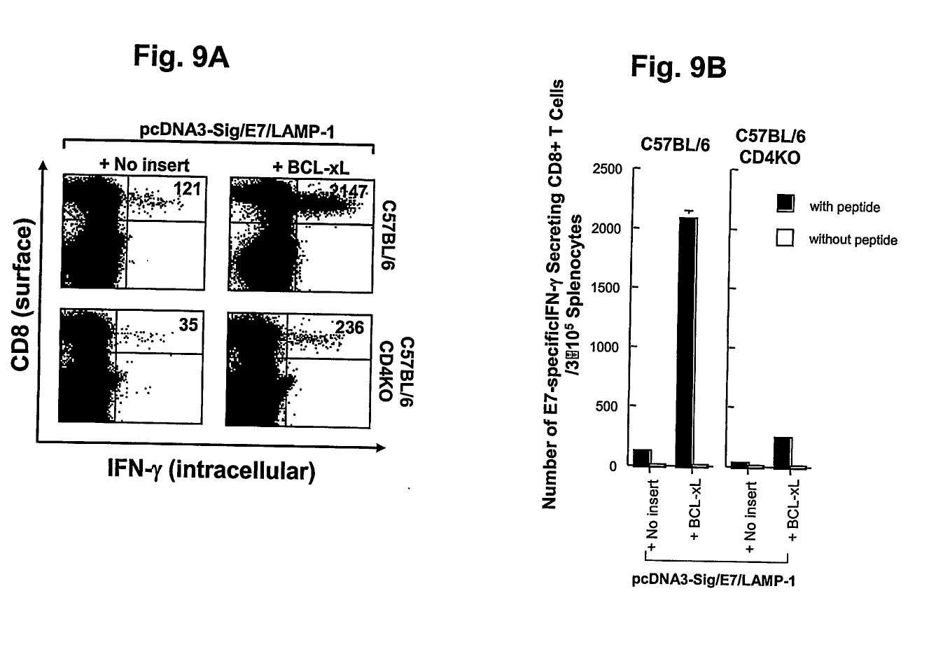

To examine whether CD4+ T cells were essential for the enhancee CD8+ T cell response, studies enumerated E7-specific CD8+ T cells generated in normal vs CD4KO C57BL/6 mice. As shown in FIGS. 9A and 9B, wild-type mice co-administered Bcl-xL with Sig/E7/LAMP-1 vaccine showed a greater E7-specific CD8+ T cell response as compared to wild-type mice vaccinated with Sig/E7/LAMP-1 mixed with empty vector. The same trend was observed when CD4KO mice received the combination of Sig/E7/LAMP-1 and Bcl-xL. When comparing CD4KO mice with wild type mice, vaccination with Sig/E7/LAMP-1+Bcl-xL resulted in an ˜10-fold greater E7-specific CD8+ T cell response in the wild-type mice. It was concluded that CD4+ T cells make an important contribution to the E7-specific CTL response.

Although the number of E7-specific CD8+ T cells generated in CD4KO mice was significantly lower than in wild types, the results demonstrated that the co-administration of Bcl-xL DNA with Sig/E7/LAMP-1 DNA in CD4KO mice was still able to generate ˜2-fold more specific CD8+ T cells vs. co-administration of Sig/E7/LAMP-1 DNA with empty pSG5 vectors in wild-type mice. According to these results, a DNA vaccine approach that includes an anti-apoptotic strategy and an intracellular targeting strategy should be more potent in generating CD8+ T cell-mediated immune responses in a CD4-depleted host when compared to the response stimulated by DNA vaccine using only an intracellular targeting strategy in an immunocompetent host.

Anti-Tumor Immunity is Enhanced by Co-Administration of pcDNA3-Sig/E7/LAMP-1 with pSG5-Bcl-xL

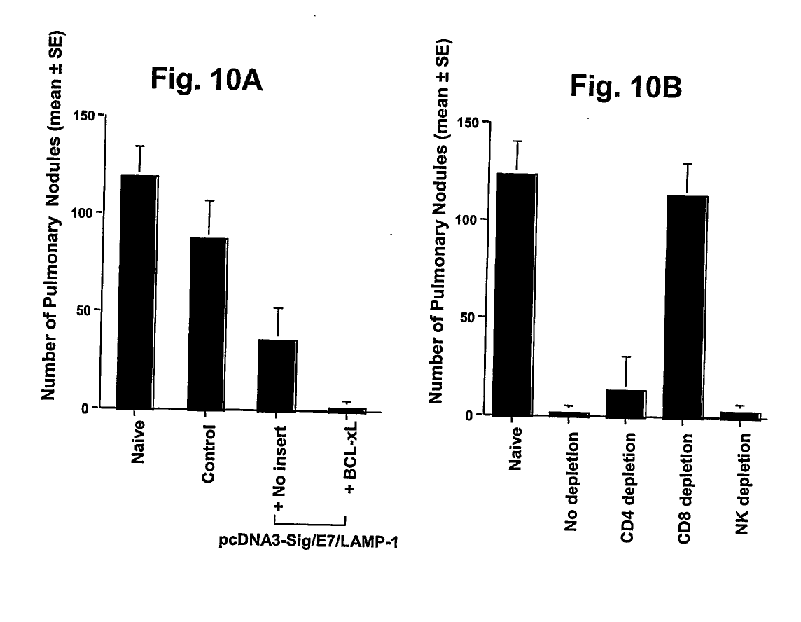

A factor vital to the success of any therapeutic vaccine, as exemplified here as an HPV therapeutic vaccine, is the ability to treat infected and/or tumor-bearing patients. To determine the therapeutic effectiveness of the present strategy, a study was conducted that tested the ability of Sig/E7/LAMP-1 mixed with Bcl-xL vs empty vector to treat established TC-1 tumor in a hematogenous spread model. As shown in FIG. 10A, mice treated with Sig/E7/LAMP-1 mixed with Bcl-xL developed significantly fewer tumor nodules than did control mice treated with Sig/E7/LAMP-1 mixed with empty vector, or naive mice. Thus co-administration of Bcl-xL DNA improves the antitumor therapeutic capacity of a DNA vaccine comprising the tumor antigen and a targeting moiety.

In a tumor protection study, antibody depletion was used to determine which subset of T cells was needed for the antitumor response. Mice were vaccinated with Sig/E7/LAMP-1 mixed with Bcl-xL and subsequently challenged with TC-1. Antibody depletion was initiated concurrently with tumor challenge. Results are in FIG. 10B. Mice depleted of CD8+ T cells displayed nearly the same degree of tumor growth as naive mice, and mice depleted of CD4+ T cells displayed slightly greater tumor growth vs. nondepleted mice. There was no effect of NK cell depletion. It was concluded that CD8+ T cells are essential for the antitumor effect, with CD4+ T cells also contributing.

Prolonged Immunity and Tumor Protection after Co-Administration of pcDNA3-Sig/E7/LAMP-1 and with pSG5-Bcl-xL

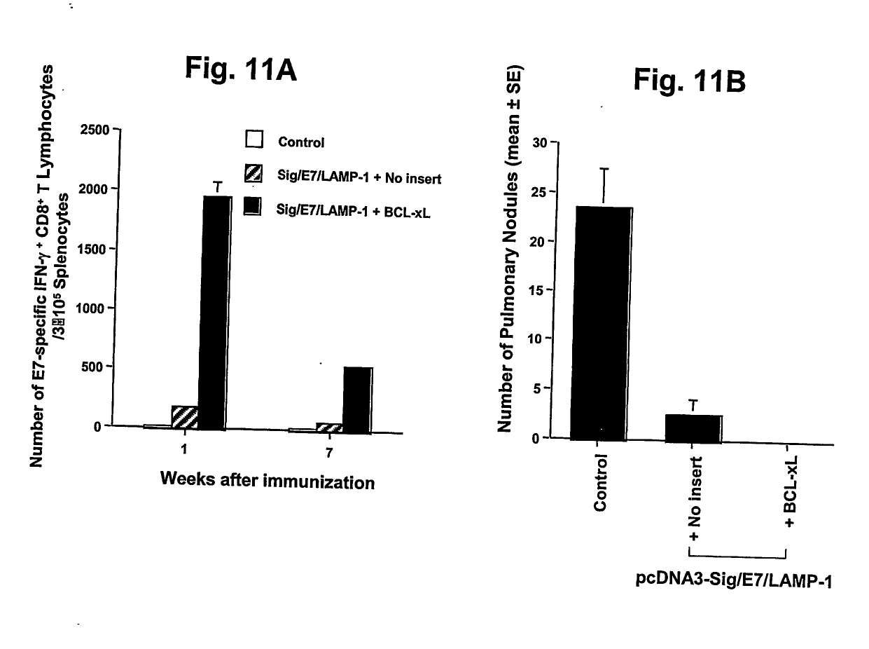

A successful protective vaccine must be able to induce a protective immune response that persists for a significant interval. To assess the ability of the present vaccination strategy to generate long-term specific CD8+ T cell immune responses and tumor protection, studies compared vaccination with Sig/E7/LAMP-1+Bcl-xL to vaccination with Sig/E7/LAMP-1+ empty vectors. Intracellular cytokine staining and flow cytometry to enumerate E7-specific CD8+ T cells was performed 1 and 7 wk after immunization. As shown in FIG. 11A, Sig/E7/LAMP-1 mixed with Bcl-xL generated an ˜7-fold higher E7-specific IFNγ CD8+ T lymphocyte response at 7 wks than Sig/E7/LAMP-1 mixed with empty vector. Thus, co-administration of the anti-apoptotic construct with the Sig/E7/LAMP-1 vaccine generated a more powerful immune response. Vaccinated mice were challenged with 10⁴ TC-1 tumor cells 7 wk after the final immunization. As shown in FIG. 11B, no tumor nodules were detectable in mice vaccinated with Sig/E7/LAMP-1 mixed with Bcl-xL, whereas mice vaccinated with Sig/E7/LAMP-1+empty vector exhibited 1.6±2.3 tumor nodules 42 days after TC-1 challenge. Therefore, co-administration of Sig/E7/LAMP-1 with Bcl-xL completely prevented tumor nodule formation 7 wk after vaccination.

Taken together, the present results indicate that a DNA vaccine that combines an intracellular targeting strategy with a strategy to prolongs DC life results in a more durable, potent and longer lasting state of antigen-specific CD8+ T cell mediated immunity that can be manifest as antitumor protection.

C. Discussion

Ther results disclosed above support the conception that a DNA vaccination strategy that combines (a) DNA encoding an antigen and (b) DNA encoding an intracellular targeting polypeptide in one vector and (c) another DNA vector that encodes a polypeptide that prolongs the life of DCs will enhancing the antigen-specific immune response thatn (a)+(b) alone

This combination strategy was shown to be effective with three (a)+(b) combinations: (i) HSP70/E7 (5), (ii) CRT/E7 (6), and (iii) Sig/E7/LAMP-1, resulting in strong and durable E7-specific CD8+ T cell responses manifest, inter alia as long-term tumor protection in vaccinated hosts. These results are attributed to prolongation of the life of DCs in the draining LN that are centrally involved in generating the immune response which is achieved by inhibition of apoptosis using the anti-apoptotic protein Bcl-xL. As a result of the combination treatment, there are more, and longer lived DCs in the LNs draining the site of immunization (8), as well as to enhanced processing of antigen due to expression of targeting polypeptide, whether it be CRT, LAMP-1, or HSP70 linked to the antigen. Thus, as discovered here, it is possible to modify DCs simultaneously in two different ways, using different means, to further enhance DNA vaccine potency.

Of the targeting strategies tested herein, Sig/E7/LAMP-1 could evoke the greatest differential in the antigen-specific CD8+ T cell response when it was co-administered with Bcl-xL DNA (FIG. 6B). This may be due to an increase in the CD4 Th cells, as Sig/E7/LAMP-1 is the only one of the constructs compared here that targets antigen the MHC class II processing pathway, activating specific CD4+ T cells more effectively than do the other constructs. An experiment using CD4KO mice demonstrated a significantly lower number of E7-specific CD8+ T cells in the absence of CD4+ cells. Thus, CD4+ T cells appear to be important in the process leading to the enhanced immunity resulting from the present strategy.

Co-administration of Bcl-xL DNA with Sig/E7/LAMP-1 DNA in CD4KO mice generated more E7-specific CD8+ T cells than did co-administration of Sig/E7/LAMP-1 DNA with pSG5 in wild-type mice, suggesting that a DNA vaccination strategy combining intracellular targeting with anti-apoptotic proteins may be useful for specific CD8+ T cell responses in individuals with a compromised immune system in which CD4+ cells are reduced significantly. This has obvious relevance to the treatment, and/or vaccination of people with HIV disease/AIDS. This CD4-depletion in this population is a likely cause of the increased severity of HPV infection and associated lesions in HIV-positive subjects (reviewed in Kuhn, L. et al., 1999, Curr. Opin. Obstet. Gynecol. 11:35; Del Mistro, A et al., 2001, Eur. J. Cancer 37:1227). Thus, this combination strategy is predicted to be useful in controlling of HPV infection and HPV-associated lesions in CD4-depleted humans.

Co-administration of DNA encoding Bcl-xL also resulted in a response characterized by higher-avidity antigen-specific CD8+ T cells. The anti-apoptotic function of Bcl-xL was deemed essential for this effect. The ability of Bcl-xL to extend DC life span would lead to prolonged DC-T cell interaction in responding LNs; the duration of DC-T cell interactions has been implicated in the generation of high-avidity specific T cells (Langenkamp et al., supra). Thus, prolonged DC life resulting from the present invention contributes directly to increased E7-specific CD8+ T cell avidity. A response characterized by high avidity CD8+ T cells is known to result in better qualitative protective (including antitumor) effects than responses mediated by low-avidity CD8+ T cells (Alexander-Miller, Mass. et al., 1996, Proc. Natl. Acad. Sci. USA 93:4102). High-avidity CD8+ T cells enhance protection by recognizing structures with low antigen density, for example, killing infected cells sooner than do low-avidity CD8+ T cells (Derby et al., supra). Earlie studies disclosed that higher-avidity CTLs may produce a stronger antitumor effects in vaccinated mice (Cheng et al., 2002, supra; Yang, S et al., 2002, J. Immunol. 169:531).

As HPV vaccine research has moved into the clinical arena, it ie increasingly important to discuss the clinical implications of newly developed HPV vaccines and strategies in anticipation of potential future clinical application. Safety is a major consideration for the clinical application of any new vaccination strategy and is especially important in this case, because increased Bcl-xL expression could be viewed as potentially hazardous to humans. This is due to the concern that the anti-apoptotic effects of Bcl-xL could interfere with the normal regulation of DC function, which could tend toward autoimmunity. However, no histological or clinical evidence of autoimmunity was observed in any of the animals used in the present studies. Another concern with new molecular vaccine is oncogenesis because Bcl-xL has been implicated in oncogenic transformation of healthy cells (Lebedeva, I et al., 2000, Cancer Res. 60:6052). One strategy to improve safety is to transfect DCs with DNA encoding factors that may indirectly enhance DC survival with a reduced concern for oncogenicity, such as TNF-related activation-induced cytokine, CD40 ligand, IL-12, and IL-15, and serine protease inhibitor 6 (Medema, J P et al., 2001, J. Exp. Med. 194:65725). Among these molecules, CD40 ligand (Mendoza, R B et al., 1997, J. Immunol. 159:5777), IL-12 (Kim, J et al., 1997, J. Immunol. 158:816), and IL-15 (Xin, K Q et al., 1999, Vaccine 17:858) have been tested a enhancers of DNA vaccine potency.

The present results encourage the application of anti-apoptotic proteins in combination with other vaccine enhancement strategies for future development of therapeutic DNA vaccines to combat HPV infection and cervical cancer.

### EXAMPLE III

DNA Encoding Serine Protease Inhibitor-6 (Serpinb9) Enhances Potency of DNA Vaccine

(This example incorporates by reference T W Kim et al., Cancer Res. 64: 400-405, 2004 Jan. 1)

A. Materials and Methods

Plasmid DNA Constructs and DNA Preparation. The generation of pcDNA3-E7, pcDNA3-CRT/E7, pcDNA3-E7/HSP70 and pcDNA3-Sig/E7/LAMP-1 are described above or in references cited above. Generation of pcDNA3-ETA(dII)/E7 was described in C F Hung et al., 2001, Cancer Res 61:3698-3703; Wu et al., WO 03/085085). For generation of pcDNA3-SPI-6, SPI-6 was first amplified with PCR using mouse cDNA as the template and a set of primers, 5′-cccgaattcatgaatactctgtctgaagga-3′ [SEQ ID NO:87]and 5′-tttggatcctggagatgagaacctgccaca-3′ [SEQ ID NO:88]. The amplified product was then cloned into the EcoR I/BamH I sites of the pcDNA3 vector.

To generate the inactive mtSPI-6 containing the P14 mutation (T327R), most of the SPI-6 ORF was amplified from pSVTf/SPI-6 (Sun, J et al., 1997, J Biol Chem 272:15434-41) using the primers 5′-ggctgctgcagcctcccggccttcctcattgat-3′ (antisense) [SEQ ID NO:89] and 5′-gcatcatgaatactctgtc-3′ (sense) [SEQ ID NO:90], and cloned into pZeroblunt (Invitrogen). The product included a naturally-occurring PstI site downstream of the primer-introduced T327R substitution. This partial ORF was cloned into the EcoRI site of pSVTf, and the full length ORF was then reconstituted by inserting a 200 bp PstI fragment containing the last part of the ORF and 3′UTR, and verified by DNA sequencing. For generation of pcDNA3-mtSPI-6, mutant SPI-6 was cut at the EcoR I/BamH I sites from pSVTf-mtSPI-6 and cloned into the EcoR I/BamH I sites of the pcDNA3 vector. The accuracy of these constructs was confirmed by DNA sequencing. The DNA was amplified in E. coli DH5α and purified as described previously. The expression of SPI-6 and mtSPI-6 in COS-7 cells transfected with DNA encoding anti-apoptotic protein was characterized by RT-PCR.

Mice. See Example I.

DNA Vaccination. See Example I. C57BL/6 mice were immunized with 2 μg of pcDNA3 encoding E7, CRT/E7, E7/HSP70, ETA(dII)/E7, or Sig/E7/LAMP-1, mixed with 2 μg of pcDNA3, pcDNA3-SPI-6, or pcDNA3-mt SPI-6. The mice received a booster with the same dose one week later.

Intracellular Cytokine Staining and Flow Cytometry Analysis. See Example I for details. Splenocytes from each vaccination group were incubated for 16 hours with either 1 μg/ml of E7 peptide containing an MHC class I epitope for detecting E7-specific CD8⁺ T cell precursors or 10 μg/ml of E7 peptide (aa 30-67) containing an MHC class II epitope for detecting E7-specific CD4⁺ T cell precursors.

In Vivo Tumor Protection and Tumor Treatment Experiments. See Example I.

Survival of Dendritic Cell Line (DC-1). An immortalized DC line (Shen, Z et al., 1997, J Immunol, 158:2723-30) was provided by Kenneth Rock (University of Massachusetts, Worcester, Mass.). Subclones were generated by the present inventors with continued passage (DC-1) that were easily transfected using Lipofectamine® 2000 (Life Technologies, Rockville, Md.). DC-1 cells (5×10⁵) were co-transfected with 1 μg of pcDNA3-E7/GFP mixed with 4 μg of pcDNA3-SPI-6, pcDNA3-mt SPI-6, pcDNA3 (no insert) after the formation of Lipofectamine®/DNA complexes. GFP+ cells were collected 16 hours later by cell sorting in a flow cytometer. GFP+ DC-1 cells (2×10⁴) were incubated with 2×10⁶ cells of an E7-specific CD8+ T cell line for 6 hours. Apoptotic dendritic cells were enumerated by Annexin V staining after gating around a population of GFP+ cells and were analyzed via flow cytometry as described above.

B. Results

Co-Administeration of DNA Encoding SPI-6 Increases CD8⁺ T Cell Responses and Anti-Tumor Effects

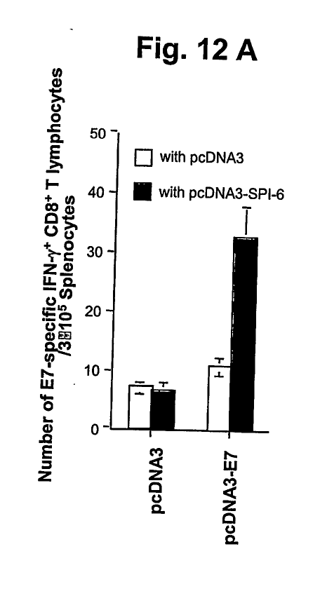

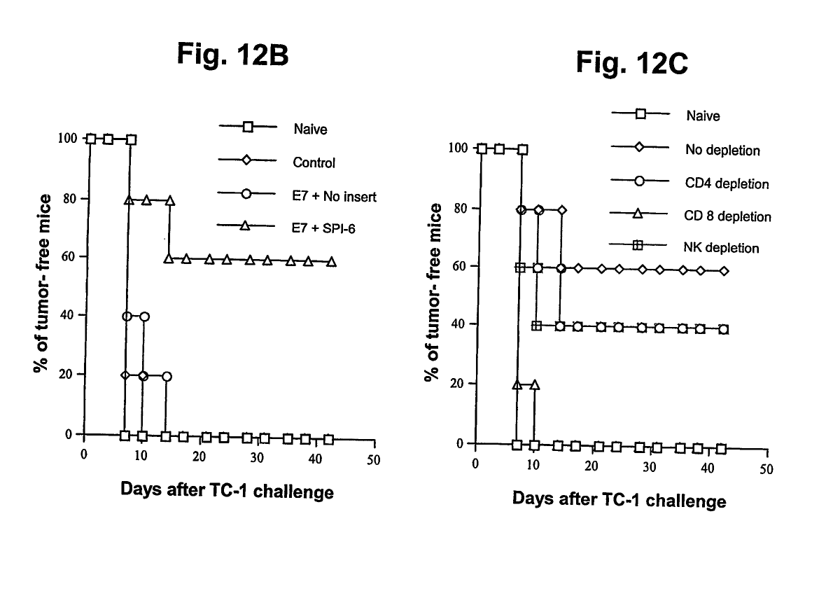

To further verify the present inventors' conception that SPI-6 will prolong DC life and enhance an immune response elicited by DNA vaccination, the an antigen-encoding pcDNA3-E7 was co-administered with control pcDNA3 or pcDNA3-SPI-6. FIG. 12A shows that inclusion of pcDNA3-SPI-6 resulted in a greater number of E7-specific IFN-γ-secreting CD8⁺ T cells (expressed per 3×10⁵ splenocyte), 32.3±5.1) compared to the control pcDNA3 (7.0±1.0) or to 5 vaccination with the antigen vector, pcDNA3-E7, alone (10.7±1.5). Thus, SPI-6 DNA can enhance antigen-specific CD8⁺ T cell responses when co-administered with antigen-encoding DNA.

To determine if the enhanced response observed above has “clinical” effects against a tumor, an in vivo tumor protection study was performed using the E7-expressing tumor TC-1.) As shown in FIG. 12B, 60% of mice vaccinated with pcDNA3-E7 co-administered with pcDNA3-SPI-6 remained tumor-free 42 days after tumor challenge. If the co-administered vector was the control pcDNA3, all mice developed tumors after only 14 days. Thus, co-administration of antigen-encoding DNA with SPI-6 DNA potentiates an anti-tumor effect against a tumor expression the appropriate antigen.

To determine which subsets of lymphocytes are important for the potentiated anti-tumor effects, an in vivo antibody depletion study was performed. Results shown in FIG. 12C) indicate depletion of CD8⁺ T cells resulted in tumor growth in all mice within two weeks. In contrast, 40% of the mice from which CD4⁺ cells or NK cells had been depleted (and 60% of control mice with sham depletion) remained tumor-free 42 days. Thus, CD8⁺ T cells play a vital effector role in this form of anti-tumor defense, whereas CD4⁺ cells and NK cells may also contribute (though the effects of depleting these two cell populations did not differ significantly different from non-depleted mice).

CD8⁺ T Cell Responses are Markedly Enhanced by Combining Intracellular Antigen-Targeting Strategies with Anti-Apoptotic Effects of SPI-6

In view of the impact of apoptosis inhibition by SPI-6 DNA shown above, it was conceived that such SPI-6 DNA co-administration would enhance responses to other improved DNA vaccination strategies, particularly those induced by chimeric vaccines comprising DNA encoding an antigen linked to DNA encoding a targeting polypeptide.

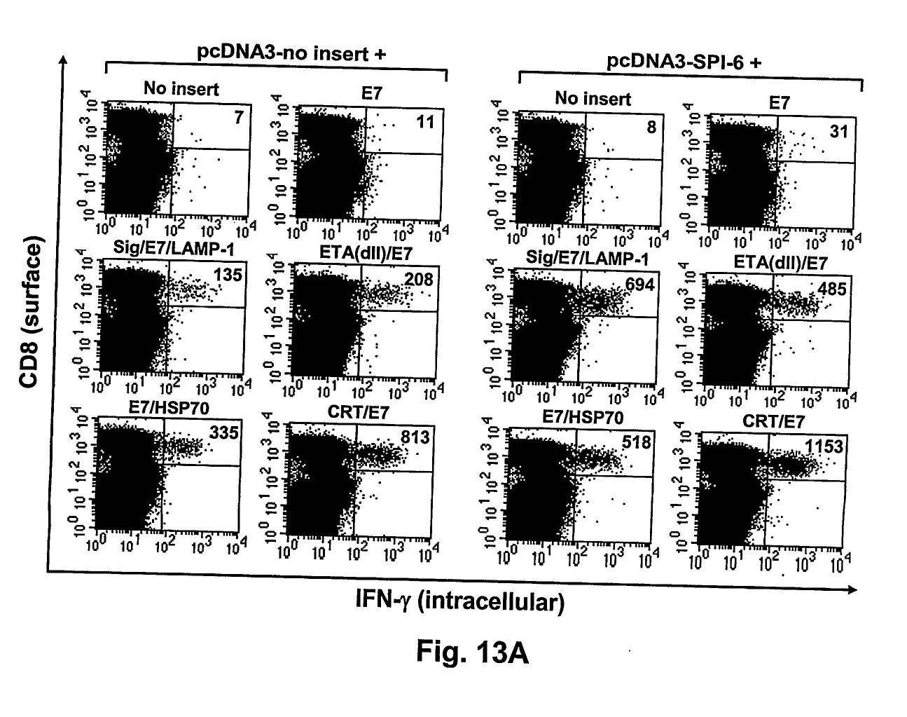

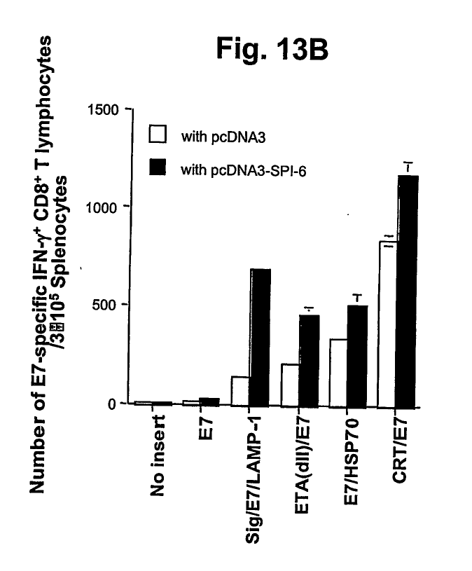

SPI-6 was co-administered with E7 linked to either ETA(dII), HSP70, CRT, or the sorting signal of LAMP-1. As depicted in FIGS. 13A and 13B, responses to the latter vaccines were further potentiated by co-administration of with DNA encoding SPI-6. Each of the constructs generated a greater number of antigen-specific CD8⁺ T cells when SPI-6 DNA was co-administered (compared to co-administeration of the control empty vector). SPI-6 DNA provoked the greatest enhancement with the Sig/E7/LAMP-1 vaccine (˜5 fold). Thus the potency of an antigen-encoding DNA vaccine that included a linked intracellular targeting polypeptide were further increased by the apoptosis-inhibiting effect produced by co-administering DNA encoding SPI-6.

Co-Administering SPI-6 with DNA Encoding Various Intracellular Targeting Polypeptides Significantly Enhances CD4⁺ Th1 but not CD4⁺ Th2 Responses

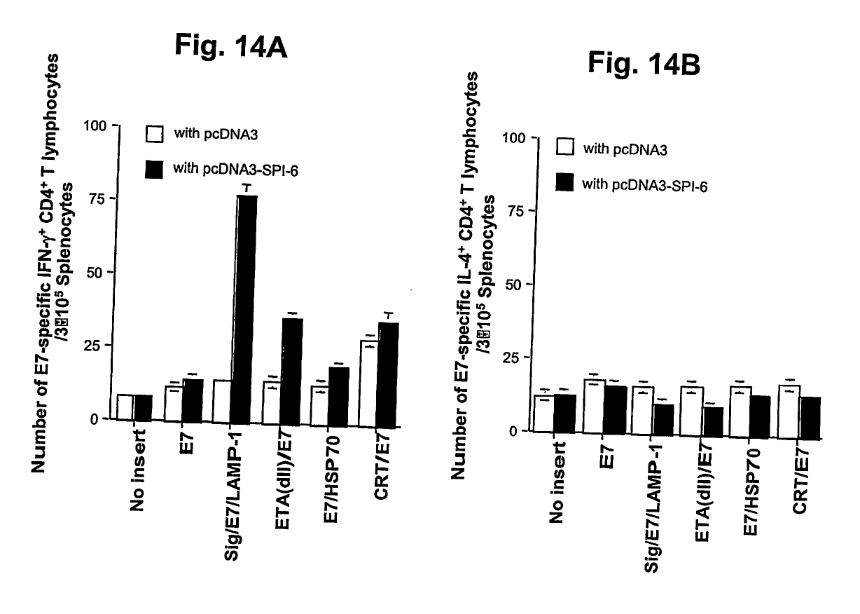

Studies of intracellular cytokine staining for IFN-γ and IL-4 were performed. As depicted in FIG. 14A co-administering DNA encoding SPI-6 with DNA encoding E7 linked to intracellular targeting polypeptides increased the E7-specific CD4+ Th1 cell response. The combination had the greates effect when the Sig/E7/LAMP-1 vaccine was used in terms of generating E7-specific IFN-γ-secreting CD4⁺ Th1 cell precursors (per 3×10⁵ splenocytes), 77.0±3.6 which was an ˜5 fold increase over the response elicited by Sig/E7/LAMP-1 co-administered with control empty vector (14.1±1.0).

As shown in FIG. 14B, co-administering the various antigen-encoding constructs with SPI-6 DNA did not increase the antigen-specific CD4⁺ Th2 immune response, measured as the frequency of E7-specific IL-4-secreting CD4⁺ T cell precursors, In fact, slight decreases in this response followed co-administration of SPI-6 DNA. It appears then that SPI-6 does not enhance Th2 CD4⁺ T cell responses. Taken together, the results indicate that vaccination with an antigen-encoding DNA co-administered with SPI-6 DNA facilitates the activation of E7-specific IFN-γ⁺ CD4⁺ Th1 cells, but does not of E7-specific IL-4⁺ CD4⁺ Th2 cells.

Co-Administering pcDNA43-Sig/E7/LAMP-1 with pcDNA3-SPI-6 to Treat Tumors

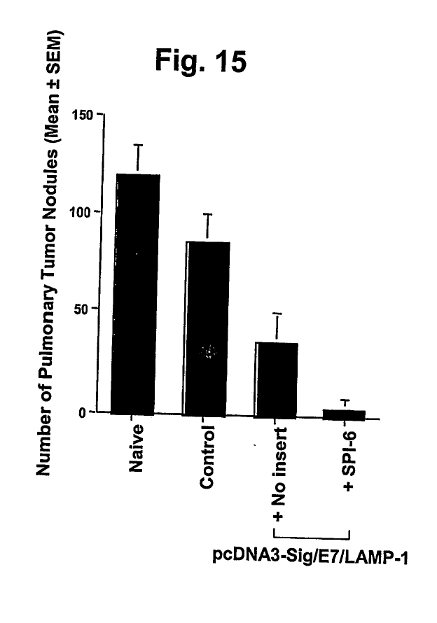

A study was done that combined the intracellular targeting benefits of the Sig/E7/LAMP-1 construct with the anti-apoptotic effect of SPI-6 DNA in generating a treatment response against an existing tumor. In view of the results presented above, Sig/E7/LAMP-1 was selected over the other chimeric constructs for this analysis. The study utilized the hematogenous spread pulmonary tumor model with TC-1 as described in the Examples above. s shown in FIG. 15, mice immunized with Sig/E7/LAMP-1 DNA co-administered with SPI-6 DNA exhibited significantly fewer pulmonary tumor nodules (3.6±5.3, P≦0.001, one-way ANOVA) compared to naive mice (118.6±15.0) or mice given Sig/E7/LAMP-1 DNA in combination with a control with empty vector (85.8±14.4). Thus, co-administeration of with SPI-6 DNA with a targeted vaccine vector, Sig/E7/LAMP-1 DNA, evoked a stronger therapeutic immune response against a tumor expressing the immunizing antigen and that this, anti-tumor response was more effective than the already improved treatment response induced by vaccination with Sig/E7/LAMP-1 DNA (vs.E7 alone).

Expression of the Anti-Apoptotic Function of SPI-6 is Required for Prolonging the Life of DCs and Enhanced Immune Responses

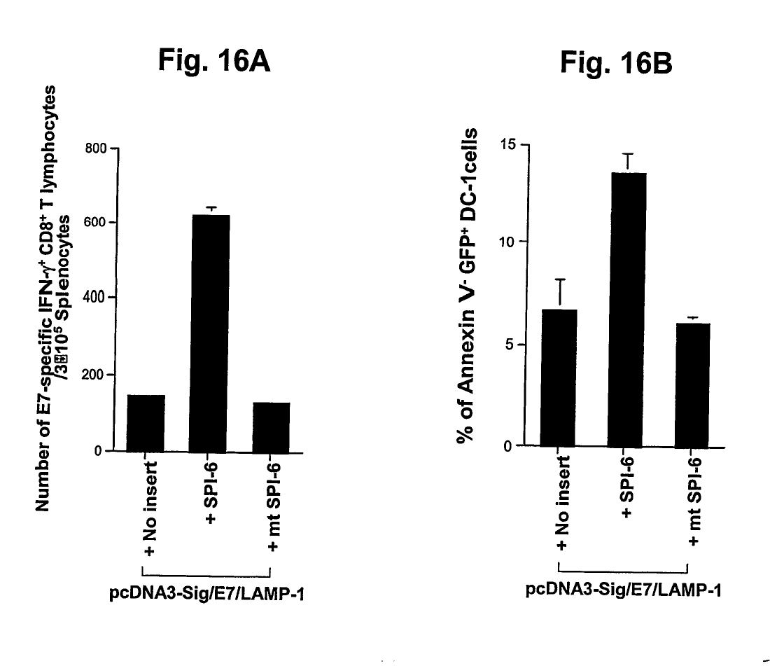

The anti-apoptotic function of a serpin can be destroyed by substituting the conserved P14 Thr with Arg (Bird, C H et al., 1998, Mol Cell Biol 18:6387-98). To confirm that the anti-apoptotic function of SPI-6 is required to prolong DC survival, an inactive P14 mutant of SPI-6 (mtSPI-6), was generated and analyzed in the above experimental system. As shown in FIG. 16A, vaccination with pcDNA3-Sig/E7/LAMP-1 co-administered with mutant SPI-6 (mtSPI-6) DNA yielded fewer E7-specific CD8⁺ T cell precursors (132.0±2.6) than did vaccination with pcDNA3-Sig/E7/LAMP-1 co-administered with pcDNA3-SPI-6 encoding wild-type active SPI-6 (620.7±22.9). Therefore, the anti-apoptotic function absent in the mutant SPI-6 is critical for the observed immune potentiating effect on the response induced by an antigen-encoding DNA vaccine composition.

To confirm that SPI-6 had the expected anti-apoptotic effects, cells of a DC line, DC-1, were transfected with E7/GFP DNA together with (i) SPI-6 DNA or (ii) empty vector, or (iii) mtSPI-6 DNA. These transfected DC-1 cells were incubated with an E7-specific CD8+ T cell line in vitro. The GFP+ DC-1 cells were subsequently stained with Annexin V to enumerate apoptotic cells. DC-1 cells that stained positively for Annexin V (i.e., apoptotic cells). As shown in FIG. 16B, the percentage of GFP+, Annexin V-negative DC-1 target cells was greater in DC-1 cells transfected with E7/GFP DNA mixed with SPI-6 DNA (13.63±0.97) than in DC-1 cells transfected with E7/GFP DNA mixed with empty vector or mtSPI-6 DNA. Thus, there were fewer apoptotic cells when SPI-6 DNA was concomitantly transfected into the cells as compared with functionally inactive mutant mtSPI-6 DNA. In fact, co-transfection with mtSPI-6 resulted in virtually the same percentage of Annexin V negative DC-1 cells as did the empty vector (6.10±0.30 vs. 6.67±1.29, suggesting that mutant SPI-6 could not prolonging DC survival.

The foregoing results prove that SPI-6 does possess anti-apoptotic function that prolongs the life of antigen-transfected DCs in vitro, and that its ability to delay apoptosis is important in enhance the immune response that is dependent upon DCs in vivo.

C. Discussion

The foregoing studies demonstrated that co-administering DNA encoding SPI-6 with antigen-encoding DNA (alone or linked to DNA encoding an additional intracellular targeting polypeptide) significantly enhances the potency of HPV-16 E7 DNA vaccines. The anti-apoptotic function of the SPI-6 is vital to this enhancement. This co-administration strategy proved effective in potentiating E7-specific CD8⁺ T cells and IFNγ-secreting CD4⁺ T cells as well as evoking markedly enhanced anti-tumor effects. Thus, it is expected that co-administering E7 DNA (or E6-DNA) with SPI-6 DNA may help to control E7-(or E6-expressing) tumors and HPV infection. It is further expected that these effects will be manifest and useful with any DNA vaccine encoding any antigen or antigenic epitope that engenders CD8+ or CD4+ T cell mediated immunity.

It is believed that the immunopotentiating effects of SPI-6 DNA occur because the anti-apoptotic protein prevents CTL-induced apoptosis of DCs. The inactive SPI-6 mutant studied above has a substitution in its proximal hinge that destroys its ability to inhibit granzyme B and prevent granzyme B-mediated apoptosis. Thus, the prolonged life of DCs brought about by SPI-6 is responsible for the effects observed.

The increased numbers of active E7-specific CD4⁺ Th1 cells described above are believed to contribute to the observed anti-tumor effect. Th1 cells stimulate the maturation of DCs via TFNγ secretion and CD40/CD40L interactions (Ridge, J P et al., 1998, Nature 393:474-78) which induces DCs to express IL-12 and to prime antigen-specific CD8+ T cells more effectively. IL-12 secretion is known to contribute to anti-tumor effects in vivo (Brunda, M J et al., 1993, J Exp Med 178:1223-30). Thus, Th1 CD4+ T cells may augment the anti-tumor effects observed above by stimulating DCs to produce IL-12, by secretion of IFNγ and by enhancing CTL activation by DCs.

As described in the earlier examples, the present inventors have transfected DCs with DNA encoding other anti-apoptotic proteins such as Bcl-xL and Bcl-2. Co-administration of DNA encoding these anti-apoptotic proteins with antigen-encoding DNA proved to be a powerful stimulus to antigen-specific CD8+ T cell responses and immunological memory. This response was also shown to be due to prolonged DC survival, resulting in enhanced antigen presentation to T cells by DCs in the LNs draining the site of antigen entry. Anti-apoptotic proteins of the Bcl-2 family (Bcl-2, Bcl-xL) were found to be the greatest enhancers of the antigen-specific cell-mediated immune response studied. The use of these anti-apoptotic proteins is associated with safety concerns because, as discussed in Example II, proteins of the Bcl-2 family are overexpressed in some cancers, and have been implicated as contributors to cellular immortalization.

In an effort to resolve such safety issues, the present inventors conceived and proved that SPI-6 would prevent CTL-induced DC death by inhibiting the perforin/granzyme B mechanism of CTL-induced apoptosis. Because it is naturally expressed in mature DCs, SPI-6 may represent a safer and effective method for enhancing DNA vaccine potency by offering a means of prolonging DC life with a lessened risk of DC immortalization (Medema et al., supra). While the Bcl-2 anti-apoptotic proteins inhibit CTL-induced apoptosis via multiple pathways (Hockenbery, D M et al, 1993, Cell 75:241-251; Cheng, E H et al., 1996, supra) SPI-6 and its human counterpart, PI-9, inhibit only the perforin/granzyme B pathway. The other major pathway, Fas-mediated apoptosis, is not affected by SPI-6 (Medema, J P et al., 2001, Proc Natl Acad Sci USA, 98:11515-20). In this way, SPI-6 represents a means for inhibiting CTL-induced apoptosis without completely depriving CTLs of their capacity to trigger death in dendritic cells.

Although use of SPI-6 alleviates certain safety concerns associated with Bcl-2 family proteins, but Bcl-2 family proteins such as Bcl-xL provide a greater enhancement of DNA vaccine potency (Example II), probably because Bcl-2 and Bcl-xL inhibit apoptosis at multiple points, whereas SPI-6 interferes solely with granzyme B activity. It is now clear that the granzyme family is composed of members other than granzyme B, raising the possibility of enhancing DNA vaccine potency by co-administration of DNAs encoding multiple granzyme inhibitor molecules with DNA encoding the antigen. Use of such a genus of inhibitors is within the scope of this invention. Since perforin is important for the apoptotic function of the granzyme family, it should be possible to further inhibit apoptosis by disrupting perforin function. Therefore, focusing on the perforin/granzyme pathway will lead the way to DNA vaccine components that can more inhibit apoptosis and be as or more stimulatory to the immune response as the Bcl-2 or Bcl-xL polypeptides.

Because a majority of cervical cancers express HPV-16 E6 and/or E7, co-administration of E6 and/or E7 DNA vaccines with SPI-6 DNA is a useful approach for the treatment of such cancers and HPV-associated cervical lesions in humans.

### EXAMPLE IV

(This example incorporates by reference T W Kim et al., Gene Ther 11: 336-342, 2004 February)

### Suicidal DNA Vaccine Potency is Enhanced by Delaying Suicidal DNA-Induced Apoptotic Cell Death

A. Materials and Methods

Plasmid DNA constructs. The generation of pcDNA3-E7 has been described previously. pSG5 plasmids encoding BCL-xL and mt 7 (mt BCL-xL) (Cheng E H et al. 1996, supra in which aa 135-137 (NWG) in the BH1 domain were changed to AIL were described previously. For generation of pcDNA3-BCL-xL, BCL-xL was cut from pSG5-BCL-xL by BglII and was cloned into the unique BamHI cloning sites of the pcDNA3.1 (−) expression vector (Invitrogen, Carlsbad, Calif., USA). For generation of pcDNA3-mt BCL-xL, mt BCL-xL was cut from pSG5-mt BCL-xL by BglII and was cloned into the unique BamHI cloning sites of the pcDNA3.1(−) expression vector. For the generation of E7/BLC-xL chimera (pcDNA-E7/BCL-xL), BCL-xL was cut from pSG5-BCLxL by BglII and was cloned into the unique BamHI cloning sites of the pcDNA3-E7. For the generation of E7/mt BLC-xL chimera (pcDNA-mt E7/BCL-xL), mt BCL-xL was cut from pSG5-mt BCL-xL by BglII and was cloned into the unique BamHI cloning sites of the pcDNA3-E7. pSCA1 vector received from Dr Rod Bremner at the University of Toronto. This pSCA1 vector contains human cytomegalovirus immediate-early gene (HCMV-IE) promoter upstream of the Semliki Forest virus replicon. The subgenomic promoter is located after the Semliki Forest virus replicon, upstream of a multiple cloning site for the insertion of genes of interest. pSCA1-E7 was reported previously (Hsu K F et al. 2001, Gene Ther 8:376-383). For the generation of pSCA1-BCL-xL, pSCA1-mt BCL-xL, pSCA1-E7/BCL-xL, and pSCA1-E7/mt BCL-xL, fragments of BCL-X, mt BCL-xL, E7/BCL-xL, or E7/mt BCL-xL were cut from pcDNA3 vectors by BamHI-PmeI and cloned into BamHI-SmaI sites of pSCA1, respectively. The accuracy of these constructs was confirmed by DNA sequencing. The DNA was amplified in Escherichia coli DH5α and purified as described previously.

Mice and murine TC-1 tumor cell line See Example I.

Survival of dendritic cell line. See Example III. Detection of dead cells was performed using propidium iodide (PI) from BD Bioscience, San Diego, Calif. according to vendor's protocol. The percent of cell death was analyzed using flow cytometry analysis by gating GFP+ cells, which represented the transfected cells. Data are expressed as percent of DC-V cell deaths.

DNA vaccination. See Example I. Mice were immunized with 2 μg of the pSCA1 encoding BCL-xL, E7, E7/BCLxL, E7/mt BCL-xL, or no insert. The mice received a booster of the same composition 1 week later.

Intracellular cytokine staining and flow cytometry Analysis. See Examples, supra.

In vivo tumor protection. See Examples, supra. C57BL/6 mice (5/group) were vaccinated via gene gun with 2 μg of pSCA1 (no insert), pSCA1-BCL-xL, pSCA1-E7, or pSCA1-E7/BCL-xL via gene gun. One week after the last vaccination, mice were challenged s.c. with 5×10⁴ TC-1 cells/mouse in the right leg and then monitored twice a week.

In vivo tumor treatment. See Examples, supra. Three days after i.v. inoculation of TC-1 tumor cells, mice were administered 2 μg of pSCA1 (no insert), pSCA1-BCL-xL, pSCA1-E7, or pSCA1-E7/BCL-xL via gene gun; mice were boosted one week later. Mice were killed and lungs were explanted on day 21 for evaluation of pulmonary nodules.

In vivo antibody depletion experiment. See Examples, supra.

Statistical analysis. See Examples supra.

B. Results

The BCL-xL Gene in a Suicidal DNA Vector Reduces Cell Death in Transfected Cells

The present inventors characterized and compared the pSCA1 plasmid-driven expression of E7/BCL-xL and E7/mt BCL-xL proteins using Western blot analysis and noted that the expression levels of wild-type and mutant forms of the proteins were equivalent.

To examine whether the linkage of BCL-xL gene to antigenic gene in a suicidal DNA vector can reduce suicidal DNA-induced cell death, the cell death of the various pSCA1 DNA-transfected cells was measured using PI. The DC variant (DC-V) cell line was selected as a model to investigate the survival of the DCs after transfecting these cells with various suicidal DNA constructs. Such immortalized clones display dendritic morphology, and many express the DC-specific markers DEC-205 and 33D1 as well as high levels of MHC molecules and costimulatory molecules (Shen et al, supra). Moreover, these cloned DCs can present exogenous antigens on both MHC class I and II molecules.

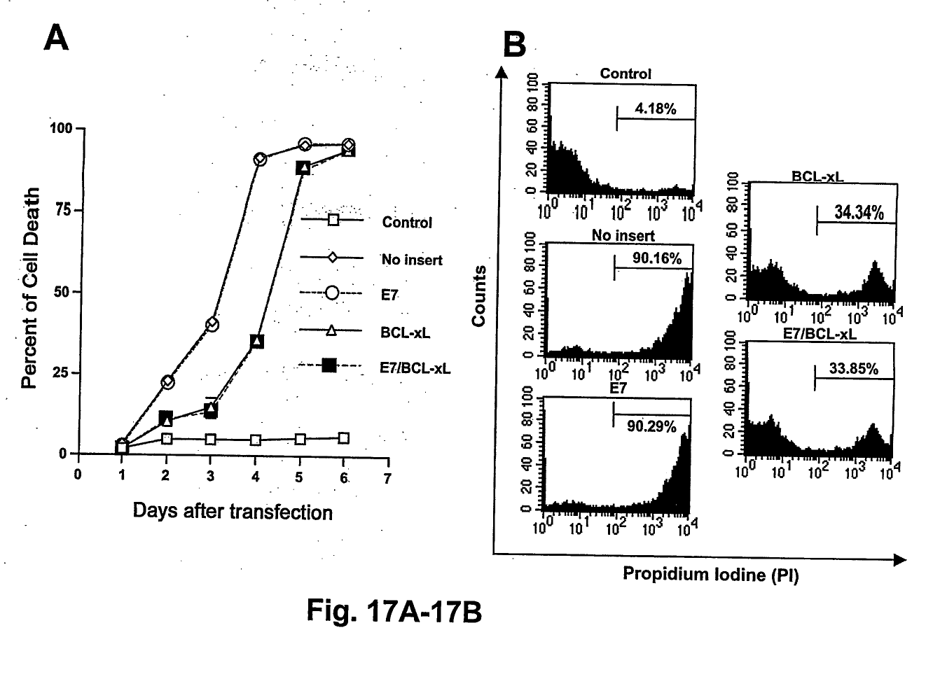

In this study, the DC-V cells were transfected with a pSCA1 construct encoding (i) E7, (ii) BCL-xL, (iii) E7/BCL-xL, (iv) E7/mt BCL-xL, or (v) no insert. pcDNA3, a plasmid vector that does not itself induce cell death, was used as a negative control. As shown in FIG. 17A, in DC-V cells transfected with pSCA1 encoding either BCL-xL or E7/BCL-xL DNA, death was delayed compared to DC-V cells transfected with pSCA1 encoding either E7 or no insert. Transfection with various of the pSCA1 vectors eventually led to cell death (by day 6 after transfection). Control transfection of DC-V cells with pcDNA3 vector did not lead to significant cell death by 6 days. These results demonstrated that the addition of BCL-xL gene to the pSCA1 DNA vector significantly delayed cell death caused by the suicidal DNA vector.

To confirm whether the above delay of cell death was due to the anti-apoptotic property of BCL-xL, a BCL-xL mutant (mt BCL-xL) lacking the anti-apoptotic function was studied. As shown in FIG. 17A, there was no significant difference in the percent of PI+ cells at day 1 among DC-V cells transfected with the various pSCA1 DNA constructs. However, by day 4 (FIG. 17B), the percent of PI+ cells among DC-V cells transfected with pSCA1 encoding E7/mt BCL-xL (91%), or no insert (90%) was significantly higher than among DC-V cells transfected with pSCA1-E7/BCL-xL (34%). These results confirmed that the mutation in BCL-xL leading to abrogation of anti-apoptotic function of BCL-xL manifested itself in this system.

The Linkage of BCL-xL to E7 in the pSCA1 Vector Significantly Enhanced the E7-Specific CD8+ T-Cell-Mediated Immune Responses in Vaccinated Mice.

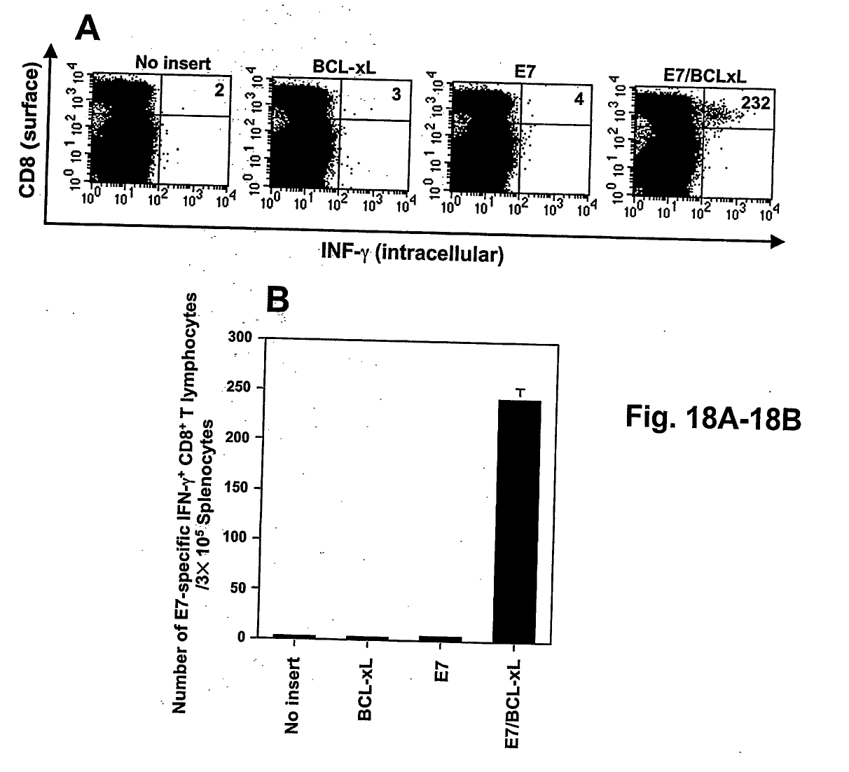

According to the inventors' conception, the delay of suicidal DNA-induced cell death due to the expression of BCL-xL would enhance the priming of antigen-specific T-cell responses when the construct was administered intradermally via gene gun. To assess the quantity of E7-specific IFNγ-secreting CD8+ T-cell precursors generated by vaccination with the various pSCA1 DNA constructs, intracellular cytokine staining followed by flow cytometry were performed. As shown in FIGS. 18A and 18B, C57BL/6 mice vaccinated with pSCA1-E7/BCL-xL generated the highest number of E7-specific IFNγ-secreting CD8+ T-cell precursors (per 3×10⁵ splenocytes) (241.7±12.7) among the vaccinated groups with more than a 50-fold increase compared to mice vaccinated with pSCA1-E7 (4.0±1.0) (P<0.01). These results also indicated that DNA encoding the E7 antigen was required since pSCA1-BCL-xL did not enhance the number of E7-specific CD8+ T cells (2.7±0.6). These results indicate that the linkage of BCL-xL to E7 in a chimeric suicidal DNA vector vaccine significantly enhanced antigen-specific CD8+ T-cell-responses.

The Anti-Apoptotic Function of BCL-xL is Important for Enhanced Immune Potentiation

To verify that the observed enhancement in the E7-specific CD8+ T cell response was due to the anti-apoptotic property of BCL-xL, a BCL-xL mutant (mt BCL-xL) that lacks anti-apoptotic function was employed. The number of E7-specific IFNγ-secreting CD8+ T cell precursors in mice vaccinated with pSCA1-E7/BCL-xL was compared with mice given pSCA1-E7/mtBCL-xL. As shown in FIGS. 18A and 18B, while vaccination with pSCA1-E7/13CL-xL suicidal DNA induced a high number of E7-specific IFNγ-secreting CD8+ T-cell precursors (per 3×10⁵ splenocytes), 251.4±12.7, vaccination with the mutant pSCA1-E7/mt BCL-xL resulted in a significantly lower number, 42.5±7.2 (P<0.001, ANOVA). Thus, the anti-apoptotic function of BCL-xL is necessary for it immunopotentiating capability when given in the form of a chimeric vaccine with antigen-encoding DNA.

The Inclusion of BCL-xL DNA with E7 DNA in the pSCA1 Vector Significantly Enhances E7-Specific Antitumor Effects

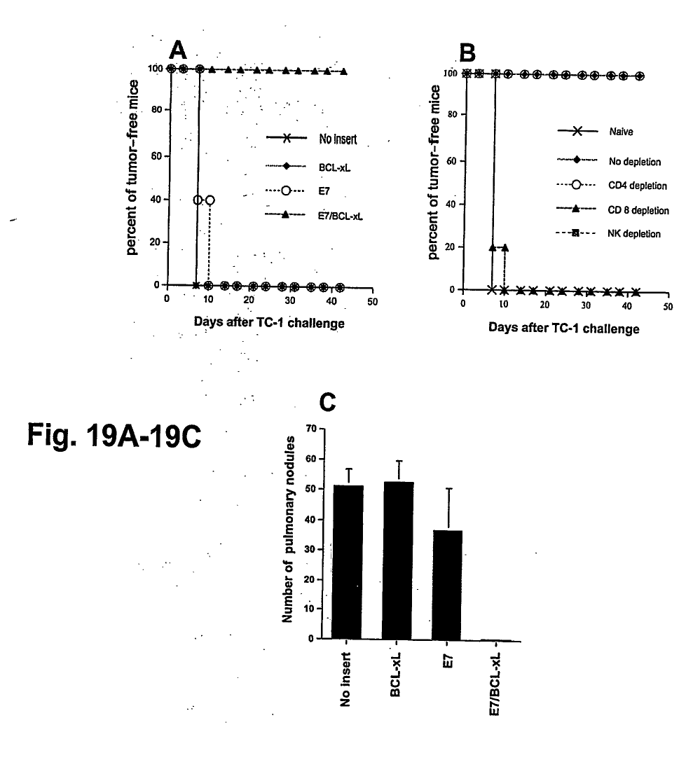

To determine if the enhanced T cell-mediated immunity noted above led to a significant E7-specific antitumor effect, studies of in vivo tumor protection were conducted using the TC-1 tumor. As shown in FIG. 19A, 100% of C57BL/6 mice receiving the pSCA1-E7/BCL-xL suicidal DNA vaccine remained tumor-free 42 days after TC-1 challenge. In contrast, all mice receiving pSCA1 (no insert), pSCA1-BCL-xL (lacking the antigen), or pSCA1-E7 suicidal DNA vectors developed tumors by day 10 after challenge. These results indicated that the linkage of BCL-xL gene to E7 DNA in a suicidal DNA vaccine significantly enhanced the E7-specific antitumor immunity.

Antibody ablation studies in vivo were used to determine which lymphocyte subsets were important for these antitumor effects. As shown in FIG. 19B, all of the mice depleted of CD8+ T cells grew tumors within 2 weeks of challenge, while none of the mice depleted of CD4+ T cells or NK cells grew tumors 42 days after TC-1 challenge. These data confirm the importance of CD8+ T cells for the antitumor effects induced by the pSCA1-E7/BCL-xL suicidal DNA vaccine.

FIG. 19C shows results of tumor treatment studies using a hematogenous spread model with TC-1 implanted i.v. Mice immunized with pSCA1-E7/BCL-xL suicidal DNA vaccine exhibited the fewest pulmonary tumor nodules (0.2±0.4, P<0.001, one-way ANOVA) compared to mice vaccinated with pSCA1 (no insert) (51.2±5.6), pSCA1-BCL-xL (52.6±7.0), or pSCA1-E7 (36.8±14.3). These results are consistent with the report of Pirtskhalaishvili G et al., 2000, J Immunol 165:1956-64, demonstrating that treatment of prostate cancer-bearing mice with BCL-xL-transduced DCs resulted in significant inhibition of tumor growth. In conclusion, the present results demonstrate that vaccination with the SCA1-E7/BCL-xL suicidal DNA vaccine induces a potent protective and therapeutic immune response against an E7-expressing tumor.

C. Discussion

The BCL-xL protein was selected for testing herein because it is considered to be one of the most potent anti-apoptotic proteins. The previous Examples show results using number of anti-apoptotic polypeptides factors to enhance DC survival and antigen-specific CD8+ T-cell immune responses when DNA encoding these polypeptides is co-administered with DNA encoding the antigen. These anti-apoptotic molecules included BCL-xL9 and BCL-2, members of the BCL-2 family of proteins; X-linked inhibitor of apoptosis protein (XIAP); and dominant-negative (dn) mutants of caspases such as dn caspase-9 and dn caspase-8 which have a mutation in the enzyme active site and serve as inhibitors of apoptosis. Results with these apoptosis inhibitors indicate that BCL-xL was most potent in enhancing antigen-specific immune responses and antitumor effects. BCL-xL, like BCL-2, localizes to outer mitochondrial membranes and prevents release of pro-apoptotic factors from mitochondria, such as cytochrome c and Smac/DIABLO. In addition, BCL-xL may inhibit apoptosis through a mitochondria-independent pathway (Medema et al., 1998, supra). Thus, BCL-xL may be able to inhibit apoptosis at multiple points along the programmed cell death pathway which explains its potency.

The anti-apoptotic function of the BCL-XL molecules is clearly needed for its observed immunological enhancement though there may be additional explanations the observed effects. For example, BCL-2 family proteins have been suggested to alter the differentiation status of cells, raising the possibility that DCs transfected with suicidal DNA encoding chimeric E7/BCL-xL molecule may lead to phenotypic changes of the transfected DCs. Those changes could include expression of MHC class I, MHC class II, or co-stimulatory molecules (B7-1, B7-2, and others). However, there were not evident changes in these molecules in DC-V cells transfected with the various pSCA-1 constructs. Alternatively, the linkage of BCL-xL to E7 may influence the processing of E7 in transfected cells. This may explain the slight increase of E7-specific CD8+ T-cell precursors in mice vaccinated with the mutant BCL vector, pSCA1-E7/mt BCL-xL, when compared mice given only the antigen-expressing vector. Irrespective of what may be learned about the BCL-xL molecule in the future, it has been established here that DNA encoding this polypeptide can be linked to antigen-encoding DNA and used to achieve enhanced antigen-specific CD8+ T-cell immune responses that have clinical significance.

Some of the safety concerns of DNA vaccines were discussed in the foregoing Examples. The use of suicidal DNA vectors significantly alleviates some of these concerns directed to possible integration of vector DNA, and also alleviates the concern about vectors encoding oncogenic proteins such as the HPV-16 E6 and E7 and the BCL-xL protein. One strategy to further improve safety is to use molecules that are anti-apoptotic yet do not have the transforming property of BCLxL. Such molecules include TRANCE (Wong B R et al., 1997, J Exp Med 186:2075-80), CD40 ligand (Esche C et al., 1999, Eur J Immunol 29: 2148-55), IL-12 (Ploemacher R E et al., 1993, Leukemia 7:1381-88), IL-15 (Bykovskaia S N et al., 1999, J Leukoc Biol 66:659-666) and SPI-6 (Example III), all of which references are incorporated by reference. Among these CD40 ligand, 31 IL-12, and IL-15 have been tested for their ability to enhance conventional naked DNA vaccine potency. The present invention includes suicidal DNA vaccines wherein DNA encoding one of these anti-apoptotic proteins is linked to antigen-encoding DNA.

It is known that that delivery of antigen to non-APCs and subsequent priming of T cells via a cross-priming mechanism may also contribute to the generation of specific CD8+ T cells. The transfection of the DNA into non-APCs, such as keratinocytes, may eventually lead to the release of encoded antigen for uptake by APCs, such as DCs, which subsequently present antigen to naive T cells. Thus, the observed enhancement of the E7-specific CD8+ T-cell response generated by suicidal DNA encoding chimeric BCL-xL/E7 may be, to some extent, related to this other antigen presentation mechanism.

In summary, the present results demonstrate pSCA1-E7/BCLxL suicidal DNA vaccine is a useful construct for induction of potent T cell immunity with fewer concerns about vector DNA integration and transformation associated with conventional DNA vaccines. Such vectors may comprise any antigen to which T cell immunity is desired, including a host of antigens present onf various tumors, viruses, virus-infected cells, bacteria, pathogenic tissues, and the like.

The references cited above are all incorporated by reference herein, whether specifically incorporated or not.

Citation of the documents herein is not intended as an admission that any of them is pertinent prior art. All statements as to the date or representation as to the contents of these documents is based on the information available to the applicant and does not constitute any admission as to the correctness of the dates or contents of these documents.

What is claimed is:

1. A nucleic acid composition useful as an immunogen, comprising a combination of
(a) first nucleic acid vector comprising a first sequence encoding an antigenic polypeptide or peptide, which first vector optionally comprises a second sequence linked to said first sequence, which second sequence encodes an immunogenicity-potentiating polypeptide (IPP);
b) a second nucleic acid vector encoding an anti-apoptotic polypeptide,
wherein, when said second vector is administered with said first vector to a subject, a T cell-mediated immune response to said antigenic polypeptide or peptide is induced that is greater in magnitude and/or duration than an immune response induced by administration of said first vector alone.

2. The composition of claim 1 wherein said first vector comprises said IPP.

3. A nucleic acid composition useful as an immunogen comprising
(a) a first nucleic acid sequence that encodes an antigenic polypeptide or peptide.
(b) optionally, fused in frame with the first nucleic acid sequence, a linker nucleic acid sequence encoding a linker peptide;
(c) a second nucleic acid sequence that is linked in frame to said first nucleic acid sequence or to said linker nucleic acid sequence and that encodes an IPP; and
(d) a third nucleic acid sequence encoding an anti-apoptotic polypeptide.

4. The composition of any of claims 1-3 wherein the IPP acts in potentiating an immune response by promoting:
(a) processing of the linked antigenic polypeptide via the MHC class I pathway or targeting of a cellular compartment that increases said processing;
(b) development, accumulation or activity of antigen presenting cells or targeting of antigen to compartments of said antigen presenting cells leading to enhanced antigen presentation;
(c) intercellular transport and spreading of the antigen; or
(d) any combination of (a)-(c).

5. The composition of claim 4 wherein the IPP is:
(a) the sorting signal of the lysosome-associated membrane protein type 1 (Sig/LAMP-1)
(b) a mycobacterial HSP70 polypeptide, the C-terminal domain thereof, or a functional homologue or derivative of said polypeptide or domain;
(c) a viral intercellular spreading protein selected from the group of herpes simplex virus-1 VP22 protein, Marek's disease virus VP22 protein or a functional homologue or derivative thereof;
(d) an endoplasmic reticulum chaperone polypeptide selected from the group of calreticulin, ER60, GRP94, gp96, or a functional homologue or derivative thereof
(e) a cytoplasmic translocation polypeptide domains of a pathogen toxin selected from the group of domain II of Pseudomonas exotoxin ETA or a functional homologue or derivative thereof;
(f) a polypeptide that targets the centrosome compartment of a cell selected from γ-tubulin or a functional homologue or derivative thereof; or
(g) a polypeptide that stimulates dendritic cell precursors or activates dendritic cell activity selected from the group of GM-CSF, Flt3-ligand extracellular domain, or a functional homologue or derivative thereof.

6. The composition of claim 1 or 3 wherein said anti-apoptotic polypeptide is selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or derivative of any of (a)-(g).

7. The composition of claim 4 wherein said anti-apoptotic polypeptide is selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or derivative of any of (a)-(g).

8. The composition of claim 5 wherein said anti-apoptotic polypeptide is selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or derivative of any of (a)-(g).

9. The composition of claim 1 or 3 wherein the antigenic peptide comprises an epitope that binds to and is presented on the cell surface by MHC class I proteins.

10. The composition of claim 9 wherein the epitope is between about 8 and about 11 amino acid residues in length.

11. The composition of claim 1 or 3 wherein the antigenic polypeptide or peptide is:
(i) is derived from a pathogen selected from the group consisting of a mammalian cell, a microorganism or a virus;
(ii) cross-reacts with an antigen of the pathogen; or
(iii) is expressed on the surface of a pathogenic cell.

12. The composition of claim 11 wherein the virus is a human papilloma virus.

13. The composition of claim 12, wherein the antigen is an HPV-16 E6 or E7 peptide.

14. The composition of claim 11 wherein the pathogen is a bacterium.

15. The composition of claim 1, wherein the antigenic polypeptide or peptide is a tumor-specific or tumor-associated antigen.

16. The composition of claim 1 wherein the first vector comprises a promoter operatively linked said first and/or said second sequence.

17. The composition of claim 3 which comprises a promoter operatively linked to one or more of said first, second and sequences.

18. The composition of claim 16, wherein the promoter is one which is expressed in an antigen presenting cell (APC).

19. The composition of claim 18, wherein the APC is a dendritic cell.

20. A particle comprising a material is suitable for introduction into a cell or an animals by particle bombardment to which is bound the first vector of claim 1 or 2.

21. A particle comprising a material is suitable for introduction into a cell or an animals by particle bombardment to which is bound the second vector of claim 1 or 2.

22. A particle comprising a material is suitable for introduction into a cell or an animals by particle bombardment to which is bound the first and the second vector of claim 1 or 2.

23. A particle comprising a material is suitable for introduction into a cell or an animals by particle bombardment to which is bound the composition of claims 3.

24. A particle comprising a material is suitable for introduction into a cell or an animals by particle bombardment to which is bound the composition of claim 4.

25. A particle comprising a material is suitable for introduction into a cell or an animals by particle bombardment to which is bound the composition of claim 5.

26. A particle comprising a material is suitable for introduction into a cell or an animals by particle bombardment to which is bound the composition of claim 6.

27. A particle comprising a material is suitable for introduction into a cell or an animals by particle bombardment to which is bound the composition of claim 7.

28. A particle comprising a material is suitable for introduction into a cell or an animals by particle bombardment to which is bound the composition of claim 8.

29. The particle of any of claims claim 20-28, wherein the material is gold.

30. A pharmaceutical composition capable of inducing or enhancing an antigen specific immune response, comprising the composition of any of claims 1-19 and a pharmaceutically acceptable carrier or excipient.

31. A pharmaceutical composition capable of inducing or enhancing an antigen specific immune response, comprising the particle of any of claims claim 20-29, and a pharmaceutically acceptable carrier or excipient.

32. A method of inducing or enhancing an antigen specific immune response in a subject comprising administering to the subject an effective amount of the composition of claim 1, 2 or 3, thereby inducing or enhancing the antigen specific immune response.

33. A method of inducing or enhancing an antigen specific immune response in a subject comprising administering to the subject an effective amount of the composition of claim 4, thereby inducing or enhancing the antigen specific immune response.

34. A method of inducing or enhancing an antigen specific immune response in a subject comprising administering to the subject an effective amount of the composition of claim 5, thereby inducing or enhancing the antigen specific immune response.

35. A method of inducing or enhancing an antigen specific immune response in a subject comprising administering to the subject an effective amount of the composition of claim 6, thereby inducing or enhancing the antigen specific immune response.

36. A method of inducing or enhancing an antigen specific immune response in a subject comprising administering to the subject an effective amount of the composition of claim 7, thereby inducing or enhancing the antigen specific immune response.

37. A method of inducing or enhancing an antigen specific immune response in a subject comprising administering to the subject an effective amount of the composition of claim 8, thereby inducing or enhancing the antigen specific immune response.

38. A method of inducing or enhancing an antigen specific immune response in a subject comprising administering to the subject an effective amount of the composition of claim 11, thereby inducing or enhancing the antigen specific immune response.

39. A method of inducing or enhancing an antigen specific immune response in a subject, comprising administering to the subject an effective amount of the composition of claim 13, thereby inducing or enhancing the antigen specific immune response.

40. A method of inducing or enhancing an antigen specific immune response in a subject, comprising administering to the subject an effective amount of the particles of claim 20, thereby inducing or enhancing the antigen specific immune response.

41. A method of inducing or enhancing an antigen specific immune response in a subject comprising administering to the subject an effective amount of the particles of claim 23, thereby inducing or enhancing the antigen specific immune response.

42. A method of inducing or enhancing an antigen specific immune response in a subject, comprising administering to the subject an effective amount of the particles of any of claims 21, 22, or 24-29, thereby inducing or enhancing the antigen specific immune response.

44. The method of claim 32, wherein the antigen specific immune response is mediated at least in part by CD8⁺ cytotoxic T lymphocytes (CTL).

45. The method of claim 33, wherein the antigen specific immune response is mediated at least in part by CD8⁺ CTL.

46. The method of claim 34, wherein the antigen specific immune response is mediated at least in part by CD8⁺ CTL.

47. The method of claim 36, wherein the antigen specific immune response is mediated at least in part by CD8⁺ CTL.

48. The method of claim 38, wherein the antigen specific immune response is mediated at least in part by CD8⁺ CTL.

49. The method of claim 39, wherein the antigen specific immune response is mediated at least in part by CD8⁺ CTL.

50. The method of claim 40, wherein the antigen specific immune response is mediated at least in part by CD8⁺ CTL.

51. The method of claim 41, wherein the antigen specific immune response is mediated at least in part by CD8⁺ CTL.

52. The method of claim 32, wherein the composition is administered to a human.

53. The method of claim 40, wherein the particles are administered to a human.

54. The method of claims 32, wherein the composition is administered intradermally.

55. The method of claims 40, wherein the particles are administered intradermally.

56. The method of claim 32 wherein the composition is administered intratumorally or peritumorally.

57. A method of increasing the numbers of CD8⁺ CTLs specific for a selected desired antigen in a subject comprising administering an effective amount of the composition of claim 1, 2 or 3 wherein the antigenic peptide comprises an epitope that binds to and is presented on the cell surface by MHC class I proteins, thereby increasing the numbers of antigen-specific CD8⁺ CTLs.

58. A method of increasing the numbers of CD8⁺ CTLs specific for a selected desired antigen in a subject comprising administering an effective amount of the composition of claim 3, wherein the antigenic peptide comprises an epitope that binds to and is presented on the cell surface by MHC class I proteins, thereby increasing the numbers of antigen-specific CD8⁺ CTLs.

59. A method of increasing the numbers of CD8⁺ CTLs specific for a selected desired antigen in a subject comprising administering an effective amount of the composition of claim 4, wherein the antigenic peptide comprises an epitope that binds to and is presented on the cell surface by MHC class I proteins, thereby increasing the numbers of antigen-specific CD8⁺ CTLs.

60. A method of increasing the numbers of CD8⁺ CTLs specific for a selected desired antigen in a subject comprising administering an effective amount of the composition of claim 5, wherein the antigenic peptide comprises an epitope that binds to and is presented on the cell surface by MHC class I proteins, thereby increasing the numbers of antigen-specific CD8⁺ CTLs.

61. A method of increasing the numbers of CD8⁺ CTLs specific for a selected desired antigen in a subject comprising administering an effective amount of the composition of claim 6, wherein the antigenic peptide comprises an epitope that binds to and is presented on the cell surface by MHC class I proteins, thereby increasing the numbers of antigen-specific CD8⁺ CTLs.

62. A method of increasing the numbers of CD8⁺ CTLs specific for a selected desired antigen in a subject comprising administering an effective amount of the composition of claim 7, wherein the antigenic peptide comprises an epitope that binds to and is presented on the cell surface by MHC class I proteins, thereby increasing the numbers of antigen-specific CD8⁺ CTLs.

63. A method of increasing the numbers of CD8⁺ CTLs specific for a selected desired antigen in a subject comprising administering an effective amount of the composition of claim 8, wherein the antigenic peptide comprises an epitope that binds to and is presented on the cell surface by MHC class I proteins, thereby increasing the numbers of antigen-specific CD8⁺ CTLs.

64. A method of increasing the numbers of CD8⁺ CTLs specific for a selected desired antigen in a subject comprising administering an effective amount of the composition of claim 11, wherein the antigenic peptide comprises an epitope that binds to and is presented on the cell surface by MHC class I proteins, thereby increasing the numbers of antigen-specific CD8⁺ CTLs.

65. A method of increasing the numbers of CD8⁺ CTLs specific for a selected desired antigen in a subject comprising administering an effective amount of the composition of claim 13, wherein the antigenic peptide comprises an epitope that binds to and is presented on the cell surface by MHC class I proteins, thereby increasing the numbers of antigen-specific CD8⁺ CTLs.

66. A method of inhibiting the growth of a tumor in a subject comprising administering an effective amount of the composition of any of claims 1-13, thereby inhibiting growth of the tumor.

67. A method of inhibiting the growth of a tumor in a subject comprising administering an effective amount of the particles of any of claims 20-29, thereby inhibiting growth of the tumor.
